(19) United States
(12) Patent Application Publication
(10) Pub. No.: US 2024/0327354 A1
(43) Pub. Date: Oct. 3, 2024

(54) GRAM-NEGATIVE BACTERIA EFFLUX PUMP INHIBITORS

(71) Applicants: INSTITUT NATIONAL DE LA SANTÉ ET DE LA RECHERCHE MÉDICALE, Paris (FR); INSTITUT PASTEUR DE LILLE, Lille Cedex (FR); UNIVERSITÉ DE LILLE, Lille (FR); CENTRE NATIONAL DE LA RECHERCHE SCIENTIFIQUE, Paris (FR); CHRU DE LILLE, Lille (FR); JOHANN WOLFGANG GOETHE-UNIVERSITÄT FRANKFURT AM MAIN, Frankfurt am Main (DE)

(72) Inventors: Anaïs VIEIRA DA CRUZ, Lille Cedex (FR); Coline PLÉ, Lille Cedex (FR); Marion FLIPO, Lille Cedex (FR); Nina COMPAGNE, Lille Cedex (FR); Nicolas WILLAND, Lille Cedex (FR); Heng-Keat TAM, Hengyang (CN); Juan Carlos JIMÉNEZ CASTELLANOS, Lille Cedex (FR); Klaas Martinus POS, Frankfurt am Main (DE); Ruben Christiaan HARTKOORN, Lille Cedex (FR); Reinke Tobias MÜLLER, Frankfurt am Main (DE)

(21) Appl. No.: 18/579,953

(22) PCT Filed: Jul. 22, 2022
(86) PCT No.: PCT/EP2022/070601

§ 371 Date: Jan. 17, 2024

(30) Foreign Application Priority Data
Jul. 23, 2021 (EP) 21306032.0

(51) Int. Cl. C07D 213/74; A61K 31/4439; A61K 31/4545; A61K 31/4709; A61K 31/495; A61K 31/496; A61K 31/498; A61K 31/55; A61K 31/551; A61K 45/06; C07D 213/80; C07D 215/38; C07D 217/22; C07D 241/44; C07D 295/073; C07D 401/04; C07D 413/04; C07D 471/04; C07D 471/08; C07D 487/08

(52) U.S. Cl. CPC C07D 213/74; A61K 31/4439; A61K 31/4545; A61K 31/4709; A61K 31/495; A61K 31/496; A61K 31/498; A61K 31/55; A61K 31/551; A61K 45/06; C07D 213/80; C07D 215/38; C07D 217/22; C07D 241/44; C07D 295/073; C07D 401/04; C07D 413/04; C07D 471/04; C07D 471/08; C07D 487/08

(57) ABSTRACT

The present invention concerns novel Gram-negative bacteria efflux pump inhibitors. It further relates to the use of Gram-negative bacteria efflux pump inhibitors to prevent and/or treat antibiotic resistance by potentiating the activity of antibiotics. Infections by multidrug resistant (MDR) Gram-negative bacteria are a major threat to global healthcare. The inventors have discovered a novel class of Resistance Nodulation cell Division-efflux pump inhibitors. The inventors tested the effects of these inhibitors on growth inhibition of different bacteria as well as their impact on the boosting of antibiotic activity in different bacteria. Particularly, the inventors tested the effects of these inhibitors on E. coli, A. baumamnii, K. pneumoniae and P. aeruginosa.

The present invention concerns novel Gram-negative bacteria efflux pump inhibitors. It further relates to the use of Gram-negative bacteria efflux pump inhibitors to prevent and/or treat antibiotic resistance by potentiating the activity of antibiotics.

### BACKGROUND

Infections by multidrug resistant (MDR) Gram-negative bacteria are a major threat to global healthcare, with R&D into novel antibiotics classified as a critical priority by the world health organization. Amongst many antibiotic-specific resistance mechanisms, multidrug efflux catalyzed by bacterial efflux pumps is a major factor defining both intrinsic and acquired multiple drug resistance phenotypes in Gram-negative bacteria. In particular, efflux pumps belonging to the Resistance Nodulation cell Division (RND) superfamily are able to extrude a plethora of chemically diverse antibiotic molecules and represent a major barrier to antibiotic efficacy and drug development (Li X Z, Plésiat P, Nikaido H. The challenge of efflux-mediated antibiotic resistance in Gram-negative bacteria. Clin Microbiol Rev. 2015, 28, 337-418. doi:10.1128/CMR.001 17-14).

RND-type efflux pumps comprise three components that assemble into tripartite complexes spanning the entire Gram-negative envelope (Du, D., Wang-Kan, X., Neuberger, A. et al. Multidrug efflux pumps: structure, function and regulation. Nat Rev Microbiol. 2018, 16, 523-539. doi.org/10.1038/s41579-018-0048-6). The canonical RND-type efflux pump is the AcrA-AcrB-TolC tripartite system from Escherichia coli, where AcrB is the inner membrane component, AcrA the periplasmic adapter protein, and TolC the outer-membrane channel.

Considering the critical role of RND pumps in innate and adaptive antibiotic resistance in Gram negative bacteria, there have been considerable efforts to discover and develop efflux pump inhibitors (EPI). Characterized EPIs include Phe-Arg β-naphthylamide (PAβN), 1-(1-naphtylmethyl)-piperazine (NMP), pyridopyrimidine (such as D13-9001), and pyranopyridine (the MBX series) that inhibit RND-pump mediated efflux and boost antibiotic activity of all known substrate drugs of the pump in Gram-negative pathogens (Opperman T J, Nguyen S T. Recent advances toward a molecular mechanism of efflux pump inhibition. Front Microbiol. 2015, 6, 421. doi: 10.3389/fmicb.2015.00421).

To identify new efflux pump inhibitors (EPIs), a phenotypic assay was used to screen a chemical library of 1280 compounds at 300 μM, in combination with a sub-active dose of pyridomycin, an antibiotic identified as a particularly good substrate of the AcrAB-TolC efflux pump.

In the present invention, the inventors have thus discovered a novel class of RND-EPI. These molecules are used to potentiate the activity of antibiotics.

### DETAILED DESCRIPTION

The present invention thus relates to a compound of formula (I):

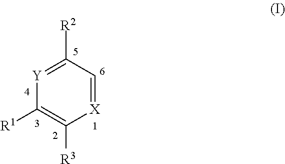

In which:
X can be chosen from:
   CH; and
   N;
Y can be chosen from:
   CH; and
   N;

X and Y not being CH at the same time;
R¹ can be chosen from:
   a halogen atom;
   a —(C₁-C₃)alkyl group;
   a —(C₁-C₃)halogenoalkyl group;
   a —(C₁-C₃)alkoxy group; or
   a nitrile group;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from 0, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
   or its pharmaceutically acceptable salts or optical isomers, racemates, diastereoisomers, enantiomers or tautomers;
with the exclusion of the following compounds:
   1-[3-chloro-5-(trifluoromethyl)-2-pyridyl]piperazine;
   1-[3-chloro-5-(trifluoromethyl)-2-pyridyl]piperazine hydrochloride;
   1-(5-bromo-3-chloro-2-pyridyl)-4-methyl-piperazine;
   1-[2-chloro-4-(trifluoromethyl)phenyl]piperazine hydrochloride;
   1-[3-chloro-5-(trifluoromethyl)-2-pyridyl]piperidin-4-amine;
   1-[3-chloro-5-(trifluoromethyl)-2-pyridyl]-1,4-diazepane;
   methyl 5-chloro-6-piperazin-1-yl-pyridine-3-carboxylate hydrochloride;
   1-(3-chloro-5-methyl-2-pyridyl)piperazine;
   1-(5-bromo-3-chloro-2-pyridyl)piperazine;
   2-chloro-3-piperazin-1-yl-quinoxaline hydrochloride;
   1-[3-bromo-5-(trifluoromethyl)-2-pyridyl]-4-methyl-piperazine;
   3-methyl-2-piperazin-1-yl-quinoline hydrochloride;
   2-piperazin-1-ylquinoline-3-carbonitrile hydrochloride;
   3-[(3R)-3-methylpiperazin-1-yl]quinoxalin-2-ol hydrochloride;
   2-chloro-3-(4-ethylpiperazin-1-yl)quinoxaline;
      3-(5-chloro-6-piperazin-1-yl-3-pyridyl)-5-methyl-1,2,4-oxadiazole;
      (1R,5S)—N-[3-chloro-5-(trifluoromethyl)-2-pyridyl]-3-azabicyclo[3.1.0]hexan-6-amine;
   2-methoxy-3-piperazin-1-yl-quinoxaline hydrochloride;
   2-chloro-6,7-dimethyl-3-piperazin-1-yl-quinoxaline;
   3-Cyan-2-(4-methyl-piperazino)-5-(pyrid-4-yl)-pyridin;
   methyl 5-chloro-6-4-methylpiperazin-1-yl) nicotinate;
   (1-[3-chloro-5-(trifluoromethyl)-2-pyridinyl]-4-methylpiperazine;
   1-(3,5-dichloro-2-pyridyl)piperazine; hydrochloride; 2-methoxy-3-(4-methylpiperazin-1-yl)quinoxaline;
   2-ethoxy-3-(4-methylpiperazin-1-yl)quinoxaline;
   2-methyl-3-(4-methylpiperazin-1-yl)quinoxaline;
   2-ethyl-3-(4-methylpiperazin-1-yl)quinoxaline;
   2-(4-methylpiperazin-1-yl)-3-(trifluoromethyl)quinoxaline;
   2-bromo-3-(4-methylpiperazin-1-yl)quinoxaline;
   2-chloro-3-(4-methyl-1,4-diazepan-1-yl)quinoxaline;
   3,6-dichloro-2-(4-methylpiperazin-1-yl)quinoxaline;
   2,6,7-trichloro-3-(4-methylpiperazin-1-yl)quinoxaline;
   1-(3-chloroquinoxalin-2-yl)-N-methyl-pyrrolidin-3-amine
   2-chloro-3-[(3S)-3-methylpiperazin-1-yl]quinoxaline hydrochloride; and
   4-bromo-1-piperazin-1-yl-isoquinoline.

The invention also relates to a pharmaceutical composition comprising a compound of formula (I) as described above and a pharmaceutically acceptable excipient.

The invention further relates to a compound of formula (I):

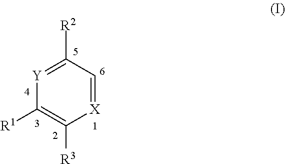

In which:
X can be chosen from:
   CH; and
   N;
Y can be chosen from:
   CH; and
   N;
R¹ can be chosen from:
   a halogen atom;
   a —(C₁-C₃)alkyl group;
   a —(C₁-C₃)halogenoalkyl group;
   a —(C₁-C₃)alkoxy group;
   a nitrile group; or
   R¹ forms together with Y a fused phenyl in positions 3 and 4;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from O, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optional a bridge of 0 to 2 carbon atoms between 2 members of the heterocycle;
      a (C₆-C₁₀)aryl group, optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
   or its pharmaceutically acceptable salts or optical isomers, racemates, diastereoisomers, enantiomers or tautomers;
for use as a medicament, in particular to prevent and/or treat antibiotic resistance.

The present invention also relates to a method for treating a bacterial infection, comprising administering to a subject in need thereof, a compound of formula (I):

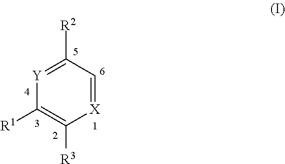

In which:
X can be chosen from:
   CH; and
   N;
Y can be chosen from:
   CH; and
   N;
R¹ can be chosen from:
   a halogen atom;
   a —(C₁-C₃)alkyl group;
   a —(C₁-C₃)halogenoalkyl group;
   a —(C₁-C₃)alkoxy group;
   a nitrile group; or
   R¹ forms together with Y a fused phenyl in positions 3 and 4;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from O, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optional a bridge of 0 to 2 carbon atoms between 2 members of the heterocycle;
      a (C₆-C₁₀)aryl group, optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
      R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;

or its pharmaceutically acceptable salts or optical isomers, racemates, diastereoisomers, enantiomers or tautomers;

in combination with an antibiotic.

Unless specified otherwise, the terms used hereabove or hereafter as regards to the compounds of formula (I) have the meaning ascribed to them below:
“halogen” refers to fluorine, chlorine, bromine or iodine atom, in particular bromine, iodine or chlorine atom.
“alkyl” represents an aliphatic-hydrocarbon group which may be straight or branched, having 1 to 6 or 1 to 4 or 1 to 3 carbon atoms in the chain (C₁-C₆)alkyl or (C₁-C₄)alkyl or (C₁-C₃)alkyl, unless specified otherwise. In particular, alkyl groups have 1 to 3 carbon atoms in the chain (C₁-C₃) alkyl. Branched means that one or more alkyl groups such as methyl, ethyl or propyl are attached to a linear alkyl chain. Exemplary alkyl groups include methyl, ethyl, n-propyl, i-propyl, n-butyl, t-butyl, 2,2-diméthylbutyl, n-pentyl, n-hexyl, in particular methyl or ethyl. As mentioned, said alkyl can be substituted by one heteroatom chosen from O, N or S (it being understood that it is one of the carbons of the alkyl group which is substituted by O, N or S).
“halogenoalkyl” represents an aliphatic-hydrocarbon group which may be straight or branched, having 1 to 3 carbon atoms in the chain (C₁-C₃)halogenoalkyl and in which one or more hydrogen atoms has been replaced by a halogen atom such as fluorine, chlorine, bromine or iodine atom, in particular by one or more fluorine atoms. Exemplary halogenoalkyl include trifluoromethyl.
“nitrile” refers to C≡N.
“alkoxy” represents an alkyl group as previously defined singular bonded to oxygen. Examples of linear or branched (C₁-C₃)alkoxy includes methoxy (CH₃O) and ethoxy (CH₃CH₂O—). Said alkoxy can be substituted by one or more fluorine atoms such as trifluoromethoxy.
“alkenyl” refers to an aliphatic hydrocarbon group containing a carbon-carbon double bond and which may be straight or branched having 2 to 6 carbon atoms in the chain (C₂-C₆)alkenyl, unless specified otherwise. Preferred alkenyl groups have 2 to 3 carbon atoms in the chain (C₂-C₃)alkenyl. Exemplary alkenyl groups include ethenyl, n-propenyl, i-propenyl, n-butenyl, i-butenyl, 2,2-dimethylbut-1-enyl, n-pentenyl, in particular propenyl. As mentioned, said alkenyl can be substituted by one heteroatom chosen from O, N or S (it being understood that it is one of the carbons of the alkyl group which is substituted by O, N or S).
“alkynyl” refers to an aliphatic hydrocarbon group containing a carbon-carbon triple bond and which may be straight or branched having 2 to 6 carbon atoms in the chain (C₂-C₆)alkynyl, unless specified otherwise. Preferred alkynyl groups have 3 to 5 carbon atoms in the chain (C₃-C₅)alkynyl. Exemplary alkynyl groups include ethynyl, propynyl, but-1-ynyl, but-2-ynyl, pent-1-ynyl. As mentioned, said alkynyl can be substituted by one heteroatom chosen from O, N or S (it being understood that it is one of the carbons of the alkyl group which is substituted by O, N or S).
“aryl” refers to an aromatic monocyclic or multicyclic hydrocarbon ring system of 6 to 10 carbon atoms, preferably of 6 carbon atoms. Exemplary aryl groups include phenyl, naphthyl, biphenyl, in particular phenyl. Said aryl or phenyl can be substituted by one or more halogen atoms such as fluorine, bromine, iodine or chlorine, in particular chlorine; one or more (C₁-C₄)alkyl or (C₁-C₃)alkyl such as a methyl, said alkyl being optionally substituted by one to 3 fluorine atoms such as trifluoromethyl; one or more (C₁-C₃)alkoxy such as methoxy, said alkoxy being optionally substituted by one or more fluorine atoms such as trifluoromethoxy, a NRR′ group, R and R′ being as defined herein, in particular by NH₂; a (4-8-membered)heterocycle having at least one N; a NH-heterocycle; an aryl of 6 to 10 carbon atoms, preferably of 6 carbon atoms, optionally substituted by a NRR′ group, R and R′ being as defined herein (such as NH₂); a NR⁷R⁸ group, R⁷ and R⁸ being as defined herein.
“heteroaryl” refers to a 5 to 12, in particular 5 to 6 aromatic mono-, bi- or multicyclic ring wherein at least one member of the ring is a hetero atom. Hetero atoms can be O, S or N, in particular N. In particular, each ring comprises from 1 to 3 hetero atoms. When bi- or multicylcic rings are comtemplated, at least one of the ring is aromatic but the other can be non aromatic, such as 1,2,3,4-tetrahydroisoquinoline and isoindoline. Examples include pyrrolyl, pyridyl, oxadiazol, thiazol, oxazol, triazol, pyrazolyl, pyrimidinyl, pyrazinyl, indolyl, imidazolyl, in particular pyridyl, triazol or oxadiazol. Said heteroaryl can be substituted by one or more halogen atoms such as fluorine, bromine, iodine or chlorine, in particular chlorine; one or more (C₁-C₄)alkyl or (C₁-C₃) alkyl such as a methyl, said alkyl being optionally substituted by one to 3 fluorine atoms such as trifluoromethyl or by a NRR′ group, R and R′ being as defined herein, in particular by methylamine, ethylamine or propylamine; one or more (C₁-C₃)alkoxy such as methoxy, said alkoxy being optionally substituted by one or more fluorine atoms such as trifluoromethoxy; a (4-8-membered)heterocycle having at least one N; a NH-heterocycle; an aryl of 6 to 10 carbon atoms, preferably of 6 carbon atoms, optionally substituted by a NRR′ group, R and R′ being as defined herein (such as methylamine or ethylamine); a NR⁷R⁸ group, R⁷ and R⁸ being as defined herein.
“—(C₁-C₃)alkyl-phenyl” or “—(C₁-C₃)alkyl-(5-6 membered)heteroaryl” means that Rₐ is linked to the oxygen atom of COO— or Rb of —N(H)Rb- is linked to the carbon of the alkyl group; in particular —(C₁-C₃)alkyl-phenyl is a benzyl, ethylphenyl or propylphenyl. Phenyl can be substituted as above mentioned, in particular by a halogen atom such as fluorine, bromine, iodine or chlorine, in particular chlorine; (C₁-C₄)alkyl or (C₁-C₃)alkyl such as a methyl, said alkyl being optionally substituted by one to 3 fluorine atoms such as trifluoromethyl; (C₁-C₃)alkoxy such as methoxy, said alkoxy being optionally substituted by one or more fluorine atoms such as trifluoromethoxy.
“—CO—NH—(C₁-C₃)alkyl-(C₆-C₁₀)aryl” means that the aryl is linked to the alkyl group by a carbon of the alkyl group; in particular —(C₁-C₃)alkyl-(C₆-C₁₀)aryl—is (C₁-C₃)alkyl-phenyl, more particularly benzyl, ethylphenyl or propylphenyl. Phenyl can be substituted as above mentioned, in particular by a halogen atom such as fluorine, bromine, iodine or chlorine, in particular chlorine; a methyl or a methoxy.
“—CONH—(C₆-C₁₀)aryl” means that the nitrogen atom of —CO—NH— is linked to a carbon of the aryl; in particular —CONH—(C₆-C₁₀)aryl is CONH-phenyl. Phenyl can be substituted as above mentioned, in particular by a NRR′ group, R and R′ being as defined herein, in particular ethylamine.
“—(C₁-C₃)alkyl-O—(C₆-C₁₀)aryl” means that the alkyl group is linked to the atom of oxygen by a carbon of the alkyl group, the oxygen atom being also linked to a carbon of the aryl group; in particular —(C₁-C₃)alkyl-O—(C₆-C₁₀)aryl—is (C₁-C₃)alkyl-O-phenyl, more particularly methyl-O-phenyl. Phenyl can be substituted as above mentioned in particular by a NRR′ group, R and R′ being as defined herein, in particular methylamine.
“—O—(C₁-C₃)alkyl-(C₆-C₁₀)aryl” means that the alkyl group is linked to the atom of oxygen by one of the carbons of the alkyl group, another carbon of the alkyl group being linked to a carbon of the aryl group; in particular —O—(C₁-C₃)alkyl-(C₆-C₁₀)aryl is —O—(C₁-C₃)alkyl-phenyl, more particularly —O-methyl-phenyl. Phenyl can be substituted as above mentioned in particular by a NRR′ group, R and R′ being as defined herein, in particular methylamine.
Rb in N(H)Rb-Ra is directly linked to the nitrogen atom.
“carbonyl” refers to C═O.
“heterocycle” or “heterocycloalkyl” refers to a saturated or partially unsaturated non aromatic stable 4 to 10-membered mono, bi or multicyclic rings which can optionally be bridged and wherein at least one member of the ring is a nitrogen atom. The bridge comprises from 0 to 2 carbon atoms between 2 members of the heterocycle. In particular, each ring comprises 1 or 2 nitrogen atoms. Suitable heterocycles are also disclosed in the Handbook of Chemistry and Physics, 76th Edition, CRC Press, Inc., 1995-1996, pages 225 to 226, the disclosure of which is hereby incorporated by reference. Examples of heterocycloalkyl include, but are not limited to piperazine, diazepane, piperidine, pyrrolidine, imidazolidine, morpholine, azetidine, diazabicyclo octanyl, diazabicycloheptanyl, azabicyclohexanyl. Said heterocycle is optionally substituted by a (C₁-C₃) alkyl, in particular methyl, or a NRR′ group, R and R′ being as defined herein, in particular NH₂.
“NH-heterocycle” means that the heterocycle is linked by a carbon or nitrogen atom of the heterocycle to the nitrogen atom of NH.
The term “substituted” generally refers to, unless specified otherwise, a substitution with one or more substituents, which may be identical or different, and which are identified herein.

The compounds of formula (I) as described herein can comprise one or more asymmetric carbon atoms. They can therefore exist in the form of enantiomers or diastereoisomers.

These enantiomers and diastereoisomers, as well as their mixtures, including racemic mixtures, form part of the invention.

The compounds of formula (I) as described herein can be provided in the form of a free base or in the form of addition salts with acids, which also form part of the invention.

These salts are advantageously prepared with pharmaceutically acceptable acids, but salts with other acids, useful for example for the purification or for the isolation of the compounds of formula (I) as described herein, also form part of the invention.

As used herein, the expression “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

As used herein, “pharmaceutically acceptable salts” refer to derivatives of the disclosed compounds wherein the parent compound is modified by making acid or base salts thereof.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like, including mono, di or tri-salts thereof; and the salts prepared from organic acids such as formic, acetic, propionic, succinic, tartaric, citric, methanesulfonic, trifluoromethanesulfonic, benzenesulfonic, trifluoroacetic, glucoronic, glutamic, benzoic, salicylic, toluenesulfonic, oxalic, fumaric, maleic, lactic and the like.

Further addition salts include ammonium salts such as tromethamine, meglumine, epolamine, etc., metal salts such as sodium, potassium, calcium, zinc or magnesium.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media like ether, dioxane, ethyl acetate, ethanol, isopropanol, or acetonitrile are preferred. Lists of suitable salts are found in Remington's Pharmaceutical Sciences, 20^{th} ed., Mack Publishing Company, Easton, P A, 2000, the disclosure of which is hereby incorporated by reference

### Compounds

As mentioned, the compounds according to the invention are compounds of formula (I):

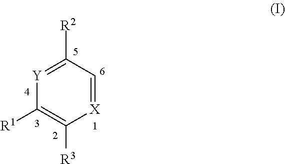

In which:
X can be chosen from:
   CH; and
   N;
Y can be chosen from:
   CH; and
   N;

X and Y not being CH at the same time;
R¹ can be chosen from:
   a halogen atom;
   a —(C₁-C₃)alkyl group;
   a —(C₁-C₃)halogenoalkyl group;
   a —(C₁-C₃)alkoxy group; or
   a nitrile group;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from 0, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
   or its pharmaceutically acceptable salts or optical isomers, racemates, diastereoisomers, enantiomers or tautomers;
with the exclusion of the following compounds:
   1-[3-chloro-5-(trifluoromethyl)-2-pyridyl]piperazine;
   1-[3-chloro-5-(trifluoromethyl)-2-pyridyl]piperazine hydrochloride;
   1-(5-bromo-3-chloro-2-pyridyl)-4-methyl-piperazine;
   1-[2-chloro-4-(trifluoromethyl)phenyl]piperazine hydrochloride;
   1-[3-chloro-5-(trifluoromethyl)-2-pyridyl]piperidin-4-amine;
   1-[3-chloro-5-(trifluoromethyl)-2-pyridyl]-1,4-diazepane;
   methyl 5-chloro-6-piperazin-1-yl-pyridine-3-carboxylate hydrochloride;
   1-(3-chloro-5-methyl-2-pyridyl)piperazine;
   1-(5-bromo-3-chloro-2-pyridyl)piperazine;
   2-chloro-3-piperazin-1-yl-quinoxaline hydrochloride;
   1-[3-bromo-5-(trifluoromethyl)-2-pyridyl]-4-methyl-piperazine;
   3-methyl-2-piperazin-1-yl-quinoline hydrochloride;
   2-piperazin-1-ylquinoline-3-carbonitrile hydrochloride;
   3-[(3R)-3-methylpiperazin-1-yl]quinoxalin-2-ol hydrochloride;
   2-chloro-3-(4-ethylpiperazin-1-yl)quinoxaline;
      3-(5-chloro-6-piperazin-1-yl-3-pyridyl)-5-methyl-1,2,4-oxadiazole;
      (1R,5S)—N-[3-chloro-5-(trifluoromethyl)-2-pyridyl]-3-azabicyclo[3.1.0]hexan-6-amine;
   2-methoxy-3-piperazin-1-yl-quinoxaline hydrochloride;
   2-chloro-6,7-dimethyl-3-piperazin-1-yl-quinoxaline;
   3-Cyan-2-(4-methyl-piperazino)-5-(pyrid-4-yl)-pyridin;
   methyl 5-chloro-6-4-methylpiperazin-1-yl) nicotinate;
   (1-[3-chloro-5-(trifluoromethyl)-2-pyridinyl]-4-methylpiperazine;
   1-(3,5-dichloro-2-pyridyl)piperazine; hydrochloride; 2-methoxy-3-(4-methylpiperazin-1-yl)quinoxaline;
   2-ethoxy-3-(4-methylpiperazin-1-yl)quinoxaline;
   2-methyl-3-(4-methylpiperazin-1-yl)quinoxaline;
   2-ethyl-3-(4-methylpiperazin-1-yl)quinoxaline;
   2-(4-methylpiperazin-1-yl)-3-(trifluoromethyl)quinoxaline;
   2-bromo-3-(4-methylpiperazin-1-yl)quinoxaline;
   2-chloro-3-(4-methyl-1,4-diazepan-1-yl)quinoxaline;
   3,6-dichloro-2-(4-methylpiperazin-1-yl)quinoxaline;
   2,6,7-trichloro-3-(4-methylpiperazin-1-yl)quinoxaline;
   1-(3-chloroquinoxalin-2-yl)-N-methyl-pyrrolidin-3-amine
   2-chloro-3-[(3S)-3-methylpiperazin-1-yl]quinoxaline hydrochloride; and
   4-bromo-1-piperazin-1-yl-isoquinoline.

In particular, said compound of formula (I) is characterized in that:
R¹ can be chosen from:
   a halogen atom;
   a —(C₁-C₃)alkyl group;
   a —(C₁-C₃)halogenoalkyl group; or
   a —(C₁-C₃)alkoxy group; and/or
R² can be chosen from:
   a halogen atom, the fluorine and chlorine atoms being excluded;
   a —(C₁-C₆)alkyl group optionally substituted by one heteroatom chosen from O, N, or S;
   a —(C₂-C₆)alkenyl group optionally substituted by one heteroatom chosen from O, N, or S;
   a —(C₂-C₆)alkynyl group optionally substituted by one heteroatom chosen from O, N, or S;
   a —(C₁-C₃)alkoxy group;
   a —(C₂-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from 0, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;

the other substituents being as defined above, and provided that:
X and Y are not N at the same time if R¹ is halogen and R³a piperazine, and
R² is not COOCH₃ if R¹ is halogen and R³a piperazine.

Still particularly:
R¹ is a halogen atom; and/or
R² can be chosen from:
   a —(C₂-C₆)alkynyl group optionally substituted by one heteroatom chosen from O, N, or S; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;

the other substituents being as defined above, and provided that:
X and Y are not N at the same time if R¹ is halogen and R³a piperazine.

Even more particularly:
X can be chosen from:
   CH; and
   N;
Y can be chosen from:
   CH; and
   N;

X and Y not being CH or N at the same time.

In a preferred embodiment, R³ is chosen from a heterocycle comprising 4 to 10 members with at least one N; in particular a piperazine; said heterocycle comprising optionally a bridge of 0 to 2 carbon atoms between 2 members of the heterocycle and being optionally substituted by a —(C₁-C₃) alkyl group or a —NRR′ group.

In particular, the compound of formula (I) is characterized in that:
X is N and Y is CH; and/or
R¹ is chosen from chlorine, bromine and iodine; and/or
R² is chosen from:
   a iodine;
   a —(C₂-C₆)alkynyl group, especially pentynyl group, optionally substituted by a —NRR′ group;
   a —(C₂-C₆)alkynyl-(C₆-C₁₀)aryl group, especially ethynyl-phenyl, optionally substituted by a —(C₁-C₃)alkyl group, said —(C₁-C₃)alkyl group being optionally substituted by a —NRR′ group;
   a —COORa group;
   a (5-6 membered)heteroaryl group having at least one heteroatom chosen from O, N, or S, especially oxadiazol; said group being optionally substituted by a (C₁-C₃)alkyl group, especially ethyl, optionally substituted by a —NRR′ group; or
   R² forms together with the carbon atom in position 6, a fused phenyl optionally substituted by
      a halogen atom, especially chlorine, bromine and iodine;
      one or more —(C₁-C₄)alkyl groups, especially methyl group;
      a (4-10 membered)heterocycle having at least one N, especially a piperazine, a pyrrolidine and a imidazolidine, said heterocycle being optionally substituted by a (C₁-C₃)alkyl group, especially methyl, a NRR′ group or a carbonyl; said —(C₁-C₃)alkyl group being optionally substituted by a NRR′ group;
      a (C₆-C₁₀)aryl group, especially phenyl, said group being optionally substituted by one or more of a halogen atom, especially fluorine and chlorine, a methyl group optionally substituted by three fluorine atoms, a methoxy group, a (C₁-C₃)alkyl group, optionally substituted by a —NRR′ group or a OH;
      a NH-heterocycle comprising 4 to 10 members with a least one N, especially a piperazine;
      a (5-12 membered)heteroaryl, especially pyridine and triazole, optionally substituted by (C₁-C₃)alkyl group optionally substituted by a —NRR′ group;
      a NR⁷R⁸ group; and/or
Ra is chosen from:
   a phenyl group optionally substituted a —(C₁-C₃)alkyl group being substituted by a NRR′ group;
R and R′, identical or different are chosen from —(C₁-C₃)alkyl group, especially methyl, and H; and/or
R³ is a piperazine optionally substituted by a methyl; and/or
R⁷ and R⁸, identical or different are chosen from H, —(C₁-C₄)alkyl group, especially ethyl, propyl and butyl, and —CO—(C₁-C₃)alkyl group, especially —CO—CH₂—; said —(C₁-C₃)alkyl group and —CO—(C₁-C₃)alkyl group being optionally substituted by —NH₂.

In particular, the compound of formula (I) is characterized in that:
X is N and Y is CH; and/or
R¹ is chosen from chlorine, bromine and iodine; and/or
R² is chosen from:
   a iodine;
   a —(C₂-C₆)alkynyl group, especially pentynyl group, optionally substituted by a-NRR′ group;
   a —COORa group;
   a (5-6 membered)heteroaryl group having at least one heteroatom chosen from 0, N, or S, especially oxadiazol; said group being optionally substituted by a (C₁-C₃)alkyl group, especially ethyl, optionally substituted by a —NRR′ group; or
   R² forms together with the carbon atom in position 6, a fused phenyl optionally substituted by
      a halogen atom, especially chlorine, bromine and iodine;
      one or more —(C₁-C₄)alkyl groups, especially methyl group;
      a (4-10 membered)heterocycle having at least one N, especially a piperazine;
      a (C₆-C₁₀)aryl group, especially phenyl, said group being optionally substituted by a (C₁-C₃)alkyl group, especially methyl, optionally substituted by a —NRR′ group;
      a NR⁷R⁸ group; and/or
Ra is chosen from:
   a phenyl group optionally substituted a —(C₁-C₃)alkyl group being substituted by a NRR′ group;
R and R′ are H; and/or
R³ is a piperazine optionally substituted by a methyl; and/or
R⁷ and R⁸, identical or different are chosen from H, —(C₁-C₃)alkyl group, especially ethyl and propyl, and —CO—(C₁-C₃)alkyl group, especially —CO—CH₂—; said —(C₁-C₃)alkyl group and —CO—(C₁-C₃)alkyl group being optionally substituted by —NH₂.

In one embodiment, said compound of formula (I) is chosen from:
1-(5-bromo-3-chloro-2-pyridyl)piperazine hydrochloride;
1-(3-chloro-5-iodo-2-pyridyl)piperazine;
1-(3-chloro-5-methyl-2-pyridyl)-4-methyl-piperazine;
1-(3-chloro-5-methyl-2-pyridyl)piperazine hydrochloride;
N-[5-chloro-6-(4-methylpiperazin-1-yl)-3-pyridyl]acetamide;
N-(5-chloro-6-piperazin-1-yl-3-pyridyl)acetamide hydrochloride;
N-(5-chloro-6-piperazin-1-yl-3-pyridyl)methane sulfonamide hydrochloride;
methyl 5-chloro-6-(4-methylpiperazin-1-yl)pyridine-3-carboxylate;
ethyl 5-chloro-6-piperazin-1-yl-pyridine-3-carboxylate hydrochloride;
benzyl 5-chloro-6-piperazin-1-yl-pyridine-3-carboxylate hydrochloride;
2-phenylethyl5-chloro-6-piperazin-1-yl-pyridine-3-carboxylate hydrochloride;
3-phenylpropyl5-chloro-6-piperazin-1-yl-pyridine-3 carboxylate hydrochloride;
p-tolylmethyl 5-chloro-6-piperazin-1-yl-pyridine-3-carboxylate hydrochloride;
(4-chlorophenyl)methyl 5-chloro-6-piperazin-1-yl-pyridine-3-carboxylate hydrochloride;
(4-methoxyphenyl)methyl 5-chloro-6-piperazin-1-yl-pyridine-3-carboxylate hydrochloride;
2-(4-chlorophenyl)ethyl 5-chloro-6-piperazin-1-yl-pyridine-3-carboxylate hydrochloride;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
3-bromo-2-piperazin-1-yl-6-(trifluoromethyl)quinoline hydrochloride;
3-bromo-6-chloro-2-piperazin-1-yl-quinoline hydrochloride;
1-[2-chloro-4-(trifluoromethyl)phenyl]-4-methyl-piperazine;
2-chloro-3-piperazin-1-yl-quinoline hydrochloride;
1-[3-bromo-5-(trifluoromethyl)-2-pyridyl]piperazine hydrochloride;
3-iodo-2-piperazin-1-yl-quinoline hydrochloride;
1-[5-iodo-3-(trifluoromethyl)-2-pyridyl]piperazine hydrochloride;
1-(3-chloro-5-iodo-2-pyridyl)piperazine;
3-chloro-2-(1,4-diazepan-1-yl)quinoline;
2-chloro-3-(3,8-diazabicyclo[3.2.1]octan-3-yl)quinoxaline hydrochloride;
N-[(1R,5S)-3-azabicyclo[3.1.0]hexan-6-yl]-3-chloro-quinolin-2-amine hydrochloride;
1-[3-chloro-5-(trifluoromethyl)-2-pyridyl]piperidin-4-amine;
1-(3-chloro-5-methoxy-2-pyridyl)piperazine hydrochloride;
(1R,5S)—N-[3-chloro-5-(trifluoromethyl)-2-pyridyl]-3-azabicyclo[3.1.0]hexan-6-amine formic acid;
3-(4-chlorophenyl)propyl 5-chloro-6-piperazin-1-yl-pyridine-3-carboxylate hydrochloride;
[3-(aminomethyl)phenyl] 5-chloro-6-piperazin-1-yl-pyridine-3-carboxylate dihydrochloride;
5-chloro-N-[(4-chlorophenyl)methyl]-6-piperazin-1-yl-pyridine-3-carboxamide hydrochloride;
N-[4-(2-aminoethyl)phenyl]-5-chloro-6-piperazin-1-yl-pyridine-3-carboxamide dihydrochloride;
2,6-dichloro-3-piperazin-1-yl-quinoline hydrochloride;
2-chloro-6-methyl-3-piperazin-1-yl-quinoline hydrochloride;
3-chloro-2-(3,8-diazabicyclo[3.2.1]octan-8-yl)quinoline hydrochloride;
2-chloro-6,7-dimethyl-3-piperazin-1-yl-quinoxaline hydrochloride;
N-[(1R,5S)-3-azabicyclo[3.1.0]hexan-6-yl]-3-chloro-quinoxalin-2-amine hydrochloride;
3-bromo-6-methyl-2-piperazin-1-yl-quinoline hydrochloride;
3-chloro-2,6-di(piperazin-1 yl)quinoline dihydrochloride;
2-methoxy-3-piperazin-1-yl-quinoxaline 2,2,2-trifluoroacetic acid;
2-chloro-3-(3,6-diazabicyclo[3.1.1]heptan-3-yl)quinoxaline trifluoromethanesulfonic acid; and
2-chloro-3-(4-ethylpiperazin-1-yl)quinoxaline hydrochloride;
4-(2-aminoethyl)-N-(5-chloro-6-piperazin-1-yl-3-pyridyl)benzamide dihydrochloride;
phenyl 5-chloro-6-piperazin-1-yl-pyridine-3-carboxylate 2,2,2-trifluoroacetic acid;
2-(5-chloro-6-piperazin-1-yl-3-pyridyl)-5-methyl-1,3,4-oxadiazole;
3-(5-chloro-6-piperazin-1-yl-3-pyridyl)-5-methyl-1,2,4-oxadiazole hydrochloride;
3-(5-chloro-6-piperazin-1-yl-3-pyridyl)prop-2-yn-1-amine 2,2,2-trifluoroacetic acid;
5-(5-chloro-6-piperazin-1-yl-3-pyridyl)pent-4-yn-1-amine 2,2,2-trifluoroacetic acid;
4-(5-chloro-6-piperazin-1-yl-3-pyridyl)but-3-yn-1-amine 2,2,2-trifluoroacetic acid;
3-chloro-8-methyl-2-piperazin-1-yl-quinoline hydrochloride;
3-chloro-6-iodo-2-piperazin-1-yl-quinoline hydrochloride;
6-bromo-3-chloro-2-piperazin-1-yl-quinoline hydrochloride;
3-chloro-2-piperazin-1-yl-1,5-naphthyridine hydrochloride;
2-chloro-6-methoxy-3-piperazin-1-yl-quinoxaline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
1-(3-chloro-2-piperazin-1-yl-6-quinolyl)piperazin-2-one dihydrochloride;
2-amino-N-(3-chloro-2-piperazin-1-yl-6-quinolyl)acetamide dihydrochloride;
[3-(5-chloro-6-piperazin-1-yl-3-pyridyl)-1,2,4-oxadiazol-5-yl]methanamine dihydrochloride;
2-[3-(5-chloro-6-piperazin-1-yl-3-pyridyl)-1,2,4-oxadiazol-5-yl]ethanamine dihydrochloride;
3-[3-(5-chloro-6-piperazin-1-yl-3-pyridyl)-1,2,4-oxadiazol-5-yl]propan-1-amine dihydrochloride;
[3-[(5-chloro-6-piperazin-1-yl-3-pyridyl)methoxy]phenyl]methanamine dihydrochloride;
[3-[(5-chloro-6-piperazin-1-yl-3-pyridyl)oxymethyl]phenyl]methanamine dihydrochloride;
3-chloro-2-piperazin-1-yl-N-(4-piperidyl)quinolin-6-amine dihydrochloride;
1-(3-chloro-2-piperazin-1-yl-6-quinolyl)piperidin-4-amine dihydrochloride;
3-chloro-6-(1,4-diazepan-1-yl)-2-piperazin-1-yl-quinoline dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
1-(3-chloro-2-piperazin-1-yl-6-quinolyl)azetidin-3-amine dihydrochloride;
3-chloro-6-(2,6-diazaspiro[3.3]heptan-2-yl)-2-piperazin-1-yl-quinoline dihydrochloride;
3-chloro-6-(3,8-diazabicyclo[3.2.1]octan-8-yl)-2-piperazin-1-yl-quinoline dihydrochloride;
3-chloro-6-(3,8-diazabicyclo[3.2.1]octan-3-yl)-2-piperazin-1-yl-quinoline dihydrochloride;
3-chloro-2-piperazin-1-yl-6-(1-piperidyl)quinoline hydrochloride;
4-(3-chloro-2-piperazin-1-yl-6-quinolyl)morpholine hydrochloride;
[1-(3-chloro-2-piperazin-1-yl-6-quinolyl)-3-piperidyl]methanamine dihydrochloride;
2-[1-(3-chloro-2-piperazin-1-yl-6-quinolyl)-3-piperidyl]ethanamine dihydrochloride;
[1-(3-chloro-2-piperazin-1-yl-6-quinolyl)pyrrolidin-2-yl]methanamine dihydrochloride
[1-(3-chloro-2-piperazin-1-yl-6-quinolyl)-2-piperidyl]methanamine dihydrochloride;
[3-(3-chloro-2-piperazin-1-yl-6-quinolyl)phenyl]methanamine dihydrochloride;
1-(3-chloro-5-ethynyl-2-pyridyl)piperazine;
3,7-dichloro-8-methyl-2-piperazin-1-yl-quinoline hydrochloride;
3-chloro-6-phenyl-2-piperazin-1-yl-quinoline hydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
3-chloro-2-piperazin-1-yl-N-[(3S)-3-piperidyl]quinolin-6-amine dihydrochloride;
N′-(3-chloro-2-piperazin-1-yl-6-quinolyl)butane-1,4-diamine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
1-[3-chloro-5-(2-phenylethynyl)-2-pyridyl]piperazine 2,2,2-trifluoroacetic acid;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride;
2-[3-(3-chloro-2-piperazin-1-yl-6-quinolyl)phenyl]propan-2-amine dihydrochloride;
2-[5-(3-chloro-2-piperazin-1-yl-6-quinolyl)triazol-1-yl]-N,N-dimethyl-ethanamine dihydrochloride;
[1-(3-chloro-2-piperazin-1-yl-6-quinolyl)imidazol-2-yl]methanamine dihydrochloride;
2-[1-(3-chloro-2-piperazin-1-yl-6-quinolyl)imidazol-2-yl]ethanamine dihydrochloride;
[1-(3-chloro-2-piperazin-1-yl-6-quinolyl)imidazol-4-yl]methanamine dihydrochloride;
2-[1-(3-chloro-2-piperazin-1-yl-6-quinolyl)imidazol-4-yl]ethanamine dihydrochloride;
[3-(3-chloro-2-piperazin-1-yl-6-quinolyl)imidazol-4-yl]methanamine dihydrochloride;
2-[3-(3-chloro-2-piperazin-1-yl-6-quinolyl)imidazol-4-yl]ethanamine dihydrochloride;
[4-(3-chloro-2-piperazin-1-yl-6-quinolyl)-1,2,4-triazol-3-yl]methanamine dihydrochloride;
2-[4-(3-chloro-2-piperazin-1-yl-6-quinolyl)-1,2,4-triazol-3-yl]ethanamine dihydrochloride;
3-(2-aminoethyl)-1-(3-chloro-2-piperazin-1-yl-6-quinolyl)pyrrolidin-2-one dihydrochloride;
1-(2-aminoethyl)-3-(3-chloro-2-piperazin-1-yl-6-quinolyl)imidazolidin-2-one dihydrochloride;
1-(3-chloro-2-piperazin-1-yl-6-quinolyl)-3-[2-(dimethylamino)ethyl]pyrrolidin-2-one dihydrochloride;
1-(3-chloro-2-piperazin-1-yl-6-quinolyl)-3-[2-(dimethylamino)ethyl]imidazolidin-2-one dihydrochloride;
[5-(3-chloro-2-piperazin-1-yl-6-quinolyl)-1,2,4-oxadiazol-3-yl]methanamine dihydrochloride;
2-[5-(3-chloro-2-piperazin-1-yl-6-quinolyl)-1,2,4-oxadiazol-3-yl]ethanamine dihydrochloride;
[3-(3-chloro-2-piperazin-1-yl-6-quinolyl)-1,2,4-oxadiazol-5-yl]methanamine dihydrochloride;
2-[3-(3-chloro-2-piperazin-1-yl-6-quinolyl)-1,2,4-oxadiazol-5-yl]ethanamine dihydrochloride;
[5-(3-chloro-2-piperazin-1-yl-6-quinolyl)-1,3,4-oxadiazol-2-yl]methanamine dihydrochloride;
2-[5-(3-chloro-2-piperazin-1-yl-6-quinolyl)-1,3,4-oxadiazol-2-yl]ethanamine dihydrochloride;
[4-(3-chloro-2-piperazin-1-yl-6-quinolyl)oxazol-2-yl]methanamine dihydrochloride;
2-[4-(3-chloro-2-piperazin-1-yl-6-quinolyl)oxazol-2-yl]ethanamine dihydrochloride;
[4-(3-chloro-2-piperazin-1-yl-6-quinolyl)thiazol-2-yl]methanamine dihydrochloride;
2-[4-(3-chloro-2-piperazin-1-yl-6-quinolyl)thiazol-2-yl]ethanamine dihydrochloride;
[4-(3-chloro-2-piperazin-1-yl-6-quinolyl)-1H-imidazol-2-yl]methanamine dihydrochloride;
2-[4-(3-chloro-2-piperazin-1-yl-6-quinolyl)-1H-imidazol-2-yl]ethanamine dihydrochloride;
[3-(3-chloro-2-piperazin-1-yl-6-quinolyl)-1H-1,2,4-triazol-5-yl]methanamine dihydrochloride;
2-[3-(3-chloro-2-piperazin-1-yl-6-quinolyl)-1H-1,2,4-triazol-5-yl]ethanamine dihydrochloride;
6-[3-(aminomethyl)phenyl]-N-[(1S,5R)-3-azabicyclo[3.1.0]hexan-6-yl]-3-chloro-quinolin-2-amine dihydrochloride;
[3-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride;
[4-(3-chloro-2-piperazin-1-yl-6-quinolyl)-2-pyridyl]methanamine dihydrochloride;
[6-(3-chloro-2-piperazin-1-yl-6-quinolyl)-2-pyridyl]methanamine dihydrochloride;
[2-(3-chloro-2-piperazin-1-yl-6-quinolyl)-4-pyridyl]methanamine dihydrochloride;
[6-(3-chloro-2-piperazin-1-yl-6-quinolyl)pyrazin-2-yl]methanamine dihydrochloride;
3-chloro-6-imidazol-1-yl-2-piperazin-1-yl-quinoline hydrochloride; and
(1S,4S)-2-[3-chloro-5-(trifluoromethyl)-2-pyridyl]-2,5-diazabicyclo[2.2.1]heptane formic acid.

In another embodiment, said compound of formula (I) is chosen from:
1-(3-chloro-5-iodo-2-pyridyl)piperazine;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
2-chloro-3-piperazin-1-yl-quinoxaline hydrochloride;
3-iodo-2-piperazin-1-yl-quinoline hydrochloride;
[3-(aminomethyl)phenyl] 5-chloro-6-piperazin-1-yl-pyridine-3-carboxylate dihydrochloride;
3-bromo-6-chloro-2-piperazin-1-yl-quinoline hydrochloride;
3-bromo-6-methyl-2-piperazin-1-yl-quinoline hydrochloride;
3-chloro-2,6-di(piperazin-1 yl)quinoline dihydrochloride;
3-chloro-6-iodo-2-piperazin-1-yl-quinoline hydrochloride;
6-bromo-3-chloro-2-piperazin-1-yl-quinoline hydrochloride;
2-[3-(5-chloro-6-piperazin-1-yl-3-pyridyl)-1,2,4-oxadiazol-5-yl]ethanamine dihydrochloride;
2-chloro-6,7-dimethyl-3-piperazin-1-yl-quinoxaline hydrochloride;
5-(5-chloro-6-piperazin-1-yl-3-pyridyl)pent-4-yn-1-amine 2,2,2-trifluoroacetic acid;
3-chloro-8-methyl-2-piperazin-1-yl-quinoline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
[3-(3-chloro-2-piperazin-1-yl-6-quinolyl)phenyl]methanamine dihydrochloride;
2-amino-N-(3-chloro-2-piperazin-1-yl-6-quinolyl)acetamide dihydrochloride.
[1-(3-chloro-2-piperazin-1-yl-6-quinolyl)-3-piperidyl]methanamine dihydrochloride;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3,7-dichloro-8-methyl-2-piperazin-1-yl-quinoline hydrochloride;
N′-(3-chloro-2-piperazin-1-yl-6-quinolyl)butane-1,4-diamine dihydrochloride;
3-chloro-2-piperazin-1-yl-N-[(3S)-3-piperidyl]quinolin-6-amine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
2-[3-(3-chloro-2-piperazin-1-yl-6-quinolyl)phenyl]propan-2-amine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride; and
[2-(3-chloro-2-piperazin-1-yl-6-quinolyl)-4-pyridyl]methanamine dihydrochloride.

Compounds provided herein can be formulated into pharmaceutical compositions, optionally by admixture with one or more pharmaceutically acceptable excipients.

The present invention thus also relates to a pharmaceutical composition comprising a compound of formula (I) as defined in this section, and a pharmaceutically acceptable excipient.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, P A, 2000.

Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration.

### Compounds for Use/Method of Treatment

As already mentioned, the present invention also relates to a compound of formula (I):

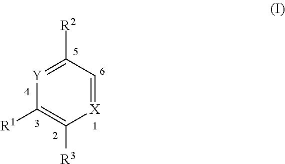

In which:
X can be chosen from:
   CH; and
   N;
Y can be chosen from:
   CH; and
   N;
R¹ can be chosen from:
   a halogen atom;
   a —(C₁-C₃)alkyl group;
   a —(C₁-C₃)halogenoalkyl group;
   a —(C₁-C₃)alkoxy group;
   a nitrile group; or
   R¹ forms together with Y a fused phenyl in positions 3 and 4;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₅)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from 0, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optional a bridge of 0 to 2 carbon atoms between 2 members of the heterocycle;
      a (C₆-C₁₀)aryl group, optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
   or its pharmaceutically acceptable salts or optical isomers, racemates, diastereoisomers, enantiomers or tautomers;
   for use as a medicament.

In one embodiment:
X can be chosen from:
   CH; and
   N;
Y can be chosen from:
   CH; and
   N;
R¹ can be chosen from:
   a halogen atom;
   a —(C₁-C₃)alkyl group;
   a —(C₁-C₃)halogenoalkyl group;
   a —(C₁-C₃)alkoxy group;
   a nitrile group; or
   R¹ forms together with Y a fused phenyl in positions 3 and 4;
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from 0, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optional a bridge of 0 to 2 carbon atoms between 2 members of the heterocycle;
      a (C₆-C₁₀)aryl group optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-6 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group;
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂;
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group.

In particular:
R¹ can be chosen from:
   a halogen atom;
   a —(C₁-C₃)alkyl group;
   a —(C₁-C₃)halogenoalkyl group;
   a —(C₁-C₃)alkoxy group; or
   a nitrile group; and/or
R² can be chosen from:
   a halogen atom, the fluorine atom being excluded;
   a —(C₁-C₆)alkyl group optionally substituted by one heteroatom chosen from O, N, or S;
   a —(C₂-C₆)alkenyl group optionally substituted by one heteroatom chosen from 0, N, or S;
   a —(C₂-C₆)alkynyl group optionally substituted by one heteroatom chosen from 0, N, or S;
   a —(C₁-C₃)alkoxy group;
   a —(C₁-C₃)halogenoalkyl group;
   a —COORa group;
   a —N(H)Rb-Ra group;
   a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
   a CONH—(C₆-C₁₀)aryl group optionally substituted by a —(C₁-C₃)alkyl group, said —(C₁-C₃)alkyl group being optionally substituted by a —NRR′ group;
   a —(C₁-C₃)alkyl-O—(C₆-C₁₀)aryl group, said group being optionally substituted by a (C₁-C₃)alkyl group optionally substituted by a —NRR′ group;
   a —O—(C₁-C₃)alkyl-(C₆-C₁₀)aryl group, said group being optionally substituted by a (C₁-C₃)alkyl group optionally substituted by a —NRR′ group;
   a (5-6 membered)heteroaryl group having at least one heteroatom chosen from O, N, or S; said group being optionally substituted by a (C₁-C₃)alkyl group optionally substituted by a —NRR′ group;
   a —CONH—(C₁-C₃)alkyl-(C₆-C₁₀)aryl group, said group being optionally substituted by a halogen atom, a methyl group or a methoxy group; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group,
Ra is chosen from:
   a —(C₁-C₆)alkyl group;
   a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
   a phenyl group optionally substituted by a —(C₁-C₃)alkyl group, said —(C₁-C₃)alkyl group being substituted by a —NRR′ group; or
   a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
   carbonyl; and
   SO₂; and/or
R³ is chosen from:
   a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
   a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group.

More particularly:
R¹ is a halogen atom; and/or
R² can be chosen from:
   a —(C₂-C₆)alkynyl group optionally substituted by one heteroatom chosen from O, N, or S; or
   R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
      one or more halogen atoms,
      one or more —(C₁-C₄)alkyl groups,
      one or more —(C₁-C₃)halogenoalkyl groups,
      one or more —(C₁-C₃)alkoxy groups optionally substituted by one to three fluorine atoms,
      a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
      a NH-heterocycle comprising 4 to 10 members with a least one N, said heterocycle comprising optionally a bridge of 0 to 2 carbon atoms between 2 members of the heterocycle;
      a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
      a NR⁷R⁸ group; and/or
X can be chosen from:
   CH; and
   N;
Y can be chosen from:
   CH; and
   N;

X and Y not being CH or N at the same time; and/or
R³ is chosen from a heterocycle comprising 4 to 10 members with at least one N; in particular a piperazine; said heterocycle comprising optionally a bridge of 0 to 2 carbon atoms between 2 members of the heterocycle and being optionally substituted by a —(C₁-C₃) alkyl group or a —NRR′ group;

Even more particularly, said compound is characterized in that:
X and Y are N or X is N and Y is CH; and/or
R¹ is a halogen atom; and/or
R² can be chosen from:
   a halogen atom, the fluorine atom being excluded;
   a —(C₂-C₆)alkynyl group optionally substituted by a —NRR′ group;
   a —COORa group;
   a (5-6 membered)heteroaryl group having at least one heteroatom chosen from 0, N, or S; said group being optionally substituted by a (C₁-C₃)alkyl group optionally substituted by a —NRR′ group; or
   R² forms together with the carbon atom in position 6, a fused phenyl optionally substituted by:
      one or more a halogen atoms;
      one or more —(C₁-C₄)alkyl groups;
      a (4-10 membered)heterocycle having at least one N, said heterocycle comprising optionally a bridge of 0 to 2 carbon atoms between 2 members of the heterocycle;
      a (C₆-C₁₀)aryl group, said group being optionally substituted by a (C₁-C₃)alkyl group optionally substituted by a —NRR′ group;
      a NR⁷R⁸ group; and/or
Ra is a phenyl group optionally substituted a —(C₁-C₃)alkyl group being substituted by a —NRR′ group; and/or
R and R′ are H; and/or
R³ is a heterocycle comprising 4 to 10 members with at least one N; said heterocycle comprising optionally a bridge of 0 to 2 carbon atoms between 2 members of the heterocycle and being optionally substituted by a methyl or —NRR′ group; and/or
R⁷ and R⁸, identical or different are chosen from H, —(C₁-C₃)alkyl group and —CO—(C₁-C₃)alkyl group; said —(C₁-C₃)alkyl group and —CO—(C₁-C₃)alkyl group being optionally substituted by —NH₂.

Even more particularly, said compound of formula (I) is characterized in that:
X and Y are N or X is N and Y is CH; and/or
R¹ is chosen from chlorine, bromine and iodine; and/or
R² is chosen from:
   a halogen atom, especially iodine;
   a —(C₂-C₆)alkynyl group, especially pentynyl group, optionally substituted by a-NRR′ group;
   a —COORa group;
   a (5-6 membered)heteroaryl group having at least one heteroatom chosen from 0, N, or S, especially oxadiazol; said group being optionally substituted by a (C₁-C₃)alkyl group, especially ethyl, optionally substituted by a —NRR′ group; or
   R² forms together with the carbon atom in position 6, a fused phenyl optionally substituted by
      a halogen atom, especially chlorine, bromine and iodine;
      one or more —(C₁-C₄)alkyl groups, especially methyl group;
      a (4-10 membered)heterocycle having at least one N, especially a piperazine;
      a (C₆-C₁₀)aryl group, especially phenyl, said group being optionally substituted by a (C₁-C₃)alkyl group, especially methyl, optionally substituted by a —NRR′ group;
      a NR⁷R⁸ group; and/or
Ra is chosen from:
   a phenyl group optionally substituted a —(C₁-C₃)alkyl group being substituted by a —NRR′ group;
R and R′ are H; and/or
   R³ is a piperazine optionally substituted by a methyl; and/or
R⁷ and R⁸, identical or different are chosen from H, —(C₁-C₃)alkyl group, especially ethyl and propyl, and —CO—(C₁-C₃)alkyl group, especially —CO—CH₂—; said —(C₁-C₃)alkyl group and —CO—(C₁-C₃)alkyl group being optionally substituted by —NH₂.

In one embodiment, said compound is chosen from:
1-[3-chloro-5-(trifluoromethyl)-2-pyridyl]-4-methyl-piperazine;
1-[3-chloro-5-(trifluoromethyl)-2-pyridyl]iperazine;
1-[3-chloro-5-(trifluoromethyl)-2-pyridyl]piperazine hydrochloride;
1-(5-bromo-3-chloro-2-pyridyl)-4-methyl-piperazine;
1-(5-bromo-3-chloro-2-pyridyl)piperazine hydrochloride;
1-(3-chloro-5-iodo-2-pyridyl)piperazine;
1-(3-chloro-5-methyl-2-pyridyl)-4-methyl-piperazine;
1-(3-chloro-5-methyl-2-pyridyl)piperazine hydrochloride;
N-[5-chloro-6-(4-methylpiperazin-1-yl)-3-pyridyl]acetamide;
N-(5-chloro-6-piperazin-1-yl-3-pyridyl)acetamide hydrochloride;
N-(5-chloro-6-piperazin-1-yl-3-pyridyl)methane sulfonamide hydrochloride;
methyl 5-chloro-6-(4-methylpiperazin-1-yl)pyridine-3-carboxylate;
methyl 5-chloro-6-piperazin-1-yl-pyridine-3-carboxylate hydrochloride;
ethyl 5-chloro-6-piperazin-1-yl-pyridine-3-carboxylate hydrochloride;
benzyl 5-chloro-6-piperazin-1-yl-pyridine-3-carboxylate hydrochloride;
2-phenylethyl5-chloro-6-piperazin-1-yl-pyridine-3-carboxylate hydrochloride;
3-phenylpropyl5-chloro-6-piperazin-1-yl-pyridine-3 carboxylate hydrochloride;
p-tolylmethyl 5-chloro-6-piperazin-1-yl-pyridine-3-carboxylate hydrochloride;
(4-chlorophenyl)methyl 5-chloro-6-piperazin-1-yl-pyridine-3-carboxylate hydrochloride;
(4-methoxyphenyl)methyl 5-chloro-6-piperazin-1-yl-pyridine-3-carboxylate hydrochloride;
2-(4-chlorophenyl)ethyl 5-chloro-6-piperazin-1-yl-pyridine-3-carboxylate hydrochloride;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
3-bromo-2-piperazin-1-yl-6-(trifluoromethyl)quinoline hydrochloride;
3-bromo-6-chloro-2-piperazin-1-yl-quinoline hydrochloride;
1-[2-chloro-4-(trifluoromethyl)phenyl]-4-methyl-piperazine;
1-[2-chloro-4-(trifluoromethyl)phenyl]piperazine hydrochloride;
2-chloro-3-piperazin-1-yl-quinoline hydrochloride;
2-chloro-3-piperazin-1-yl-quinoxaline hydrochloride;
1-[3-bromo-5-(trifluoromethyl)-2-pyridyl]-4-methyl-piperazine;
1-[3-bromo-5-(trifluoromethyl)-2-pyridyl]piperazine hydrochloride;
3-iodo-2-piperazin-1-yl-quinoline hydrochloride;
3-methyl-2-piperazin-1-yl-quinoline hydrochloride;
2-piperazin-1-ylquinoline-3-carbonitrile hydrochloride;
1-[5-iodo-3-(trifluoromethyl)-2-pyridyl]piperazine hydrochloride;
1-(3-chloro-5-iodo-2-pyridyl)piperazine;
1-[3-chloro-5-(trifluoromethyl)-2-pyridyl]-1,4-diazepane;
3-chloro-2-(1,4-diazepan-1-yl)quinoline;
2-chloro-3-(3,8-diazabicyclo[3.2.1]octan-3-yl)quinoxaline hydrochloride;
3-[(3R)-3-methylpiperazin-1-yl]quinoxalin-2-ol hydrochloride;
2-chloro-3-[(3S)-3-methylpiperazin-1-yl]quinoxaline hydrochloride;
N-[(1R,5S)-3-azabicyclo[3.1.0]hexan-6-yl]-3-chloro-quinolin-2-amine hydrochloride;
1-[3-chloro-5-(trifluoromethyl)-2-pyridyl]piperidin-4-amine;
1-(3-chloro-5-methoxy-2-pyridyl)piperazine hydrochloride;
(1R,5S)—N-[3-chloro-5-(trifluoromethyl)-2-pyridyl]-3-azabicyclo[3.1.0]hexan-6-amine formic acid;
3-(4-chlorophenyl)propyl 5-chloro-6-piperazin-1-yl-pyridine-3-carboxylate hydrochloride;
[3-(aminomethyl)phenyl] 5-chloro-6-piperazin-1-yl-pyridine-3-carboxylate dihydrochloride;
5-chloro-N-[(4-chlorophenyl)methyl]-6-piperazin-1-yl-pyridine-3-carboxamide hydrochloride;
N-[4-(2-aminoethyl)phenyl]-5-chloro-6-piperazin-1-yl-pyridine-3-carboxamide dihydrochloride;
2,6-dichloro-3-piperazin-1-yl-quinoline hydrochloride;
2-chloro-6-methyl-3-piperazin-1-yl-quinoline hydrochloride;
3-chloro-2-(3,8-diazabicyclo[3.2.1]octan-8-yl)quinoline hydrochloride;
2-chloro-6,7-dimethyl-3-piperazin-1-yl-quinoxaline hydrochloride;
N-[(1R,5S)-3-azabicyclo[3.1.0]hexan-6-yl]-3-chloro-quinoxalin-2-amine hydrochloride;
3-bromo-6-methyl-2-piperazin-1-yl-quinoline hydrochloride;
3-chloro-2,6-di(piperazin-1 yl)quinoline dihydrochloride;
2-methoxy-3-piperazin-1-yl-quinoxaline 2,2,2-trifluoroacetic acid;
2-chloro-3-(3,6-diazabicyclo[3.1.1]heptan-3-yl)quinoxaline trifluoromethanesulfonic acid;
2-chloro-3-(4-ethylpiperazin-1-yl)quinoxaline hydrochloride;
4-(2-aminoethyl)-N-(5-chloro-6-piperazin-1-yl-3-pyridyl)benzamide dihydrochloride;
phenyl 5-chloro-6-piperazin-1-yl-pyridine-3-carboxylate 2,2,2-trifluoroacetic acid;
3-(5-chloro-6-piperazin-1-yl-3-pyridyl)-5-methyl-1,2,4-oxadiazole hydrochloride;
3-(5-chloro-6-piperazin-1-yl-3-pyridyl)prop-2-yn-1-amine 2,2,2-trifluoroacetic acid;
5-(5-chloro-6-piperazin-1-yl-3-pyridyl)pent-4-yn-1-amine 2,2,2-trifluoroacetic acid;
4-(5-chloro-6-piperazin-1-yl-3-pyridyl)but-3-yn-1-amine 2,2,2-trifluoroacetic acid;
3-chloro-8-methyl-2-piperazin-1-yl-quinoline hydrochloride;
3-chloro-6-iodo-2-piperazin-1-yl-quinoline hydrochloride;
6-bromo-3-chloro-2-piperazin-1-yl-quinoline hydrochloride;
3-chloro-2-piperazin-1-yl-1,5-naphthyridine hydrochloride;
2-chloro-6-methoxy-3-piperazin-1-yl-quinoxaline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
1-(3-chloro-2-piperazin-1-yl-6-quinolyl)piperazin-2-one dihydrochloride;
2-amino-N-(3-chloro-2-piperazin-1-yl-6-quinolyl)acetamide dihydrochloride;
[3-(5-chloro-6-piperazin-1-yl-3-pyridyl)-1,2,4-oxadiazol-5-yl]methanamine dihydrochloride;
2-[3-(5-chloro-6-piperazin-1-yl-3-pyridyl)-1,2,4-oxadiazol-5-yl]ethanamine dihydrochloride;
3-[3-(5-chloro-6-piperazin-1-yl-3-pyridyl)-1,2,4-oxadiazol-5-yl]propan-1-amine dihydrochloride;
[3-[(5-chloro-6-piperazin-1-yl-3-pyridyl)methoxy]phenyl]methanamine dihydrochloride;
[3-[(5-chloro-6-piperazin-1-yl-3-pyridyl)oxymethyl]phenyl]methanamine dihydrochloride;
3-chloro-2-piperazin-1-yl-N-(4-piperidyl)quinolin-6-amine dihydrochloride;
1-(3-chloro-2-piperazin-1-yl-6-quinolyl)piperidin-4-amine dihydrochloride;
3-chloro-6-(1,4-diazepan-1-yl)-2-piperazin-1-yl-quinoline dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
1-(3-chloro-2-piperazin-1-yl-6-quinolyl)azetidin-3-amine dihydrochloride;
3-chloro-6-(2,6-diazaspiro[3.3]heptan-2-yl)-2-piperazin-1-yl-quinoline dihydrochloride
3-chloro-6-(3,8-diazabicyclo[3.2.1]octan-8-yl)-2-piperazin-1-yl-quinoline dihydrochloride;
3-chloro-6-(3,8-diazabicyclo[3.2.1]octan-3-yl)-2-piperazin-1-yl-quinoline dihydrochloride;
3-chloro-2-piperazin-1-yl-6-(1-piperidyl)quinoline hydrochloride;
4-(3-chloro-2-piperazin-1-yl-6-quinolyl)morpholine hydrochloride;
[1-(3-chloro-2-piperazin-1-yl-6-quinolyl)-3-piperidyl]methanamine dihydrochloride;
2-[1-(3-chloro-2-piperazin-1-yl-6-quinolyl)-3-piperidyl]ethanamine dihydrochloride;
[1-(3-chloro-2-piperazin-1-yl-6-quinolyl)pyrrolidin-2-yl]methanamine dihydrochloride;
[1-(3-chloro-2-piperazin-1-yl-6-quinolyl)-2-piperidyl]methanamine dihydrochloride;
[3-(3-chloro-2-piperazin-1-yl-6-quinolyl)phenyl]methanamine dihydrochloride;
1-(3-chloro-5-ethynyl-2-pyridyl)piperazine;
3,7-dichloro-8-methyl-2-piperazin-1-yl-quinoline hydrochloride;
3-chloro-6-phenyl-2-piperazin-1-yl-quinoline hydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
3-chloro-2-piperazin-1-yl-N-[(3S)-3-piperidyl]quinolin-6-amine dihydrochloride;
N′-(3-chloro-2-piperazin-1-yl-6-quinolyl)butane-1,4-diamine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
1-[3-chloro-5-(2-phenylethynyl)-2-pyridyl]piperazine 2,2,2-trifluoroacetic acid;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride;
2-[3-(3-chloro-2-piperazin-1-yl-6-quinolyl)phenyl]propan-2-amine dihydrochloride;
2-[5-(3-chloro-2-piperazin-1-yl-6-quinolyl)triazol-1-yl]-N,N-dimethyl-ethanamine dihydrochloride;
[1-(3-chloro-2-piperazin-1-yl-6-quinolyl)imidazol-2-yl]methanamine dihydrochloride;
2-[1-(3-chloro-2-piperazin-1-yl-6-quinolyl)imidazol-2-yl]ethanamine dihydrochloride;
[1-(3-chloro-2-piperazin-1-yl-6-quinolyl)imidazol-4-yl]methanamine dihydrochloride;
2-[1-(3-chloro-2-piperazin-1-yl-6-quinolyl)imidazol-4-yl]ethanamine dihydrochloride;
[3-(3-chloro-2-piperazin-1-yl-6-quinolyl)imidazol-4-yl]methanamine dihydrochloride;
2-[3-(3-chloro-2-piperazin-1-yl-6-quinolyl)imidazol-4-yl]ethanamine dihydrochloride;
[4-(3-chloro-2-piperazin-1-yl-6-quinolyl)-1,2,4-triazol-3-yl]methanamine dihydrochloride;
2-[4-(3-chloro-2-piperazin-1-yl-6-quinolyl)-1,2,4-triazol-3-yl]ethanamine dihydrochloride;
3-(2-aminoethyl)-1-(3-chloro-2-piperazin-1-yl-6-quinolyl)pyrrolidin-2-one dihydrochloride;
1-(2-aminoethyl)-3-(3-chloro-2-piperazin-1-yl-6-quinolyl)imidazolidin-2-one dihydrochloride;
1-(3-chloro-2-piperazin-1-yl-6-quinolyl)-3-[2-(dimethylamino)ethyl]pyrrolidin-2-one dihydrochloride;
1-(3-chloro-2-piperazin-1-yl-6-quinolyl)-3-[2-(dimethylamino)ethyl]imidazolidin-2-one dihydrochloride;
[5-(3-chloro-2-piperazin-1-yl-6-quinolyl)-1,2,4-oxadiazol-3-yl]methanamine dihydrochloride;
2-[5-(3-chloro-2-piperazin-1-yl-6-quinolyl)-1,2,4-oxadiazol-3-yl]ethanamine dihydrochloride;
[3-(3-chloro-2-piperazin-1-yl-6-quinolyl)-1,2,4-oxadiazol-5-yl]methanamine dihydrochloride;
2-[3-(3-chloro-2-piperazin-1-yl-6-quinolyl)-1,2,4-oxadiazol-5-yl]ethanamine dihydrochloride;
[5-(3-chloro-2-piperazin-1-yl-6-quinolyl)-1,3,4-oxadiazol-2-yl]methanamine dihydrochloride;
2-[5-(3-chloro-2-piperazin-1-yl-6-quinolyl)-1,3,4-oxadiazol-2-yl]ethanamine dihydrochloride;
[4-(3-chloro-2-piperazin-1-yl-6-quinolyl)oxazol-2-yl]methanamine dihydrochloride;
2-[4-(3-chloro-2-piperazin-1-yl-6-quinolyl)oxazol-2-yl]ethanamine dihydrochloride;
[4-(3-chloro-2-piperazin-1-yl-6-quinolyl)thiazol-2-yl]methanamine dihydrochloride;
2-[4-(3-chloro-2-piperazin-1-yl-6-quinolyl)thiazol-2-yl]ethanamine dihydrochloride;
[4-(3-chloro-2-piperazin-1-yl-6-quinolyl)-1H-imidazol-2-yl]methanamine dihydrochloride;
2-[4-(3-chloro-2-piperazin-1-yl-6-quinolyl)-1H-imidazol-2-yl]ethanamine dihydrochloride;
[3-(3-chloro-2-piperazin-1-yl-6-quinolyl)-1H-1,2,4-triazol-5-yl]methanamine dihydrochloride;
2-[3-(3-chloro-2-piperazin-1-yl-6-quinolyl)-1H-1,2,4-triazol-5-yl]ethanamine dihydrochloride;
6-[3-(aminomethyl)phenyl]-N-[(1S,5R)-3-azabicyclo[3.1.0]hexan-6-yl]-3-chloro-quinolin-2-amine dihydrochloride;
[3-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride;
[4-(3-chloro-2-piperazin-1-yl-6-quinolyl)-2-pyridyl]methanamine dihydrochloride;
[6-(3-chloro-2-piperazin-1-yl-6-quinolyl)-2-pyridyl]methanamine dihydrochloride;
[2-(3-chloro-2-piperazin-1-yl-6-quinolyl)-4-pyridyl]methanamine dihydrochloride;
[6-(3-chloro-2-piperazin-1-yl-6-quinolyl)pyrazin-2-yl]methanamine dihydrochloride;
3-chloro-6-imidazol-1-yl-2-piperazin-1-yl-quinoline hydrochloride; and
(1S,4S)-2-[3-chloro-5-(trifluoromethyl)-2-pyridyl]-2,5-diazabicyclo[2.2.1]heptane formic acid.
In another embodiment, said compound is chosen from:
1-(3-chloro-5-iodo-2-pyridyl)piperazine;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
2-chloro-3-piperazin-1-yl-quinoxaline hydrochloride;
3-iodo-2-piperazin-1-yl-quinoline hydrochloride;
[3-(aminomethyl)phenyl] 5-chloro-6-piperazin-1-yl-pyridine-3-carboxylate dihydrochloride;
3-bromo-6-chloro-2-piperazin-1-yl-quinoline hydrochloride;
3-bromo-6-methyl-2-piperazin-1-yl-quinoline hydrochloride;
3-chloro-2,6-di(piperazin-1 yl)quinoline dihydrochloride;
3-chloro-6-iodo-2-piperazin-1-yl-quinoline hydrochloride;
6-bromo-3-chloro-2-piperazin-1-yl-quinoline hydrochloride;
2-[3-(5-chloro-6-piperazin-1-yl-3-pyridyl)-1,2,4-oxadiazol-5-yl]ethanamine dihydrochloride;
2-chloro-6,7-dimethyl-3-piperazin-1-yl-quinoxaline hydrochloride;
5-(5-chloro-6-piperazin-1-yl-3-pyridyl)pent-4-yn-1-amine 2,2,2-trifluoroacetic acid;
3-chloro-8-methyl-2-piperazin-1-yl-quinoline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
[3-(3-chloro-2-piperazin-1-yl-6-quinolyl)phenyl]methanamine dihydrochloride;
2-amino-N-(3-chloro-2-piperazin-1-yl-6-quinolyl)acetamide dihydrochloride.
[1-(3-chloro-2-piperazin-1-yl-6-quinolyl)-3-piperidyl]methanamine dihydrochloride;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3,7-dichloro-8-methyl-2-piperazin-1-yl-quinoline hydrochloride;
N′-(3-chloro-2-piperazin-1-yl-6-quinolyl)butane-1,4-diamine dihydrochloride;
3-chloro-2-piperazin-1-yl-N-[(3S)-3-piperidyl]quinolin-6-amine dihydrochloride;
[1-(3-chloro-2-piperazin-1-yl-6-quinolyl)-4-piperidyl]methanamine dihydrochloride
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
2-[3-(3-chloro-2-piperazin-1-yl-6-quinolyl)phenyl]propan-2-amine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride; and
[2-(3-chloro-2-piperazin-1-yl-6-quinolyl)-4-pyridyl]methanamine dihydrochloride.

Said compound can be used to treat a bacterial infection, when administered in combination with an antibiotic.

In particular, the compound of formula (I) as defined in this section allows to treat in combination with antibiotics, both antibiotic sensitive and antibiotic resistant bacteria.

“Antibiotic resistance” is well known in the art. Bacteria can acquire resistance, but can also be innately resistant to antibiotic molecules. Antibiotic efflux pumps can be involved in both these processes: Basal efflux pump expression can make bacteria innately resistant to some antibiotics, while mutations leading to the overexpression of these pumps can lead to acquired resistance. Compounds according to the invention act on both these forms of resistance.

As a consequence, compounds according to the invention can be used to prevent and/or treat infections by bacteria with innate and/or acquired antibiotic resistance.

Infections such as pneumonia, bronchitis, ear infections, meningitis, urinary tract infections, septicemia and sexually transmitted diseases can be cited as examples.

In particular, said compound is used to prevent and/or treat Gram-negative bacteria with innate or acquired antibiotic resistance.

In one embodiment, said compound is used to prevent and/or treat subjects afflicted by infections caused by Gram-negative bacteria with innate and acquired antibiotic resistance.

“Gram-negative bacteria” as used herein has the common meaning known in the art.

Escherichia coli (E. coli), Salmonella, Shigella, and other Enterobacteriaceae, Pseudomonas, Moraxella, Helicobacter, Campylobacter, Stenotrophomonas, Bdellovibrio, acetic acid bacteria, Legionella, cyanobacteria, spirochaetes, green sulfur, and green non-sulfur bacteria, Neisseria gonorrhoeae, Neisseria meningitidis, Moraxella catarrhalis, Haemophilus influenza, Klebsiella pneumoniae, Legionella pneumophila, Pseudomonas aeruginosa, Proteus mirabilis, Enterobacter cloacae, Serratia marcescens, Helicobacter pylori, Salmonella enteitidis, Salmonella typhi and Acinetobacter baumannii can be cited as examples.

In particular, said Gram negative bacteria is chosen from E. coli., K. pneumoniae and other Enterobacteriaceae, A. baumannii, P. aeruginosa, Neisseria gonorrhoeae, Shigella species.

More particularly, said compound is a Gram-negative bacteria efflux pump inhibitor. “Bacteria efflux pumps” are well known in the art. Efflux pumps are bacterial transport proteins which are involved in extrusion of substrates from the bacteria into the external environment. In the context of the invention, efflux pumps belonging to the Resistance Nodulation cell Division (RND) superfamily, in particular in Gram-negative bacteria, are contemplated. RND pumps amongst Gram-negative bacteria are highly conserved, and many efflux pump inhibitors show broad spectrum RND pump inhibition as binding pockets interactions are conserved. Such is the case for the residues interacting with the compounds here presented.

In particular, compounds of formula (I) are able to bind to the transmembrane domain thereby allosterically impacting the conformational protomer cycling and drug efflux process.

The present invention also relates to a method for treating a bacterial infection, comprising administering to a subject in need thereof, a compound of formula (I) as defined in this section, in combination with an antibiotic.

In particular and as mentioned above, the compound of formula (I) is a Gram-negative bacteria efflux pump inhibitor.

More particularly, the method according to the present invention allows to prevent and/or treat Gram-negative bacteria when given in combination with antibiotics.

Even more particularly, said Gramnegative bacteria is chosen from E. coli., K. pneumoniae and other Enterobacteriaceae, A. baumannii, P. aeruginosa, Neisseria gonorrhoeae and Shigella species.

The terms “treat”, “treating”, “treated” or “treatment”, refer to therapeutic treatment wherein the object is to eliminate or lessen antibiotic resistance. Beneficial or desired clinical results include, but are not limited to, elimination of resistance, alleviation of resistance, diminishment of extent of condition, stabilized (i.e., not worsening) state of condition, delay or slowing of progression of the condition. In particular, as used in the context of the invention, the treatment of antibiotic resistance refers to the elimination or reduction of the phenomena of resistance.

The terms “prevent”, “prevention”, “preventing” or “prevented”, as used in the context of the present invention, refer to the prevention of the onset, recurrence or spread of the antibiotic resistance, or of one or more symptoms thereof. In certain embodiments, the terms refer to the treatment with or administration of a compound provided herein prior to the onset of resistance, particularly to patients at risk of antibiotic resistance. The terms encompass the inhibition or reduction of the resistance. Subjects with familial history of an infection associated with antibiotic resistance, in particular are candidates for preventive regimens in certain embodiments. In addition, subjects who have a history of recurring symptoms and/or resistances are also potential candidates for the prevention. In this regard, the term “prevention” may be interchangeably used with the term “prophylactic treatment”.

In particular, the subject in need of a prevention and/or treatment against antibiotic resistance is a subject afflicted with a disease caused by bacteria, in particular Gram-negative bacteria as described herein.

In the context of the present invention, the identification of the subjects who are in need of treatment of herein-described conditions is conducted as above mentioned and is well within the ability and knowledge of the man skilled in the art. A clinician skilled in the art can readily identify, by the above mentioned technics, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease and/or bacteria involved; the degree of involvement or the severity of the disease and/or resistance; the response of the individual subject; the particular compound administered; the mode of administration; the bioavailability characteristic of the preparation administered; the dose regimen selected; the use of concomitant medication; and other relevant circumstances.

As used herein, an “effective amount” refers to an amount which is effective in reducing, eliminating, treating or controlling the antibiotic resistance. The term “controlling” is intended to refer to all processes wherein there may be a slowing, interrupting, arresting, or stopping of the progression of the antibiotic resistance, but does not necessarily indicate a total elimination of all disease and condition symptoms, and is intended to include prophylactic treatment and chronic use.

The term “patient” or “subject” refers to a warm-blooded animal such as a mammal, in particular a human, male or female, unless otherwise specified, which is afflicted with, or has the potential to be afflicted by antibiotic resistance, as described herein.

The amount of the compound according to the invention, which is required to achieve the desired biological effect, will vary depending upon a number of factors, including the dosage of the drug to be administered, the chemical characteristics (e.g. hydrophobicity) of the compounds employed, the potency of the compounds, the type of resistance, the state of resistance in the patient, and the route of administration.

Compounds provided herein can be formulated into pharmaceutical compositions, optionally by admixture with one or more pharmaceutically acceptable excipients.

Such compositions may be prepared for use in oral administration, particularly in the form of tablets or capsules, in particular orodispersible (lyoc) tablets; or parenteral administration, particularly in the form of liquid solutions, suspensions or emulsions.

It may be prepared by any of the methods well known in the pharmaceutical art, for example, as described in Remington: The Science and Practice of Pharmacy, 20^{th} ed.; Gennaro, A. R., Ed.; Lippincott Williams & Wilkins: Philadelphia, P A, 2000. Pharmaceutically compatible binding agents and/or adjuvant materials can be included as part of the composition. Oral compositions will generally include an inert diluent carrier or an edible carrier. They can be administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate.

Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like.

Examples of modes of administration include parenteral e.g. subcutaneous, intramuscular, intravenous, intradermal, as well as oral administration.

### Process of Preparation

The present invention is also concerned with the process of preparation of the compounds of formula (I) as described herein.

The compounds and process of the present invention may be prepared in a number of ways well-known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art.

It will be appreciated that the compounds of the present invention may contain one or more asymmetrically substituted carbon atoms, and may be isolated in optically active or racemic forms. Thus, all chiral, diastereomeric, racemic forms, isomeric forms of a structure are intended, unless the specific stereochemistry or isomeric form is specifically indicated.

It is well-known in the art how to prepare and isolate such optically active forms.

For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxyl, amino, imino, thio or carboxy groups, where these are desired in the final product, to avoid their unwanted participation in the reactions.

Conventional protecting groups may be used in accordance with standard practice, for examples see T. W. Greene and P. G. M. Wuts in Protective Groups in Organic Chemistry, 4th ed. (2007), John Wiley & Sons Inc., 1999; J. F. W. McOmie in Protective Groups in Organic Chemistry, Plenum Press, 1973.

The compound thus prepared may be recovered from the reaction mixture by conventional means. For example, the compounds may be recovered by distilling off the solvent from the reaction mixture or, if necessary, after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography.

The reactions can be carried out by the skilled person by applying or adapting the methods illustrated in the examples hereinafter.

In particular, compounds of formula (I) can be prepared according to protocols 1 to 5, as mentioned in Part A of the experimental part below.

Further, the process of the invention may also comprise the additional step of isolating the compound of formula (I). This can be done by the skilled person by any of the known conventional means, such as the recovery methods described above.

Generally, the starting products are commercially available mainly from Fisher scientific, Fluorochem, Enamine or Sigma-Aldrich or other typical chemicals supplier or may be obtained by applying or adapting any known methods or those described in the examples.

In the context of the present invention, it should be understood that “a compound for use for the prevention or treatment of” is equivalent to “the use of a compound for the prevention or treatment of” and to “the use of a compound for the manufacture of a medicament for the prevention or treatment of”.

The invention will be further illustrated by the following examples.

### EXAMPLES

### Part A—Synthesis of Compounds According to the Invention

### Preparation of Compounds of Formula (I)

### Protocol 1

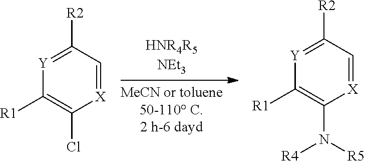

The chlorinated derivative (0.2-1.3 mmol, 1 eq), appropriate amine (1.2-5.6 eq) and NEt₃ (1.3-2.2 eq) were dissolved in MeCN or toluene (0.8-5 mL). The mixture was heated at 50° C., 80° C. or 110° C. for 2 h to 6 days, cooled to room temperature and then purified by flash or reverse chromatography.

### Protocol 2

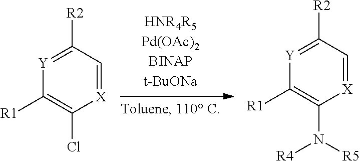

The chlorinated derivative (0.2-2.2 mmol, 1 eq), appropriate amine (1.5-3.2 eq), t-BuONa (1.4-2.5 eq), BINAP (0.03-0.08 eq), Pd(OAc)₂ (0.02-0.07 eq) were dissolved in toluene (0.6-3.0 mL) under argon. The mixture was heated at 110° C. for 2 h to 3 days, cooled to room temperature, dried under vacuum and purified by flash or reverse chromatography.

### Protocol 3

The appropriate Boc-protected compound (0.04-0.7 mmol, 1 eq) was dissolved in 1,4-dioxane (0.3-3.0 mL) and HCl 4N in 1,4-dioxane (8-28 eq) was added. The mixture was stirred at room temperature for 3 h to 5 days. The mixture was either filtered under vacuum and rinsed with petroleum ether and MeOH if the compound precipitated, or the mixture was evaporated under vacuum.

### Protocol 4

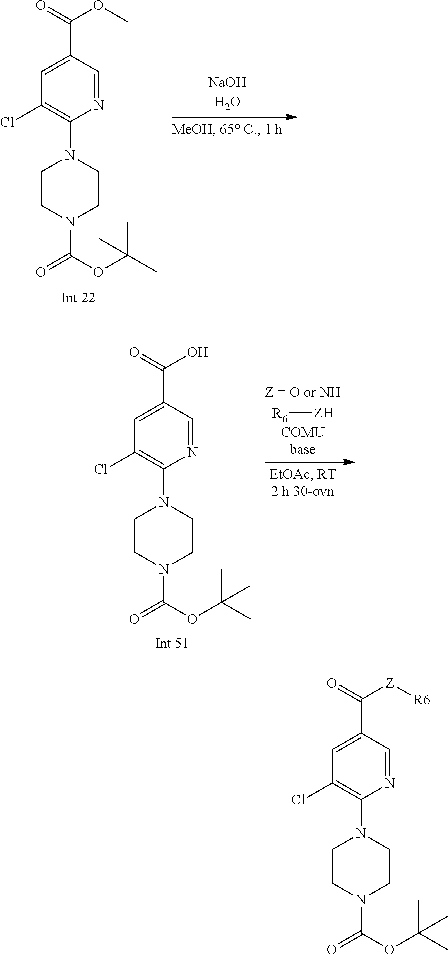

Intermediate 22 (4.86 mmol, 1 eq), NaOH (8.0 eq) and 1.5 mL water were stirred in 20 mL methanol at 65° C. for 1 hour. The reaction was diluted with water and then acidified with an aqueous solution of HCl 1N. The aqueous layer was extracted three times with EtOAc. The combined organic layers were washed with brine, dried over MgSO₄ and evaporated under reduced pressure to give Intermediate 51.

Intermediate 51 (0.30-0.50 mmol, 1 eq) was stirred at RT with NEt₃ or K₂CO₃ (2-2.5 eq) and COMU (1.5-2.5 eq) for 10 min in EtOAc (1.5 mL). Then, the corresponding alcohol or amine was added (1.5 eq) and the reaction was allowed to stir at RT for 1 h to 24 h. If the product precipitated, it was isolated by filtration. Otherwise the reaction was washed once with an aqueous solution of HCl 1N, twice with a saturated solution of NaHCO₃ and the product was extracted with EtOAc. The combined organic layers were washed with brine, dried over MgSO4 and evaporated under vacuum. The intermediate was purified by reverse or normal phase chromatography.

### Protocol 5

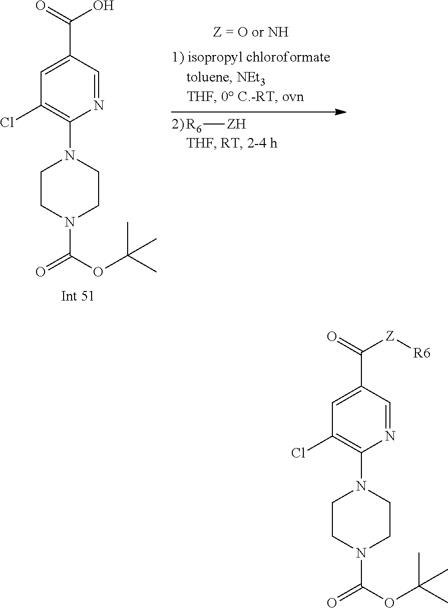

A 1 M solution of isopropyl chloroformate in toluene (1.5 eq) was added in 3 mL of anhydrous THF under argon. Intermediate 51 (1.7 mmol, 1 eq) and NEt₃ (1.2 eq) were dissolved in 3 mL of anhydrous THF and added dropwise to the isopropyl chloroformate solution at 0° C. The reaction was allowed to warm to RT and stirred overnight. A solution of saturated NaHCO₃ was added and the product was extracted twice with EtOAc. The organic layer was washed once with a saturated solution of NaHCO₃, once with brine, dried over MgSO₄ and evaporated under reduced pressure. A solution of the previous compound (0.4-0.5 mmol, 1 eq) dissolved in 1 mL anhydrous THF was added dropwise at 0° C. to a solution of the corresponding alcohol or amine (1.2-1.5 eq) and tBuOK or Na₂CO₃ (1.6-2.0 eq) dissolved in 1 mL anhydrous THF or water at 0° C. The mixture was stirred at RT for 2-4 h. The reaction was diluted with EtOAc and then washed once with a 1N HCl aqueous solution and twice with a saturated solution of NaHCO₃. The organic layers were combined, washed with a saturated solution of NaCl, dried over MgSO₄ and evaporated under vacuum. The intermediate was purified by flash chromatography.

### Intermediates

Intermediates 1-4, 70: To a solution of 3-ethoxyacryloyl chloride (1.0 eq) in THF (4.0-9.3 mL) at 0° C., was added the corresponding aniline (4.0-9.4 mmol, 1 eq) and pyridine (1.5-1.6 eq). The mixture was stirred overnight at room temperature. The reaction was quenched with water, extracted thrice with EtOAc, washed with brine, dried over MgSO₄, and then evaporated under reduced pressure. The crude product was purified by flash chromatography.

| Int | Structure | Physical data |
|---|---|---|
| 1 | 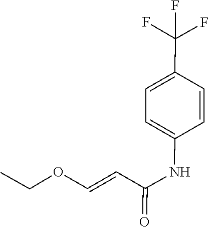 [SMILES: CCO/C=C/C(=O)Nc1ccc(C(F)(F)F)cc1] | (E)-3-ethoxy-N-[4-(trifluoromethyl)phenyl]prop-2- enamide Purification by flash chromatography (cyclohexane/EtOAc 100/0-70/30); Yield: 95%; ¹H NMR (300 MHz, CD₂Cl₂): δ 1.34 (t, J = 7.1 Hz, 3H), 3.95 (q, J = 7.1 Hz, 2H), 5.35 (d, J = 12.1 Hz, 1H), 7.33 (s, 1H), 7.54-7.57 (m, 2H), 7.61-7.71 (m, 3H) ppm; [ES + MS] m/z 260 (MH⁺). |
| 2 | 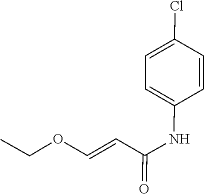 | (E)-N-(4-chlorophenyl)-3-ethoxy-prop-2-enamide Yield: 83%; ¹H NMR (300 MHz, CD₂Cl₂): δ 1.33 (t, J = 7.0 Hz, 3H), 3.94 (q, J = 7.0 Hz, 2H), 5.29-5.33 (m, 1H), 7.11 (s, 1H), 7.27 (d, J = 8.8 Hz, 2H), 7.50 (d, J = 8.8 Hz, 2H), 7.60 (d, J = 12.0 Hz, 1H) ppm; [ES + MS] m/z 226 (MH⁺). |
| 3 | 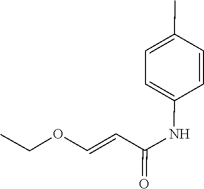 | (E)-3-ethoxy-N-(p-tolyl)prop-2-enamide Purification by flash chromatography (cyclohexane/EtOAc 100/0-70/30); Yield: 94%; ¹H NMR (300 MHz, CD₂Cl₂): δ 1.32 (t, J = 7.1 Hz, 3H), 2.30 (s, 3H), 3.91 (q, J = 7.1 Hz, 2H), 5.32 (d, J = 12.1 Hz, 1H), 7.11 (d, J = 8.2 Hz, 2H), 7.17 (s, 1H), 7.39 (d, J = 8.3 Hz, 2H), 7.58 (d, J = 12.1 Hz, 1H) ppm; [ES + MS] m/z 206 (MH⁺). |
| 4 | 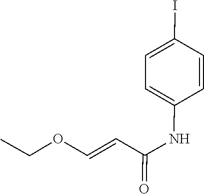 | (E)-3-ethoxy-N-(4-iodophenyl)prop-2-enamide Purification by flash chromatography (cyclohexane/EtOAc 100/0-60/40); Yield: 88%; ¹H NMR (300 MHz, CD₂Cl₂): δ 1.27 (t, J = 7.0 Hz, 3H), 3.95 (d, J = 7.0 Hz, 2H), 5.49 (d, J = 12.3 Hz, 1H), 7.43-7.51 (m, 3H), 7.61 (d, J = 8.8 Hz, 2H), 9.82 (s, 1H) ppm; [ES + MS] m/z 318 (MH⁺) |
| 70 | 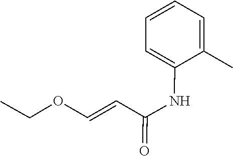 | (E)-3-ethoxy-N-(o-tolyl)prop-2-enamide Purification by flash chromatography (cyclohexane/EtOAc 100/0-70/30); Yield: 68%; ¹H NMR (300 MHz, CD₂Cl₂): δ 1.32 (t, J = 7.1 Hz, 3H), 2.23 (s, 3H), 3.88 (q, J = 7.1 Hz, 2H), 5.41 (d, J = 12.1 Hz, 1H), 7.03-7.21 (m, 3H), 7.24 (s, 1H), 7.58 (d, J = 12.1 Hz, 1H), 7.65-7.70 (m, 1H) ppm; [ES + MS] m/z 206 (MH⁺). |

Intermediates 5-8, 71: The corresponding starting intermediate (0.88-7.1 mmol, 1 eq) was added by small portion at 0° C. to concentrated sulfuric acid (28 eq.). The resulting mixture was stirred at room temperature for 2 h-3 h. The suspension was cooled at 0° C. and quenched with a solution of Na₂CO₃ until pH=7-8, extracted thrice with EtOAc, washed with brine, dried over MgSO₄, and then evaporated under reduced pressure.

| | | Starting | Physical |
|---|---|---|---|
| Int | Structure | Int. | data |
| 5 | 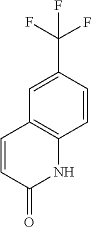 [SMILES: O=c1ccc2cc(C(F)(F)F)ccc2[nH]1] | 1 | 6-(trifluoromethyl)-1H-quinolin-2-one Yield: 90%; ¹H NMR (300 MHz, CD₂Cl₂): δ 6.77 (d, J = 9.6 Hz, 1H), 7.50 (d, J = 8.3 Hz, 1H), 7.74 (dd, J = 1.8, 8.7 Hz, 1H), 7.86-7.90 (m, 2H), 12.47 (s, 1H) ppm; [ES + MS] m/z 214 (MH⁺). |
| 6 | 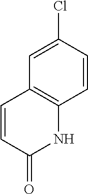 [SMILES: O=c1ccc2cc(Cl)ccc2[nH]1] | 2 | 6-chloro-1H-quinolin-2-one Yield: 77%; ¹H NMR (300 MHz, DMSO-d6): δ 6.56 (d, J = 9.6 Hz, 1H), 7.30 (d, J = 8.8 Hz, 1H), 7.52 (dd, J = 2.4, 8.8 Hz, 1H), 7.78 (d, J = 2.4 Hz, 1H), 7.87 (d, J = 9.5 Hz, 1H), 11.87 (s, 1H) ppm; [ES + MS] m/z 180 (MH⁺). |
| 7 | 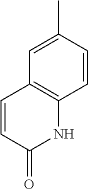 | 3 | 6-methyl-1H-quinolin-2-one Yield: 57%; ¹H NMR (300 MHz, DMSO-d₆): δ ¹H NMR (300 MHz, DMSO-d6): δ 2.32 (s, 3H), 6.46 (d, J = 9.5 Hz, 1H), 7.20 (d, J = 8.3 Hz, 1H), 7.31 (dd, J= 1.3, 8.3 Hz, 1H), 7.43 (s, 1H), 7.81 (d, J = 9.5 Hz, 1H), 11.66 (br s, 1H) ppm; [ES + MS] m/z 160 (MH⁺). |
| 8 | 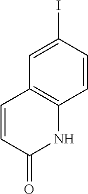 [SMILES: O=c1ccc2cc(I)ccc2[nH]1] | 4 | 6-iodo-1H-quinolin-2-one Yield: 74%; ¹H NMR (300 MHz, DMSO-d6): δ ¹H NMR (300 MHz, DMSO-d6): δ 6.51 (d, J = 9.6 Hz, 1H), 7.11 (d, J = 8.6 Hz, 1H), 7.76 (dd, J = 2.0, 8.6 Hz, 1H), 7.84 (d, J = 9.6 Hz, 1H), 8.06 (d, J = 1.9 Hz, 1H), 11.83 (br s, 1H) ppm. ppm; [ES + MS] m/z 272 (MH⁺). |
| 71 | 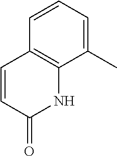 | 70 | 8-methyl-1H-quinolin-2-one Yield: 88%; ¹H NMR (300 MHz, CD₂Cl₂): δ 2.50 (s, 3H), 6.60 (d, J = 9.5 Hz, 1H), 7.12 (t, J = 7.6 Hz, 1H), 7.36 (d, J = 7.3 Hz, 1H), 7.42 (d, J = 7.8 Hz, 1H), 7.77 (d, J = 9.5 Hz, 1H), 9.86 (br s, 1H) ppm; [ES + MS] m/z 160 (MH⁺). |

Intermediate 9-12, 72-74, 122: To a solution of the corresponding starting intermediate (0.9-4.8 mmol) in anhydrous DMF (2.4-13.0 mL) was added NBS or NCS (1.1-2.5 eq). The mixture was stirred at 60° C. for 1 h-overnight. The solution was cooled to room temperature, quenched with water, extracted thrice with EtOAc, washed with brine, dried over MgSO₄, evaporated under reduced pressure and then purified by flash chromatography.

| Int | Structure | Starting Int. | Physical data |
|---|---|---|---|
| 9 | 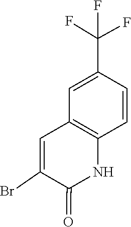 [SMILES: O=c1[nH]c2ccc(C(F)(F)F)cc2cc1Br] | 5 | 3-bromo-6-(trifluoromethyl)-1H-quinolin-2- one Purification by flash chromatography (cyclohexane/EtOAc 100/0-70/30); Yield: 73%; ¹H NMR (300 MHz, CD₂Cl₂): δ 7.57 (dd, J = 0.6, 8.6 Hz, 1H), 7.75-7.80 (m, 1H), 7.82- 7.84 (m, 1H), 8.28 (s, 1H), 12.32 (s, 1H) ppm; [ES + MS] m/z 292 (MH⁺). |
| 10 | 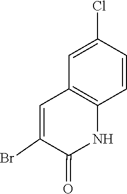 [SMILES: O=c1[nH]c2ccc(Cl)cc2cc1Br] | 6 | 3-bromo-6-chloro-1H-quinolin-2-one Purification by flash chromatography (cyclohexane/EtOAc 100/0-40/60); Yield: 53%; ¹H NMR (300 MHz, DMSO-d₆): δ 7.33 (d, J = 8.8 Hz, 1H), 7.58 (dd, J = 2.4, 8.8 Hz, 1H), 7.79 (d, J = 2.3 Hz, 1H), 8.48 (s, 1H), 12.39 (s, 1H) ppm; [ES + MS] m/z 258 (MH⁺). |
| 11 | 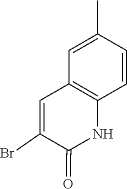 | 7 | 3-bromo-6-methyl-1H-quinolin-2-one Purification by flash chromatography (cyclohexane/EtOAc 100/0-40/60); Yield: 88%; ¹H NMR (300 MHz, DMSO-d₆): δ 2.34 (s, 3H), 7.23 (d, J = 8.3 Hz, 1H), 7.38 (ddd, J = 0.5, 2.0, 8.4 Hz, 1H), 7.44-7.46 (m, 1H), 8.42 (s, 1H), 12.2 (br s, 1H) ppm; [ES + MS] m/z 238 (MH⁺). |
| 12 | 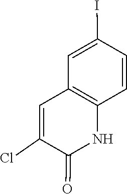 [SMILES: O=c1[nH]c2ccc(I)cc2cc1Cl] | 8 | 3-chloro-6-iodo-1H-quinolin-2-one Purification by flash chromatography (cyclohexane/EtOAc 100/0-30/70); Yield: 82%; ¹H NMR (300 MHz, DMSO-d₆): δ 7.14 (d, J = 8.7 Hz, 1H), 7.81 (dd, J = 2.0, 8.6 Hz, 1H), 8.08 (d, J = 2.0 Hz, 1H), 8.25 (s, 1H), 12.38 (s, 1H) ppm; [ES + MS] m/z 306 (MH⁺). |
| 72 | 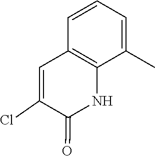 | 71 | 6-chloro-8-methyl-1H-quinolin-2-one Not isolated, [ES + MS] m/z 194 (MH⁺) |
| 73 | 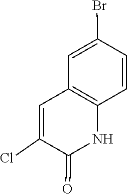 [SMILES: O=c1[nH]c2ccc(Br)cc2cc1Cl] | commercial | 6-bromo-3-chloro-1H-quinolin-2-one Purification by flash chromatography (cyclohexane/EtOAc 100/0-30/70); Yield: 53%; ¹H NMR (300 MHz, DMSO-d₆): δ 7.28 (d, J = 8.8 Hz, 1H), 7.68 (dd, J = 2.3, 8.8 Hz, 1H), 7.92 (d, J = 2.2 Hz, 1H), 8.28 (s, 1H) ppm; [ES + MS] m/z 258 (MH⁺). |
| 74 | 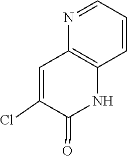 [SMILES: O=c1[nH]c2cccnc2cc1Cl] | commercial | 3-chloro-1H-1,5-naphthyridin-2-one Purification by flash chromatography (DCM/MeOH 100/0-90/10); Yield: 48%; ¹H NMR (300 MHz, DMSO-d₆): δ 7.28 (dd, J = 4.5, 8.4 Hz, 1H), 7.70-7.74 (m, 1H), 8.28 (d, J = 0.5 Hz, 1H), 8.52 (dd, J = 1.4, 4.4 Hz, 1H), 12.47 (s, 1H) ppm; [ES + MS] m/z 181 (MH⁺). |
| 122 | 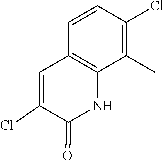 | commercial | 3,7-dichloro-8-methyl-1H-quinolin-2-one Not isolated, [ES + MS] m/z 228 (MH⁺) |

Intermediates 13-16, 75-77, 123: To the corresponding starting intermediate (0.37-2.04 mmol) was added POCl₃ (28-32 eq). The mixture was stirred at 100° C. for 10 min-1 h, then reaction mixture was poured on ice, diluted in DCM, basified with NaOH, extracted thrice with DCM. The organic phase was washed with brine, dried over MgSO₄ and then evaporated under reduced pressure.

| Int | Structure | Starting Int. | Physical data |
|---|---|---|---|
| 13 | 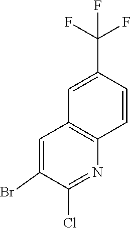 [SMILES: FC(F)(F)c1ccc2nc(Cl)c(Br)cc2c1] | 9 | 3-bromo-2-chloro-6- (trifluoromethyl)quinoline Yield: 94%; ¹H NMR (300 MHz, CD₂Cl₂): δ 7.98 (dd, J = 1.8, 8.8 Hz, 1H), 8.15-8.19 (m, 2H), 8.64 (s, 1H) ppm. |
| 14 | 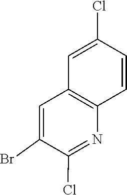 [SMILES: Clc1ccc2nc(Cl)c(Br)cc2c1] | 10 | 3-bromo-2,6-dichloro-quinoline Yield: 96%; ¹H NMR (300 MHz, CD₂Cl₂): δ 7.70 (dd, J = 2.3, 9.0 Hz, 1H), 7.77 (d, J = 2.3 Hz, 1H), 7.92 (dt, J = 0.6, 9.0 Hz, 1H), 8.38 (s, 1H) ppm. |
| 15 | 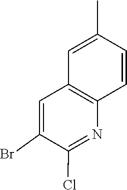 | 11 | 3-bromo-2-chloro-6-methyl-quinoline Yield: 78%; ¹H NMR (300 MHz, CD₂Cl₂): δ 2.54 (s, 3H), 7.54-7.56 (m, 1H), 7.62 (dd, J = 1.8, 8.6 Hz, 1H), 7.88 (d, J = 8.6 Hz, 1H), 8.39 (s, 1H) ppm. |
| 16 | 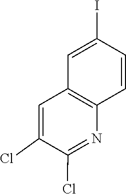 [SMILES: Clc1cc2cc(I)ccc2nc1Cl] | 12 | 2,3-dichloro-6-iodo-quinoline Yield: 85%; ¹H NMR (300 MHz, DMSO-d6): δ 7.76 (d, J = 8.9 Hz, 1H), 8.10 (dd, J = 2.0, 8.9 Hz, 1H), 8.50 (d, J = 2.0 Hz, 1H), 8.73 (s, 1H) ppm. |
| 75 | 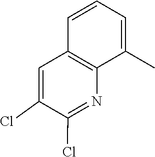 | 72 | 2,3-dichloro-8-methyl-quinoline Not isolated |
| 76 | 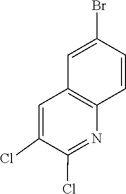 [SMILES: Clc1cc2cc(Br)ccc2nc1Cl] | 73 | 6-bromo-2,3-dichloro-quinoline Yield: 81%; ¹H NMR (300 MHz, CD₂Cl₂): δ 7.82 (dd, J = 2.0, 9.0 Hz, 1H), 7.87 (d, J = 8.9 Hz, 1H), 7.97 (d, J = 1.4 Hz, 1H), 8.20 (s, 1H, 4CH) ppm. |
| 77 | 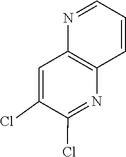 [SMILES: Clc1cc2ncccc2nc1Cl] | 74 | 2,3-dichloro-1,5-naphthyridine Yield: 36%; ¹H NMR (300 MHz, CD₂Cl₂): δ 7.28 (dd, J = 4.2, 8.6 Hz, 1H), 8.31 (ddd, J = 0.8, 1.7, 8.6 Hz, 1H), 8.51 (d, J = 0.7 Hz, 1H), 8.98 (dd, J = 1.6, 4.2 Hz, 1H) ppm. |
| 123 | 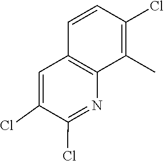 | 122 | 2,3,7-trichloro-8-methyl-quinoline Not isolated |

Intermediates 17-35, 78-81, 124 were prepared according to Protocol 1.

| Int | Structure | Physical data |
|---|---|---|
| 17 | 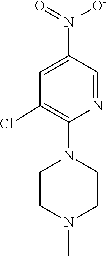 [SMILES: CN1CCN(c2ncc([N+](=O)[O-])cc2Cl)CC1] | 1-(3-chloro-5-nitro-2-pyridyl)-4-methyl-piperazine Purification by flash chromatography (DCM/MeOH 100/0- 95/05); Yield: 94%; ¹H NMR (300 MHz, CD₂Cl₂): δ 2.30 (s, 3H), 2.51 (t, J = 5.0 Hz, 4H), 3.73 (t, J = 5.0 Hz, 4H), 8.31 (d, J = 2.4 Hz, 1H), 8.93 (d, J = 2.4 Hz, 1H) ppm; [ES + MS] m/z 257 (MH⁺). |
| 18 | 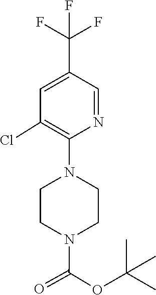 [SMILES: CC(C)(C)OC(=O)N1CCN(c2ncc(C(F)(F)F)cc2Cl)CC1] | tert-butyl-4-[3-chloro-5-(trifluoromethyl)-2- pyridyl]piperazine-1-carboxylate Purification by flash chromatography (cyclohexane/EtOAc 100/0-90/10); Yield: 68%; ¹H NMR (300 MHz, CD₂Cl₂): δ 1.46 (s, 9H), 3.43-3.47 (m, 4H), 3.53-3.57 (m, 4H), 7.80 (dd, J = 0.5, 2.2 Hz, 1H), 8.39-3.41 (m, 1H) ppm; [ES + MS] m/z 366 (MH⁺). |
| 19 | 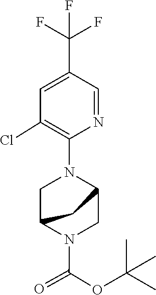 | tert-butyl (1S,4S)-5-[3-chloro-5-(trifluoromethyl)-2- pyridyl]-2,5-diazabicyclo[2.2.1]heptane-2-carboxylate Purification by flash chromatography (cyclohexane/EtOAc 100/0-90/10); Yield: 93%; ¹H NMR (300 MHz, CD₂Cl₂): δ 1.41 (s, 9H), 1.89-1.91 (m, 2H), 3.40 (dd, J = 2.0, 10.2 Hz, 1H), 3.51-3.66 (m, 2H), 4.00 (d, J = 10.0 Hz, 1H), 4.53 (d, J = 30.0 Hz, 1H), 5.01-5.02 (m, 1H), 7.66-7.67 (m, 1H), 8.25- 8,26 (m, 1H) ppm; [ES + MS] m/z 378 (MH⁺). |
| 20 | 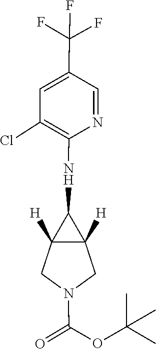 [SMILES: CC(C)(C)OC(=O)N1C[C@@H]2[C@H](C1)[C@H]2Nc1ncc(C(F)(F)F)cc1Cl] | tert-butyl (1R,5S)-6-[[3-chloro-5-(trifluoromethyl)-2- pyridyl]amino]-3-azabicyclo[3.1.0]hexane-3-carboxylate Purification by flash chromatography (cyclohexane/EtOAc 100/0-80/20); Yield: 91%; ¹H NMR (300 MHz, CD₂Cl₂): δ 1.43 (s, 9H), 1.71-1.74 (m, 2H), 2.57 (q, J = 2.2 Hz, 1H), 3.41 (dt, J = 2.2, 10.9 Hz, 2H), 3.67-3.72 (m, 2H), 5.55 (br s, 1H), 7.65-7.66 (m, 1H), 8.34-8.35 (m, 1H) ppm; [ES + MS] m/z 378 (MH⁺). |
| 21 | 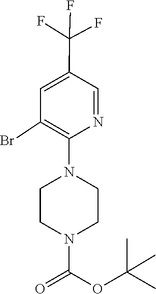 [SMILES: CC(C)(C)OC(=O)N1CCN(c2ncc(C(F)(F)F)cc2Br)CC1] | tert-butyl 4-[3-bromo-5-(trifluoromethyl)-2- pyridyl]piperazine-1-carboxylate Purification by flash chromatography (cyclohexane/EtOAc 100/0-80/20); Yield: 69%; [ES + MS] m/z 410 (MH⁺). |
| 22 | 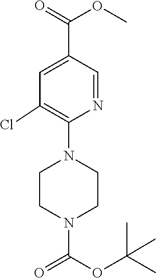 [SMILES: COC(=O)c1cnc(N2CCN(C(=O)OC(C)(C)C)CC2)c(Cl)c1] | tert-butyl 4-(3-chloro-5-methoxycarbonyl-2- pyridyl)piperazine-1-carboxylate Purification by flash chromatography (cyclohexane/EtOAc 100/0-80/20); Yield: 86%; [ES + MS] m/z 356 (MH⁺). |
| 23 | 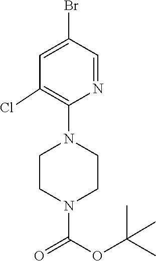 [SMILES: CC(C)(C)OC(=O)N1CCN(c2ncc(Br)cc2Cl)CC1] | tert-butyl 4-(5-bromo-3-chloro-2-pyridyl)piperazine-1- carboxylate Purification by flash chromatography (DCM/MeOH 100/0- 95/05); Yield: 85%; [ES + MS] m/z 376 (MH⁺). |
| 24 | 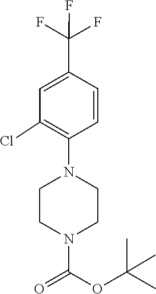 [SMILES: CC(C)(C)OC(=O)N1CCN(c2ccc(C(F)(F)F)cc2Cl)CC1] | tert-butyl 4-[2-chloro-4- (trifluoromethyl)phenyl]piperazine-1-carboxylate Purification by flash chromatography (DCM/MeOH 100/0- 95/05); Yield: 35%; ¹H NMR (300 MHz, CD₂Cl₂): δ 1.50 (s, 9H), 3.08 (t, J = 5.0 Hz, 4H), 3.62 (t, J = 5.0 Hz, 4H), 7.14 (d, J = 8.6 Hz, 1H), 7.51-7.55 (m, 1H), 7.67 (d, J = 2.2 Hz, 1H) ppm; [ES + MS] m/z 365 (MH⁺). |
| 25 | 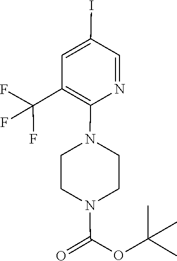 [SMILES: CC(C)(C)OC(=O)N1CCN(c2ncc(I)cc2C(F)(F)F)CC1] | tert-butyl 4-[5-iodo-3-(trifluoromethyl)-2- pyridyl]piperazine-1-carboxylate Treatment with HCl 1N and extraction with EtOAc. The organic layer was washed with brine, dried over MgSO₄, and evaporated under reduced pressure; Yield: 89%; ¹H NMR (300 MHz, CD₂Cl₂): δ 1.45 (s, 9H), 3.20-3.26 (m, 4H), 3.48-3.54 (m, 4H), 8.16 (qd, J = 0.4, 2.3 Hz, 1H), 8.58 (J = 0.7, 2.2 Hz, 1H) ppm; [ES + MS] m/z 458 (MH⁺). |
| 26 | 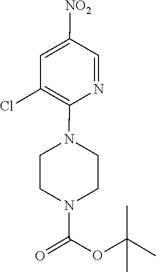 [SMILES: CC(C)(C)OC(=O)N1CCN(c2ncc([N+](=O)[O-])cc2Cl)CC1] | tert-butyl 4-(3-chloro-5-nitro-2-pyridyl)piperazine-1- carboxylate Yield: 99%; ¹H NMR (300 MHz, CD₂Cl₂): δ 1.46 (s, 9H), 3.53- 3.59 (m, 4H), 3.63-3.69 (m, 4H), 8.34 (d, J = 2.4 Hz, 1H), 8.95 (d, J = 2.4 Hz, 1H) ppm; [ES + MS] m/z 341 (MH⁺). |
| 27 | 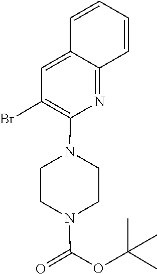 [SMILES: CC(C)(C)OC(=O)N1CCN(c2nc3ccccc3cc2Br)CC1] | tert-butyl 4-(3-bromo-2-quinolyl)piperazine-1- carboxylate Purification by flash chromatography (cyclohexane/EtOAc 100/0-90/10); Yield: 92%; ¹H NMR (300 MHz, CD₂Cl₂): δ 1.47 (s, 9H), 3.38-3.41 (m, 4H), 3.60-3.63 (m, 4H), 7.38-7.43 (m, 1H), 7.61-7.67 (m, 2H), 7.80-7.83 (m, 1H), 8.31 (s, 1H) ppm. [ES + MS] m/z 392 (MH⁺). |
| 28 | 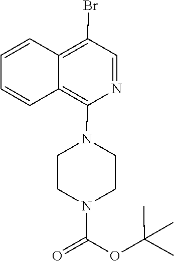 [SMILES: CC(C)(C)OC(=O)N1CCN(c2ncc(Br)c3ccccc23)CC1] | tert-butyl 4-(4-bromo-1-isoquinolyl)piperazine-1- carboxylate Purification by flash chromatography (cyclohexane/EtOAc 100/0-90/10); Yield: 87%; ¹H NMR (300 MHz, CD₂Cl₂): δ 1.47 (s, 9H), 3.30-3.34 (m, 4H), 3.64-3.68 (m, 4H), 7.61 (ddd, J = 1.2, 7.0, 8.2 Hz, 1H), 7.76 (ddd, J = 1.2, 7.0, 8.2 Hz, 1H), 8.09-8.14 (m, 2H), 8.30 (s, 1H) ppm. [ES + MS] m/z 392 (MH⁺). tert-butyl 4-(3-chloroquinoxalin-2-yl)piperazine-1- carboxylate |
| 29 | 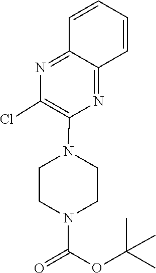 [SMILES: CC(C)(C)OC(=O)N1CCN(c2nc3ccccc3nc2Cl)CC1] | Yield: 100%; ¹H NMR (300 MHz, CD₂Cl₂): δ 1.46 (s, 9H), 3.46-3.52 (m, 4H), 3.59-3.64 (m, 4H), 7.53-7.59 (m, 1H), 7.64-7.69 (m, 1H), 7.81-7.88 (m, 2H) ppm; [ES + MS] m/z 349 (MH⁺). |
| 30 | 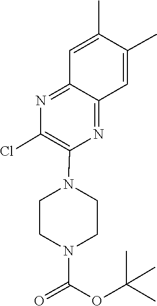 [SMILES: Cc1cc2nc(Cl)c(N3CCN(C(=O)OC(C)(C)C)CC3)nc2cc1C] | tert-butyl 4-(3-chloro-6,7-dimethyl-quinoxalin-2- yl)piperazine-1-carboxylate Purification by flash chromatography (cyclohexane/EtOAc 100/0-80/20); Yield: 79%; ¹H NMR (300 MHz, CD₂Cl₂): δ 1.47 (s, 9H), 2.43 (s, 3H), 2.44 (s, 3H), 3.42-3.46 (m, 4H), 3.59-3.63 (m, 4H), 7.60 (s, 1H), 7.61 (s, 1H) ppm; [ES + MS] m/z 377 (MH⁺). |
| 31 | 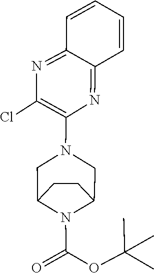 [SMILES: CC(C)(C)OC(=O)N1C2CCC1CN(c1nc3ccccc3nc1Cl)C2] | tert-butyl 3-(3-chloroquinoxalin-2-yl)-3,8- diazabicyclo[3.2.1]octane-8-carboxylate Purification by flash chromatography (cyclohexane/EtOAc 100/0-90/10); Yield: 86%; ¹H NMR (300 MHz, CD₂Cl₂): δ 1.89-2.13 (m, 4H), 3.20 (d, J = 12.5 Hz, 2H), 3.89 (d, J = 11.9 Hz, 2H), 4.35 (s, 2H), 7.54 (ddd, J = 1.5, 6.9, 8.3 Hz, 1H), 7.65 (ddd, J = 1.5, 6.9, 8.4 Hz, 1H), 7.80 (ddd, J = 0.4, 1.4, 8.3 Hz, 1H), 7.85 (ddd, J = 0.4, 1.4, 8.3 Hz, 1H) ppm; [ES + MS] m/z 375 (MH⁺). |
| 32 | 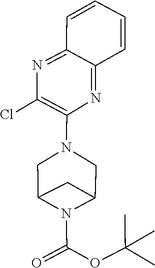 [SMILES: CC(C)(C)OC(=O)N1C2CC1CN(c1nc3ccccc3nc1Cl)C2] | tert-butyl 3-(3-chloroquinoxalin-2-yl)-3,6- diazabicyclo[3.1.1]heptane-6-carboxylate Purification by flash chromatography (cyclohexane/EtOAc 100/0-80/20); Yield: 73%; ¹H NMR (300 MHz, CD₂Cl₂): δ 1.35 (s, 9H), 1.54-1.58 (m, 1H), 2.57-2.66 (m, 1H), 4.02 (d, J = 12.5 Hz, 2H), 4.25 (d, J = 6.2 Hz, 2H), 4.49 (d, J = 12.6 Hz, 2H), 7.44 (ddd, J = 1.5, 7.0, 8.3 Hz, 1H), 7.60 (ddd, J = 1.5, 7.0, 8.4 Hz, 1H), 7.73 (dd, J = 1.4, 8.3 Hz, 1H), 7.79 (dd, J = 1.5, 8.3 Hz, 1H) ppm; [ES + MS] m/z 361 (MH⁺). |
| 33 | 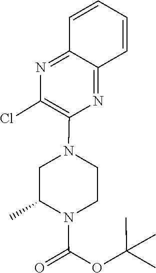 [SMILES: C[C@@H]1CN(c2nc3ccccc3nc2Cl)CCN1C(=O)OC(C)(C)C] | tert-butyl (2R)-4-(3-chloroquinoxalin-2-yl)-2-methyl- piperazine-1-carboxylate Purification by flash chromatography (cyclohexane/EtOAc 100/0-80/20); Yield: 71%; ¹H NMR (300 MHz, CD₂Cl₂): δ 1.35 (d, J = 6.8 Hz, 3H), 1.43 (s, 1H), 1.47 (s, 9H), 2.96 (ddd, J = 3.8, 13.0, 16.8 Hz, 1H), 3.08 (dd, J = 3.8, 13.0 Hz, 1H), 3.34 (ddd, J = 3.1, 12.8, 15.6 Hz, 1H), 3.92-4.03 (m, 2H), 4.37-4.43 (m, 1H), 7.56 (ddd, J = 1.5, 7.0, 8.3 Hz, 1H), 7.67 (ddd, J = 1.5, 7.0, 8.4 Hz, 1H), 7.83 (ddd, J = 0.5, 1.5, 8.3 Hz, 1H), 7.86 (ddd, J = 0.5, 1.5, 8.3 Hz, 1H) ppm; [ES + MS] m/z 363 (MH⁺). |
| 34 | 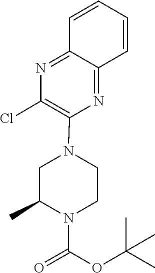 [SMILES: C[C@H]1CN(c2nc3ccccc3nc2Cl)CCN1C(=O)OC(C)(C)C] | tert-butyl (2S)-4-(3-chloroquinoxalin-2-yl)-2-methyl- piperazine-1-carboxylate Purification by flash chromatography (cyclohexane/EtOAc 100/0-90/10); Yield: 82%; ¹H NMR (300 MHz, CD₂Cl₂): δ 1.35 (d, J = 6.7 Hz, 3H), 1.47 (s, 9H), 2.96 (dt, J = 3.8, 13.0 Hz, 1H), 3.08 (dd, J = 3.7, 12.8 Hz, 1H), 3.34 (dt, J = 3.5, 13.4 Hz, 1H), 3.91-4.05 (m, 3H), 4.34-4.46 (m, 1H), 7.56 (ddd, J = 1.5, 7.0, 8.4 Hz, 1H), 7.66 (ddd, J = 1.5, 7.0, 8.4 Hz, 1H), 7.82 (ddd, J = 0.6, 1.5, 8.3 Hz, 1H), 7.86 (ddd, J = 1.5, 7.0, 8.3 Hz, 1H) ppm; [ES + MS] m/z 363 (MH⁺). |
| 35 | 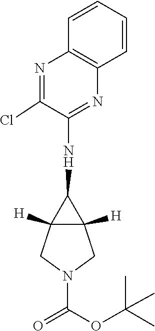 [SMILES: CC(C)(C)OC(=O)N1C[C@@H]2[C@H](C1)[C@H]2Nc1nc2ccccc2nc1Cl] | tert-butyl (1S,5R)-6-[(3-chloroquinoxalin-2-yl)amino]-3- azabicyclo[3.1.0]hexane-3-carboxylate Purification by flash chromatography (cyclohexane/EtOAc 100/0-70/30); Yield: 65%; ¹H NMR (300 MHz, CD₂Cl₂): δ 1.45 (s, 9H), 1.79-1.81 (m, 2H), 2.66 (q, J = 2.3 Hz, 1H), 3.43-3.49 (m, 2H), 3.77 (t, J = 10.8 Hz, 2H), 5.77 (s, 1H), 7.43 (ddd, J = 1.5, 7.0, 8.3 Hz, 1H), 7.62 (ddd, J = 1.5, 7.0, 8.3 Hz, 1H), 7.77-7.78 (m, 1H), 7.79-7.80 (m, 1H) ppm; [ES + MS] m/z 361 (MH⁺). |
| 78 | 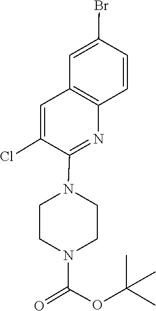 [SMILES: CC(C)(C)OC(=O)N1CCN(c2nc3ccc(Br)cc3cc2Cl)CC1] | tert-butyl 4-(6-bromo-3-chloro-2-quinolyl)piperazine-1- carboxylate Purification by flash chromatography (cyclohexane/EtOAc 100/0-40/60); Yield: 59%; ¹H NMR (300 MHz, CD₂Cl₂): δ 1.47 (s, 9H), 3.41-3.45 (m, 4H), 3.58-3.62 (m, 4H), 7.67-7.68 (m, 2H), 7.80-7.81 (m, 1H), 7.99 (s, 1H) ppm; [ES + MS] m/z 426 (MH⁺). |
| 79 | 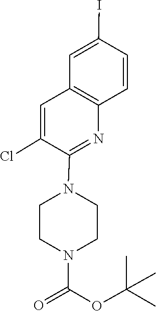 [SMILES: CC(C)(C)OC(=O)N1CCN(c2nc3ccc(I)cc3cc2Cl)CC1] | tert-butyl 4-(3-chloro-6-iodo-2-quinolyl)piperazine-1- carboxylate Purification by flash chromatography (cyclohexane/EtOAc 100/0-40/60); Yield: 78%; ¹H NMR (300 MHz, CD₂Cl₂): δ 1.47 (s, 9H), 3.41-3.45 (m, 4H), 3.58-3.62 (m, 4H), 7.54 (d, J = 8.8 Hz, 1H), 7.84 (dd, J = 2.0, 8.8 Hz, 1H), 7.96 (s, 1H), 8.03 (d, J = 2.0 Hz, 1H) ppm; [ES + MS] m/z 474 (MH⁺). |
| 80 | 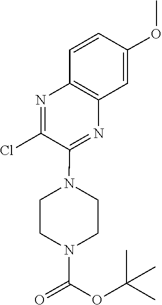 [SMILES: COc1ccc2nc(Cl)c(N3CCN(C(=O)OC(C)(C)C)CC3)nc2c1] | tert-butyl 4-(3-chloro-7-methoxy-quinoxalin-2- yl)piperazine-1-carboxylate Purification by flash chromatography (cyclohexane/EtOAc 100/0-80/20); Yield: 85%; ¹H NMR (300 MHz, CD₂Cl₂): δ 1.47 (s, 9H), 3.46-3.50 (m, 4H), 3.60-3.63 (m, 4H), 3.93 (s, 3H), 7.17 (dd, J = 2.5, 4.5 Hz, 1H), 7.21 (d, J = 2.80 Hz, 1H), 7.73 (d, J = 8.9 Hz, 1H) ppm; [ES + MS] m/z 379 (MH⁺). |
| 81 | 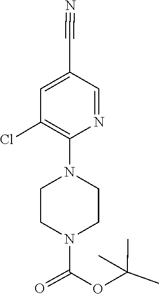 [SMILES: CC(C)(C)OC(=O)N1CCN(c2ncc(C#N)cc2Cl)CC1] | tert-butyl 4-(3-chloro-5-cyano-2-pyridyl)piperazine-1- carboxylate Yield: 94%; ¹H NMR (300 MHz, CD₂Cl₂): δ 1.46 (s, 9H), 3.54 (s, 8H), 7.77 (d, J = 2.0 Hz, 1H), 8.39 (d, J = 1.9 Hz, 1H) ppm; [ES + MS] m/z 323 (MH⁺). |
| 124 | 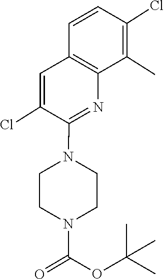 [SMILES: Cc1c(Cl)ccc2cc(Cl)c(N3CCN(C(=O)OC(C)(C)C)CC3)nc12] | tert-butyl 4-(3,7-dichloro-8-methyl-2- quinolyl)piperazine-1-carboxylate Purification by flash chromatography (cyclohexane/EtOAc 100/0-90/10); Yield: 7% over 3 steps from 122; ¹H NMR (300 MHz, CD₂Cl₂): δ 1.48 (s, 9H), 2.74 (s, 3H), 3.46-3.50 (m, 4H), 3.60-3.64 (m, 4H), 7.35 (d, J = 8.7 Hz, 1H), 7.44 (d, J = 8.7 Hz, 1H), 8.02 (s, 1H) ppm; [ES + MS] m/z 396 (MH⁺). |

Intermediates 36-50 and 82 were prepared according to Protocol 2.

| Int | Structure | Physical data |
|---|---|---|
| 36 | 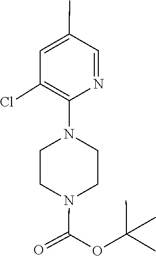 [SMILES: Cc1cnc(N2CCN(C(=O)OC(C)(C)C)CC2)c(Cl)c1] | tert-butyl 4-(3-chloro-5-methyl-2-pyridyl)piperazine-1 carboxylate Purification by flash chromatography (cyclohexane/EtOAc 100/0-80/20); Yield: 58%; [ES + MS] m/z 312 (MH⁺). |
| 37 | 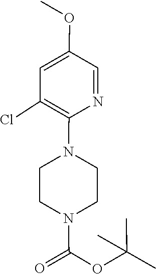 [SMILES: COc1cnc(N2CCN(C(=O)OC(C)(C)C)CC2)c(Cl)c1] | tert-butyl 4-(3-chloro-5-methoxy-2-pyridyl)piperazine-1- carboxylate Purification by flash chromatography (cyclohexane/EtOAc 100/0-80/20); Yield: 55%; ¹H NMR (300 MHz, CD₂Cl₂): δ 1.45 (s, 9H), 3.09-3.15 (m, 4H), 3.51-3.57 (m, 4H), 3.80 (s, 3H), 7.28 (d, J = 2.7 Hz, 1H), 7.91 (d, J = 2.7 Hz, 1H) ppm; [ES + MS] m/z 328 (MH⁺). |
| 38 | 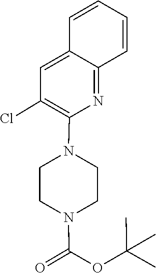 [SMILES: CC(C)(C)OC(=O)N1CCN(c2nc3ccccc3cc2Cl)CC1] | tert-butyl 4-(3-chloro-2-quinolyl)piperazine-1- carboxylate Purification by flash chromatography (cyclohexane/EtOAc 100/0-90/10); Yield: 40%; ¹H NMR (300 MHz, CD₂Cl₂): δ 1.47 (s, 9H), 3.40-3.45 (m, 4H), 3.59-3.63 (m, 4H), 7.40 (ddd, J = 1.2, 7.0, 8.1 Hz, 1H), 7.62 (ddd, J = 1.5, 7.0, 8.5 Hz, 1H), 7.65-7.68 (m, 1H), 7.79-7.83 (m, 1H), 8.08 (s, 1H) ppm; [ES + MS] m/z 348 (MH⁺). |
| 39 | 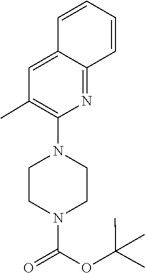 [SMILES: Cc1cc2ccccc2nc1N1CCN(C(=O)OC(C)(C)C)CC1] | tert-butyl 4-(3-methyl-2-quinolyl)piperazine-1- carboxylate Purification by flash chromatography (cyclohexane/EtOAc 100/0-90/10); Yield: 36%; [ES + MS] m/z 328 (MH⁺). |
| 40 | 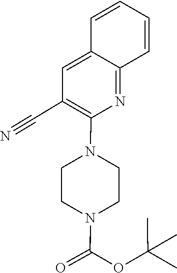 [SMILES: CC(C)(C)OC(=O)N1CCN(c2nc3ccccc3cc2C#N)CC1] | tert-butyl 4-(3-cyano-2-quinolyl)piperazine-1-carboxylate Purification by flash chromatography (cyclohexane/EtOAc 100/0-80/20); Yield: 56%; ¹H NMR (300 MHz, CD₂Cl₂): δ 1.47 (s, 9H), 3.62-3.64 (m, 8H), 7.41 (ddd, J = 1.5, 6.5, 8.2 Hz, 1H), 7.69-7.80 (m, 3H), 8.42 (s, 1H), ppm; [ES + MS] m/z 339 (MH⁺). |
| 41 | 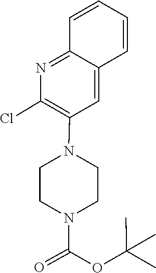 [SMILES: CC(C)(C)OC(=O)N1CCN(c2cc3ccccc3nc2Cl)CC1] | tert-butyl 4-(2-chloro-3-quinolyl)piperazine-1- carboxylate Purification by flash chromatography (cyclohexane/EtOAc 100/0-90/10); Yield: 7%; ¹H NMR (300 MHz, CD₂Cl₂): δ 1.48 (s, 9H), 3.09 (t, J = 4.9 Hz, 4H), 3.64 (t, J = 4.9 Hz, 4H), 7.53 (ddd, J = 1.4, 7.0, 8.1 Hz, 1H), 7.59-7.69 (m, 2H), 7.77 (dd, J = 1.0, 7.0 Hz, 1H), 7.91 (td, J = 0.6, 8.3 Hz, 1H) ppm; [ES + MS] m/z 348 (MH⁺). |
| 42 | 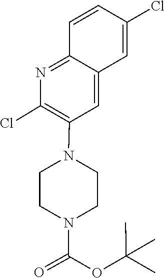 [SMILES: CC(C)(C)OC(=O)N1CCN(c2cc3cc(Cl)ccc3nc2Cl)CC1] | tert-butyl 4-(2,6-dichloro-3-quinolyl)piperazine-1- carboxylate Purification by flash chromatography (cyclohexane/EtOAc 100/0-90/10); Yield: 13%; ¹H NMR (300 MHz, CD₂Cl₂): δ 1.47 (s, 9H), 3.09 (t, J = 4.9 Hz, 4H), 3.64 (t, J = 4.9 Hz, 4H), 7.52 (s, 1H), 7.55 (d, J = 2.3 Hz, 1H), 7.73 (d, J = 2.3 Hz, 1H), 7.86 (d, J = 8.9 Hz, 1H) ppm; [ES + MS] m/z 382 (MH⁺). |
| 43 | 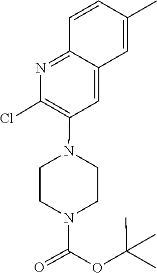 [SMILES: Cc1ccc2nc(Cl)c(N3CCN(C(=O)OC(C)(C)C)CC3)cc2c1] | tert-butyl 4-(2-chloro-6-methyl-3-quinolyl)piperazine-1- carboxylate Purification by flash chromatography (cyclohexane/EtOAc 100/0-80/20); Yield: 12%; ¹H NMR (300 MHz, CD₂Cl₂): δ 1.48 (s, 9H), 2.51 (s, 3H), 3.07 (t, J = 4.9 Hz, 4H), 3.63 (t, J = 4.9 Hz, 4H), 7.45 (dd, J = 1.8, 8.6 Hz, 1H), 7.52-7.54 (m, 1H), 7.55 (s, 1H), 7.79 (d, J = 8.5 Hz, 1H) ppm; [ES + MS] m/z 362 (MH⁺). |
| 44 | 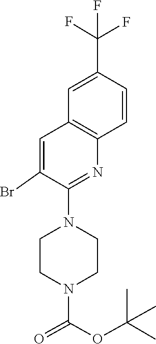 [SMILES: CC(C)(C)OC(=O)N1CCN(c2nc3ccc(C(F)(F)F)cc3cc2Br)CC1] | tert-butyl 4-[3-bromo-6-(trifluoromethyl)-2- quinolyl]piperazine-1-carboxylate Purification by flash chromatography (cyclohexane/EtOAc 100/0-90/10); Yield: 73%; ¹H NMR (300 MHz, CD₂Cl₂): δ 1.47 (s, 9H), 3.46-3.50 (m, 4H), 3.60-3.64 (m, 4H), 7.79 (dd, J = 2.0, 8.9 Hz, 1H), 7.90 (dd, J = 0.6, 8.9 Hz, 1H), 7.95-7.96 (m, 1H), 8.37 (s, 1H) ppm; [ES + MS] m/z 460 (MH⁺). |
| 45 | 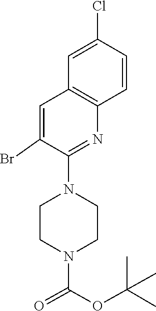 [SMILES: CC(C)(C)OC(=O)N1CCN(c2nc3ccc(Cl)cc3cc2Br)CC1] | tert-butyl 4-(3-bromo-6-chloro-2-quinolyl)piperazine-1- carboxylate Purification by flash chromatography (cyclohexane/EtOAc 100/0-90/10); Yield: 39%; ¹H NMR (300 MHz, CD₂Cl₂): δ 1.48 (s, 9H), 3.37-3.42 (m, 4H), 3.56-3.63 (m, 4H), 7.52-7.57 (m, 1H), 7.59-7.61 (m, 1H), 7.71-7.75 (m, 1H), 8.19 (s, 1H) ppm; [ES + MS] m/z 426 (MH⁺). |
| 46 | 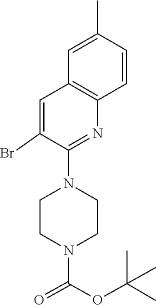 [SMILES: Cc1ccc2nc(N3CCN(C(=O)OC(C)(C)C)CC3)c(Br)cc2c1] | tert-butyl 4-(3-bromo-6-methyl-2-quinolyl)piperazine-1- carboxylate Purification by flash chromatography (cyclohexane/EtOAc 100/0-80/20); Yield: 14%; ¹H NMR (300 MHz, CD₂Cl₂): δ 1.47 (s, 9H), 2.48 (s, 3H), 3.34-3.38 (m, 4H), 3.59-3.63 (m, 4H), 7.42-7.43 (m, 1H), 7.47 (dd, J = 1.8, 8.6 Hz, 1H), 7.70 (d, J = 8.5 Hz, 1H), 8.22 (s, 1H) ppm; [ES + MS] m/z 406 (MH⁺). |
| 47 | 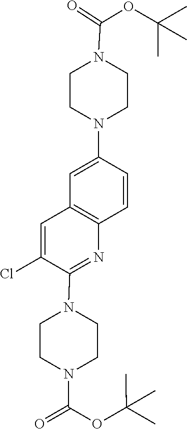 [SMILES: CC(C)(C)OC(=O)N1CCN(c2ccc3nc(N4CCN(C(=O)OC(C)(C)C)CC4)c(Cl)cc3c2)CC1] | tert-butyl 4-[2-(4-tert-butoxycarbonylpiperazin-1-yl)-3- chloro-6-quinolyl]piperazine-1-carboxylate Purification by flash chromatography (cyclohexane/EtOAc 100/0-80/20); Yield: 17%; ¹H NMR (300 MHz, CD₂Cl₂): δ 1.47 (s, 9H), 1.47 (s, 9H), 3.17-3.21 (m, 4H), 3.31-3.35 (m, 4H), 3.57-3.61 (m, 8H), 6.92 (d, J = 2.7 Hz, 1H), 7.39 (dd, J = 2.7, 9.2 Hz, 1H), 7.71 (d, J = 9.2 Hz, 1H), 7.95 (s, 1H) ppm; [ES + MS] m/z 532 (MH⁺). |
| 48 | 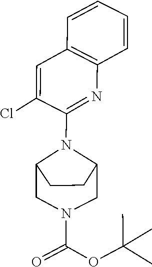 [SMILES: CC(C)(C)OC(=O)N1CC2CCC(C1)N2c1nc2ccccc2cc1Cl] | tert-butyl 8-(3-chloro-2-quinolyl)-3,8- diazabicyclo[3.2.1]octane-3-carboxylate Purification by flash chromatography (cyclohexane/EtOAc 100/0-90/10); Yield: 18%; ¹H NMR (300 MHz, CD₂Cl₂): δ 1.46 (s, 9H), 1.77-1.82 (m, 2H), 1.97-2.03 (m, 2H), 3.24 (d, J = 12.3 Hz, 1H), 3.36 (d, J = 12.3 Hz, 1H), 3.82 (d, J = 12.6 Hz, 1H), 3.90 (d, J = 11.5 Hz, 1H), 4.66 (d, J = 10.4 Hz, 2H), 7.35 (ddd, J = 1.2, 7.0, 8.1 Hz, 1H), 7.58 (ddd, J = 1.6, 7.0, 8.4 Hz, 1H), 7.61-7.65 (m, 1H), 7.73-7.76 (m, 1H), 8.05 (s, 1H) ppm; [ES + MS] m/z 374 (MH⁺). |
| 49 | 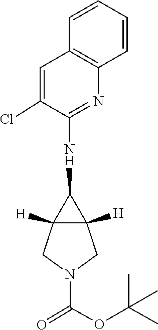 [SMILES: CC(C)(C)OC(=O)N1C[C@@H]2[C@H](C1)[C@H]2Nc1nc2ccccc2cc1Cl] | tert-butyl (1S,5R)-6-[(3-chloro-2-quinolyl)amino]-3- azabicyclo[3.1.0]hexane-3-carboxylate Purification by flash chromatography (cyclohexane/EtOAc 100/0-70/30); Yield: 57%; ¹H NMR (300 MHz, CD₂Cl₂): δ 1.46 (s, 9H), 1.76-1.78 (m, 2H), 2.66-2.68 (m, 1H), 3.43-3.49 (m, 2H), 3.74-3.81 (m, 2H), 5.58 (s, 1H), 7.27 (ddd, J = 1.0, 6.9, 8.0 Hz, 1H), 7.53-7.59 (m, 2H), 7.74-7.78 (m, 1H), 7.91 (s, 1H) ppm; [ES + MS] m/z 360 (MH⁺). |
| 50 | 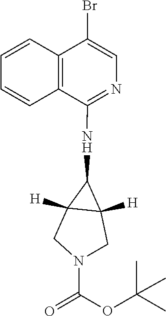 [SMILES: CC(C)(C)OC(=O)N1C[C@@H]2[C@H](C1)[C@H]2Nc1ncc(Br)c2ccccc12] | tert-butyl (1S,5R)-6-[(4-bromo-1-isoquinolyl)amino]-3- azabicyclo[3.1.0]hexane-3-carboxylate Purification by flash chromatography (cyclohexane/EtOAc 100/0-80/20); Yield: 37%; ¹H NMR (300 MHz, CD₂Cl₂): δ 1.44 (s, 9H), 1.75-1.77 (m, 2H), 2.65-2.67 (m, 1H), 3.44 (dd, J = 4.0, 10.8 Hz, 2H), 3.74 (d, J = 10.8 Hz, 2H), 5.68 (s, 1H), 7.55 (ddd, J = 1.1, 7.1, 8.2 Hz, 1H), 7.70-7.77 (m, 2H), 8.04 (d, J = 8.5 Hz, 1H), 8.19 (s, 1H) ppm; [ES + MS] m/z 404 (MH⁺). |
| 82 | 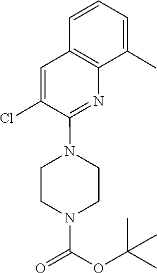 [SMILES: Cc1cccc2cc(Cl)c(N3CCN(C(=O)OC(C)(C)C)CC3)nc12] | tert-butyl 4-(3-chloro-8-methyl-2-quinolyl)piperazine-1- carboxylate Purification by flash chromatography (cyclohexane/EtOAc 100/0-80/20); Yield: 16% over 3 steps; ¹H NMR (300 MHz, CD₂Cl₂): δ 1.48 (s, 9H), 2.67 (s, 3H), 3.45-3.48 (m, 4H), 3.60- 3.65 (m, 4H), 7.26-7.31 (m, 1H), 7.46-7.52 (m, 2H), 8.05 (s, 1H) ppm; [ES + MS] m/z 362 (MH⁺). |

Intermediates 51-60 and 83 were prepared according to Protocol 4.

| Int | Structure | Physical data |
|---|---|---|
| 51 | 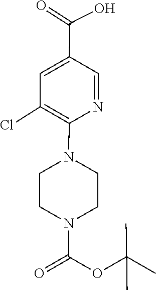 [SMILES: CC(C)(C)OC(=O)N1CCN(c2ncc(C(=O)O)cc2Cl)CC1] | 6-(4-tert-butoxycarbonylpiperazin-1-yl)-5- chloro-pyridine-3-carboxylic acid Yield: 100%; ¹H NMR (300 MHz, DMSO-d₆): δ 1.41 (s, 9H), 3.39-3.50 (m, 8H), 8.09 (d, J = 2.0 Hz, 1H), 8.66 (d, J = 2.0 Hz, 1H), 13.19 (s, 1H) ppm; [ES + MS] m/z 342 (MH⁺). |
| 52 | 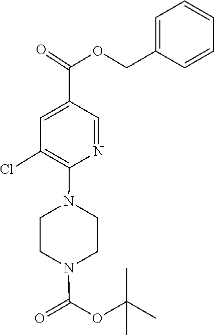 [SMILES: CC(C)(C)OC(=O)N1CCN(c2ncc(C(=O)OCc3ccccc3)cc2Cl)CC1] | tert-butyl 4-(5-benzyloxycarbonyl-3- chloro-2-pyridyl)piperazine-1- carboxylate Purification by flash chromatography (cyclohexane/EtOAc 100/0-90/10) and by reverse phase chromatography (MeCN 0.1% formic acid/water 0.1% formic acid 20/80- 100/0); Yield: 50%; ¹H NMR (300 MHz, CD₂Cl₂): δ 1.46 (s, 9H), 3.46-3.57 (m, 8H), 7.33-7.47 (m, 5H), 8.14 (d, J = 2.0 Hz, 1H), 8.75 (d, J = 2.0 Hz, 1H) ppm; [ES + MS] m/z 432 (MH⁺). |
| 53 | 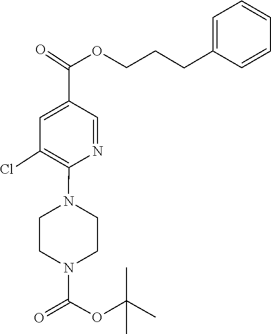 [SMILES: CC(C)(C)OC(=O)N1CCN(c2ncc(C(=O)OCCCc3ccccc3)cc2Cl)CC1] | tert-butyl 4-[3-chloro-5-(3- phenylpropoxycarbonyl)-2- pyridyl]piperazine-1-carboxylate Purification by reverse phase chromatography (MeCN 0.1% formic acid/water 0.1% formic acid 20/80-100/0); Yield: 68%; ¹H NMR (300 MHz, CD₂Cl₂): δ 1.46 (s, 9H), 2.02-2.13 (m, 2H), 2.77 (t, J = 7.5 Hz, 2H), 3.46-3.58 (m, 8H), 4.30 (t, J = 6.5 Hz, 2H), 7.14-7.33 (m, 5H), 8.09 (d, J = 2.0 Hz, 1H), 8.72 (d, J = 2.0 Hz, 1H) ppm; [ES + MS] m/z 460 (MH⁺). |
| 54 | 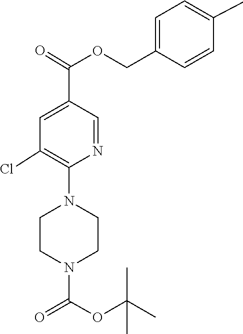 [SMILES: Cc1ccc(COC(=O)c2cnc(N3CCN(C(=O)OC(C)(C)C)CC3)c(Cl)c2)cc1] | tert-butyl 4-[3-chloro-5-(p- tolylmethoxycarbonyl)-2- pyridyl]piperazine-1-carboxylate Purification by reverse phase chromatography (MeCN 0.1% formic acid/water 0.1% formic acid 20/80-100/0); Yield: 51%; ¹H NMR (300 MHz, CD₂Cl₂): δ 1.46 (s, 9H), 2.35 (s, 3H), 3.46-3.57 (m, 8H), 5.28 (s, 2H), 7.20 (d, J = 7.8 Hz, 2H), 7.32 (d, J = 7.9 Hz, 2H), 8.14 (d, J = 2.0 Hz, 1H), 8.74 (d, J = 2.0 Hz, 1H) ppm; [ES + MS] m/z 446 (MH⁺). |
| 55 | 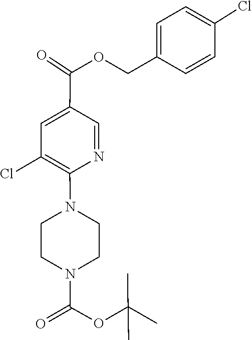 [SMILES: CC(C)(C)OC(=O)N1CCN(c2ncc(C(=O)OCc3ccc(Cl)cc3)cc2Cl)CC1] | tert-butyl 4-[3-chloro-5-[(4- chlorophenyl)methoxycarbonyl]-2- pyridyl]piperazine-1-carboxylate Purification by flash chromatography (cyclohexane/EtOAc 100/0-70/30); Yield: 37%; ¹H NMR (300 MHz, CD₂Cl₂): δ 1.45 (s, 9H), 3.46-3.58 (m, 8H), 5.30 (s, 2H), 7.35- 7.41 (m, 4H), 8.13 (d, J = 2.0 Hz, 1H), 8.74 (d, J = 2.0 Hz, 1H) ppm; [ES + MS] m/z 466 (MH⁺). |
| 56 | 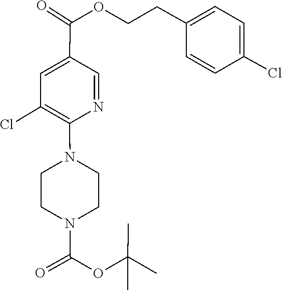 [SMILES: CC(C)(C)OC(=O)N1CCN(c2ncc(C(=O)OCCc3ccc(Cl)cc3)cc2Cl)CC1] | tert-butyl 4-[3-chloro-5-[2-(4- chlorophenyl)ethoxycarbonyl]-2- pyridyl]piperazine-1-carboxylate Purification by reverse phase chromatography (MeCN 0.1% formic acid/water 0.1% formic acid 20/80-100/0); Yield: 58%; ¹H NMR (300 MHz, CD₂Cl₂): δ 1.46 (s, 9H), 3.03 (t, J = 7.0 Hz, 2H), 3.46- 3.58 (m, 8H), 4.47 (t, J = 6.7 Hz, 2H), 7.21- 7.32 (m, 4H), 8.08 (d, J = 2.0 Hz, 1H), 8.67 (d, J = 2.0 Hz, 1H) ppm; [ES + MS] m/z 480 (MH⁺). |
| 57 | 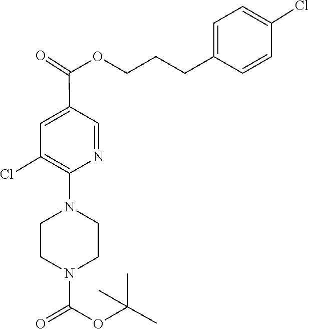 [SMILES: CC(C)(C)OC(=O)N1CCN(c2ncc(C(=O)OCCCc3ccc(Cl)cc3)cc2Cl)CC1] | tert-butyl 4-[3-chloro-5-[3-(4- chlorophenyl)propoxycarbonyl]-2- pyridyl]piperazine-1-carboxylate Purification by reverse phase chromatography (MeCN 0.1% formic acid/water 0.1% formic acid 20/80-100/0); Yield: 55%; ¹H NMR (300 MHz, CD₂Cl₂): 1.46 (s, 9H), 2.01-2.11 (m, 2H), 2.75 (t, J = 7.1 Hz, 2H), 3.47-3.58 (m, 8H), 4.29 (t, J = 6.6 Hz, 2H), 7.16 (d, J = 8.4 Hz, 2H), 7.26 (d, J = 8.4 Hz, 2H), 8.08 (d, J = 2.0 Hz, 1H), 8.70 (d, J = 2.0 Hz, 1H) ppm; [ES + MS] m/z 494 (MH⁺). |
| 58 | 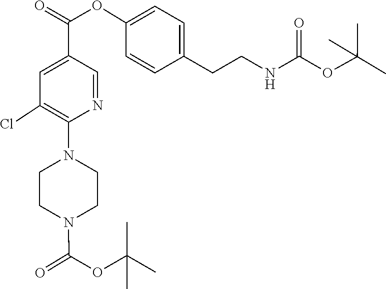 [SMILES: CC(C)(C)OC(=O)NCCc1ccc(OC(=O)c2cnc(N3CCN(C(=O)OC(C)(C)C)CC3)c(Cl)c2)cc1] | tert-butyl 4-[5-[4-[2-(tert- butoxycarbonylamino)ethyl]phenoxy] carbonyl-3-chloro-2-pyridyl]piperazine-1- carboxylate Purification by flash chromatography (cyclohexane/EtOAc 100/0-70/30); Yield: 46%; ¹H NMR (300 MHz, CD₂Cl₂): δ 1.42 (s, 9H), 1.47 (s, 9H), 2.81 (t, J = 7.0 Hz, 2H), 3.36 (q, J = 6.8 Hz, 2H), 3.57 (s, 8H), 4.64 (br s, 1H), 7.11-7.16 (m, 2H), 7.24-7.29 (m, 2H), 8.26 (d, J = 2.1 Hz, 1H), 8.87 (d, J = 2.0 Hz, 1H) ppm; [ES + MS] m/z 561 (MH⁺). |
| 59 | 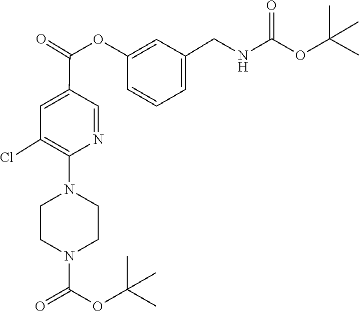 [SMILES: CC(C)(C)OC(=O)NCc1cccc(OC(=O)c2cnc(N3CCN(C(=O)OC(C)(C)C)CC3)c(Cl)c2)c1] | tert-butyl 4-[5-[3-[(tert- butoxycarbonylamino)methyl]phenoxy] carbonyl-3-chloro-2-pyridyl]piperazine-1- carboxylate Purification by flash chromatography (cyclohexane/EtOAc 100/0-70/30); Yield: 65%; ¹H NMR (300 MHz, CD₂Cl₂): δ 1.44 (s, 9H), 1.47 (s, 9H), 3.57 (s, 8H), 4.33 (d, J = 6.3 Hz, 2H), 5.02 (br s, 1H), 7.08-7.14 (m, 2H), 7.21 (d, J = 7.7 Hz, 1H), 7.40 (t, J = 7.9 Hz, 1H), 8.26 (d, J = 2.1 Hz, 1H), 8.87 (d, J = 2.0 Hz, 1H) ppm; [ES + MS] m/z 547 (MH⁺). |
| 60 | 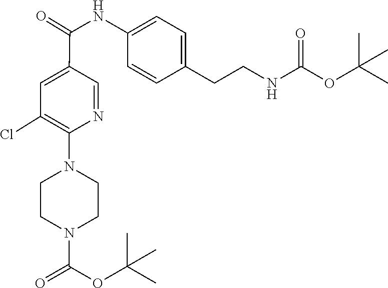 [SMILES: CC(C)(C)OC(=O)NCCc1ccc(NC(=O)c2cnc(N3CCN(C(=O)OC(C)(C)C)CC3)c(Cl)c2)cc1] | tert-butyl 4-[5-[[4-[2-(tert- butoxycarbonylamino)ethyl]phenyl] carbamoyl]-3-chloro-2-pyridyl]piperazine-1- carboxylate Yield: 70%; ¹H NMR (300 MHz, CD₂Cl₂): δ 1.41 (s, 9H), 1.46 (s, 9H), 2.77 (t, J = 7.1 Hz, 2H), 3.33 (q, J = 6.6 Hz, 2H), 3.44-3.49 (m, 4H), 3.54-3.59 (m, 4H), 4.61 (br s, 1H), 7.20 (d, J = 8.5 Hz, 2H), 7.55 (d, J = 8.5 Hz, 2H), 7.81 (br s, 1H), 8.10 (d, J = 2.1 Hz, 1H), 8.62 (d, J = 2.2 Hz, 1H) ppm; [ES + MS] m/z 560 (MH⁺). |
| 83 | 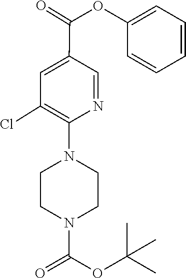 [SMILES: CC(C)(C)OC(=O)N1CCN(c2ncc(C(=O)Oc3ccccc3)cc2Cl)CC1] | tert-butyl 4-(3-chloro-5-phenoxycarbonyl- 2-pyridyl)piperazine-1-carboxylate Purification by flash chromatography (cyclohexane/EtOAc 100/0-80/20); Yield: 78%; ¹H NMR (300 MHz, CD₂Cl₂): δ 1.47 (s, 9H), 3.57 (s, 8H), 7.18-7.23 (m, 2H), 7.26- 7.33 (m, 1H), 7.41-7.48 (m, 2H), 8.28 (d, J = 2.0 Hz, 1H), 8.88 (d, J = 2.0 Hz, 1H) ppm; [ES + MS] m/z 418 (MH⁺). |

Intermediates 61-63 were prepared according to Protocol 5.

| Int | Structure | Physical data |
|---|---|---|
| 61 | 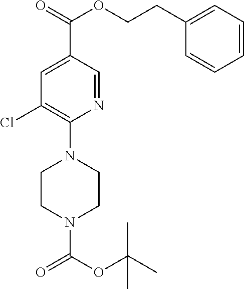 [SMILES: CC(C)(C)OC(=O)N1CCN(c2ncc(C(=O)OCCc3ccccc3)cc2Cl)CC1] | tert-butyl 4-[3-chloro-5-(2- phenylethoxycarbonyl)-2-pyridyl]piperazine- 1-carboxylate Purification by flash chromatography (cyclohexane/EtOAc 100/0-90/10); Yield: 36%; [ES+ MS] m/z 446 (MH⁺). |
| 62 | 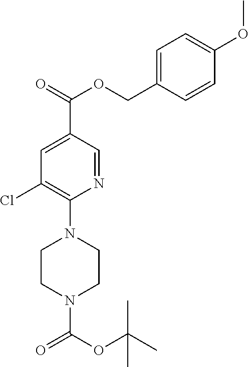 [SMILES: COc1ccc(COC(=O)c2cnc(N3CCN(C(=O)OC(C)(C)C)CC3)c(Cl)c2)cc1] | tert-butyl 4-[3-chloro-5-[(4- methoxyphenyl)methoxycarbonyl]-2- pyridyl]piperazine-1-carboxylate Purification by flash chromatography (cyclohexane/EtOAc 100/0-85/15); Yield: 19%; [ES+ MS] m/z 462 (MH⁺). |
| 63 | 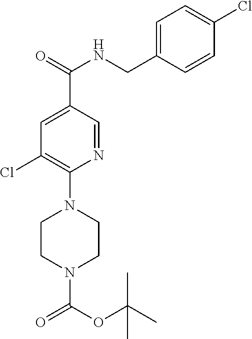 [SMILES: CC(C)(C)OC(=O)N1CCN(c2ncc(C(=O)NCc3ccc(Cl)cc3)cc2Cl)CC1] | tert-butyl 4-[3-chloro-5-[(4- chlorophenyl)methylcarbamoyl]-2- pyridyl]piperazine-1-carboxylate Purification by flash chromatography (cyclohexane/EtOAc 100/0-65/35); Yield: 47%; ¹H NMR (300 MHz, CD₂Cl₂): δ 1.45 (s, 9H), 3.39- 3.47 (m, 4H), 3.50-3.57 (m, 4H), 4.56 (d, J = 6.2 Hz, 2H), 6.50 (t, J = 5.7 Hz, 1H), 7.26-7.35 (m, 4H), 8.01 (d, J = 2.6 Hz, 1H), 8.52 (d, J = 2.6 Hz, 1H) ppm; [ES+ MS] m/z 465 (MH⁺). |

### Intermediate 64: tert-butyl 4-(3-chloro-5-ethoxycarbonyl-2-pyridyl)piperazine-1-carboxylate

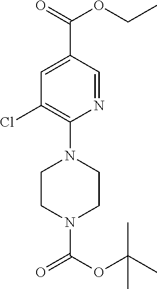 [SMILES: CCOC(=O)c1cnc(N2CCN(C(=O)OC(C)(C)C)CC2)c(Cl)c1]

Intermediate 51 (0.28 mmol) was dissolved in anhydrous DMF (1 mL) and potassium carbonate (2 eq) was added. The reaction mixture was stirred 5 minutes at room temperature and then iodoethane (50 eq) was added. The reaction mixture was stirred overnight at 100° C. and then cooled to room temperature. The mixture was poured into a separating funnel with EtOAc. The organic phase was washed twice with HCl 1N, once with brine, dried over MgSO4 and then evaporated under reduced pressure. The residue was purified by flash chromatography (cyclohexane/EtOAc 100/0-90/10) to give the title compound. Yield: 40%; ¹H NMR (300 MHz, CD₂Cl₂): δ 1.40 (t, J=7.1 Hz, 3H), 1.50 (s, 9H), 3.50-3.54 (m, 4H), 3.57-3.61 (m, 4H), 4.37 (q, J=7.1 Hz, 2H), 8.17 (d, J=2.0 Hz, 1H), 8.76 (d, J=2.0 Hz, 1H) ppm; [ES+MS] m/z 370 (MH⁺).

### Intermediate 65: tert-butyl 4-(5-amino-3-chloro-2-pyridyl)piperazine-1-carboxylate

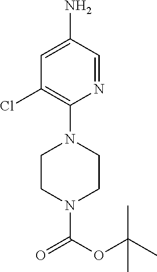 [SMILES: CC(C)(C)OC(=O)N1CCN(c2ncc(N)cc2Cl)CC1]

Intermediate 26 (1 mmol, 1 eq), tBuOK (1.2 eq) and Bis(pinacolato)diboron (3.1 eq) were heated at 110° C. in iPrOH for 4 h. The reaction was evaporated under reduced pressure and the product was purified by flash chromatography (DCM/MeOH 100/0-95/5) to give the title compound. Yield: 100%; ¹H NMR (300 MHz, CD₂Cl₂): δ 1.45 (s, 9H), 3.03-3.08 (m, 4H), 3.48-3.69 (m, 6H), 7.06 (d, J=2.6 Hz, 1H), 7.70 (d, J=2.7 Hz, 1H) ppm; [ES+MS] m/z 313 (MH⁺).

### Intermediate 66: tert-butyl 4-(5-acetamido-3-chloro-2-pyridyl)piperazine-1-carboxylate

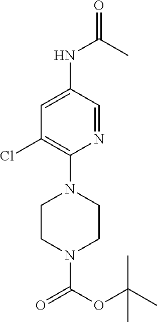 [SMILES: CC(=O)Nc1cnc(N2CCN(C(=O)OC(C)(C)C)CC2)c(Cl)c1]

A mixture of Intermediate 65 (0.12 mmol, 1 eq) and 480 μL acetyl acetate was stirred at RT for 3 days. The reaction was washed with saturated NaHCO₃ and extracted with EtOAc. The organic layers were combined, washed with brine, dried over MgSO₄ and evaporated under reduced pressure. The product was purified by flash chromatography (DCM/MeOH 100/0-95/5) to give the title compound. Yield: 38%; ¹H NMR (300 MHz, CD₂Cl₂): δ 1.46 (s, 9H), 2.13 (s, 3H), 3.16-3.23 (m, 4H), 3.50-3.58 (m, 4H), 7.37 (s, 1H), 8.09 (d, J=2.5 Hz, 1H), 8.15 (d, J=2.5 Hz, 1H) ppm; [ES+MS] m/z 355 (MH⁺).

### Intermediate 67: tert-butyl 4-[3-chloro-5-(methanesulfonamido)-2-pyridyl]piperazine-1-carboxylate

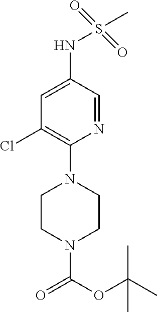 [SMILES: CC(C)(C)OC(=O)N1CCN(c2ncc(NS(C)(=O)=O)cc2Cl)CC1]

A mixture of Intermediate 65 (0.47 mmol, 1 eq) and methanesulfonyl chloride (1.5 eq) in 1 mL anhydrous pyridine was stirred at RT for 2 h. Water was added and the product was extracted with EtOAc. The combined organic layers were washed with brine, dried over MgSO₄ and evaporated under reduced pressure. The crude was purified by flash chromatography (cyclohexane/EtOAc 100/0-60/40) to give the title compound. Yield: 100%; ¹H NMR (300 MHz, CD₂Cl₂): δ 1.46 (s, 9H), 2.99 (s, 3H), 3.24-3.30 (m, 4H), 3.52-3.58 (m, 4H), 6.52 (s, 1H), 7.66 (d, J=2.5 Hz, 1H), 8.07 (d, J=2.5 Hz, 1H) ppm; [ES+MS] m/z 391 (MH⁺).

### Intermediate 68: tert-butyl 4-(3-iodo-2-quinolyl)piperazine-1-carboxylate

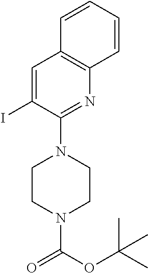 [SMILES: CC(C)(C)OC(=O)N1CCN(c2nc3ccccc3cc2I)CC1]

A dry 10 mL tube was charged with Intermediate 27 (0.17 mmol, 1 eq), CuI (0.08 eq) and sodium iodide (2.0 eq). The tube was purged with argon for 30 min then 1,4-dioxane (2.0 mL) and a solution of (1R,2R)—N1,N2-dimethylcyclohexane-1,2-diamine in 1,4-dioxane (0.1 eq) were added. The mixture was heated at 110° C. for 3 days, CuI (0.66 eq) and (1R,2R)—N1,N2-dimethylcyclohexane-1,2-diamine (0.2 eq), were added by small portion until completed reaction. After 21 days, the reaction was quenched with water, extracted twice with EtOAc. The organic layer was washed with brine, dried over MgSO₄ and then evaporated under reduced pressure. The crude product was purified by flash chromatography (cyclohexane/EtOAc 100/0-90/10) to give the title compound. Yield: 30%; [ES+MS] m/z 440 (MH⁺).

### Intermediate 69: tert-butyl 4-(3-methoxyquinoxalin-2-yl)piperazine-1-carboxylate

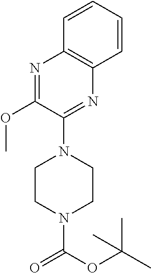 [SMILES: COc1nc2ccccc2nc1N1CCN(C(=O)OC(C)(C)C)CC1]

Intermediate 29 (0.29 mmol, 1 eq) and K₂CO₃ (4 eq) were heated at 65° C. in 1 mL MeOH for 1 h 30. The reaction mixture was cooled to RT then to 0° C. Cold water was added and the precipitate was filtered to give the desired compound. Yield: 74%; ¹H NMR (300 MHz, CD₂Cl₂): δ 1.47 (s, 9H), 3.54-3.59 (m, 4H), 3.62-3.68 (m, 4H), 4.10 (s, 3H), 7.36-7.47 (m, 2H), 7.68 (dd, J=2.4, 7.1 Hz, 2H) ppm; [ES+MS] m/z 345 (MH⁺).

Intermediates 84-86: The tert-butyl 4-(3-chloro-6-iodo-2-quinolyl)piperazine-1-carboxylate 79 (0.2 mmol, 1 eq), appropriate amine (1.5-3.2 eq), Cs₂CO₃ (1.4-2.8 eq), Xantphos (0.08-0.09 eq), Pd₂dbas (0.04-0.07 eq) were dissolved in 1,4-dioxane (0.8-1.0 mL) under argon. The mixture was heated at 100° C. for overnight to 2 days, cooled to room temperature, dried under vacuum and purified by flash chromatography.

| Int | Structure | Physical data |
|---|---|---|
| 84 | 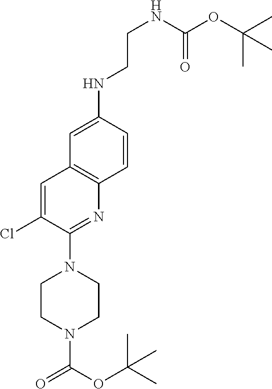 [SMILES: CC(C)(C)OC(=O)NCCNc1ccc2nc(N3CCN(C(=O)OC(C)(C)C)CC3)c(Cl)cc2c1] | tert-butyl 4-[6-[2-(tert- butoxycarbonylamino)ethylamino]-3-chloro-2- quinolyl]piperazine-1-carboxylate Purification by flash chromatography (cyclohexane/EtOAc 100/0-70/30); Yield: 43%; ¹H NMR (300 MHz, CD₂Cl₂): δ 1.44 (s, 9H), 1.47 (s, 9H), 3.27-3.31 (m, 6H), 3.41 (q, J = 5.8 Hz, 2H), 3.57-3.61 (m, 4H), 4.41 (s, 1H), 4.87 (s, 1H), 6.61 (d, J = 2.6 Hz, 1H), 7.02 (dd, J = 2.6, 9.0 Hz, 1H), 7.60 (d, J = 9.0 Hz, 1H), 7.88 (m, 1H) ppm; [ES+ MS] m/z 506 (MH⁺). |
| 85 | 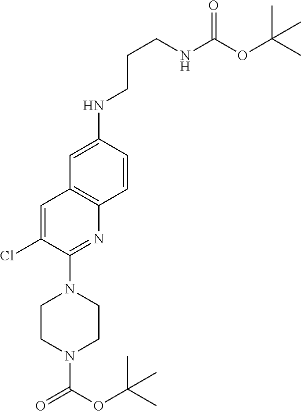 [SMILES: CC(C)(C)OC(=O)NCCCNc1ccc2nc(N3CCN(C(=O)OC(C)(C)C)CC3)c(Cl)cc2c1] | tert-butyl 4-[6-[3-(tert- butoxycarbonylamino)propylamino]-3-chloro-2- quinolyl]piperazine-1-carboxylate Purification by flash chromatography (cyclohexane/EtOAc 100/0-60/40); Yield: 43%; ¹H NMR (300 MHz, CD₂Cl₂): δ 1.43 (s, 9H), 1.47 (s, 9H), 3.20-3.31 (m, 9H), 3.57-3.61 (m, 5H), 4.29 (s, 1H), 4.70 (s, 1H), 6.61 (d, J = 2.6 Hz, 1H), 7.03 (dd, J = 2.6, 9.0 Hz, 1H), 7.60 (d, J = 9.0 Hz, 1H), 7.87 (s, 1H) ppm; [ES+ MS] m/z 520 (MH⁺). |
| 86 | 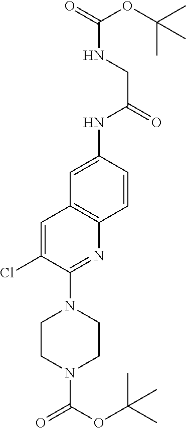 [SMILES: CC(C)(C)OC(=O)NCC(=O)Nc1ccc2nc(N3CCN(C(=O)OC(C)(C)C)CC3)c(Cl)cc2c1] | tert-butyl 4-[6-[[2-(tert- butoxycarbonylamino)acetyl]amino]-3-chloro-2- quinolyl]piperazine-1-carboxylate Purification by flash chromatography (cyclohexane/EtOAc 100/0-50/50); Yield: 63%; ¹H NMR (300 MHz, CD₂Cl₂): δ 1.47 (s, 9H), 1.48 (s, 9H), 3.36-3.40 (m, 4H), 3.58-3.62 (m, 4H), 3.94 (d, J = 6.0 Hz, 2H), 5.36 (br s, 1H), 7.54 (dd, J = 2.4, 9.0 Hz, 1H), 7.74 (d, J = 9.0 Hz, 1H), 8.00 (s, 1H), 8.06 (d, J = 1.8 Hz, 1H), 8.40 (br s, 1H) ppm; [ES+ MS] m/z 520 (MH⁺). |

### Intermediate 87: tert-butyl 4-(2,3-dichloro-6-quinolyl)-3-oxo-piperazine-1-carboxylate

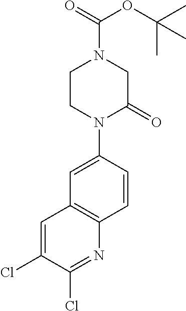 [SMILES: CC(C)(C)OC(=O)N1CCN(c2ccc3nc(Cl)c(Cl)cc3c2)C(=O)C1]

The 2,3-dichloro-6-iodo-quinoline 16 (0.6 mmol, 1 eq), tert-butyl 3-oxopiperazine-1-carboxylate (1.0 eq), Cs₂CO₃ (1.4 eq), Xantphos (0.04 eq), Pd₂dba₃ (0.02 eq) were dissolved in 1,4-dioxane (2.5 mL) under argon. The mixture was heated at 100° C. overnight, cooled to room temperature, dried under vacuum. The crude product was purified by flash chromatography (cyclohexane/EtOAc 100/0-50/50) to give the title compound. Yield: 84%; ¹H NMR (300 MHz, CD₂Cl₂): δ 1.49 (s, 9H), 3.81-3.84 (m, 4H), 4.25 (s, 2H), 7.70 (d, J=2.3 Hz, 1H), 7.75 (dd, J=2.4, 9.0 Hz, 1H), 8.00 (d, J=9.0 Hz, 1H), 8.26 (s, 1H) ppm; [ES+MS] m/z 396 (MH⁺).

### Intermediate 88: tert-butyl 4-[2-(4-tert-butoxycarbonylpiperazin-1-yl)-3-chloro-6-quinolyl]-3-oxo-piperazine-1-carboxylate

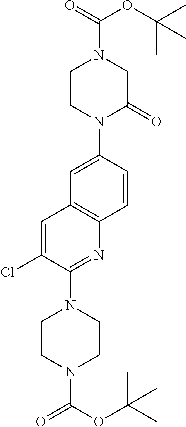 [SMILES: CC(C)(C)OC(=O)N1CCN(c2nc3ccc(N4CCN(C(=O)OC(C)(C)C)CC4=O)cc3cc2Cl)CC1]

The tert-butyl 4-(2,3-dichloro-6-quinolyl)-3-oxo-piperazine-1-carboxylate 87 (0.4 mmol, 1 eq), tert-butyl piperazine-1-carboxylate (1.5 eq), t-BuONa (1.4 eq), BINAP (0.02 eq), Pd(OAc)₂ (0.03 eq) were dissolved in toluene (1.8 mL) under argon. The mixture was heated at 110° C. overnight, cooled to room temperature, dried under vacuum. The crude product was purified by flash chromatography (cyclohexane/EtOAc 100/0-20/80) to give the title compound. Yield: 26%; ¹H NMR (300 MHz, CD₂Cl₂): δ 1.47 (s, 9H), 1.49 (s, 9H), 3.41-3.46 (m, 4H), 3.59-3.63 (m, 4H), 3.79 (s, 4H), 4.22 (s, 2H), 7.54-7.58 (m, 2H), 7.82 (dd, J=0.7, 9.6 Hz, 1H), 8.05 (s, 1H) ppm; [ES+MS] m/z 546 (MH⁺).

### Intermediate 89: tert-butyl 4-[5-[[4-[2-(tert-butoxycarbonylamino)ethyl]benzoyl]amino]-3-chloro-2-pyridyl]piperazine-1-carboxylate

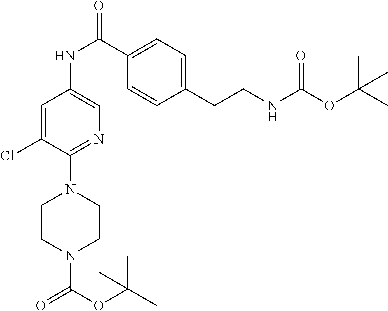 [SMILES: CC(C)(C)OC(=O)NCCc1ccc(C(=O)Nc2cnc(N3CCN(C(=O)OC(C)(C)C)CC3)c(Cl)c2)cc1]

4-[2-(tert-butoxycarbonylamino)ethyl]benzoic acid (1.5 eq) was stirred at RT with NEt₃ (3 eq) and COMU (3 eq) for 10 min in EtOAc (2 mL). Then, a solution of intermediate 65 in 1 mL EtOAc was added (0.50 mmol, 1 eq) and the reaction was allowed to stir at RT for 48 h. The reaction was washed once with an aqueous solution of HCl 1N, twice with a saturated solution of NaHCO₃ and the product was extracted with EtOAc. The combined organic layers were washed with brine, dried over MgSO₄ and evaporated under vacuum. The crude product was purified by reverse phase chromatography (MeOH/H₂O 10/90-100/0) to give the title compound. Yield: 25%; ¹H NMR (300 MHz, CD₂Cl₂): δ 1.41 (s, 9H), 1.46 (s, 9H), 2.86 (t, J=6.9 Hz, 2H), 3.22-3.26 (m, 4H), 3.37 (q, J=6.7 Hz, 2H), 3.53-3.58 (m, 4H), 4.61 (brs, 1H), 7.34 (d, J=8.2 Hz, 2H), 7.81 (d, J=8.2 Hz, 2H), 7.86 (brs, 1H), 8.23 (d, J=2.5 Hz, 1H), 8.30 (d, J=2.4 Hz, 1H) ppm; [ES+MS] m/z 560 (MH⁺).

### Intermediate 90: tert-butyl 4-[3-chloro-5-(5-methyl-1,3,4-oxadiazol-2-yl)-2-pyridyl]piperazine-1-carboxylate

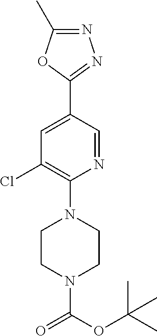 [SMILES: Cc1nnc(-c2cnc(N3CCN(C(=O)OC(C)(C)C)CC3)c(Cl)c2)o1]

Intermediate 51 (0.14 mmol, 1 eq), acetohydrazide (1.4 eq), NEt₃ (5 eq) and T3P 50% in EtOAc (3.5 eq) were put into reaction in 500 μL EtOAc at 80° C. The reaction was stopped after 48 h in total. It was diluted with EtOAc, washed twice with water and twice with NaHCO₃ sat. The organic layer was then washed with brine, dried over MgSO₄ and evaporated under reduced pressure. The crude product was purified by flash chromatography (cyclohexane/EtOAc 100/0-60/40) to give the title compound. Yield: 43%; ¹H NMR (300 MHz, CD₂Cl₂): δ 1.46 (s, 9H), 2.57 (s, 3H), 3.44-3.50 (m, 4H), 3.53-3.59 (m, 4H), 8.18 (d, J=2.1 Hz, 1H), 8.73 (d, J=2.1 Hz, 1H) ppm; [ES+MS] m/z 380 (MH⁺).

### Intermediate 91: tert-butyl 4-[3-chloro-5-[(Z)—N′-hydroxycarbamimidoyl]-2-pyridyl]piperazine-1-carboxylate

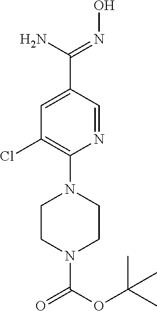 [SMILES: CC(C)(C)OC(=O)N1CCN(c2ncc(/C(N)=N/O)cc2Cl)CC1]

Intermediate 81 (0.47 mmol, 1 eq), hydroxylamine hydrochloride (1.5 eq) and NEt₃ (1.6 eq) were heated at ref lux in 1 mL ethanol absolute for 1 h. The solvent was evaporated under reduced pressure and the residue dissolved in EtOAc. It was washed with water twice. The organic layer was then washed with brine, dried over MgSO₄ and evaporated under reduced pressure to give the title compound. Yield: 98%; ¹H NMR (300 MHz, CD₂Cl₂): δ 1.46 (s, 9H), 3.32-3.38 (i, 4H), 3.53-3.57 (m, 4H), 4.81 (brs, 2H), 6.75 (brs, 1H), 7.86 (d, J=2.1 Hz, 18H), 8.38 (d, J=2.1 Hz, 1H) ppm; [ES+MS] m/z 356 (MH⁺).

Intermediate 92-95: To a mixture of intermediate 91 (0.30-0.50 mmol, 1 eq), the corresponding carboxylic acid (1.1 eq) and NEt₃ (5 eq) in 2 mL EtOAc, was added dropwise T3 50% in EtOAc (3.5 eq). The reaction was heated at 80° C. for 16-24 h. The reaction was diluted with EtOAc, washed twice with water and twice with a saturated solution of NaHCO₃. The organic layer was then washed with brine, dried over MgSO₄ and evaporated under reduced pressure.

| Int | Structure | Physical data |
|---|---|---|
| 92 | 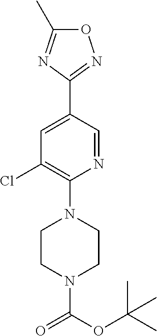 [SMILES: Cc1nc(-c2cnc(N3CCN(C(=O)OC(C)(C)C)CC3)c(Cl)c2)no1] | tert-butyl 4-[3-chloro-5-(5-methyl-1,2,4-oxadiazol-3-yl)-2- pyridyl]piperazine-1-carboxylate Purification by flash chromatography (cyclohexane/EtOAc 100/0-70/30); Yield: 33%; ¹H NMR (300 MHz, CD₂Cl₂): δ 1.46 (s, 9H), 2.63 (s, 3H), 3.41-3.46 (m, 4H), 3.54-3.59 (m, 4H), 8.22 (d, J = 2.0 Hz, 1H), 8.78 (d, J = 2.0 Hz, 1H) ppm; [ES+ MS] m/z 380 (MH⁺). |
| 93 | 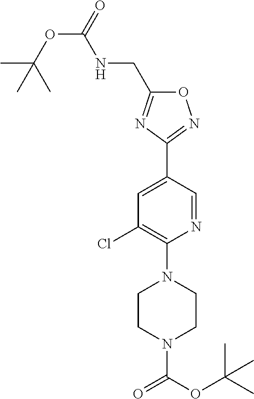 [SMILES: CC(C)(C)OC(=O)NCc1nc(-c2cnc(N3CCN(C(=O)OC(C)(C)C)CC3)c(Cl)c2)no1] | tert-butyl 4-[5-[5-[(tert-butoxycarbonylamino)methyl]- 1,2,4-oxadiazol-3-yl]-3-chloro-2-pyridyl]piperazine-1- carboxylate Purification by flash chromatography (cyclohexane/EtOAc 100/0-70/30); Yield : 27%; ¹H NMR (300 MHz, CD₂Cl₂): δ 1.46 (s, 18H), 3.43-3.48 (m, 4H), 3.54-3.59 (m, 4H), 4.59 (d, J = 6.0 Hz, 2H), 5.27 (brs, 1H), 8.22 (d, J = 2.0 Hz, 1H), 8.79 (d, J = 2.0 Hz, 1H) ppm; [ES+ MS] m/z 495 (MH⁺). |
| 94 | 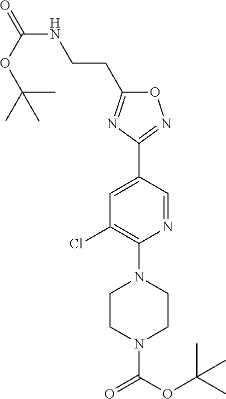 [SMILES: CC(C)(C)OC(=O)NCCc1nc(-c2cnc(N3CCN(C(=O)OC(C)(C)C)CC3)c(Cl)c2)no1] | tert-butyl 4-[5-[5-[2-(tert-butoxycarbonylamino)ethyl]- 1,2,4-oxadiazol-3-yl]-3-chloro-2-pyridyl]piperazine-1- carboxylate Purification by flash chromatography (cyclohexane/EtOAc 100/0-70/30); Yield : 66%; ¹H NMR (300 MHz, CD₂Cl₂): δ 1.41 (s, 9H), 1.46 (s, 9H), 3.12 (t, J = 6.3 Hz, 2H), 3.42-3.48 (m, 4H), 3.54-3.65 (m, 6H), 5.07 (brs, 1H), 8.23 (d, J = 2.1 Hz, 1H), 8.81 (d, J = 2.1 Hz, 1H) ppm; [ES+ MS] m/z 509 (MH⁺). |
| 95 | 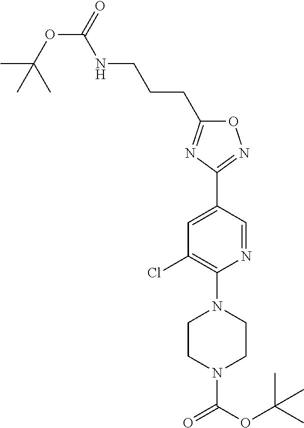 [SMILES: CC(C)(C)OC(=O)NCCCc1nc(-c2cnc(N3CCN(C(=O)OC(C)(C)C)CC3)c(Cl)c2)no1] | tert-butyl 4-[5-[5-[3-(tert-butoxycarbonylamino)propyl]- 1,2,4-oxadiazol-3-yl]-3-chloro-2-pyridyl]piperazine-1- carboxylate Purification by flash chromatography (cyclohexane/EtOAc 100/0-70/30) and reverse phase flash chromatography (MeOH/water 10/90-100/0); Yield : 41%; ¹H NMR (300 MHz, CD₂Cl₂): δ 1.41 (s, 9H), 1.46 (s, 9H), 2.04 (p, J = 7.2 Hz, 2H), 2.98 (t, J = 7.5 Hz, 2H), 3.24 (q, J = 6.6 Hz, 2H), 3.42-3.46 (m, 4H), 3.54-3.59 (m, 4H), 4.77 (brs, 1H), 8.22 (d, J = 2.0 Hz, 1H), 8.80 (d, J = 2.0 Hz, 1H) ppm; [ES+ MS] m/z 523 (MH⁺). |

### Intermediate 96: tert-butyl 4-(3-chloro-5-iodo-2-pyridyl)piperazine-1-carboxylate

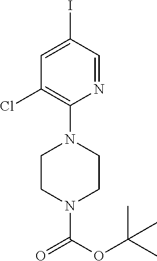 [SMILES: CC(C)(C)OC(=O)N1CCN(c2ncc(I)cc2Cl)CC1]

A round bottom flask was charged with CuI (5 mol %), sodium iodide (2 eq) and intermediate 23 (4.0 mmol, 1 eq). The flask was purged with argon for 30 min then dry dioxane (8.7 mL) and trans-N1,N2-dimethylcyclohexane-1,2-diaminedioxane (0.1 eq) were added. The solution was heated at 110° C. under argon. 5 mol % CuI, 2 eq NaI and 0.1 eq trans-N1,N2-dimethylcyclohexane-1,2-diaminedioxane were added after 7, 11, 13 and 15 days. 5 mL dioxane were also added after 12 days of reaction. The reaction was stopped after 16 days in total. The reaction mixture was diluted with EtOAc and was washed twice with H₂O. The organic layer was then washed with brine, dried over MgSO₄ and evaporated under reduced pressure. The crude was purified by flash chromatography (cyclohexane/EtOAc 100/0-70/30) to give the title compound. Yield: 77%; ¹H NMR (300 MHz, CD₂Cl₂): δ 1.45 (s, 9H), 3.25-3.30 (m, 4H), 3.50-3.56 (m, 4H), 7.88 (d, J=2.0 Hz, 1H), 8.33 (d, J=2.0 Hz, 1H) ppm; [ES+MS] m/z 424 (MH⁺).

Intermediates 97-99, 109, 111: In a tube, were added intermediate 96 (0.19-0.91 mmol, 1 eq), the corresponding alkyne (1.0-2.3 eq), Pd(PPh₃)₂Cl₂ (0.02-0.18 eq) and CuI (0.06-0.6 eq). The tube was purged with argon for 30 min, then anhydrous MeCN (1.0-4.0 mL) and NEt₃ (12-20 eq) were added. The mixture was heated at 50° C. by conventional heating or 100° C. under microwave irradiation for 1-56 h. The reaction mixture was filtered on a celite plug and diluted with EtOAc. The organic layer was washed twice with 1N HCl aqueous solution, once with water and once with brine. It was then dried over MgSO₄, filtered and the solvent was removed under reduced pressure. The crude was purified by flash chromatography (cyclohexane/EtOAc 100/0-80/20) or reverse phase flash chromatography (MeOH or MeCN/water 10/90-100/0).

| Int | Structure | Physical data |
|---|---|---|
| 97 | 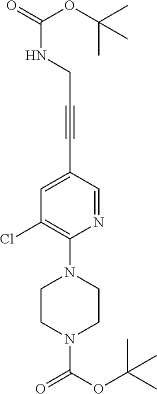 [SMILES: CC(C)(C)OC(=O)NCC#Cc1cnc(N2CCN(C(=O)OC(C)(C)C)CC2)c(Cl)c1] | tert-butyl 4-[5-[3-(tert-butoxycarbonylamino)prop-1- ynyl]-3-chloro-2-pyridyl]piperazine-1-carboxylate Yield: 54%; 1H NMR (300 MHz, CD₂Cl₂): δ 1.44 (s, 9H), 1.45 (s, 9H), 3.30-3.35 (m, 4H), 3.51-3.56 (m, 4H), 4.11 (d, J = 5.9 Hz, 2H), 4.89 (brs, 1H), 7.62 (d, J = 2.0 Hz, 1H), 8.20 (d, J = 2.0 Hz, 1H) ppm; [ES+ MS] m/z 451 (MH⁺). |
| 98 | 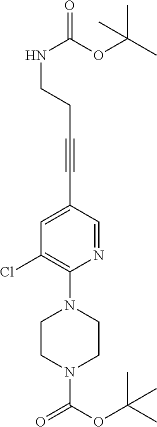 [SMILES: CC(C)(C)OC(=O)NCCC#Cc1cnc(N2CCN(C(=O)OC(C)(C)C)CC2)c(Cl)c1] | tert-butyl 4-[5-[4-(tert-butoxycarbonylamino)but-1-ynyl]- 3-chloro-2-pyridyl]piperazine-1-carboxylate Yield: 87%; ¹H NMR (300 MHz, CD₂Cl₂): δ 1.43 (s, 9H), 1.45 (s, 9H), 2.59 (t, J = 6.5 Hz, 2H), 3.28-3.35 (m, 6H), 3.51-3.56 (m, 4H), 4.87 (brs, 1H), 7.61 (d, J = 2.0 Hz, 1H), 8.18 (d, J = 2.0 Hz, 1H) ppm; [ES+ MS] m/z 465 (MH⁺). |
| 99 | 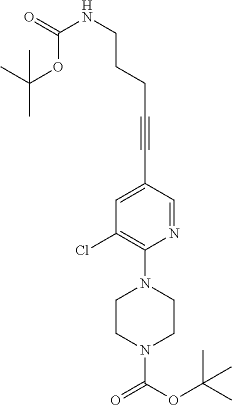 [SMILES: CC(C)(C)OC(=O)NCCCC#Cc1cnc(N2CCN(C(=O)OC(C)(C)C)CC2)c(Cl)c1] | tert-butyl 4-[5-[5-(tert-butoxycarbonylamino)pent-1- ynyl]-3-chloro-2-pyridyl]piperazine-1-carboxylate Yield: 74%; ¹H NMR (300 MHz, CD₂Cl₂): δ 1.43 (s, 9H), 1.45 (s, 9H), 1.76 (p, J = 6.8 Hz, 2H), 2.45 (t, J = 7.0 Hz, 2H), 3.23 (q, J = 6.6 Hz, 2H), 3.28-3.33 (m, 4H), 3.51-3.56 (m, 4H), 4.72 (brs, 1H), 7.60 (d, J = 2.0 Hz, 1H), 8.17 (d, J = 2.0 Hz, 1H) ppm; [ES+ MS] m/z 479 (MH⁺). |
| 109 | 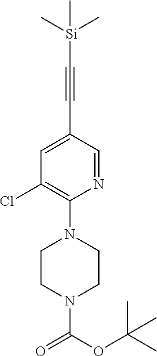 [SMILES: CC(C)(C)OC(=O)N1CCN(c2ncc(C#C[Si](C)(C)C)cc2Cl)CC1] | tert-butyl 4-[3-chloro-5-(2-trimethylsilylethynyl)-2- pyridyl]piperazine-1-carboxylate Yield: 68%; ¹H NMR (300 MHz, CD₂Cl₂): δ 0.24 (s, 9H), 1.45 (s, 9H), 3.32-3.37 (m, 4H), 3.51-3.56 (m, 4H), 7.64 (d, J = 1.9 Hz, 1H), 8.22 (d, J = 2.0 Hz, 1H) ppm; [ES+ MS] m/z 394 (MH⁺). |
| 111 | 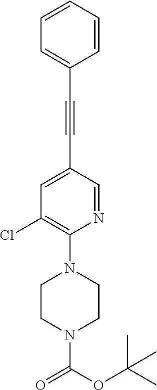 [SMILES: CC(C)(C)OC(=O)N1CCN(c2ncc(C#Cc3ccccc3)cc2Cl)CC1] | tert-butyl 4-[3-chloro-5-(2-phenylethynyl)-2-pyridyl] piperazine-1-carboxylate Yield: 93%; ¹H NMR (300 MHz, CD₂Cl₂): δ 1.46 (s, 9H), 3.33- 3.40 (m, 4H), 3.52-3.59 (m, 4H), 7.34-7.40 (m, 3H), 7.50-7.55 (m, 2H), 7.74 (d, J = 2.0 Hz, 1H), 8.31 (d, J = 2.0 Hz, 1H) ppm; [ES+ MS] m/z 398 (MH⁺). |

### Intermediate 100: tert-butyl 4-[3-chloro-5-(hydroxymethyl)-2-pyridyl]piperazine-1-carboxylate

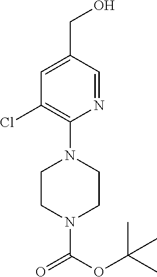 [SMILES: CC(C)(C)OC(=O)N1CCN(c2ncc(CO)cc2Cl)CC1]

A tube containing intermediate 22 (1.50 mmol, 1 eq) was purged under argon for 15 min. Then, 6 mL dry tetrahydrofuran were added and the reaction was cooled at 0° C. LiBH₄ 2.0 M in THF (4 eq) was added, the reaction was allowed to warm up at RT and was stirred for 3 days. The reaction mixture was quenched with EtOAc and washed twice with water. The aqueous layer was then extracted with EtOAc. The combined organic layers were washed with brine, dried over MgSO₄ and evaporated under reduced pressure. The crude was purified by flash chromatography (cyclohexane/EtOAc 100/0-50/50) to give the title compound. Yield: 43%; ¹H NMR (300 MHz, CD₂Cl₂): δ 1.46 (s, 9H), 3.24-3.29 (m, 4H), 3.52-3.57 (m, 4H), 4.60 (s, 2H), 7.66 (d, J=2.1 Hz, 1H), 8.13 (d, J=2.1 Hz, 1H) ppm; [ES+MS]m/z 328 (MH⁺).

### Intermediate 101: tert-butyl 4-[3-chloro-5-(methylsulfonyloxymethyl)-2-pyridyl]piperazine-1-carboxylate

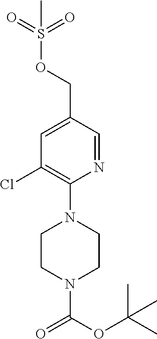 [SMILES: CC(C)(C)OC(=O)N1CCN(c2ncc(COS(C)(=O)=O)cc2Cl)CC1]

Intermediate 100 (0.27 mmol, 1 eq) was dissolved in dichloromethane (1 mL), cooled at 0° C. and methanesulfonyl chloride (2 eq) was added. The reaction mixture was allowed to warm up at RT and was stirred for 2 h. The reaction was quenched with water and extracted with DCM to give the title compound. Yield: 84%.

### Intermediate 102: tert-butyl 4-[5-[[3-[(tert-butoxycarbonylamino)methyl]phenoxy]methyl]-3-chloro-2-pyridyl]piperazine-1-carboxylate

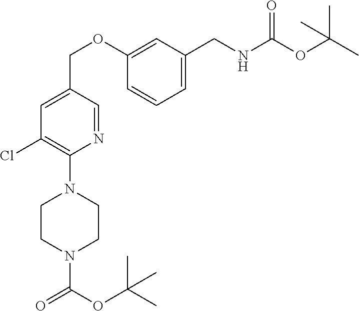 [SMILES: CC(C)(C)OC(=O)NCc1cccc(OCc2cnc(N3CCN(C(=O)OC(C)(C)C)CC3)c(Cl)c2)c1]

tert-butyl N-[(3-hydroxyphenyl)methyl]carbamate (1.2 eq) and cesium carbonate (1.3 eq) were heated at 70° C. in dry DMF (1.3 mL). After 10 min, a solution of intermediate 101 (0.23 mmol, 1 eq) in dry DMF (1 mL) was added and the reaction was heated for 4 h. The DMF was evaporated under reduced pressure, the residue dissolved in EtOAc and washed twice with an aqueous solution of HCl 1N. The organic layer was then washed with brine, dried over MgSO₄ and evaporated under reduced pressure. The crude was purified by flash chromatography (cyclohexane/EtOAc 100/0-70/30) to give the title compound. Yield: 59%; ¹H NMR (300 MHz, CD₂Cl₂): δ 1.44 (s, 9H), 1.46 (s, 9H), 3.27-3.32 (m, 4H), 3.52-3.59 (m, 4H), 4.26 (d, J=6.1 Hz, 2H), 4.97 (s, 2H), 6.82-6.92 (m, 3H), 7.26 (t, J=8.0 Hz, 1H), 7.72 (d, J=2.1 Hz, 1H), 8.22 (d, J=2.1 Hz, 1H) ppm; [ES+MS] m/z 533 (MH⁺).

### Intermediate 103: tert-butyl N-[[3-[(5,6-dichloro-3-pyridyl)oxymethyl]phenyl]methyl]carbamate

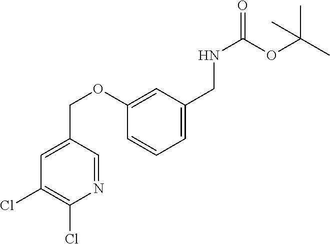 [SMILES: CC(C)(C)OC(=O)NCc1cccc(OCc2cnc(Cl)c(Cl)c2)c1]

5,6-dichloropyridin-3-ol (0.44 mmol, 1 eq), tert-butyl N-[[3-(bromomethyl)phenyl]methyl]carbamate (2 eq) and K₂CO₃ (2.5 eq) were heated at 80° C. in DMF (1.2 mL) for 1 h. The reaction was diluted with EtOAc and washed with water twice. The organic layer was then washed with brine, dried over MgSO₄ and evaporated under reduced pressure. The crude was purified by flash chromatography (cyclohexane/EtOAc 100/0-80/20) and by reverse phase chromatography (MeCN 0.1% formic acid/water 0.1% formic acid 10/90-100/0). Yield: 74%; ¹H NMR (300 MHz, CD₂Cl₂): δ 1.44 (s, 9H), 4.31 (d, J=6.2 Hz, 2H), 4.98 (brs, 1H), 5.09 (s, 2H), 7.26-7.40 (m, 4H), 7.43 (d, J=2.8 Hz, 1H), 8.05 (d, J=2.8 Hz, 1H) ppm; [ES+MS] m/z 383 (MH⁺).

### Intermediate 104: tert-butyl 4-[5-[[3-[(tert-butoxycarbonylamino)methyl]phenyl]methoxy]-3-chloro-2-pyridyl]piperazine-1-carboxylate

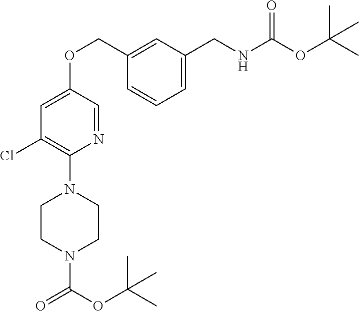 [SMILES: CC(C)(C)OC(=O)NCc1cccc(COc2cnc(N3CCN(C(=O)OC(C)(C)C)CC3)c(Cl)c2)c1]

In a tube, intermediate 103 (0.12 mmol, 1 eq), boc-piperazine (1.8 eq), Pd(OAc)₂ (4 mol %), BINAP (4 mol %), and tBuONa (1.7 eq) were purged under argon for 15 min. Then, dry toluene (0.2 mL) was added and the mixture was heated at 110° C. overnight. The reaction mixture was filtered on a plug of celite and rinsed with EtOAc. It was concentrated under reduced pressure and the residue was purified by flash chromatography (cyclohexane/EtOAc 100/0-70/30) to give the title compound. Yield: 67%; ¹H NMR (300 MHz, CD₂Cl₂): δ 1.44 (s, 9H), 1.46 (s, 9H), 3.10-3.16 (m, 4H), 3.51-3.57 (m, 4H), 4.30 (d, J=6.1 Hz, 2H), 5.00-5.08 (m, 3H), 7.24-7.39 (m, 5H), 7.95 (d, J=2.7 Hz, 1H) ppm; [ES+MS] m/z 533 (MH⁺).

### Intermediate 105: tert-butyl 4-[6-[3-[(tert-butoxycarbonylamino)methyl]phenyl]-3-chloro-2-quinolyl]piperazine-1-carboxylate

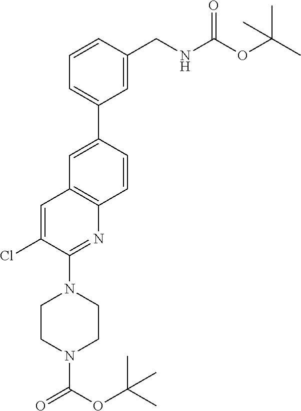 [SMILES: CC(C)(C)OC(=O)NCc1cccc(-c2ccc3nc(N4CCN(C(=O)OC(C)(C)C)CC4)c(Cl)cc3c2)c1]

The tert-butyl 4-(6-bromo-3-chloro-2-quinolyl)piperazine-1-carboxylate 78 (0.2 mmol, 1 eq), [3-[(tert-butoxycarbonylamino)methyl]phenyl]boronic acid (1.0 eq), K₂CO₃ (1.6 eq) were dissolved in a mixture of DME/EtOH/H₂O (2/1/2) (5 mL). Then was added Pd(PPh₃)₂Cl₂ (0.1 eq.), the suspension was heated at 90° C. under argon for 1 h. The reaction was diluted with CH₂Cl₂ and washed with water twice. The organic layer was then washed with brine, dried over MgSO₄ and evaporated under reduced pressure. The crude product was purified by flash chromatography (cyclohexane/EtOAc 100/0-70/30) to give the title compound.

Yield: 55%; ¹H NMR (300 MHz, CD₂Cl₂): δ 1.46 (s, 9H), 1.48 (s, 9H), 3.43-3.47 (m, 4H), 3.60-3.64 (m, 4H), 4.38 (d, J=9.3 Hz, 2H), 5.04 (br s, 1H), 7.28-7.32 (m, 1H), 7.42-7.48 (m, 1H), 7.58-7.62 (m, 2H), 7.85-7.89 (m, 3H), 8.13 (s, 1H) ppm; [ES+MS] m/z 553 (MH⁺).

Intermediates 106-107, 120-121, 125-131: The tert-butyl 4-(6-bromo-3-chloro-2-quinolyl)piperazine-1-carboxylate 78 (0.1-0.4 mmol, 1 eq), appropriate amine (1.5-3 eq), Cs₂CO₃ (1.3-1.5 eq), Xantphos (0.04-0.10 eq), Pd₂dba₃ (0.02-0.04 eq) were dissolved in 1,4-dioxane (0.7-4.0 mL) under argon. The mixture was heated at 100° C. for overnight, cooled to room temperature, filtered on celite, dried under vacuum and purified by flash chromatography.

| Int | Structure | Physical data |
|---|---|---|
| 106 | 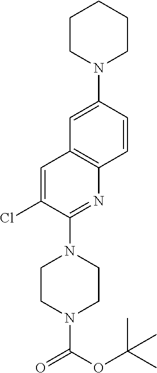 [SMILES: CC(C)(C)OC(=O)N1CCN(c2nc3ccc(N4CCCCC4)cc3cc2Cl)CC1] | tert-butyl 4-[3-chloro-6-(1-piperidyl)-2- quinolyl]piperazine-1-carboxylate Purification by flash chromatography (cyclohexane/EtOAc 100/0-85/15); Yield: 56%; ¹H NMR (300 MHz, CD₂Cl₂): δ 1.47 (s, 9H), 1.58-1.66 (m, 2H), 1.70-1.76 (m, 4H), 3.20-3.24 (m, 4H), 3.30-3.34 (m, 4H), 3.58-3.62 (m, 4H), 6.90 (d, J = 2.7 Hz, 1H), 7.75 (dd, J = 2.8, 9.3 Hz, 1H), 7.67 (d, J = 9.3 Hz, 1H), 7.92 (s, 1H) ppm; [ES+ MS] m/z 431 (MH⁺). |
| 107 | 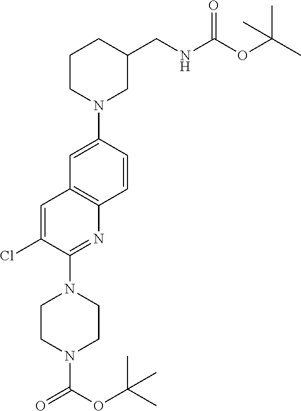 [SMILES: CC(C)(C)OC(=O)NCC1CCCN(c2ccc3nc(N4CCN(C(=O)OC(C)(C)C)CC4)c(Cl)cc3c2)C1] | tert-butyl 4-[6-[3-[(tert- butoxycarbonylamino)methyl]-1-piperidyl]-3- chloro-2-quinolyl]piperazine-1-carboxylate Purification by flash chromatography (cyclohexane/EtOAc 100/0-70/30); Yield: 42%; ¹H NMR (300 MHz, CD₂Cl₂): δ 1.44 (s, 9H), 1.47 (s, 9H), 1.58-1.75 (m, 2H), 1.80-1.89 (m, 3H), 2.51-2.59 (m, 1H), 2.74-2.83 (m, 1H), 3.10 (t, J = 6.4 Hz, 2H), 3.30- 3.34 (m, 4H), 3.57-3.67 (m, 6H), 4.73 (br s, 1H), 6.91 (d, J = 2.7 Hz, 1H), 7.39 (dd, J = 2.7, 9.3 Hz, 1H), 7.67 (d, J = 9.2 Hz, 1H), 7.92 (s, 1H) ppm; [ES+ MS] m/z 560 (MH⁺). |
| 120 | 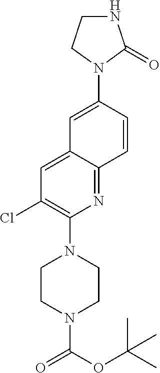 [SMILES: CC(C)(C)OC(=O)N1CCN(c2nc3ccc(N4CCNC4=O)cc3cc2Cl)CC1] | tert-butyl 4-[3-chloro-6-(2-oxoimidazolidin-1-yl)-2- quinolyl]piperazine-1-carboxylate Purification by flash chromatography (DCM/EtOAc 100/0-60/40); Yield: 35%; ¹H NMR (300 MHz, CD₂Cl₂): δ 1.47 (s, 9H), 3.35-3.41 (m, 4H), 3.57-3.64 (m, 6H), 3.98-4.05 (m, 2H), 4.69 (s, 1H), 7.56 (d, J = 2.5 Hz, 1H), 7.77 (d, J = 9.2 Hz, 1H), 8.03 (s, 1H), 8.13 (dd, J = 2.6, 9.3 Hz, 1H) ppm; [ES+ MS] m/z 432 (MH⁺). |
| 121 | 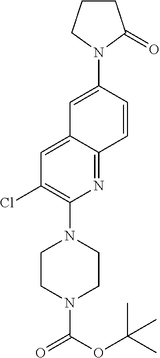 [SMILES: CC(C)(C)OC(=O)N1CCN(c2nc3ccc(N4CCCC4=O)cc3cc2Cl)CC1] | tert-butyl 4-[3-chloro-6-(2-oxopyrrolidin-1-yl)-2- quinolyl]piperazine-1-carboxylate Purification by flash chromatography (DCM/EtOAc 100/0-70/30); Yield: 82%; ¹H NMR (300 MHz, CD₂Cl₂): δ 1.47 (s, 9H), 2.19 (p, J = 7.6 Hz, 2H), 2.60 (t, J = 7.9 Hz, 2H), 3.37-3.43 (m, 4H), 3.58-3.63 (m, 4H), 3.94 (t, J = 7.0 Hz, 2H), 7.77-7.82 (m, 2H), 8.03-8.08 (m, 2H) ppm; [ES+ MS] m/z 431 (MH⁺). |
| 125 | 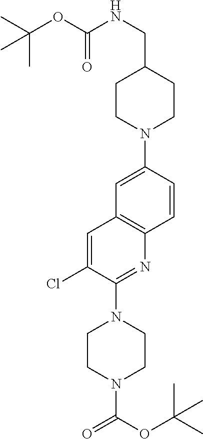 [SMILES: CC(C)(C)OC(=O)NCC1CCN(c2ccc3nc(N4CCN(C(=O)OC(C)(C)C)CC4)c(Cl)cc3c2)CC1] | tert-butyl 4-[6-[4-[(tert- butoxycarbonylamino)methyl]-1-piperidyl]-3- chloro-2-quinolyl]piperazine-1-carboxylate Purification by flash chromatography (cyclohexane/EtOAc 100/0-70/30); Yield: 53%; ¹H NMR (300 MHz, CD₂Cl₂): δ 1.43 (s, 9H), 1.47 (s, 9H), 1.53-1.65 (m, 2H), 1.79-1.86 (m, 2H), 2.75 (td, J = 2.3, 12.2 Hz, 2H), 3.04 (d, J = 6.4 Hz, 2H), 3.30-3.34 (m, 4H), 3.57-3.61 (m, 4H), 3.74-3.80 (m, 2H), 4.70 (s, 1H), 6.91 (d, J = 2.6 Hz, 1H), 7.39 (dd, J = 2.7, 9.3 Hz, 1H), 7.67 (d, J = 9.3 Hz, 1H), 7.93 (s, 1H) ppm; [ES+ MS] m/z 560 (MH⁺). |
| 126 | 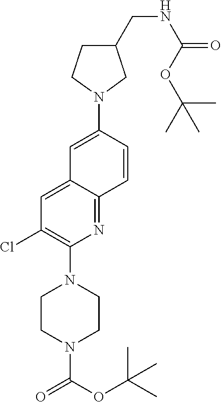 [SMILES: CC(C)(C)OC(=O)NCC1CCN(c2ccc3nc(N4CCN(C(=O)OC(C)(C)C)CC4)c(Cl)cc3c2)C1] | tert-butyl 4-[6-[3-[(tert- butoxycarbonylamino)methyl]pyrrolidin-1-yl]-3- chloro-2-quinolyl]piperazine-1-carboxylate Purification by flash chromatography (cyclohexane/EtOAc 100/0-80/20); Yield: 86%; ¹H NMR (300 MHz, CD₂Cl₂): δ 1.43 (s, 9H), 1.47 (s, 9H), 1.74-1.86 (m, 1H), 2.11-2.22 (m, 1H), 2.50-2.60 (m, 1H), 3.08-3.52 (m, 10H), 3.57-3.61 (m, 4H), 4.78 (s, 1H), 6.53 (d, J = 2.6 Hz, 1H), 7.09 (dd, J = 2.7, 9.2 Hz, 1H), 7.67 (d, J = 9.2 Hz, 1H), 7.89 (s, 1H) ppm; [ES+ MS] m/z 546 (MH⁺). |
| 127 | 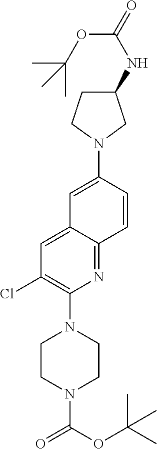 [SMILES: CC(C)(C)OC(=O)N[C@@H]1CCN(c2ccc3nc(N4CCN(C(=O)OC(C)(C)C)CC4)c(Cl)cc3c2)C1] | tert-butyl 4-[6-[(3R0-3-(tert- butoxycarbonylamino)pyrrolidin-1-yl]-3-chloro-2- quinolyl]piperazine-1-carboxylate Purification by flash chromatography (cyclohexane/EtOAc 100/0-80/20); Yield: 46%; ¹H NMR (300 MHz, CD₂Cl₂): δ 1.43 (s, 9H), 1.47 (s, 9H), 1.92-2.02 (m, 1H), 2.26-2.37 (m, 1H), 3.21-3.68 (m, 12H), 4.34 (s, 1H), 4.81 (s, 1H), 6.55 (d, J = 2.7 Hz, 1H), 7.10 (dd, J = 2.7, 9.3 Hz, 1H), 7.69 (d, J = 9.2 Hz, 1H), 7.90 (s, 1H) ppm; [ES+ MS] m/z 532 (MH⁺). |
| 128 | 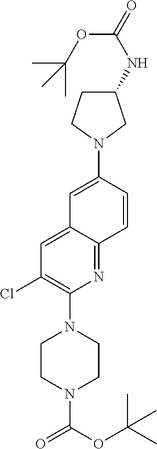 [SMILES: CC(C)(C)OC(=O)N[C@H]1CCN(c2ccc3nc(N4CCN(C(=O)OC(C)(C)C)CC4)c(Cl)cc3c2)C1] | tert-butyl 4-[6-[(3S)-3-(tert- butoxycarbonylamino)pyrrolidin-1-yl]-3-chloro-2- quinolyl]piperazine-1-carboxylate Purification by flash chromatography (cyclohexane/EtOAc 100/0-70/30); Yield: 81%; ¹H NMR (300 MHz, CD₂Cl₂): δ 1.43 (s, 9H), 1.47 (s, 9H), 1.92-2.03 (m, 1H), 2.26-2.38 (m, 1H), 3.21-3.31 (m, 5H), 3.37-3.67 (m, 7H), 4.36 (s, 1H), 4.81 (s, 1H), 6.55 (d, J = 2.6 Hz, 1H), 7.09 (dd, J = 2.7, 9.2 Hz, 1H), 7.68 (d, J = 9.1 Hz, 1H), 7.90 (s, 1H) ppm; [ES+ MS] m/z 532 (MH⁺). |
| 129 | 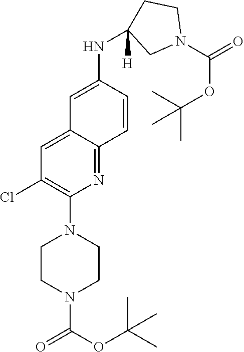 [SMILES: CC(C)(C)OC(=O)N1CCN(c2nc3ccc(N[C@@H]4CCN(C(=O)OC(C)(C)C)C4)cc3cc2Cl)CC1] | tert-butyl 4-[6-[[(3R)-1-tert- butoxycarbonylpyrrolidin-3-yl]amino]-3-chloro-2- quinolyl]piperazine-1-carboxylate Purification by flash chromatography (cyclohexane/EtOAc 100/0-70/30); Yield: 51%; ¹H NMR (300 MHz, CD₂Cl₂): δ 1.43 (s, 9H), 1.47 (s, 9H), 1.89-1.98 (m, 1H), 2.21-2.29 (m, 1H), 3.25-3.32 (m, 5H), 3.44-3.48 (m, 2H), 3.58-3.61 (m, 4H), 3.72 (s, 1H), 4.10 (s, 2H), 6.62 (d, J = 2.6 Hz, 1H), 7.01 (dd, J = 2.6, 9.0 Hz, 1H), 7.62 (d, J = 9.0 Hz, 1H), 7.89 (s, 1H) ppm; [ES+ MS] m/z 532 (MH⁺). |
| 130 | 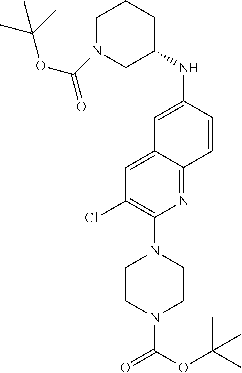 [SMILES: CC(C)(C)OC(=O)N1CCN(c2nc3ccc(N[C@H]4CCCN(C(=O)OC(C)(C)C)C4)cc3cc2Cl)CC1] | tert-butyl 4-[6-[(1-tert-butoxycarbonyl-3- piperidyl)amino]-3-chloro-2-quinolyl]piperazine-1- carboxylate Purification by flash chromatography (cyclohexane/EtOAc 100/0-70/30); Yield: 50%; ¹H NMR (300 MHz, CD₂Cl₂): δ 1.43 (s, 9H), 1.47 (s, 9H) 1.53-1.79 (m, 3H), 1.99-2.07 (m, 1H), 2.90-3.00 (m, 1H), 3.07-3.15 (m, 1H), 3.29-3.31 (m, 4H), 3.43-3.50 (m, 1H), 3.57-3.62 (m, 4H), 3.64-3.72 (m, 1H), 3.98- 4.05 (m, 2H), 6.67 (d, J = 2.3 Hz, 1H), 7.02 (dd, J = 2.6, 9.0 Hz, 1H), 7.62 (d, J = 9.0 Hz, 1H), 7.87 (s, 1H) ppm; [ES+ MS] m/z 546 (MH⁺). |
| 131 | 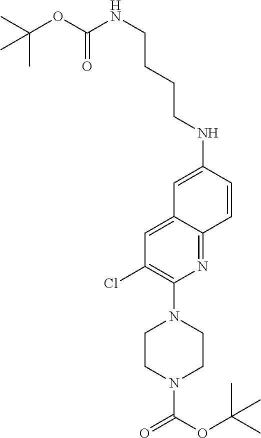 [SMILES: CC(C)(C)OC(=O)NCCCCNc1ccc2nc(N3CCN(C(=O)OC(C)(C)C)CC3)c(Cl)cc2c1] | tert-butyl 4-[6-[4-(tert- butoxycarbonylamino)butylamino]-3-chloro-2- quinolyl]piperazine-1-carboxylate Purification by flash chromatography (cyclohexane/EtOAc 100/0-80/20); Yield: 18%; ¹H NMR (300 MHz, CD₂Cl₂): 0 1.42 (s, 9H), 1.47 (s, 9H), 1.59-1.73 (m, 4H), 3.11-3.23 (m, 4H), 3.26- 3.30 (m, 4H), 3.57-3.61 (m, 4H), 4.04 (br s, 1H), 4.64 (br s, 1H), 6.59 (d, J = 2.6 Hz, 1H), 7.02 (dd, J = 2.6, 9.0 Hz, 1H), 7.59 (d, J = 9.0 Hz, 1H), 7.88 (s, 1H) ppm; [ES+ MS] m/z 534 (MH⁺). |

### Intermediate 108: tert-butyl 4-(3-chloro-1,5-naphthyridin-2-yl)piperazine-1-carboxylate

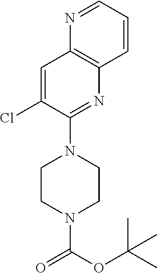 [SMILES: CC(C)(C)OC(=O)N1CCN(c2nc3cccnc3cc2Cl)CC1]

The 2,3-dichloro-1,5-naphthyridine 77 (0.3 mmol, 1 eq), tert-butyl piperazine-1-carboxylate (1.5 eq), t-BuONa (1.5 eq), SPhos (0.08 eq), Pd(OAc)₂ (0.04 eq) were dissolved in toluene (0.7 mL) under argon. The mixture was heated at 110° C. overnight, cooled to room temperature, dried under vacuum. The crude product was purified by flash chromatography (cyclohexane/EtOAc 100/0-50/50) to give the title compound. Yield: 21%; ¹H NMR (300 MHz, CD₂Cl₂): δ 1.47 (s, 9H), 3.45-3.49 (m, 4H), 3.60-3.64 (m, 4H), 7.28 (dd, J=4.2, 8.5 Hz, 1H), 8.11 (ddd, J=0.8, 1.6, 8.5 Hz, 1H), 8.28 (d, J=0.7 Hz, 1H), 8.98 (dd, J=1.6, 4.2 Hz, 1H) ppm; [ES+MS] m/z 349 (MH⁺).

### Intermediate 110: tert-butyl 4-(3-chloro-5-ethynyl-2-pyridyl)piperazine-1-carboxylate

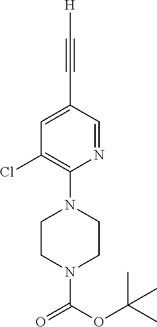 [SMILES: C#Cc1cnc(N2CCN(C(=O)OC(C)(C)C)CC2)c(Cl)c1]

TBAF 1M in THF (1.1 eq) was added to a solution of intermediate 109 (0.08-0.61 mmol) in 0.3-2.5 mL THF. The reaction was allowed to stir at room temperature overnight. The reaction was diluted with EtOAc and washed twice with water. The organic layer was then washed with brine, dried over MgSO₄ and evaporated under reduced pressure to give the title compound. Yield: 92%; ¹H NMR (300 MHz, CD₂Cl₂): δ 1.46 (s, 9H), 3.21 (s, 1H), 3.32-3.39 (m, 4H), 3.51-3.57 (m, 4H), 7.68 (d, J=2.0 Hz, 1H), 8.26 (d, J=2.0 Hz, 1H) ppm; [ES+MS] m/z 322 (MH⁺).

Intermediates 112-114: In a tube, were added intermediate 110 (0.23-0.62 mmol), Pd(PPh₃)₂Cl₂ (2-3 mol %), CuI (7-10 mol %) and the corresponding iodo derivative (1.4 eq). Then TEA (14 eq) and 0.9-2.5 mL anhydrous MeCN were added. The reaction was flushed under argon for 30 min and was heated at 100° C. under microwaves for 1 h. The reaction was filtered on a celite plug. The filtrate was diluted with EtOAc and was washed three times with water and once with brine. It was then dried over MgSO₄, filtered and the solvent was removed under reduced pressure. The crude was purified by flash chromatography.

| Int | Structure | Physical data |
|---|---|---|
| 112 | 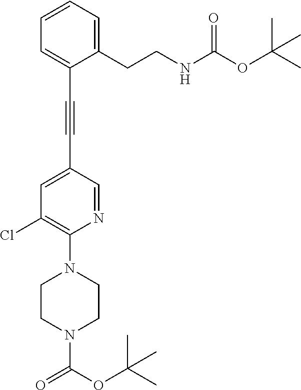 [SMILES: CC(C)(C)OC(=O)NCCc1ccccc1C#Cc1cnc(N2CCN(C(=O)OC(C)(C)C)CC2)c(Cl)c1] | tert-butyl 4-[5-[2-[2-[2-(tert- butoxycarbonylamino)ethyl]phenyl]ethynyl]-3- chloro-2-pyridyl]piperazine-1-carboxylate Purification by reverse phase flash chromatography (water/MeCN 90/10-0/100); Yield: 56%; ¹H NMR (300 MHz, CD₂Cl₂): δ 1.39 (s, 9H), 1.46 (s, 9H), 3.02 (t, J = 7.0 Hz, 2H), 3.33-3.46 (m, 6H), 3.53-3.58 (m, 4H), 4.68 (brs, 1H), 7.20-7.34 (m, 3H), 7.51 (dd, J = 1.4, 8.0 Hz, 1H), 7.85 (brs, 1H), 8.34 (d, J = 2.0 Hz, 1H) ppm; [ES+ MS] m/z 541 (MH⁺). |
| 113 | 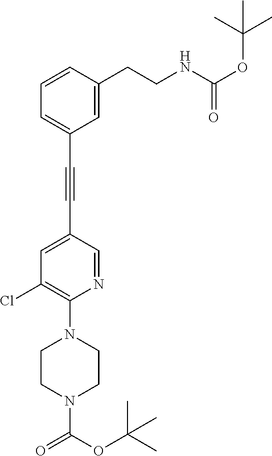 [SMILES: CC(C)(C)OC(=O)NCCc1cccc(C#Cc2cnc(N3CCN(C(=O)OC(C)(C)C)CC3)c(Cl)c2)c1] | tert-butyl 4-[5-[2-[3-[2-(tert- butoxycarbonylamino)ethyl]phenyl]ethynyl]-3- chloro-2-pyridyl]piperazine-1-carboxylate Purification by flash chromatography (cyclohexane/EtOAc 100/0-80/20); Yield: 52%; ¹H NMR (300 MHz, CD₂Cl₂): δ 1.41 (s, 9H), 1.46 (s, 9H), 2.79 (t, J = 7.0 Hz, 2H), 3.31-3.40 (m, 6H), 3.52-3.59 (m, 4H), 4.60 (brs, 1H), 7.20 (td, J = 1.4, 7.5 Hz, 1H), 7.31 (t, J = 7.8 Hz, 1H), 7.36-7.40 (m, 2H), 7.73 (d, J = 2.0 Hz, 1H), 8.31 (d, J = 2.0 Hz, 1H) ppm; [ES+ MS] m/z 541 (MH⁺). |
| 114 | 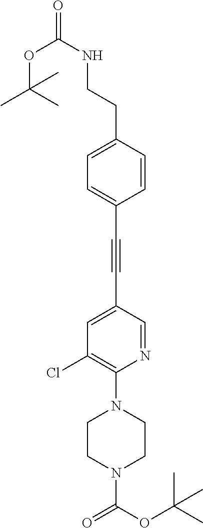 [SMILES: CC(C)(C)OC(=O)NCCc1ccc(C#Cc2cnc(N3CCN(C(=O)OC(C)(C)C)CC3)c(Cl)c2)cc1] | tert-butyl 4-[5-[2-[4-[2-(tert- butoxycarbonylamino)ethyl]phenyl]ethynyl]-3- chloro-2-pyridyl]piperazine-1-carboxylate Purification by flash chromatography (cyclohexane/EtOAc 100/0-80/20); Yield: 76%; ¹H NMR (300 MHz, CD₂Cl₂): δ 1.41 (s, 9H), 1.46 (s, 9H), 2.80 (t, J = 7.1 Hz, 2H), 3.29-3.40 (m, 6H), 3.52-3.59 (m, 4H), 4.59 (brs, 1H), 7.21 (d, J = 8.2 Hz, 2H), 7.46 (d, J = 7.3 Hz, 2H), 7.73 (d, J = 2.0 Hz, 1H), 8.30 (d, J = 2.0 Hz, 1H) ppm; [ES+ MS] m/z 541 (MH⁺). |

### Intermediate 115: tert-butyl 4-[3-chloro-6-(2-trimethylsilylethynyl)-2-quinolyl]piperazine-1-carboxylate

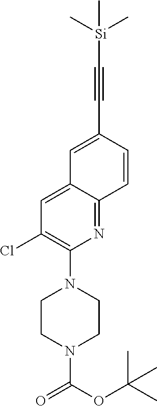 [SMILES: CC(C)(C)OC(=O)N1CCN(c2nc3ccc(C#C[Si](C)(C)C)cc3cc2Cl)CC1]

In a tube, were added intermediate 79 (1.07-2.01 mmol), Pd(PPh₃)₂Cl₂ (2 mol %) and CuI (6-10 mol %). The tube was purged with argon for 15 min, then ethynyl(trimethyl)silane (1.5 eq), TEA (14 eq) and 4-8 mL anhydrous MeCN were added. The reaction was heated at 100° C. under microwaves for 2 h. The reaction was filtered on a celite plug. The filtrate was diluted with EtOAc and washed twice with water. The aqueous layer was extracted once with EtOAc. The combined organic layers were washed with brine, dried over MgSO₄, filtered and the solvent was removed under reduced pressure. The crude was purified by reverse phase flash chromatography (MeCN/water 10/90-100/0) to give the title compound.

Yield: 78%; ¹H NMR (300 MHz, CD₂Cl₂): δ 0.27 (s, 9H), 1.47 (s, 9H), 3.42-3.48 (m, 4H), 3.57-3.63 (m, 4H), 7.62 (dd, J=1.8, 8.7 Hz, 1H), 7.72 (d, J=8.7 Hz, 1H), 7.77 (d, J=1.7 Hz, 1H), 8.01 (s, 1H) ppm; [ES+MS] m/z 444 (MH⁺).

### Intermediate 116: tert-butyl 4-(3-chloro-6-ethynyl-2-quinolyl)piperazine-1-carboxylate

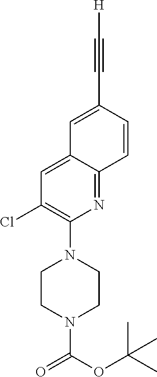 [SMILES: C#Cc1ccc2nc(N3CCN(C(=O)OC(C)(C)C)CC3)c(Cl)cc2c1]

TBAF 1M in THF (1.1 eq) was added to a solution of intermediate 115 (0.82-1.76 mmol) in 4-10 mL THF. The reaction was allowed to stir at RT for 1 h-3 h. The reaction was diluted with EtOAc and washed twice with water. The aqueous layer was extracted once with EtOAc. The combined organic layers were then washed with brine, dried over MgSO₄ and evaporated under reduced pressure to give the title compound. Yield: 100%; ¹H NMR (300 MHz, CD₂Cl₂): δ 1.47 (s, 9H), 3.21 (s, 1H), 3.43-3.49 (m, 4H), 3.58-3.63 (m, 4H), 7.65 (dd, J=1.8, 8.7 Hz, 1H), 7.74 (d, J=8.8 Hz, 1H), 7.81 (d, J=1.8 Hz, 1H), 8.03 (s, 1H) ppm; [ES+MS] m/z 372 (MH⁺).

### Intermediate 117: tert-butyl 4-[6-[1-[3-(tert-butoxycarbonylamino)propyl]triazol-4-yl]-3-chloro-2-quinolyl]piperazine-1-carboxylate

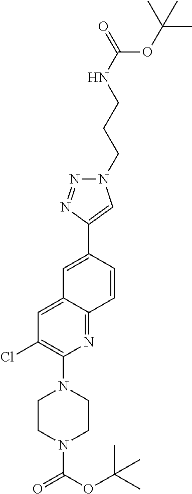 [SMILES: CC(C)(C)OC(=O)NCCCn1cc(-c2ccc3nc(N4CCN(C(=O)OC(C)(C)C)CC4)c(Cl)cc3c2)nn1]

To a tube charged with a freshly prepared solution of 1M sodium ascorbate (0.1 eq), CuSO₄·5H₂O (8 mol %), and TBTA (3 mol %) in THF/H₂O (3:1, 2.5 mL) was added intermediate 116 (0.40 mmol) and 3-azidopropan-1-amine (1 eq). The reaction mixture was stirred at RT for 2 h 30. The mixture was then washed twice with a saturated solution of K₂CO₃ and extracted three times with EtOAc. The combined organic layers were then washed with brine, dried over MgSO₄ and evaporated under reduced pressure. The residue was then dissolved in 1.5 mL of a 2:1 DCM/MeOH mixture and Boc2O (2 eq) was added at 0° C. After 48 h, the solvent was evaporated and the residue purified by reverse phase flash chromatography (water/MeCN 90/10-0/100) to give the title compound. Yield: 19%; ¹H NMR (300 MHz, CD₂Cl₂): δ 1.43 (s, 9H), 1.47 (s, 9H), 2.13 (p, J=6.6 Hz, 2H), 3.18 (q, J=6.4 Hz, 2H), 3.42-3.48 (m, 4H), 3.59-3.65 (m, 4H), 4.48 (t, J=6.8 Hz, 2H), 4.78 (brs, 1H), 7.86 (d, J=8.7 Hz, 1H), 8.00 (brs, 1H), 8.05 (dd, J=2.0, 8.7 Hz, 1H), 8.13 (s, 1H), 8.16 (d, J=1.9 Hz, 1H) ppm; [ES+MS] m/z 572 (MH⁺).

### Intermediate 118: tert-butyl 4-[6-[1-[2-(tert-butoxycarbonylamino)ethyl]triazol-4-yl]-3-chloro-2-quinolyl]piperazine-1-carboxylate

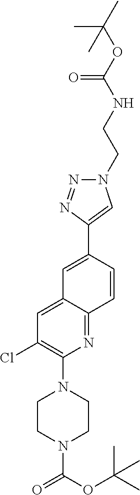 [SMILES: CC(C)(C)OC(=O)NCCn1cc(-c2ccc3nc(N4CCN(C(=O)OC(C)(C)C)CC4)c(Cl)cc3c2)nn1]

To a tube charged with a freshly prepared solution of 1M sodium ascorbate (0.1 eq), CuSO₄·5H₂O (9 mol %), and TBTA (Tris((1-benzyl-4-triazolyl)methyl)amine, 3 mol %) in THF/H₂O (3:1, 2.0 d mL) was added intermediate 116 (0.27 mmol), 2-azidoethanamine; hydrochloride (1 eq) and TEA (1 eq. The reaction mixture was stirred at RT for 2 h. The mixture was then washed twice with a saturated solution of K₂CO₃ and extracted once with EtOAc. The combined organic layers were then washed with brine, dried over MgSO₄ and evaporated under reduced pressure. The residue was dissolved in 0.6 mL DCM and Boc₂O (1 eq) was added at 0° C. After 4 h 30, the solvent was evaporated and the residue was purified by flash chromatography (DCM/EtOAc 100/0-70/30) to give the title compound. Yield: 87%; ¹H NMR (300 MHz, CD₂Cl₂): δ 1.42 (s, 9H), 1.47 (s, 9H), 3.41-3.48 (m, 4H), 3.58-3.71 (m, 6H), 4.53 (t, J=5.7 Hz, 2H), 4.98 (brs, 1H), 7.85 (d, J=8.7 Hz, 1H), 7.90 (s, 1H), 8.04 (dd, J=2.0, 8.7 Hz, 1H), 8.10 (s, 1H), 8.13 (d, J=1.8 Hz, 1H) ppm; [ES+MS] m/z 558 (MH⁺).

### Intermediate 119: tert-butyl 4-[3-chloro-6-[1-[2-(dimethylamino)ethyl]triazol-4-yl]-2-quinolyl]piperazine-1-carboxylate

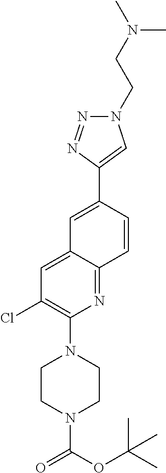 [SMILES: CN(C)CCn1cc(-c2ccc3nc(N4CCN(C(=O)OC(C)(C)C)CC4)c(Cl)cc3c2)nn1]

To a tube charged with a freshly prepared solution of sodium ascorbate 1M (0.1 eq), CuSO₄·5H₂O (0.14 eq), TBTA (3 mol %) and TEA (1 eq) in THF/H₂O (3:1, 2.0 mL) was added intermediate 116 (0.22 mmol) and 2-azido-N,N-dimethyl-ethanamine; hydrochloride (1 eq). The reaction mixture was stirred at RT for 20 h. The mixture was then washed twice with a saturated solution of K₂CO₃ and extracted once with EtOAc. The combined organic layers were then washed with brine, dried over MgSO₄ and evaporated under reduced pressure. The crude was then purified by reverse phase chromatography (water 0.1% formic acid/MeCN 0.1% formic acid 10/90-0/100) to give the title compound. Yield: 61%; ¹H NMR (300 MHz, CD₂Cl₂): δ 1.47 (s, 9H), 2.30 (s, 6H), 2.79 (t, J=6.1 Hz, 2H), 3.41-3.47 (m, 4H), 3.59-3.64 (m, 4H), 4.49 (t, J=6.1 Hz, 2H), 7.85 (d, J=8.7 Hz, 1H), 8.04-8.08 (m, 2H), 8.13 (s, 1H), 8.16 (d, J=1.9 Hz, 1H) ppm; [ES+MS] m/z 486 (MH⁺).

Intermediates 132-135: The tert-butyl 4-(6-bromo-3-chloro-2-quinolyl)piperazine-1-carboxylate 78 (0.2-0.6 mmol, 1 eq), appropriate phenylboronic acid (1.0 eq) and K₂CO₃ (1.6 eq) were dissolved in DME/EtOH/H₂O (2/1/2, 5.0-10.0 mL) under argon. Then was added Pd(PPh₃)₂Cl₂ (0.09-0.1 eq.), the suspension was heated at 90° C. under argon for 3 h to overnight. The reaction was quenched with water, extracted twice with CH₂Cl₂. The organic layer was washed with brine, dried over MgSO₄, evaporated under reduced pressure and purified by flash chromatography.

| Int | Structure | Physical data |
|---|---|---|
| 132 | 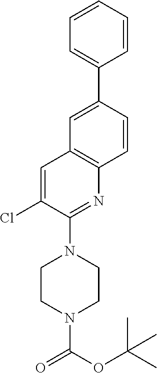 [SMILES: CC(C)(C)OC(=O)N1CCN(c2nc3ccc(-c4ccccc4)cc3cc2Cl)CC1] | tert-butyl 4-(3-chloro-6-phenyl-2- quinolyl)piperazine-1-carboxylate Purification 2 times by flash chromatography (cyclohexane/EtOAc 100/0-90/10); Yield: 27%; ¹H NMR (300 MHz, CD₂Cl₂): δ 1.48 (s, 9H), 3.43-3.47 (m, 4H), 3.61-3.64 (m, 4H), 7.35-7.41 (m, 1H), 7.46- 7.52 (m, 2H), 7.68-7.72 (m, 2H), 7.86-7.92 (m, 3H), 8.14 (s, 1H) ppm; [ES+ MS] m/z 424 (MH⁺). |
| 133 | 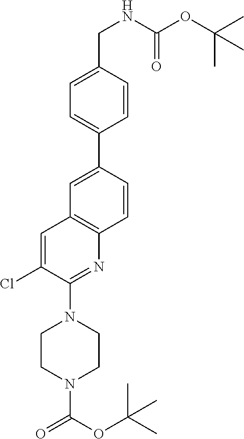 [SMILES: CC(C)(C)OC(=O)NCc1ccc(-c2ccc3nc(N4CCN(C(=O)OC(C)(C)C)CC4)c(Cl)cc3c2)cc1] | tert-butyl 4-[6-[4-[(tert- butoxycarbonylamino)methyl]phenyl]-3-chloro- 2-quinolyl]piperazine-1-carboxylate Purification by flash chromatography (cyclohexane/EtOAc 100/0-80/20); Yield: 55%; ¹H NMR (300 MHz, CD₂Cl₂): δ 1.47 (s, 9H), 1.49 (s, 9H), 3.43-3.46 (m, 4H), 3.61-3.64 (m, 4H), 4.35 (d, J = 6.1 Hz, 2H), 5.13 (br s, 1H), 7.39 (d, J = 8.2 Hz, 2H), 7.65 (d, J = 8.2 Hz, 2H), 7.83-7.87 (m, 3H), 8.10 (s, 1H) ppm; [ES+ MS] m/z 553 (MH⁺). |
| 134 | 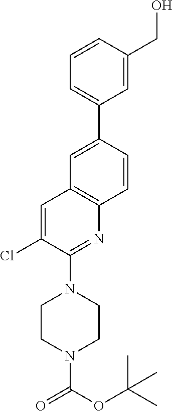 [SMILES: CC(C)(C)OC(=O)N1CCN(c2nc3ccc(-c4cccc(CO)c4)cc3cc2Cl)CC1] | tert-butyl 4-[3-chloro-6-[3- (hydroxymethyl)phenyl]-2-quinolyl]piperazine- 1-carboxylate Purification by flash chromatography (cyclohexane/EtOAc 100/0-60/40); Yield: 29%; ¹H NMR (300 MHz, CD₂Cl₂): δ 1.48 (s, 9H), 1.91 (t, 1H), 3.43-3.47 (m, 4H), 3.61-3.64 (m, 4H), 4.77 (d, J = 5.7 Hz, 2H), 7.38 (d, J = 7.7 Hz, 1H), 7.48 (t, J = 7.6 Hz, 1H), 7.62 (td, J = 1.6, 8.0 Hz, 1H), 7.70-7.72 (m, 1H), 7.86-7.93 (m, 3H), 8.13 (s, 1H) ppm; [ES+ MS] m/z 454 (MH⁺). |
| 135 | 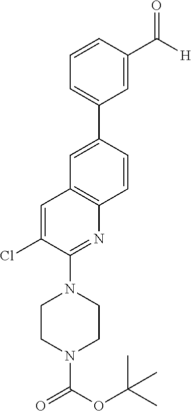 [SMILES: CC(C)(C)OC(=O)N1CCN(c2nc3ccc(-c4cccc(C=O)c4)cc3cc2Cl)CC1] | tert-butyl 4-[3-chloro-6-(3-formylphenyl)-2- quinolyl]piperazine-1-carboxylate Purification by flash chromatography (cyclohexane/EtOAc 100/0-70/30); Yield: 69%; ¹H NMR (300 MHz, CD₂Cl₂): δ 1.48 (s, 9H), 3.45-3.49 (m, 4H), 3.61-3.65 (m, 4H), 7.67 (t, J = 7.4 Hz, 1H), 7.87-7.93 (m, 4H), 7.96-8.00 (m, 1H), 8.15 (s, 1H), 8.20 (t, J = 1.5 Hz, 1H), MS] m/z 452 10.11 (s, 1H) ppm; [ES+ (MH⁺). |

Intermediates 136-137: Tert-butyl 4-[3-chloro-6-(3-formylphenyl)-2-quinolyl]piperazine-1-carboxylate 135 (0.13-0.22 mmol) was dissolved in anhydrous MeOH (0.6-0.9 mL) under argon atmosphere and MeNH₂ or Me₂NH (2 M in THF, 1.1-1.8 eq.) was added. The solution was stirred at room temperature for 24 h, then NaBH₄ (2.0-2.1 eq.) was added at 0. The suspension was stirred at room temperature overnight and was quenched with water at 0° C. The suspension was stirred at room temperature for 1 h then extracted twice with CH₂Cl₂. The organic layer was washed with brine, dried over MgSO₄ and evaporated under reduced pressure.

| Int | Structure | Physical data |
|---|---|---|
| 136 | 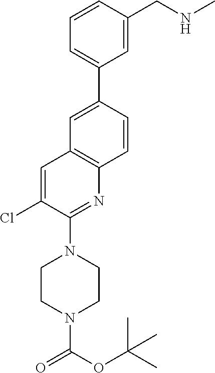 [SMILES: CNCc1cccc(-c2ccc3nc(N4CCN(C(=O)OC(C)(C)C)CC4)c(Cl)cc3c2)c1] | tert-butyl 4-[3-chloro-6-[3- (methylaminomethyl)phenyl]-2- quinolyl]piperazine-1-carboxylate Yield: 90%; ¹H NMR (300 MHz, CD₂Cl₂): δ 1.48 (s, 9H), 2.46 (s, 3H), 3.43-3.47 (m, 4H), 3.60-3.64 (m, 4H), 3.82 (s, 2H), 7.32-7.36 (m, 1H), 7.44 (t, J = 7.6 Hz, 1H), 7.58 (td, J = 1.5, 7.8 Hz, 1H), 7.67-7.68 (m, 1H), 7.85-7.93 (m, 3H), 8.13 (s, 1H) ppm; [ES+ MS] m/z 467 (MH⁺). |
| 137 | 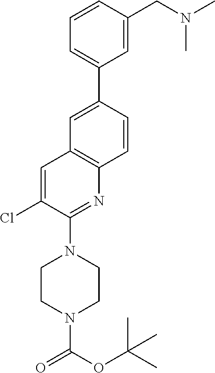 [SMILES: CN(C)Cc1cccc(-c2ccc3nc(N4CCN(C(=O)OC(C)(C)C)CC4)c(Cl)cc3c2)c1] | tert-butyl 4-[3-chloro-6-[3- [(dimethylamino)methyl]phenyl]-2- quinolyl]piperazine-1-carboxylate Yield: 58%; ¹H NMR (300 MHz, CD₂Cl₂): δ 1.48 (s, 9H), 2.27 (s, 6H), 3.42-3.47 (m, 4H), 3.53 (s, 2H), 3.60-3.64 (m, 4H), 7.33 (td, J = 1.6, 7.4 Hz, 1H), 7.43 (t, J = 7.6 Hz, 1H), 7.60 (td, J = 1.5, 7.8 Hz, 1H), 7.67-7.68 (m, 1H), 7.85-7.94 (m, 3H), 8.13 (s, 1H) ppm; [ES+ MS] m/z 481 (MH⁺). |

### Intermediate 138: tert-butyl 4-[6-[3-[(tert-butoxycarbonylamino)methyl]phenyl]-3-chloro-2-quinolyl]piperazine-1-carboxylate

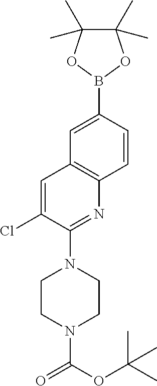 [SMILES: CC(C)(C)OC(=O)N1CCN(c2nc3ccc(B4OC(C)(C)C(C)(C)O4)cc3cc2Cl)CC1]

In a tube was added tert-butyl 4-(6-bromo-3-chloro-2-quinolyl)piperazine-1-carboxylate 78 (2.34 mmol, 1.0 eq.), bis(pinacolato)diboron (2.52 mmol, 1.1 eq.), K₂CO₃ (7.37 mmol, 3.1 eq.), Pd(dppf)Cl₂ (0.07 mmol, 0.03 eq.), dissolved in 50 mL of dioxane. The mixture was degassed for 40 minutes then heated to 100° C. After one night, Bis(pinacolato)diboron (0.41 mmol, 0.2 eq.), K₂CO₃ (2.25 mmol, 1.1 eq.) and Pd(dppf)Cl₂ (0.02 mmol, 0.01 eq.) were added then the mixture was stirred for 3 h. The reaction mixture was then washed with water and then extracted thrice with dichloromethane. The organic layers were washed with a saturated NaCl solution, dried over MgSO₄ and evaporated under vacuum. The crude product was purified by flash chromatography (cyclohexane/EtOAc 100/0-90/10) to give the title compound 138. Yield: 73%; ¹H NMR (300 MHz, CD₂Cl₂): δ 1.36 (s, 12H), 1.47 (s, 9H), 3.44-3.47 (m, 4H), 3.59-3.62 (m, 4H), 7.76 (d, J=8.4 Hz, 1H), 7.93 (dd, J=1.4, 8.4 Hz, 1H), 8.08 (s, 1H), 8.11 (br s, 1H) ppm; [ES+MS] m/z 474 (MH⁺).

Intermediates 139-152, 203-204: LiAlH₄ (1 M in THF, 2.0 eq.) was dissolved in THF or Et₂O (1.0-5.0 mL) then was added dropwise a solution of AlCl₃ (2.0 eq.) in THF/Et₂O (2:1, 3:1 or 5:1, 3.0-6.0 mL) or Et₂O (3.0 mL). The solution was stirred at room temperature for 20 m. Then the appropriate benzonitrile (1.2-2.8 mmol, 1.0 eq.) in THF (1.0-4.0 mL) was added dropwise. The suspension was stirred at room temperature for 1 h-3 h, then was quenched with water at 0° C. and stirred at room temperature for 1 h-overnight. The reaction was quenched with NH₃ in H₂O, extracted twice with CH₂Cl₂. The organic layer was washed with brine, dried over MgSO₄ and evaporated under reduced pressure.

| Int | Structure | Physical data |
|---|---|---|
| 139 | 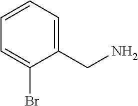 | (2-bromophenyl)methanamine Not isolated, ¹H NMR (300 MHz, CD₂Cl₂): δ 3.88 (s, 2H), 7.12 (td, J = 1.8, 7.8 Hz, 1H), 7.31 (td, J = 1.3, 7.4 Hz, 1H), 7.41 (dd, J = 1.6, 7.6 Hz, 1H), 7.54 (dd, J = 1.2, 7.9 Hz, 1H) ppm; [ES+ MS] m/z 186 (MH⁺). |
| 140 | 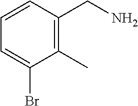 | (3-bromo-2-methyl-phenyl)methanamine Not isolated, [ES+ MS] m/z 200 (MH⁺). |
| 141 | 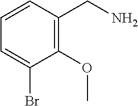 | (3-bromo-2-methoxy-phenyl)methanamine Not isolated, [ES+ MS] m/z 216 (MH⁺). |
| 142 | 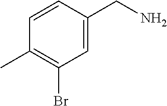 | (3-bromo-4-methyl-phenyl)methanamine Not isolated, [ES+ MS] m/z 200 (MH⁺). |
| 143 | 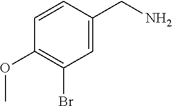 | (3-bromo-4-methoxy-phenyl)methanamine Not isolated, [ES+ MS] m/z 216 (MH⁺). |
| 144 | 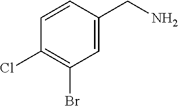 | (3-bromo-4-chloro-phenyl)methanamine Not isolated, [ES+ MS] m/z 220 (MH⁺). |
| 145 | 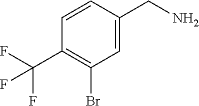 [SMILES: NCc1ccc(C(F)(F)F)c(Br)c1] | [3-bromo-4- (trifluoromethyl)phenyl]methanamine Not isolated, [ES+ MS] m/z 254 (MH⁺). |
| 146 | 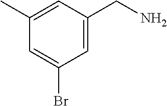 | (3-bromo-5-methyl-phenyl)methanamine Not isolated, [ES+ MS] m/z 200 (MH⁺). |
| 147 | 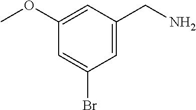 | (3-bromo-5-methoxy-phenyl)methanamine Not isolated, [ES+ MS] m/z 216 (MH⁺). |
| 148 | 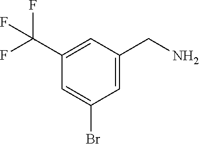 | [3-bromo-5- (trifluoromethyl)phenyl]methanamine Not isolated, [ES+ MS] m/z 254 (MH⁺). |
| 149 | 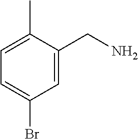 | (5-bromo-2-methyl-phenyl)methanamine Not isolated, [ES+ MS] m/z 200 (MH⁺). |
| 150 | 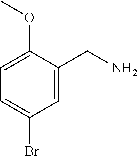 | (5-bromo-2-methoxy-phenyl)methanamine Not isolated, [ES+ MS] m/z 216 (MH⁺). |
| 151 | 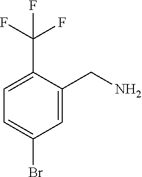 [SMILES: NCc1cc(Br)ccc1C(F)(F)F] | [5-bromo-2- (trifluoromethyl)phenyl]methanamine Not isolated, [ES+ MS] m/z 254 (MH⁺). |
| 152 | 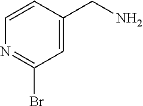 | (2-bromo-4-pyridyl)methanamine Not isolated, [ES+ MS] m/z 187 (MH⁺). |
| 203 | 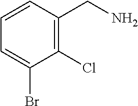 | (3-bromo-2-chloro-phenyl)methanamine Not isolated, [ES+ MS] m/z 220 (MH⁺). |
| 204 | 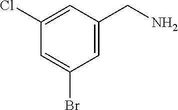 | (3-bromo-5-chloro-phenyl)methanamine Not isolated, [ES+ MS] m/z 220 (MH⁺). |

Intermediates 153-168, 205-206: The bromobenzylamine derivative (0.7-1.9 mmol, 1.0 eq.) was dissolved in CH₂Cl₂ (1.0-3.0 mL) then a solution of di-tert-butyl dicarbonate (1.0-3.0 eq.) in CH₂Cl₂ (2.0-3.0 mL) was added dropwise at 0° C. The reaction was stirred at room temperature for 1 h-2 days. The crude product was evaporated under reduced pressure and purified by flash or reverse chromatography.

| | | Starting | |
|---|---|---|---|
| Int | Structure | material | Physical data |
| 153 | 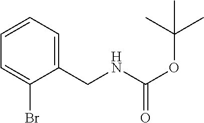 [SMILES: CC(C)(C)OC(=O)NCc1ccccc1Br] | 139 | tert-butyl N-[(2- bromophenyl)methyl]carbamate Yield: 62% over 2 steps; 1H NMR (300 MHz, CD₂Cl₂): δ 1.42 (s, 9H), 4.35 (d, J = 6.2 Hz), 5.08 (br s), 7.12-7.19 (m, 1H), 7.31 (td, J = 1.2, 7.6 Hz, 1H), 7.35-7.39 (m, 1H), 7.54 (dd, J = 1.1, 7.9 Hz, 1H) ppm; [ES+ MS] m/z 286 (MH⁺). |
| 154 | 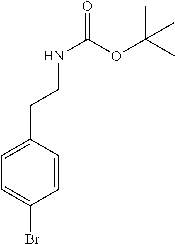 [SMILES: CC(C)(C)OC(=O)NCCc1ccc(Br)cc1] | commercial | tert-butyl N-[2-(4- bromophenyl)ethyl]carbamate Purification by flash chromatography (cyclohexane/EtOAc 100/0-70/30); Yield: 88%; ¹H NMR (300 MHz, CD₂Cl₂): δ 1.40 (s, 9H), 2.74 (t, J = 7.0 Hz, 2H), 3.31 (q, J = 6.6 Hz, 2H), 4.58 (br s, 1H), 7.09 (d, J = 8.4 Hz, 2H), 7.43 (d, J = 8.4 Hz, 2H) ppm; [ES+ MS] m/z 300 (MH⁺). |
| 155 | 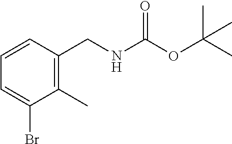 [SMILES: Cc1c(Br)cccc1CNC(=O)OC(C)(C)C] | 140 | tert-butyl N-[(3-bromo-2-methyl- phenyl)methyl]carbamate Purification by reverse chromatography (MeOH/H2O 10/90-100/0); Yield: 48% over 2 steps; ¹H NMR (300 MHz, CD₂Cl₂): δ 1.43 (s, 9H), 2.39 (s, 3H), 4.31 (m, 2H), 4.82 (br s, 1H), 7.04 (t, J = 7.9 Hz, 1H), 7.21 (d, J = 7.6 Hz, 1H), 7.48 (d, J = 7.1 Hz, 1H) ppm; not ionisable |
| 156 | 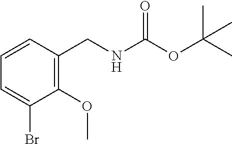 [SMILES: COc1c(Br)cccc1CNC(=O)OC(C)(C)C] | 141 | tert-butyl N-[(3-bromo-2-methoxy- phenyl)methyl]carbamate Purification by reverse phase chromatography (MeCN/H₂O/0.1% HCOOH 10/90-100/0); Yield: 32% over 2 steps; ¹H NMR (300 MHz, CD₂Cl₂): δ 1.42 (s, 9H), 3.84 (s, 3H), 4.34 (d, J = 6.1 Hz, 2H), 5.02 (br s, 1H), 6.99 (t, J = 7.8 Hz, 1H), 7.25 (dd, J = 1.6, 7.7 Hz, 1H), 7.47 (dd, J = 1.6, 7.8 Hz, 1H) ppm; not ionisable |
| 157 | 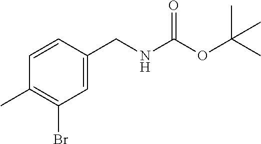 [SMILES: Cc1ccc(CNC(=O)OC(C)(C)C)cc1Br] | 142 | tert-butyl N-[(3-bromo-4-methyl- phenyl)methyl]carbamate Purification by flash chromatography (cyclohexane/EtOAc 100/0-80/20); Yield: 76% over 2 steps; ¹H NMR (300 MHz, CD₂Cl₂): δ 1.43 (s, 9H), 2.36 (s, 3H), 4.22 (d, J = 6.1 Hz, 2H), 4.93 (br s, 1H), 7.13 (dd, J = 1.6, 7.7 Hz, 1H), 7.20 (d, J = 7.7 Hz, 1H), 7.44 (d, J = 1.6 Hz, 1H) ppm; not ionisable |
| 158 | 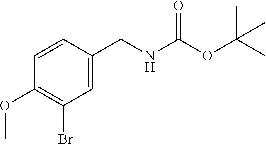 [SMILES: COc1ccc(CNC(=O)OC(C)(C)C)cc1Br] | 143 | tert-butyl N-[(3-bromo-4-methoxy- phenyl)methyl]carbamate Purification by flash chromatography (cyclohexane/EtOAc 100/0-80/20); Yield: 78% over 2 steps; ¹H NMR (300 MHz, CD₂Cl₂): δ 1.43 (s, 9H), 3.86 (s, 3H), 4.19 (d, J = 6.1 Hz, 2H), 4.92 (br s, 1H), 6.88 (d, J = 8.4 Hz, 1H), 7.20 (dd, J = 2.2, 8.4 Hz, 1H), 7.46 (d, J = 2.2 Hz, 1H) ppm; not ionisable |
| 159 | 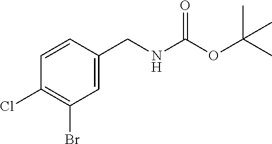 [SMILES: CC(C)(C)OC(=O)NCc1ccc(Cl)c(Br)c1] | 144 | tert-butyl N-[(3-bromo-4-chloro- phenyl)methyl]carbamate Purification by flash chromatography (cyclohexane/EtOAc 100/0-80/20); Yield: 76% over 2 steps; ¹H NMR (300 MHz, CD₂Cl₂): δ 1.43 (s, 9H), 4.24 (d, J = 6.4 Hz, 2H), 4.99 (br s, 1H), 7.18 (dd, J = 2.2, 8.3 Hz, 1H), 7.41 (d, J = 8.4 Hz, 1H), 7.55 (d, J = 1.9 Hz, 1H) ppm; not ionisable |
| 160 | 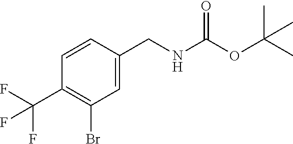 [SMILES: CC(C)(C)OC(=O)NCc1ccc(C(F)(F)F)c(Br)c1] | 145 | tert-butyl N-[[3-bromo-4- (trifluoromethyl)phenyl]methyl]carbamate Purification by flash chromatography (cyclohexane/EtOAc 100/0-80/20); Yield: 67% over 2 steps; ¹H NMR (300 MHz, CD₂Cl₂): δ 1.44 (s, 9H), 4.32 (d, J = 6.1 Hz, 2H), 5.07 (br s, 1H), 7.32-7.36 (m, 1H), 7.64-7.67 (m, 2H) ppm; not ionisable |
| 161 | 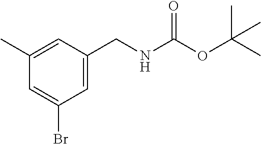 [SMILES: Cc1cc(Br)cc(CNC(=O)OC(C)(C)C)c1] | 146 | tert-butyl N-[(3-bromo-5-methyl- phenyl)methyl]carbamate Purification by flash chromatography (cyclohexane/EtOAc 100/0-80/20); Yield: 69% over 2 steps; ¹H NMR (300 MHz, CD₂Cl₂): δ 1.44 (s, 9H), 2.31 (s, 3H), 4.22 (d, J = 6.2 Hz, 2H), 4.94 (br s, 1H), 7.02-7.03 (m, 1H), 7.21-7.24 (m, 2H) ppm; not ionisable |
| 162 | 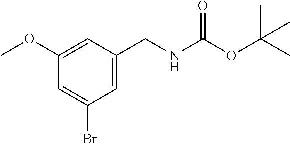 [SMILES: COc1cc(Br)cc(CNC(=O)OC(C)(C)C)c1] | 147 | tert-butyl N-[(3-bromo-5-methoxy- phenyl) methyl]carbamate Purification by flash chromatography (cyclohexane/EtOAc 100/0-80/20); Yield: 70% over 2 steps; ¹H NMR (300 MHz, CD₂Cl₂): δ 1.44 (s, 9H), 3.78 (s, 3H), 4.23 (d, J = 6.2 Hz, 2H), 4.98 (br s, 1H), 6.76-6.77 (m, 1H), 6.95 (m, 1H), 7.00-7.01 (m, 1H) ppm; not ionisable |
| 163 | 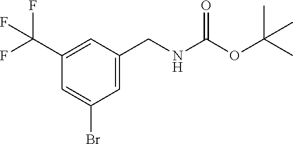 [SMILES: CC(C)(C)OC(=O)NCc1cc(Br)cc(C(F)(F)F)c1] | 148 | tert-butyl N-[[3-bromo-5- (trifluoromethyl)phenyl]methyl]carbamate Purification by flash chromatography (cyclohexane/EtOAc 100/0-80/20); Yield: 55% over 2 steps; ¹H NMR (300 MHz, CD₂Cl₂): δ 1.44 (s, 9H), 4.33 (d, J = 6.1 Hz, 2H), 5.06 (br s, 1H), 7.48-7.49 (m, 1H), 7.64-7.66 (m, 1H), 7.66-7.68 (m, 1H) ppm; not ionisable |
| 164 | 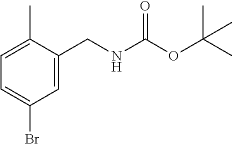 [SMILES: Cc1ccc(Br)cc1CNC(=O)OC(C)(C)C] | 149 | tert-butyl N-[(5-bromo-2-methyl- phenyl)methyl]carbamate Purification by flash chromatography (cyclohexane/EtOAc 100/0-80/20); Yield: 70% over 2 steps; ¹H NMR (300 MHz, CD₂Cl₂): δ 1.44 (s, 9H), 2.25 (s, 3H), 4.24 (d, J = 6.0 Hz, 2H), 4.84 (br s, 1H), 7.05 (d, J = 8.1 Hz, 1H), 7.29 (dd, J = 2.1, 8.1 Hz, 1H), 7.37 (d, J = 1.7 Hz, 1H) ppm; not ionisable |
| 165 | 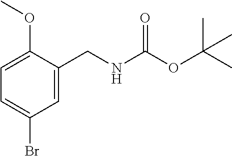 [SMILES: COc1ccc(Br)cc1CNC(=O)OC(C)(C)C] | 150 | tert-butyl N-[(5-bromo-2-methoxy- phenyl)methyl]carbamate Purification by flash chromatography (cyclohexane/EtOAc 100/0-80/20); Yield: 69% over 2 steps; ¹H NMR (300 MHz, CD₂Cl₂): δ 1.42 (s, 9H), 3.18 (s, 3H), 4.22 (d, J = 6.3 Hz, 2H), 5.02 (br s, 1H), 6.77 (m, 1H), 7.35 (m, 2H) ppm; not ionisable |
| 166 | 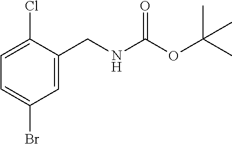 [SMILES: CC(C)(C)OC(=O)NCc1cc(Br)ccc1Cl] | commercial | tert-butyl N-[(5-bromo-2-chloro- phenyl) methyl]carbamate Yield: 95%; ¹H NMR (300 MHz, CD₂Cl₂): δ 1.44 (s, 9H), 4.33 (d, J = 6.8 Hz, 2H), 5.06 (br s, 1H), 7.24 (d, J = 8.2 Hz, 1H), 7.36 (dd, J = 2.4, 8.4 Hz, 1H), 7.51 (d, J = 2.3 Hz, 1H) ppm; not ionisable |
| 167 | 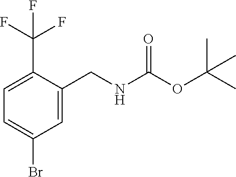 [SMILES: CC(C)(C)OC(=O)NCc1cc(Br)ccc1C(F)(F)F] | 151 | tert-butyl N-[[5-bromo-2- (trifluoromethyl)phenyl]methyl]carbamate Purification by flash chromatography (CH₂Cl₂/EtOAc 100/0-70/30); Yield: 73% over 2 steps; ¹H NMR (300 MHz, CD₂Cl₂): δ 1.45 (s, 9H), 4.45 (d, J = 6.1 Hz, 2H), 5.01 (br s, 1H), 7.52-7.53 (m, 2H), 7.72 (s, 1H) ppm; not ionisable |
| 168 | 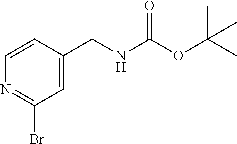 [SMILES: CC(C)(C)OC(=O)NCc1ccnc(Br)c1] | 152 | tert-butyl N-[(2-bromo-4- pyridyl)methyl]carbamate Purification by reverse chromatography (MeOH/H₂O 10/90-100/0); Yield: 13% over 2 steps; ¹H NMR (300 MHz, CD₂Cl₂): δ 1.44 (s, 9H), 4.28 (d, J = 6.4 Hz, 2H), 5.04 (br s, 1H), 7.16-7.18 (m, 1H), 7.40-7.41 (m, 1H), 8.28 (d, J = 5.1 Hz, 1H) ppm; [ES+ MS] m/z 287 (MH⁺). |
| 205 | 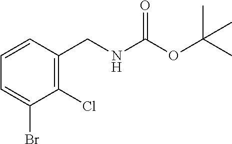 [SMILES: CC(C)(C)OC(=O)NCc1cccc(Br)c1Cl] | 203 | tert-butyl N-[(3-bromo-2-chloro- phenyl)methyl]carbamate Purification by flash chromatography (CH₂Cl₂/EtOAc 100/0-80/20); Yield: 38% over 2 steps; ¹H NMR (300 MHz, CD₂Cl₂): δ 1.43 (s, 9H), 4.39 (d, J = 6.3 Hz, 2H), 5.10 (br s, 1H), 7.15 (t, J = 7.8 Hz, 1H), 7.32-7.36 (m, 1H), 7.57 (dd, J = 1.6, 8.0 Hz, 1H) ppm; not ionisable |
| 206 | 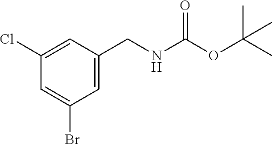 [SMILES: CC(C)(C)OC(=O)NCc1cc(Cl)cc(Br)c1] | 204 | tert-butyl N-[(3-bromo-5-chloro- phenyl)methyl]carbamate Purification by flash chromatography (CH₂Cl₂/EtOAc 100/0-80/20); Yield: 73% over 2 steps; ¹H NMR (300 MHz, CD₂Cl₂): δ 1.44 (s, 9H), 4.24 (d, J = 6.2 Hz, 2H), 5.01 (br s, 1H), 7.22-7.24 (m, 1H), 7.33-7.34 (m, 1H), 7.42-7.43 (m, 1H) ppm; not ionisable |

Intermediates 169-177: To a solution of bromobenzylamine derivative (1.0-1.3 mmol, 1.0 eq.) in TH/H₂O (1:1, 1.9-2.2 mL) was added di-tert-butyl dicarbonate (1.5-1.6 eq.) and NaHCO₃ (2.0-2.1 eq.). The reaction was stirred at room temperature for 1 h-3 h. The reaction was quenched with water, extracted twice with EtOAc. The organic layer was washed with brine, dried over MgSO₄, evaporated under reduced pressure and purified by flash chromatography.

| Int | Structure | Physical data |
|---|---|---|
| 169 | 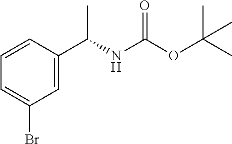 [SMILES: C[C@H](NC(=O)OC(C)(C)C)c1cccc(Br)c1] | tert-butyl N-[(1S)-1-(3- bromophenyl)ethyl]carbamate Purification by flash chromatography (cyclohexane/EtOAc 100/0-90/10); Yield: 90%; ¹H NMR (300 MHz, CD₂Cl₂): δ 1.39 (s, 9H), 1.41 (s, 3H), 4.69 (br s, 1H), 4.87 (br s, 1H), 7.19-7.26 (m, 2H), 7.38 (dt, J = 2.0, 6.9 Hz, 1H), 7.44-7.46 (m, 1H) ppm; [ES+ MS] m/z 300 (MH⁺) |
| 170 | 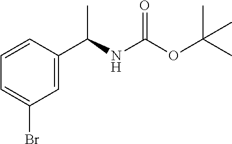 [SMILES: C[C@@H](NC(=O)OC(C)(C)C)c1cccc(Br)c1] | tert-butyl N-[(1R)-1-(3- bromophenyl)ethyl]carbamate Purification by flash chromatography (cyclohexane/EtOAc 100/0-90/10); Yield: 100%; ¹H NMR (300 MHz, CD₂Cl₂): δ 1.39 (s, 9H), 1.41 (s, 3H), 4.69 (br s, 1H), 4.87 (br s, 1H), 7.19-7.26 (m, 2H), 7.38 (dt, J = 2.0, 6.9 Hz, 1H), 7.44-7.46 (m, 1H) ppm; [ES+ MS] m/z 300 (MH⁺). |
| 171 | 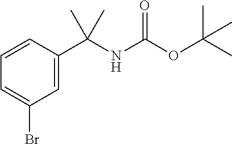 [SMILES: CC(C)(C)OC(=O)NC(C)(C)c1cccc(Br)c1] | tert-butyl N-[1-(3-bromophenyl)-1-methyl- ethyl]carbamate Purification by flash chromatography (cyclohexane/EtOAc 100/0-90/10); Yield: 35%; ¹H NMR (300 MHz, CD₂Cl₂): δ 1.36 (s, 9H), 1.57 (s, 6H), 5.02 (br s, 1H), 7.20 (t, J = 7.8 Hz, 1H), 7.31-7.37 (m, 2H), 7.53 (t, J = 1.9 Hz, 1H) ppm; [ES+ MS] m/z 314 (MH⁺) |
| 172 | 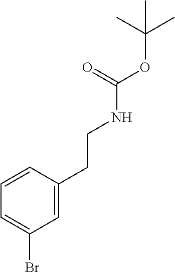 [SMILES: CC(C)(C)OC(=O)NCCc1cccc(Br)c1] | tert-butyl N-[2-(3-bromophenyl)ethyl]carbamate Purification by flash chromatography (cyclohexane/EtOAc 100/0-90/10); Yield: 81%; ¹H NMR (300 MHz, CD₂Cl₂): δ 1.41 (s, 9H), 2.76 (t, J = 7.0 Hz, 2H), 3.32 (q, J = 6.8 Hz, 2H), 4.58 (br s, 1H), 7.13-7.22 (m, 2H), 7.34-38 (m, 2H) ppm; [ES+ MS] m/z 300 (MH⁺). |
| 173 | 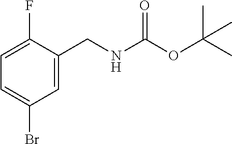 [SMILES: CC(C)(C)OC(=O)NCc1cc(Br)ccc1F] | tert-butyl N-[(5-bromo-2-fluoro- phenyl)methyl]carbamate Purification by flash chromatography (cyclohexane/EtOAc 100/0-90/10); Yield: 88%; ¹H NMR (300 MHz, CD₂Cl₂): δ 1.43 (s, 9H), 4.30 (d, J = 6.0 Hz, 2H), 4.99 (br s, 1H), 6.95 (dd, J = 8.8, 9.6 Hz, 1H), 7.34-7.40 (m, 1H), 7.46 (dd, J = 2.5, 6.6 Hz, 1H) ppm; [ES+ MS] m/z 304 (MH⁺). |
| 174 | 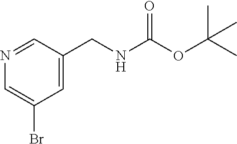 [SMILES: CC(C)(C)OC(=O)NCc1cncc(Br)c1] | tert-butyl N-[(5-bromo-3- pyridyl)methyl]carbamate Purification by flash chromatography (cyclohexane/EtOAc 100/0-70/30); Yield: 89%; ¹H NMR (300 MHz, CD₂Cl₂): δ 1.43 (s, 9H), 4.28 (d, J = 6.1 Hz, 2H), 5.02 (br s, 1H), 7.77-7.79 (m, 1H), 8.43 (d, J = 1.7 Hz, 1H), 8.55 (d, J = 2.1 Hz, 1H) ppm; [ES+ MS] m/z 287 (MH⁺). |
| 175 | 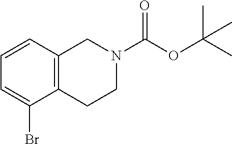 [SMILES: CC(C)(C)OC(=O)N1CCc2c(Br)cccc2C1] | tert-butyl 5-bromo-3,4-dihydro-1H-isoquinoline-2- carboxylate Purification by flash chromatography (cyclohexane/EtOAc 100/0-90/10); Yield: 93%; ¹H NMR (300 MHz, CD₂Cl₂): δ 1.46 (s, 9H), 2.83 (d, J = 6.0 Hz, 2H), 3.64 (d, J = 6.0 Hz, 2H), 4.55 (s, 2H), 7.02-7.10 (m, 2H), 7.41-7.45 (m, 1H) ppm; [ES+ MS] m/z 312 (MH⁺). |
| 176 | 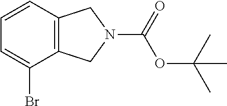 [SMILES: CC(C)(C)OC(=O)N1Cc2cccc(Br)c2C1] | tert-butyl 4-bromoisoindoline-2-carboxylate Purification by flash chromatography (cyclohexane/EtOAc 100/0-90/10); Yield: 87%; ¹H NMR (300 MHz, CD₂Cl₂): δ 1.50 (s, 9H), 4.60-4.62 (m, 2H), 4.72-4.74 (m, 2H), 7.13-7.24 (m, 2H), 7.41 (d, J = 8.3 Hz, 1H) ppm; not ionisable |
| 177 | 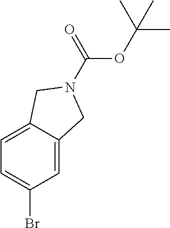 [SMILES: CC(C)(C)OC(=O)N1Cc2ccc(Br)cc2C1] | tert-butyl 5-bromoisoindoline-2-carboxylate Purification by flash chromatography (cyclohexane/EtOAc 100/0-90/10); Yield: 83%; ¹H NMR (300 MHz, CD₂Cl₂): δ 1.49 (s, 9H), 4.58-4.62 (m, 4H), 7.11-7.17 (dd, J = 8.4, 11.3 Hz, 1H), 7.38- 7.43 (m, 2H) ppm; not ionisable |

Intermediates 178-183: The tert-butyl 4-[3-chloro-6-(4,4,5,5-tetramethyl-1,3,2-dioxaborolan-2-yl)-2-quinolyl]piperazine-1-carboxylate 138 (0.25-32 mmol, 1 eq), appropriate phenylbromide (1.0-1.1 eq) and K₂CO₃ (1.6-1.7 eq) were dissolved in DME/EtOH/H₂O (2/1/2, 5.0 mL) under argon. Then was added Pd(PPh₃)₂Cl₂ (0.1 eq.), the suspension was heated at 90° C. under argon for 3 h. The reaction was quenched with water, extracted twice with CH₂Cl₂. The organic layer was then washed with brine, dried over MgSO₄ and evaporated under reduced pressure and purified by flash chromatography.

| | | Phenyl | |
|---|---|---|---|
| Int | Structure | bromide | Physical data |
| 178 | 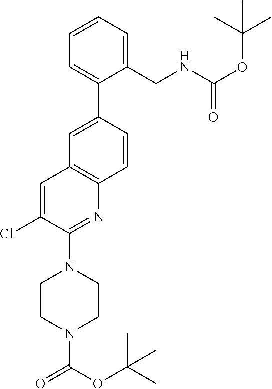 [SMILES: CC(C)(C)OC(=O)NCc1ccccc1-c1ccc2nc(N3CCN(C(=O)OC(C)(C)C)CC3)c(Cl)cc2c1] | 153 | tert-butyl 4-[6-[2-[(tert- butoxycarbonylamino)methyl]phenyl]- 3-chloro-2-quinolyl]piperazine-1- carboxylate Purification by flash chromatography (cyclohexane/EtOAc 100/0-80/20); Yield: 68%; ¹H NMR (300 MHz, CD₂Cl₂): δ 1.43 (s, 9H), 1.48 (s, 9H), 3.43-3.47 (m, 4H), 3.61-3.65 (m, 4H), 4.26 (d, J = 5.9 Hz, 2H), 4.82 (br s, 1H), 7.29-7.48 (m, 4H), 7.56-7.60 (m, 2H), 7.85 (dd, J = 0.6, 9.2 Hz, 1H), 8.09 (s, 1H) ppm; [ES+ MS] m/z 553 (MH⁺). |
| 179 | 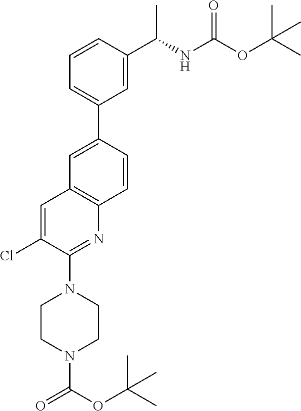 [SMILES: C[C@H](NC(=O)OC(C)(C)C)c1cccc(-c2ccc3nc(N4CCN(C(=O)OC(C)(C)C)CC4)c(Cl)cc3c2)c1] | 169 | tert-butyl 4-[6-[3-[(1S)-1-(tert- butoxycarbonylamino)ethyl]phenyl]-3- chloro-2-quinolyl]piperazine-1- carboxylate Purification by flash chromatography (cyclohexane/EtOAc 100/0-80/20); Yield: 47%; ¹H NMR (300 MHz, CD₂Cl₂): δ 1.41 (s, 9H), 1.48 (s, 9H), 1.57 (s, 3H), 3.43- 3.47 (m, 4H), 3.60-3.65 (m, 4H), 4.82 (br s, 1H), 4.98 (br s, 1H), 7.32 (d, J = 7.7 Hz, 1H), 7.45 (t, J = 7.6 Hz, 1H), 7.56-7.63 (m, 2H), 7.86-7.89 (m, 3H), 8.13 (s, 1H) ppm; [ES+ MS] m/z 567 (MH⁺). |
| 180 | 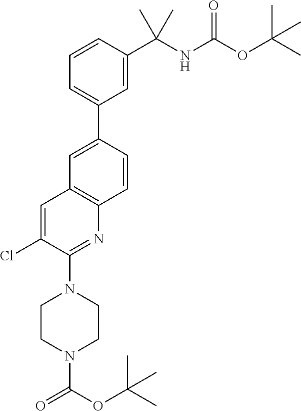 [SMILES: CC(C)(C)OC(=O)NC(C)(C)c1cccc(-c2ccc3nc(N4CCN(C(=O)OC(C)(C)C)CC4)c(Cl)cc3c2)c1] | 171 | tert-butyl 4-[6-[3-[1-(tert- butoxycarbonylamino)-1-methyl- ethyl]phenyl]-3-chloro-2- quinolyl]piperazine-1-carboxylate Purification by flash chromatography (cyclohexane/EtOAc 100/0-80/20); Yield: 60%; ¹H NMR (300 MHz, CD₂Cl₂): δ 1.43 (s, 9H), 1.48 (s, 9H), 1.66 (s, 6H), 3.43- 3.47 (m, 4H), 3.60-3.65 (m, 4H), 5.11 (br s, 1H), 7.42-7.47 (m, 2H), 7.53-7.57 (m, 1H), 7.70-7.72 (m, 1H), 7.85-7.89 (m, 3H), 8.13 (s, 1H) ppm; [ES+ MS] m/z 581 (MH⁺). |
| 181 | 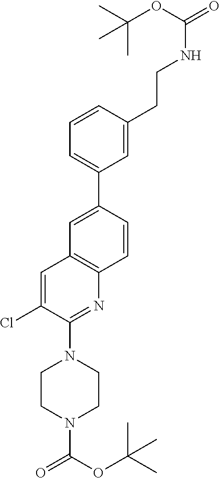 [SMILES: CC(C)(C)OC(=O)NCCc1cccc(-c2ccc3nc(N4CCN(C(=O)OC(C)(C)C)CC4)c(Cl)cc3c2)c1] | 172 | tert-butyl 4-[6-[3-[2-(tert- butoxycarbonylamino)ethyl]phenyl]-3- chloro-2-quinolyl]piperazine-1- carboxylate Purification by flash chromatography (cyclohexane/EtOAc 100/0-80/20); Yield: 44%; ¹H NMR (300 MHz, CD₂Cl₂): δ 1.40 (s, 9H), 1.48 (s, 9H), 2.85-2.91 (m, 2H), 3.37-3.47 (m, 6H), 3.60-3.64 (m, 4H), 4.65 (br s, 1H), 7.21-7.25 (m, 1H), 7.42 (d, J = 7.6 Hz, 1H), 7.53-7.58 (m, 2H), 7.86-7.90 (m, 3H), 8.32 (s, 1H) ppm; [ES+ MS] m/z 567 (MH⁺). |
| 182 | 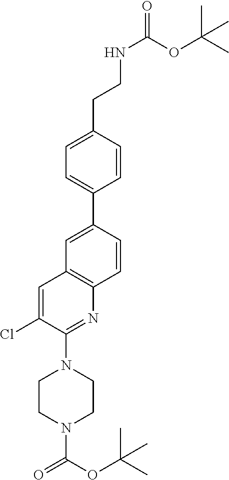 [SMILES: CC(C)(C)OC(=O)NCCc1ccc(-c2ccc3nc(N4CCN(C(=O)OC(C)(C)C)CC4)c(Cl)cc3c2)cc1] | 154 | tert-butyl 4-[6-[4-[2-(tert- butoxycarbonylamino)ethyl]phenyl]-3- chloro-2-quinolyl]piperazine-1- carboxylate Purification by flash chromatography (cyclohexane/EtOAc 100/0-70/30); Yield: 28%; ¹H NMR (300 MHz, CD₂Cl₂): δ 1.42 (s, 9H), 1.48 (s, 9H), 2.84 (t, J = 7.0 Hz, 2H), 3.35-3.47 (m, 6H), 3.60-3.64 (m, 4H), 4.64 (br s, 1H), 7.32 (d, J = 8.3 Hz, 2H), 7.65 (d, J = 8.3 Hz, 2H), 7.84-7.88 (m, 3H), 8.13 (s, 1H) ppm; [ES+ MS] m/z 567 (MH⁺). |
| 183 | 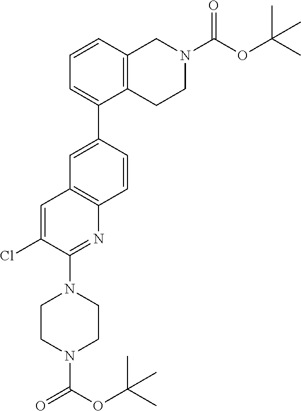 [SMILES: CC(C)(C)OC(=O)N1CCN(c2nc3ccc(-c4cccc5c4CCN(C(=O)OC(C)(C)C)C5)cc3cc2Cl)CC1] | | tert-butyl 5-[2-(4-tert- butoxycarbonylpiperazin-1-yl)-3- chloro-6-quinolyl]-3,4-dihydro-1H- isoquinoline-2-carboxylate Purification by flash chromatography (cyclohexane/EtOAc 100/0-80/20); Yield: 37%; ¹H NMR (300 MHz, CD₂Cl₂): δ 1.47 (s, 9H), 1.48 (s, 9H), 2.74 (t, J = 5.8 Hz, 2H), 3.43-3.47 (m, 4H), 3.51 (t, J = 5.8 Hz, 2H), 3.60-3.65 (m, 4H), 4.63 (s, 2H), 7.16- 7.31 (m, 3H), 7.56-7.60 (m, 2H), 7.85 (dd, J = 0.6, 9.1 Hz, 1H), 8.10 (s, 1H) ppm; [ES+ MS] m/z 579 (MH⁺). |

Intermediates 184-202, 207-208: The tert-butyl 4-[3-chloro-6-(4,4,5,5-tetramethyl-1,3,2-dioxaborolan-2-yl)-2-quinolyl]piperazine-h-carboxylate 138 (0.11-0.16 mmol, 1 eq), appropriate phenylbromide (1.1-1.7 eq) were dissolved in 1,4-dioxane (0.9-1.4 mL). Then Na₂CO₃ (2 M in water, 0.9-1.4 mL) and Pd(PPh₃)₄ (0.1 eq.) were added and the suspension was heated under microwave irradiation at 120 00 for 30 min. The reaction was filtered in a plug of celite, quenched with water, extracted twice with EtOAc. The organic layer was then washed with brine, dried over MgSO₄, evaporated under reduced pressure and purified by flash or reverse chromatography.

| | | Phenyl | |
|---|---|---|---|
| Int | Structure | bromide | Physical data |
| 184 | 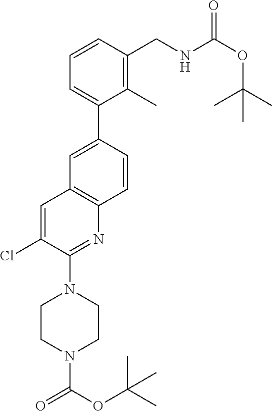 [SMILES: Cc1c(CNC(=O)OC(C)(C)C)cccc1-c1ccc2nc(N3CCN(C(=O)OC(C)(C)C)CC3)c(Cl)cc2c1] | 155 | tert-butyl 4-[6-[3-[(tert- butoxycarbonylamino)methyl]-2-methyl- phenyl]-3-chloro-2-quinolyl]piperazine-1- carboxylate Purification by reverse chromatography (MeOH/H₂O 10/90-100/0); Yield: 33%; ¹H NMR (300 MHz, CD₂Cl₂): δ 1.45 (s, 9H), 1.48 (s, 9H), 2.20 (s, 3H), 3.43-3.46 (m, 4H), 3.61- 3.64 (m, 4H), 4.36 (d, J = 5.8 Hz, 2H), 4.88 (br s, 1H), 7.20-7.25 (m, 2H), 7.28-7.21 (m, 1H), 7.55-7.58 (m, 2H) 7.84 (dd, J = 0.8, 9.2 Hz, 1H), 8.10 (s, 1H) ppm; [ES+ MS] m/z 567 (MH⁺). |
| 185 | 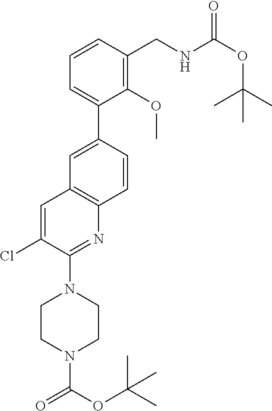 [SMILES: COc1c(CNC(=O)OC(C)(C)C)cccc1-c1ccc2nc(N3CCN(C(=O)OC(C)(C)C)CC3)c(Cl)cc2c1] | 156 | tert-butyl 4-[6-[3-[(tert- butoxycarbonylamino)methyl]-2-methoxy- phenyl]-3-chloro-2-quinolyl]piperazine-1- carboxylate Purification by flash chromatography (cyclohexane/EtOAc 100/0-80/20); Yield: 92%; [ES+ MS] m/z 583 (MH⁺) |
| 186 | 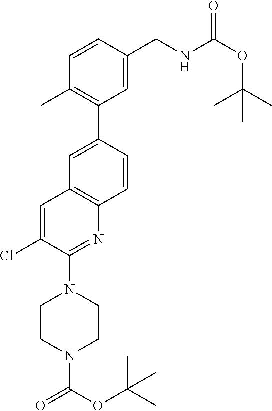 [SMILES: Cc1ccc(CNC(=O)OC(C)(C)C)cc1-c1ccc2nc(N3CCN(C(=O)OC(C)(C)C)CC3)c(Cl)cc2c1] | 157 | tert-butyl 4-[6-[5-[(tert- butoxycarbonylamino)methyl]-2-methyl- phenyl]-3-chloro-2-quinolyl]piperazine-1- carboxylate Purification by flash chromatography (cyclohexane/EtOAc 100/0-80/20); [ES+ MS] m/z 567 (MH⁺) |
| 187 | 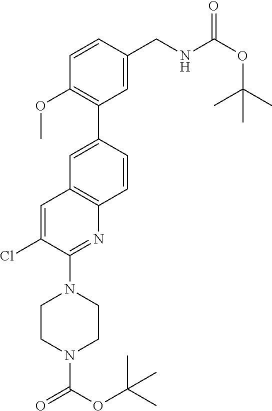 [SMILES: COc1ccc(CNC(=O)OC(C)(C)C)cc1-c1ccc2nc(N3CCN(C(=O)OC(C)(C)C)CC3)c(Cl)cc2c1] | 158 | tert-butyl 4-[6-[5-[(tert- butoxycarbonylamino)methyl]-2-methoxy- phenyl]-3-chloro-2-quinolyl]piperazine-1- carboxylate Purification by reverse chromatography (MeOH/H₂O 10/90-100/0); Yield: 57%; ¹H NMR (300 MHz, CD₂Cl₂): δ 1.44 (s, 9H), 1.48 (s, 9H), 3.42-3.46 (m, 4H), 3.61-3.64 (m, 4H), 3.82 (s, 3H), 4.30 (d, J = 5.9 Hz, 2H), 4.95 (br s, 1H), 6.99 (d, J = 8.3 Hz, 1H), 7.21-7.26 (m, 2H), 7.77-7.81 (m, 3H), 8.10 (s, 1H) ppm; [ES+ MS] m/z 583 (MH⁺). |
| 188 | 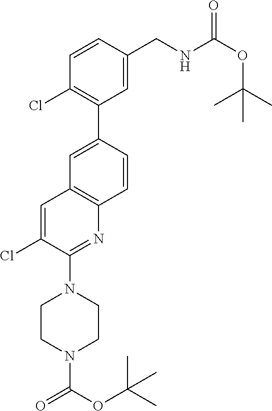 [SMILES: CC(C)(C)OC(=O)NCc1ccc(Cl)c(-c2ccc3nc(N4CCN(C(=O)OC(C)(C)C)CC4)c(Cl)cc3c2)c1] | 159 | tert-butyl 4-[6-[5-[(tert- butoxycarbonylamino)methyl]-2-chloro- phenyl]-3-chloro-2-quinolyl]piperazine-1- carboxylate Purification by flash chromatography (cyclohexane/EtOAc 100/0-80/20); [ES+ MS] m/z 587 (MH⁺). |
| 189 | 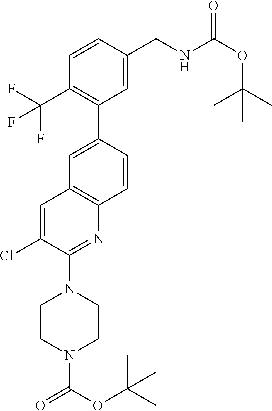 [SMILES: CC(C)(C)OC(=O)NCc1ccc(C(F)(F)F)c(-c2ccc3nc(N4CCN(C(=O)OC(C)(C)C)CC4)c(Cl)cc3c2)c1] | 160 | tert-butyl 4-[6-[5-[(tert- butoxycarbonylamino)methyl]-2- (trifluoromethyl)phenyl]-3-chloro-2- quinolyl]piperazine-1-carboxylate Purification by flash chromatography (cyclohexane/EtOAc 100/0-80/20); [ES+ MS] m/z 621 (MH⁺). |
| 190 | 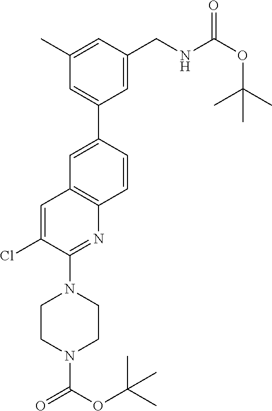 [SMILES: Cc1cc(CNC(=O)OC(C)(C)C)cc(-c2ccc3nc(N4CCN(C(=O)OC(C)(C)C)CC4)c(Cl)cc3c2)c1] | 161 | tert-butyl 4-[6-[3-[(tert- butoxycarbonylamino)methyl]-5-methyl- phenyl]-3-chloro-2-quinolyl]piperazine-1- carboxylate Purification by flash chromatography (cyclohexane/EtOAc 100/0-80/20); Yield: 81%; ¹H NMR (300 MHz, CD₂Cl₂): δ 1.45 (s, 9H), 1.48 (s, 9H), 2.43 (s, 3H), 3.43-3.46 (m, 4H), 3.60-3.64 (m, 4H), 4.34 (d, J = 6.1 Hz, 2H), 7.11-7.13 (m, 1H), 7.40-7.41 (m, 2H), 7.85-7.86 (m, 1H) 7.87-7.88 (m, 2H), 8.12 (s, 1H) ppm; [ES+ MS] m/z 567 (MH⁺) |
| 191 | 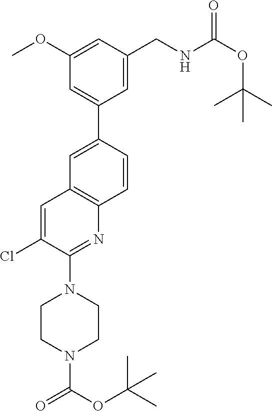 [SMILES: COc1cc(CNC(=O)OC(C)(C)C)cc(-c2ccc3nc(N4CCN(C(=O)OC(C)(C)C)CC4)c(Cl)cc3c2)c1] | 162 | tert-butyl 4-[6-[3-[(tert- butoxycarbonylamino)methyl]-5-methoxy- phenyl]-3-chloro-2-quinolyl]piperazine-1- carboxylate Purification by flash chromatography (cyclohexane/EtOAc 100/0-80/20); Yield: 98%; ¹H NMR (300 MHz, CD₂Cl₂): δ 1.46 (s, 9H), 1.48 (s, 9H), 3.43-3.46 (m, 4H), 3.60-3.64 (m, 4H), 3.87 (s, 3H), 4.35 (d, J = 6.1 Hz, 2H), 5.03 (br s, 1H), 6.85-6.86 (m, 1H), 7.10-7.12 (m, 1H), 7.18-7.20 (m, 1H), 7.84-7.87 (m, 3H), 8.12 (s, 1H) ppm; [ES+ MS] m/z 583 (MH⁺) |
| 192 | 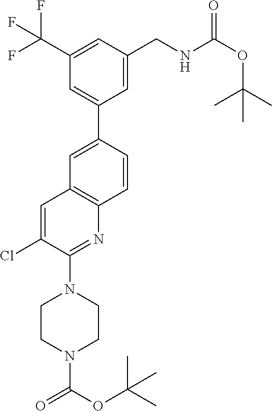 [SMILES: CC(C)(C)OC(=O)NCc1cc(-c2ccc3nc(N4CCN(C(=O)OC(C)(C)C)CC4)c(Cl)cc3c2)cc(C(F)(F)F)c1] | 163 | tert-butyl 4-[6-[3-[(tert- butoxycarbonylamino)methyl]-5- (trifluoromethyl)phenyl]-3-chloro-2- quinolyl]piperazine-1-carboxylate Purification by flash chromatography (cyclohexane/EtOAc 100/0-80/20); Yield: 64%; ¹H NMR (300 MHz, CD₂Cl₂): δ 1.43 (s, 9H), 1.48 (s, 9H), 3.45-3.48 (m, 4H), 3.61-3.64 (m, 4H), 4.44 (d, J = 6.1 Hz, 2H), 5.13 (br s, 1H), 7.55-7.57 (m, 1H), 7.81-7.89 (m, 5H), 8.14 (s, 1H) ppm; [ES+ MS] m/z 621 (MH⁺). |
| 193 | 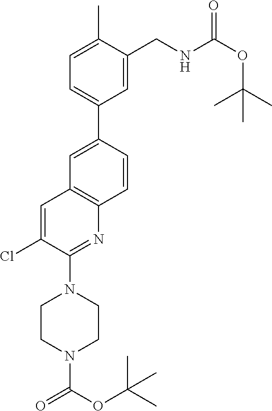 [SMILES: Cc1ccc(-c2ccc3nc(N4CCN(C(=O)OC(C)(C)C)CC4)c(Cl)cc3c2)cc1CNC(=O)OC(C)(C)C] | 164 | tert-butyl 4-[6-[3-[(tert- butoxycarbonylamino)methyl]-4-methyl- phenyl]-3-chloro-2-quinolyl]piperazine-1- carboxylate Purification by flash chromatography (cyclohexane/EtOAc 100/0-80/20); Yield: 82%; ¹H NMR (300 MHz, CD₂Cl₂): δ 1.46 (s, 9H), 1.49 (s, 9H), 2.40 (s, 3H), 3.42-3.45 (m, 4H), 3.60-3.64 (m, 4H), 4.36 (d, J = 5.8 Hz, 2H), 5.01 (br s, 1H), 7.27 (d, J = 7.8 Hz, 1H), 7.51 (dd, J = 1.9, 7.8 Hz, 1H), 7.56 (d, J = 1.6 Hz, 1H), 7.80-7.82 (m, 1H), 7.85-7.86 (m, 2H), 8.10 (s, 1H) ppm; [ES+ MS] m/z 567 (MH⁺) |
| 194 | 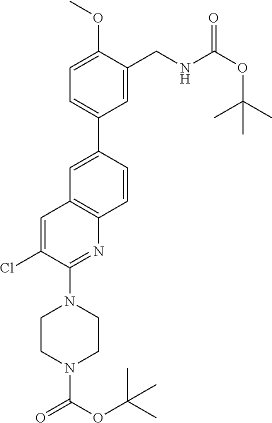 [SMILES: COc1ccc(-c2ccc3nc(N4CCN(C(=O)OC(C)(C)C)CC4)c(Cl)cc3c2)cc1CNC(=O)OC(C)(C)C] | 165 | tert-butyl 4-[6-[3-[(tert- butoxycarbonylamino)methyl]-4-methoxy- phenyl]-3-chloro-2-quinolyl]piperazine-1- carboxylate Purification by flash chromatography (cyclohexane/EtOAc 100/0-80/20); Yield: 69%; ¹H NMR (300 MHz, CD₂Cl₂): δ 1.43 (s, 9H), 1.48 (s, 9H), 3.42-3.45 (m, 4H), 3.60-3.64 (m, 4H), 3.90 (s, 3H), 4.34 (d, J = 6.1 Hz, 2H), 5.13 (br s, 1H), 6.99-7.02 (m, 1H), 7.59-7.62 (m, 2H), 7.80-7.82 (m, 1H), 7.85-7.86 (m, 2H), 8.12 (s, 1H) ppm; [ES+ MS] m/z 583 (MH⁺) |
| 195 | 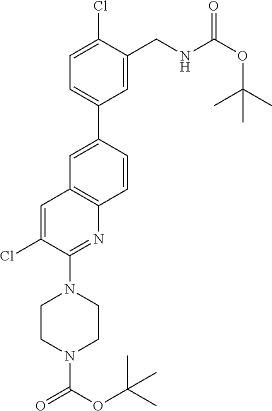 [SMILES: CC(C)(C)OC(=O)NCc1cc(-c2ccc3nc(N4CCN(C(=O)OC(C)(C)C)CC4)c(Cl)cc3c2)ccc1Cl] | 166 | tert-butyl 4-[6-[3-[(tert- butoxycarbonylamino)methyl]-4-chloro- phenyl]-3-chloro-2-quinolyl]piperazine-1- carboxylate Purification by flash chromatography (cyclohexane/EtOAc 100/0-80/20); Yield: 67%; ¹H NMR (300 MHz, CD₂Cl₂): δ 1.43 (s, 9H), 1.48 (s, 9H), 3.43-3.47 (m, 4H), 3.60-3.64 (m, 4H), 4.45 (d, J = 6.2 Hz, 2H), 5.15 (br s, 1H), 7.46 (d, J = 8.3 Hz, 1H), 7.55 (dd, J = 8.1, 2.4 Hz, 1H), 7.70 (d, J = 2.1 Hz 1H), 7.85-7.87 (m, 3H), 8.12 (s, 1H) ppm; [ES+ MS] m/z 587 (MH⁺). |
| 196 | 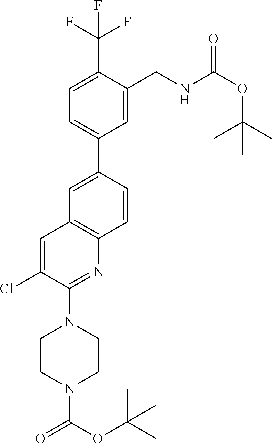 [SMILES: CC(C)(C)OC(=O)NCc1cc(-c2ccc3nc(N4CCN(C(=O)OC(C)(C)C)CC4)c(Cl)cc3c2)ccc1C(F)(F)F] | 167 | tert-butyl 4-[6-[3-[(tert- butoxycarbonylamino)methyl]-4- (trifluoromethyl)phenyl]-3-chloro-2- quinolyl]piperazine-1-carboxylate Purification by flash chromatography (cyclohexane/EtOAc 100/0-80/20); [ES+ MS] m/z 621 (MH⁺) |
| 197 | 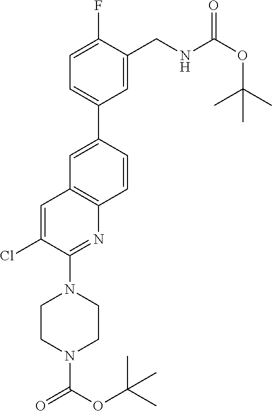 [SMILES: CC(C)(C)OC(=O)NCc1cc(-c2ccc3nc(N4CCN(C(=O)OC(C)(C)C)CC4)c(Cl)cc3c2)ccc1F] | 173 | tert-butyl 4-[6-[3-[(tert- butoxycarbonylamino)methyl]-4-fluoro- phenyl]-3-chloro-2-quinolyl]piperazine-1- carboxylate Purification by flash chromatography (cyclohexane/EtOAc 100/0-80/20); Yield: 60%; ¹H NMR (300 MHz, CD₂Cl₂): δ 1.44 (s, 9H), 1.48 (s, 9H), 3.43-3.47 (m, 4H), 3.60-3.64 (m, 4H), 4.42 (d, J = 6.2 Hz, 2H), 5.06 (br s, 1H), 7.16 (dd, J = 8.6, 9.8 Hz, 1H), 7.55-7.61 (m, 1H), 7.65 (dd, J = 2.4, 7.7 Hz, 1H), 7.81- 7.89 (m, 3H), 8.12 (s, 1H) ppm; [ES+ MS] m/z 571 (MH⁺). |
| 198 | 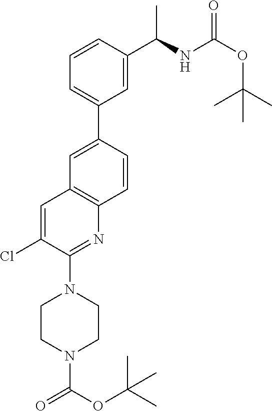 [SMILES: C[C@@H](NC(=O)OC(C)(C)C)c1cccc(-c2ccc3nc(N4CCN(C(=O)OC(C)(C)C)CC4)c(Cl)cc3c2)c1] | 170 | tert-butyl 4-[6-[3-[(1R)-1-(tert- butoxycarbonylamino)ethyl]phenyl]-3- chloro-2-quinolyl]piperazine-1-carboxylate Purification by flash chromatography (cyclohexane/EtOAc 100/0-80/20); Yield: 65%; ¹H NMR (300 MHz, CD₂Cl₂): δ 1.41 (s, 9H), 1.48 (s, 9H), 1.55 (s, 3H), 3.43-3.47 (m, 4H), 3.60-3.65 (m, 4H), 4.82 (br s, 1H), 4.97 (br s, 1H), 7.32 (td, J = 1.4, 7.7 Hz, 1H), 7.45 (t, J = 7.6 Hz, 1H), 7.56-7.63 (m, 2H), 7.86- 7.89 (m, 3H), 8.13 (s, 1H) ppm; [ES+ MS] m/z 567 (MH⁺). |
| 199 | 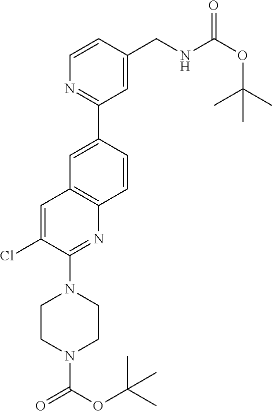 [SMILES: CC(C)(C)OC(=O)NCc1ccnc(-c2ccc3nc(N4CCN(C(=O)OC(C)(C)C)CC4)c(Cl)cc3c2)c1] | 168 | tert-butyl 4-[6-[4-[(tert- butoxycarbonylamino)methyl]-2-pyridyl]- 3-chloro-2-quinolyl]piperazine-1- carboxylate Purification by flash chromatography (cyclohexane/EtOAc 100/0-80/20); Yield: 90%; ¹H NMR (300 MHz, CD₂Cl₂): δ 1.48 (s, 9H), 1.54 (s, 9H), 3.47-3.48 (m, 4H), 3.61-3.64 (m, 4H), 4.40 (d, J = 6.2 Hz, 2H), 5.12 (br s, 1H, NH), 7.19 (dd, J = 1.5, 7.1 Hz, 1H), 7.76- 7.77 (m, 1H), 7.88 (m, 1H), 8.16 (s, 1H), 8.28- 8.33 (m, 2H), 8.64 (d, J = 5.1 Hz, 1H) ppm; [ES+ MS] m/z 554 (MH⁺) |
| 200 | 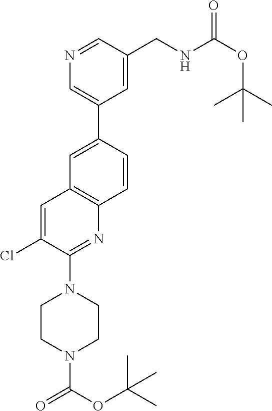 [SMILES: CC(C)(C)OC(=O)NCc1cncc(-c2ccc3nc(N4CCN(C(=O)OC(C)(C)C)CC4)c(Cl)cc3c2)c1] | 174 | tert-butyl 4-[6-[5-[(tert- butoxycarbonylamino)methyl]-3-pyridyl]- 3-chloro-2-quinolyl]piperazine-1- carboxylate Purification by flash chromatography (cyclohexane/EtOAc 100/0-80/20); Yield: 66%; ¹H NMR (300 MHz, CD₂Cl₂): δ): 1.45 (s, 9H), 1.48 (s, 9H), 3.44-3.49 (m, 4H), 3.59-3.65 (m, 4H), 4.39 (d, J = 6.3 Hz, 2H), 7.84-7.94 (m, 4H), 7.84-7.94 (m, 1H), 8.14 (s, 1H), 8.87 (d, J = 2.2 Hz, 1H) ppm; [ES+ MS] m/z 554 (MH⁺). |
| 201 | 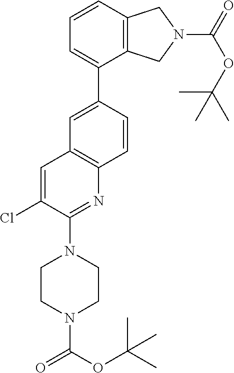 [SMILES: CC(C)(C)OC(=O)N1CCN(c2nc3ccc(-c4cccc5c4CN(C(=O)OC(C)(C)C)C5)cc3cc2Cl)CC1] | 176 | tert-butyl 4-[2-(4-tert- butoxycarbonylpiperazin-1-yl)-3-chloro-6- quinolyl]isoindoline-2-carboxylate Purification by flash chromatography (cyclohexane/EtOAc 100/0-80/20); Yield: 72%; ¹H NMR (300 MHz, CD₂Cl₂): δ 1.43 (s, 9H), 1.48 (s, 9H), 3.44-3.48 (m, 4H), 3.61-3.65 (m, 4H), 4.69-4.76 (m, 4H), 7.25-7.43 (m, 3H), 7.67-7.72 (m, 2H), 7.85-7.90 (m, 1H), 8.12 (d, J = 2.3 Hz, 1H) ppm; [ES+ MS] m/z 565 (MH⁺). |
| 202 | 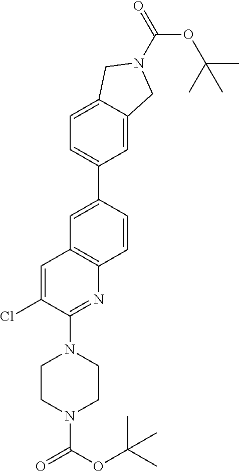 [SMILES: CC(C)(C)OC(=O)N1CCN(c2nc3ccc(-c4ccc5c(c4)CN(C(=O)OC(C)(C)C)C5)cc3cc2Cl)CC1] | 177 | tert-butyl 5-[2-(4-tert- butoxycarbonylpiperazin-1-yl)-3-chloro-6- quinolyl]isoindoline-2-carboxylate Purification by flash chromatography (cyclohexane/EtOAc 100/0-80/20); Yield: 47%; ¹H NMR (300 MHz, CD₂Cl₂): δ 1.48 (s, 9H), 1.51 (s, 9H), 3.43-3.46 (m, 4H), 3.61-3.64 (m, 4H), 4.69-4.73 (m, 4H), 7.33-7.40 (m, 1H), 7.54-7.62 (m, 2H), 7.83-7.89 (m, 3H), 8.13 (s, 1H) ppm; [ES+ MS] m/z 565 (MH⁺). |
| 207 | 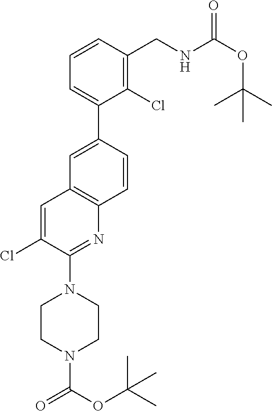 [SMILES: CC(C)(C)OC(=O)NCc1cccc(-c2ccc3nc(N4CCN(C(=O)OC(C)(C)C)CC4)c(Cl)cc3c2)c1Cl] | 205 | tert-butyl 4-[6-[3-[(tert- butoxycarbonylamino)methyl]-2-chloro- phenyl]-3-chloro-2-quinolyl]piperazine-1- carboxylate Purification by flash chromatography (cyclohexane/EtOAc 100/0-80/20); [ES+ MS] m/z 587 (MH⁺). |
| 208 | 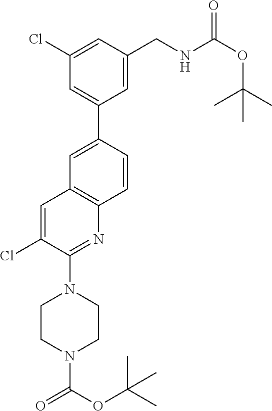 [SMILES: CC(C)(C)OC(=O)NCc1cc(Cl)cc(-c2ccc3nc(N4CCN(C(=O)OC(C)(C)C)CC4)c(Cl)cc3c2)c1] | 206 | tert-butyl 4-[6-[3-[(tert- butoxycarbonylamino)methyl]-5-chloro- phenyl]-3-chloro-2-quinolyl]piperazine-1- carboxylate Purification by flash chromatography (cyclohexane/EtOAc 100/0-80/20); [ES+ MS] m/z 587 (MH⁺). |

### EXAMPLES

Examples 1-9 were prepared according to Protocol 1.

| Ex | Structure | Physical data |
|---|---|---|
| 1 | 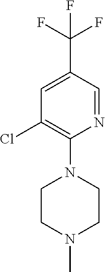 [SMILES: CN1CCN(c2ncc(C(F)(F)F)cc2Cl)CC1] | 1-[3-chloro-5-(trifluoromethyl)-2-pyridyl]-4-methyl- piperazine Purification by reverse chromatography (MeCN/H₂O 10/90- 100/0); Yield: 60%; ¹H NMR (300 MHz, CD₂Cl₂): δ 2.30 (s, 3H), 2.52 (t, J = 4.9 Hz, 4H), 3.52 (t, J = 4.9 Hz, 4H), 7.76- 7.77 (m, 1H), 8.37-8.39 (m, 1H) ppm; ¹³C NMR (75 MHz, CD₂Cl₂): δ 46.3, 48.9, 55.2, 119.7 (q, ²J_{CF} = 33 Hz), 121.0, 124.0 (q, ¹J_{CF} = 271 Hz), 136.3 (q, ³J_{CF} = 3.4 Hz), 143.3 (q, ³J_{CF} = 4.3 Hz), 160.4 ppm; [ES+ MS] m/z 280 (MH⁺). |
| 2 | 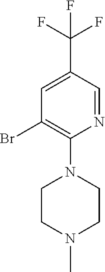 [SMILES: CN1CCN(c2ncc(C(F)(F)F)cc2Br)CC1] | 1-[3-bromo-5-(trifluoromethyl)-2-pyridyl]-4-methyl- piperazine Purification by flash chromatography (DCM/MeOH 100/0- 95/05); Yield: 91%; ¹H NMR (300 MHz, CD₂Cl₂): δ 2.30 (s, 3H), 2.52 (t, J = 4.9 Hz, 4H), 3.48 (t, J = 5.0 Hz, 4H), 7.96- 7.97 (m, 1H), 8.42-8.43 (m, 1H) ppm; ¹³C NMR (75 MHz, CD₂Cl₂): δ 46.3, 49.4, 55.2, 110.4, 120.2 (q, ²J_{CF} = 33 Hz), 123.8 (q, ¹J_{CF} = 272 Hz), 139.8 (q, ⁴J_{CF} = 3.0 Hz), 143.9 (q, ⁴J_{CF} = 4.1 Hz), 161.7 ppm; [ES+ MS] m/z 324 (MH⁺). |
| 3 | 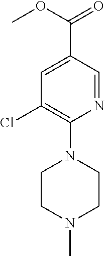 [SMILES: COC(=O)c1cnc(N2CCN(C)CC2)c(Cl)c1] | methyl 5-chloro-6-(4-methylpiperazin-1-yl)pyridine-3- carboxylate Purification by flash chromatography (DCM/MeOH 100/0- 95/05); Yield: 52%; ¹H NMR (300 MHz, CD₂Cl₂): δ 2.29 (s, 3H), 2.51 (t, J = 4.9 Hz, 4H), 3.55 (t, J = 5.0 Hz, 4H), 3.86 (s, 3H), 8.09 (d, J = 2.0 Hz, 1H), 8.69 (d, J = 2.0 Hz, 1H) ppm; ¹³C NMR (75 MHz, CD₂Cl₂): δ 46.3, 48.8, 52.3, 55.3, 119.6, 120.1, 140.0, 147.9, 160.4, 165.4 ppm; [ES+ MS] m/z 270 (MH⁺). |
| 4 | 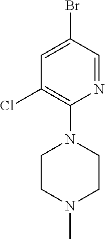 | 1-(5-bromo-3-chloro-2-pyridyl)-4-methyl-piperazine Purification by flash chromatography (DCM/MeOH 100/0- 93/7); Yield: 72%; ¹H NMR (300 MHz, CD₂Cl₂): δ 2.29 (s, 3H), 2.51 (t, J = 4.9 Hz, 4H), 3.33 (t, J = 5.0 Hz, 4H), 7.72 (d, J = 2.2 Hz, 1H), 8.19 (d, J = 2.2 Hz, 1H) ppm; ¹³C NMR (75 MHz, CD₂Cl₂): δ 46.3, 49.2, 55.3, 111.5, 123.0, 141.0, 146.8, 157.6 ppm; [ES+ MS] m/z 290 (MH⁺). |
| 5 | 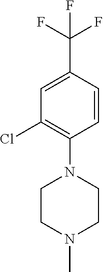 [SMILES: CN1CCN(c2ccc(C(F)(F)F)cc2Cl)CC1] | 1-[2-chloro-4-(trifluoromethyl)phenyl]-4-methyl- piperazine Purification by flash chromatography (DCM/MeOH 100/0- 95/05); Yield: 71%; ¹H NMR (300 MHz, CD₂Cl₂): δ 2.32 (s, 3H), 2.57 (t, J = 4.5 Hz, 4H), 3.12 (t, J = 4.6 Hz, 4H), 7.12 (dd, J = 0.5, 8.4 Hz, 1H), 7.46-7.50 (m, 1H), 7.60-7.62 (m, 1H) ppm; ¹³C NMR (75 MHz, CD₂Cl₂): δ 46.2, 51.2, 55.4, 120.8, 124.2 (q, ¹J_{CF} = 271 Hz), 125.1 (q, ⁴J_{CF} = 3.5 Hz), 125.2 (q, ²J_{CF} = 33 Hz), 128.1 (q, ⁴J_{CF} = 3.6 Hz), 128.8, 153.0 ppm; [ES+ MS] m/z 279 (MH⁺). |
| 6 | 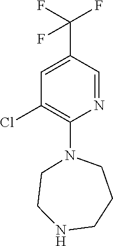 [SMILES: FC(F)(F)c1cnc(N2CCCNCC2)c(Cl)c1] | 1-[3-chloro-5-(trifluoromethyl)-2-pyridyl]-1,4-diazepane Purification by flash chromatography (DCM/MeOH 100/0- 90/10); Yield: 77%. ¹H NMR (300 MHz, CD₂Cl₂): δ 1.86-1.95 (m, 2H), 2.14 (br s, 1H), 2.85-2.90 (m, 2H), 3.03-3.08 (m, 2H), 3.78-3.87 (m, 4H), 7.68-7.70 (m, 1H), 8.27-8.29 (m, 1H). ¹³C NMR (75 MHz, CD₂Cl₂): δ 31.1, 48.4, 49.8, 50.5, 54.1, 116.5, 117.1 (q, ²J_{CF} = 33 Hz), 124.2 (q, ¹J_{CF} = 271 Hz), 136.9 (q, ³J_{CF} = 3 Hz), 143.0 (q, ³J_{CF} = 4 Hz), 158.7. [ES+ MS] m/z 280 (MH⁺). |
| 7 | 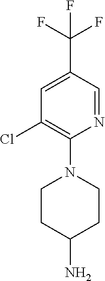 [SMILES: NC1CCN(c2ncc(C(F)(F)F)cc2Cl)CC1] | 1-[3-chloro-5-(trifluoromethyl)-2-pyridyl]piperidin-4- amine Purification by flash chromatography (DCM/MeOH 2% Et₃N 100/0-90/10); Yield: 78%; ¹H NMR (300 MHz, DMSO-d₆): δ 1.45-1.58 (m, 2H), 1.89-1.94 (m, 2H), 2.93-3.08 (m, 3H), 3.93-3.98 (m, 2H), 5.23 (br s, 2H), 8.15-8.16 (m, 1H), 8.52- 8.53 (m, 1H); ¹³C NMR (75 MHz, DMSO-d₆): δ 33.1, 47.7, 48.5, 119.0 (q, ²J_{CF} = 32 Hz), 120.6, 124.3 (q, ¹J_{CF} = 271 Hz), 137.1 (q, ³J_{CF} = 3 Hz), 143.9 (q, ³J_{CF} = 4 Hz), 160.5; [ES+ MS] m/z 280 (MH⁺). |
| 8 | 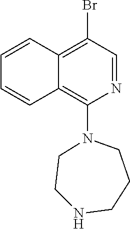 [SMILES: Brc1cnc(N2CCCNCC2)c2ccccc12] | 4-bromo-1-(1,4-diazepan-1-yl)isoquinoline Purification by reverse chromatography (MeOH/H₂O 10/90- 100/0); Yield: 49%. ¹H NMR (300 MHz, CD₂Cl₂): δ 1.95-2.00 (m, 2H), 3.00-3.05 (m, 2H), 3.09-3.13 (m, 2H), 3.69-3.75 (m, 4H), 7.53 (ddd, J = 1.2, 6.9, 8.3 Hz, 1H), 7.71 (ddd, J = 1.1, 6.9, 8.3 Hz, 1H), 8.04 (d, J = 8.4 Hz, 1H), 8.10 (dd, J = 0.5, 8.4 Hz, 1H), 8.18 (s, 1H) ppm; ¹³C NMR (75 MHz, CD₂Cl₂): δ 31.4, 48.6, 49.8, 53.4, 56.1, 110.5, 122.7, 126.4, 126.7, 130.9, 136.9, 142.1, 161.2 ppm. [ES+ MS] m/z 306 (MH⁺) |
| 9 | 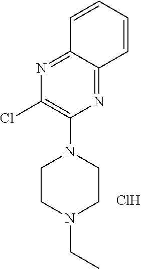 [SMILES: CCN1CCN(c2nc3ccccc3nc2Cl)CC1 ; Cl] | 2-chloro-3-(4-ethylpiperazin-1- yl)quinoxaline;hydrochloride Purification by reverse phase flash chromatography (MeOH/H₂O 10/90-100/0). Addition of HCl 4N in dioxane to form the hydrochloride salt, recuperation by filtration. Yield: 46%. ¹H NMR (300 MHz, DMSO-d₆): δ 1.29 (t, J = 7.4 Hz, 3H), 3.11-3.27 (m, 4H), 3.39-3.50 (m, 2H), 3.62 (d, J = 11.9 Hz, 2H), 4.18 (d, J = 13.8 Hz, 2H), 7.69 (ddd, J = 1.5, 6.9, 8.3 Hz, 1H), 7.79 (ddd, J = 1.5, 7.0, 8.4 Hz, 1H), 7.89 (ddd, J = 0.5, 1.6, 8.3 Hz, 1H), 7.94 (ddd, J = 0.5, 1.5, 8.3 Hz, 1H), 10.63 (brs, 1H) ppm; ¹³C NMR (75 MHz, DMSO-d₆): δ 8.9, 45.7, 49.9, 50.7, 126.9, 127.4, 128.2, 130.8, 138.0, 139.2, 141.1, 151.3 ppm. [ES+ MS] m/z 277 (MH⁺) |

Examples 10-11 were prepared according to Protocol 2.

| Ex | Structure | Physical data |
|---|---|---|
| 10 | 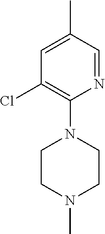 | 1-(3-chloro-5-methyl-2-pyridyl)-4-methyl-piperazine Purification by flash chromatography (DCM/MeOH 100/0- 93/7); Yield: 61%; ¹H NMR (300 MHz, CD₂Cl₂): δ 2.22 (s, 3H), 2.30 (s, 3H), 2.53 (t, J = 4.8 Hz, 4H), 3.26 (t, J = 4.8 Hz, 4H), 7.42-7.44 (m, 1H), 7.97-7.99 (m, 1H) ppm; ¹³C NMR (75 MHz, CD₂Cl₂): δ 17.2, 46.3, 49.6, 55.4, 122.8, 128.3, 139.8, 146.1, 157.0 ppm; [ES+ MS] m/z 226 (MH⁺). |
| 11 | 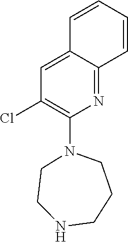 [SMILES: Clc1cc2ccccc2nc1N1CCCNCC1] | 3-chloro-2-(1,4-diazepan-1-yl)quinoline Purification by reverse chromatography (H₂O/MeOH 90/10- 0/100); Yield: 48%; ¹H NMR (300 MHz, CD₂Cl₂): δ 1.92-2.02 (m, 3H), 2.93-2.98 (m, 2H), 3.10-3.14 (m, 2H), 3.76-3.83 (m, 4H), 7.29 (ddd, J = 1.1, 7.0, 8.0 Hz, 1H), 7.52-7.61 (m, 2H), 7.70 (d, J = 8.4 Hz, 1H), 8.00 (s, 1H) ppm; ¹³C NMR (75 MHz, DMSO-d₆): δ 31.5, 48.4, 50.1, 51.3, 121.2, 124.1, 125.2, 126.6, 127.1, 129.9, 138.2, 145.8, 156.7 ppm; [ES+ MS] m/z 262 (MH⁺). |

Examples 12-78, 93-137 were prepared according to Protocol 3.

| Ex | Structure | Physical data |
|---|---|---|
| 12 | 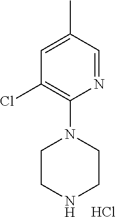 | 1-(3-chloro-5-methyl-2-pyridyl)piperazine; hydrochloride Yield: 98%; ¹H NMR (300 MHz, DMSO-d₆): δ 2.22 (s, 3H), 3.15-3.20 (m, 4H), 3.42 (t, J = 4.7 Hz, 4H), 7.73 (d, J = 1.0 Hz, 1H), 8.09 (d, J = 1.0 Hz, 1H), 9.21 (br s, 2H) ppm; ¹³C NMR (75 MHz, DMSO-d6): δ 16.6, 42.5, 45.8, 121.7, 129.2, 139.8, 145.7, 154.8 ppm; [ES+ MS] m/z 212 (MH⁺). |
| 13 | 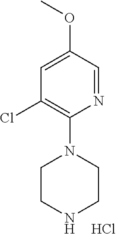 | 1-(3-chloro-5-methoxy-2- pyridyl)piperazine;hydrochloride Yield: 72%; ¹H NMR (300 MHz, DMSO-d₆): δ 3.14-3.22 (m, 4H), 3.30-3.36 (m, 4H), 3.80 (s, 3H), 7.61 (d, J = 2.7 Hz, 1H), 8.02 (d, J = 2.7 Hz, 1H), 9.50 (br s, 2H). ¹³C NMR (75 MHz, DMSO-d₆): δ 42.7, 46.2, 56.4, 122.8, 125.0, 132.8, 151.0, 152.4; [ES+ MS] m/z 228 (MH⁺). |
| 14 | 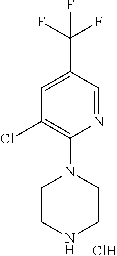 [SMILES: FC(F)(F)c1cnc(N2CCNCC2)c(Cl)c1 ; Cl] | 1-[3-chloro-5-(trifluoromethyl)-2-pyridyl]piperazine; hydrochloride Yield: 97%; ¹H NMR (300 MHz, DMSO-d₆): δ 3.18-3.23 (m, 4H), 3.66-3.71 (m, 4H), 8.27 (dd, J = 0.5, 1.9 Hz, 1H), 8.60- 8.61 (m, 1H), 9.59 (br s, 2H) ppm; ¹³C NMR (75 MHz, DMSO-d6): 42.3, 45.0, 119.5 (q, ²J_{CF} = 33 Hz), 120.6, 123.3 (q, ¹J_{CF} = 272 Hz), 136.5 (q, ³J_{CF} = 3.1 Hz), 143.2 (q, ³J_{CF} = 4.3 Hz), 159.0 ppm; [ES+ MS] m/z 266 (MH⁺). |
| 14′ | 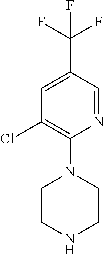 [SMILES: FC(F)(F)c1cnc(N2CCNCC2)c(Cl)c1] | 1-[3-chloro-5-(trifluoromethyl)-2-pyridyl]piperazine ¹H NMR (300 MHz, DMSO-d₆): δ 2.80-2.83 (m, 4H), 3.34- 3.38 (m, 4H), 8.14-8.16 (m, 1H), 8.52-8.54 (m, 1H); ¹³C NMR (75 MHz, DMSO-d₆): δ 45.5, 49.6, 118.0 (q, ²J_{CF} = 33 Hz), 119.7, 123.5 (q, ¹J_{CF} = 270 Hz), 136.1 (q, ³J_{CF} = 3 Hz), 143.0 (q, ³J_{CF} = 4 Hz), 159.8 |
| 15 | 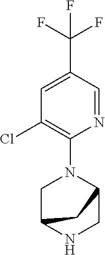 | (1S,4S)-2-[3-chloro-5-(trifluoromethyl)-2-pyridyl]-2,5- diazabicyclo[2.2.1]heptane;formic acid Purification by reverse phase flash chromatography (MeCN/water pH 9.2); Yield: 10%; ¹H NMR (300 MHz, DMSO-d₆): δ 1.83 (d, J = 10.2 Hz, 1H), 1.95 (d, J = 10.2 Hz, 1H), 3.14 (d, J = 9.8 Hz, 1H), 3.23 (d, J = 9.8 Hz, 1H), 3.66 (d, J = 10.2 Hz, 1H), 3.96 (d, J = 10.2 Hz, 1H), 4.08 (s, 1H), 4.90 (s, 1H), 7.56 (br s, 2H), 8.03-8.04 (m, 1H), 8.40-8.41 (m, 1H); ¹³C NMR (75 MHz, DMSO-d₆): δ 35.5, 50.7, 57.0, 57.1, 58.6, 115.5, 116.0 (q, ²J_{CF} = 32 Hz), 124.2 (q, ¹J_{CF} = 271 Hz), 136.4 (q, ³J_{CF} = 3 Hz), 143.6 (q, ³J_{CF} = 4 Hz), 156.4; [ES+ MS] m/z 278 (MH⁺). |
| 16 | 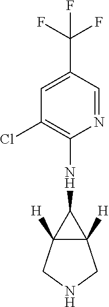 [SMILES: FC(F)(F)c1cnc(N[C@H]2[C@@H]3CNC[C@@H]32)c(Cl)c1] | (1R,5S)-N-[3-chloro-5-(trifluoromethyl)-2-pyridyl]-3- azabicyclo[3.1.0]hexan-6-amine;formic acid Purified by reverse phase chromatography (MeCN 0.1% formic acid/water 0.1% formic acid 10/90-100/0); Yield: 58%; ¹H NMR (300 MHz, DMSO-d₆): δ 1.86-1.88 (m, 2H), 2.80-2.83 (m, 1H), 3.09-3.11 (d, J = 11.3 Hz, 2H), 3.23 (d, J = 11.3 Hz, 2H), 6.45 (br s, 2H), 7.36-7.37 (d, J = 3.0 Hz, 1H), 7.96-7.97 (m, 1H), 8.38-8.40 (m, 1H); ¹³C NMR (75 MHz, DMSO-d₆): δ 25.3, 32.7, 47.8, 114.7 (q, ²J_{CF} = 32 Hz), 114.8, 124.5 (q, ¹J_{CF} = 271 Hz), 133.4 (q, ³J_{CF} = 3 Hz), 144.3 (q, ³J_{CF} = 4 Hz), 157.0; [ES+ MS] m/z 278 (MH⁺). |
| 17 | 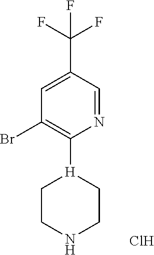 | 1-[3-bromo-5-(trifluoromethyl)-2-pyridyl]piperazine; hydrochloride Yield: 90%; ¹H NMR (300 MHz, DMSO-d₆): δ 3.18-3.22 (m, 4H), 3.63-3.67 (m, 4H), 8.40 (dd, J = 0.5, 2.2 Hz, 1H), 8.64- 8.66 (m, 1H), 9.66 (br s, 2H) ppm; ¹³C NMR (75 MHz, DMSO-d6): 42.3, 45.6, 110.5, 120.0 (q, ²J_{CF} = 33 Hz), 123.2 (q, ¹J_{CF} = 273 Hz), 139.9 (q, ³J_{CF} = 2.4 Hz), 143.7 (q, ³J_{CF} = 4.1 Hz), 160.4 ppm; [ES+ MS] m/z 310 (MH⁺). |
| 18 | 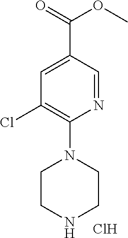 [SMILES: COC(=O)c1cnc(N2CCNCC2)c(Cl)c1 ; Cl] | methyl 5-chloro-6-piperazin-1-yl-pyridine-3- carboxylate; hydrochloride Yield: 82%; ¹H NMR (300 MHz, DMSO-d₆): δ 3.18-3.23 (m, 4H), 3.71 (t, J = 4.9 Hz, 4H), 3.85 (s, 3H), 8.16 (d, J = 2.0 Hz, 1H), 8.71 (d, J = 2.0 Hz, 1H), 9.55 (br s, 2H) ppm; ¹³C NMR (75 MHz, DMSO-d6): 42.4, 45.0, 52.3, 119.8, 120.1, 139.5, 147.2, 158.9, 164.2 ppm; [ES+ MS] m/z 256 (MH⁺). |
| 19 | 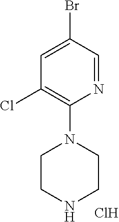 [SMILES: Clc1cc(Br)cnc1N1CCNCC1 ; Cl] | 1-(5-bromo-3-chloro-2- pyridyl)piperazine;hydrochloride Yield: 99%; ¹H NMR (300 MHz, DMSO-d₆): δ 3.15-3.20 (m, 4H), 3.47-3.52 (m, 4H), 8.18 (d, J = 2.2 Hz, 1H), 8.37 (d, J = 2.2 Hz, 1H), 9.63 (br s, 2H) ppm; ¹³C NMR (75 MHz, DMSO-d6): 42.4, 45.4, 112.4, 122.5, 141.0, 146.6, 155.9 ppm; [ES+ MS] m/z 276 (MH⁺). |
| 20 | 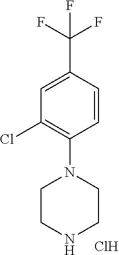 [SMILES: FC(F)(F)c1ccc(N2CCNCC2)c(Cl)c1 ; Cl] | 1-[2-chloro-4-(trifluoromethyl)phenyl]piperazine; hydrochloride Yield: 91%; ¹H NMR (300 MHz, DMSO-d₆): δ 3.21-3.26 (m, 4H), 3.28-3.33 (m, 4H), 7.37 (d, J = 8.2 Hz, 1H), 7.67-7.70 (m, 1H), 7.83 (d, J = 2.0 Hz, 1H), 9.50 (br s, 2H) ppm; ¹³C NMR (75 MHz, DMSO-d6): 42.8, 47.3, 121.5, 123.6 (q, ¹J_{CF} = 270 Hz), 124.4 (q, ²J_{CF} = 32 Hz), 125.3 (q, ³J_{CF} = 3.8 Hz), 127.4 (q, ³J_{CF} = 3.8 Hz), 127.7, 151.3 ppm; [ES+ MS] m/z 265 (MH⁺). |
| 21 | 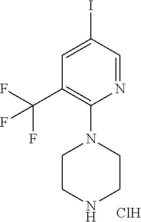 [SMILES: FC(F)(F)c1cc(I)cnc1N1CCNCC1 ; Cl] | 1-[5-iodo-3-(trifluoromethyl)-2-pyridyl]piperazine; hydrochloride Yield: 65%; ¹H NMR (300 MHz, DMSO-d₆): δ 3.10-3.24 (m, 4H), 3.34-3.46 (m, 4H), 8.37 (d, J = 2.0 Hz, 1H), 8.76 (d, J = 2.0 Hz, 1H), 9.52 (s, 2H) ppm; ¹³C NMR (75 MHz, DMSO- d6): 42.5, 47.1, 85.5, 117.5 (q, ²J_{CF} = 32 Hz), 122.8 (q, ¹J_{CF} = 273 Hz), 144.8 (q, ³J_{CF} = 4.9 Hz), 157.3 ppm; [ES+ MS] m/z 358 (MH⁺). |
| 22 | 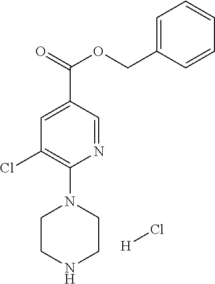 | benzyl 5-chloro-6-piperazin-1-yl-pyridine-3- carboxylate;hydrochloride Yield: 17%; ¹H NMR (300 MHz, DMSO-d₆): δ 3.17-3.26 (m, 4H), 3.66-3.76 (m, 4H), 5.35 (s, 2H), 7.31-7.51 (m, 5H), 8.18 (d, J = 2.0 Hz, 1H), 8.75 (d, J = 2.0 Hz, 1H), 9.38 (br s, 2H); ¹³C NMR (75 MHz, DMSO-d₆): δ 42.9, 45.5, 66.8, 120.2, 120.5, 128.4, 128.6, 129.0, 136.3, 139.9, 147.8, 159.4, 164.0; [ES+ MS] m/z 332 (MH⁺). |
| 23 | 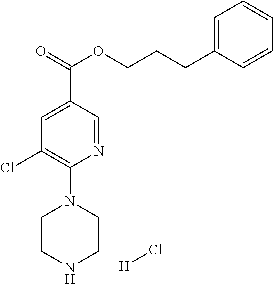 | 3-phenylpropyl 5-chloro-6-piperazin-1-yl-pyridine-3- carboxylate;hydrochloride Yield: 93%; ¹H NMR (300 MHz, DMSO-d₆): δ 2.01 (m, 2H), 2.72 (t, J = 7.4 Hz), 3.18-3.24 (m, 4H), 3.71 (t, J = 4.8 Hz, 4H), 4.26 (t, J = 6.3 Hz), 7.13-7.31 (m, 5H), 8.09 (d, J = 2.0 Hz, 1H), 8.70 (d, J = 2.0 Hz, 1H), 9.54 (br s, 2H); ¹³C NMR (75 MHz, DMSO-d₆): δ 29.7, 31.6, 42.4, 45.1, 64.4, 119.8, 120.3, 125.9, 128.3, 139.4, 141.2, 147.2, 159.0, 163.7; [ES+ MS] m/z 360 (MH⁺). |
| 24 | 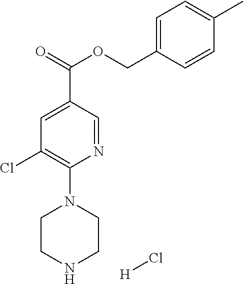 | p-tolylmethyl 5-chloro-6-piperazin-1-yl-pyridine-3- carboxylate;hydrochloride Yield: 80%; ¹H NMR (300 MHz, DMSO-d₆): δ 2.30 (s, 3H), 3.16-3.24 (m, 4H), 3.71 (t, J = 4.7 Hz, 4H), 5.30 (s, 2H), 7.20 (d, J = 7.8 Hz, 2H), 7.35 (d, J = 7.9 Hz, 2H), 8.15 (d, J = 2.0 Hz, 1H), 8.73 (d, J = 2.1 Hz, 1H), 9.51 (br s, 2H); ¹³C NMR (75 MHz, DMSO-d₆): δ 20.8, 42.4, 45.0, 66.3, 119.7, 120.1, 128.1, 129.1, 132.9, 137.5, 139.5, 147.3, 159.0, 163.5; [ES+ MS] m/z 346 (MH⁺). |
| 25 | 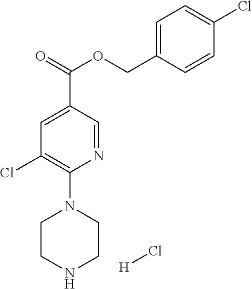 | (4-chlorophenyl)methyl 5-chloro-6-piperazin-1-yl- pyridine-3-carboxylate;hydrochloride Yield: 23%; ¹H NMR (300 MHz, DMSO-d₆): δ 3.22 (t, J = 4.6 Hz, 4H), 3.69 (t, J = 4.6 Hz, 4H), 5.34 (s, 2H), 7.44-7.53 (m, 4H), 8.19 (d, J = 1.9 Hz, 1H), 8.76 (d, J = 1.9 Hz, 1H), 9.17 (br s, 2H); ¹³C NMR (75 MHz, DMSO-d₆): δ 42.6, 45.2, 65.6, 119.7, 119.9, 128.5, 129.9, 132.8, 134.9, 139.6, 147.4, 159.0, 163.5; [ES+ MS] m/z 366 (MH⁺). |
| 26 | 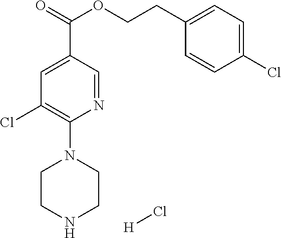 | 2-(4-chlorophenyl)ethyl 5-chloro-6-piperazin-1-yl- pyridine-3-carboxylate;hydrochloride Yield: 60%; ¹H NMR (300 MHz, DMSO-d₆): δ 3.02 (t, J = 6.7 Hz, 2H), 3.18-3.23 (m, 4H), 3.70 (t, J = 4.7 Hz, 4H), 4.46 (t, J = 6.5 Hz, 2H), 7.34-7.37 (m, 4H), 8.08 (d, J = 2.1 Hz, 1H), 8.64 (d, J = 2.0 Hz, 1H), 9.43 (br s, 2H); ¹³C NMR (75 MHz, DMSO-d₆): δ 33.5, 42.4, 45.1, 65.2, 119.8, 120.1, 128.3, 130.8, 131.1, 137.2, 139.4, 147.1, 158.9, 163.5; [ES+ MS] m/z 380 (MH⁺). |
| 27 | 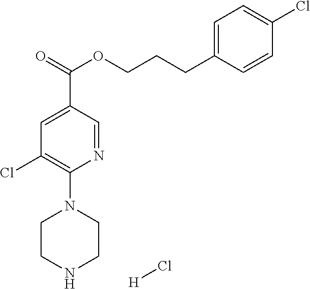 | 3-(4-chlorophenyl)propyl 5-chloro-6-piperazin-1-yl- pyridine-3-carboxylate;hydrochloride Yield: 97%; ¹H NMR (300 MHz, DMSO-d₆): δ 1.96-2.06 (m, 2H), 2.73 (t, J = 7.3 Hz, 2H), 3.19-3.25 (m, 4H), 3.70 (t, J = 4.6 Hz, 4H), 4.26 (t, J = 6.3 Hz, 2H), 7.25-7.34 (m, 4H), 8.08 (d, J = 2.0 Hz, 1H), 8.67 (d, J = 2.0 Hz, 1H), 9.36 (br s, 2H); ¹³C NMR (75 MHz, DMSO-d₆): δ 29.5, 31.0, 42.5, 45.1, 64.4, 119.8, 120.2, 128.2, 130.3, 130.5, 139.4, 140.4, 147.2, 158.9, 163.6; [ES+ MS] m/z 394 (MH⁺). |
| 28 | 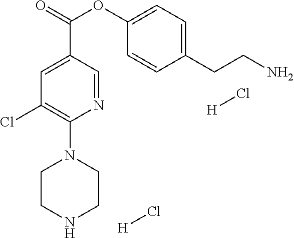 | [4-(2-aminoethyl)phenyl] 5-chloro-6-piperazin-1-yl- pyridine-3-carboxylate;dihydrochloride Yield: 97%; ¹H NMR (300 MHz, DMSO-d₆): δ 2.91-3.08 (m, 4H), 3.18-3.25 (m, 4H), 3.78 (t, J = 4.8 Hz, 4H), 7.22-7.27 (m, 2H), 7.34-7.39 (m, 2H), 8.19-8.25 (m, 3H), 8.32 (d, J = 2.1 Hz, 1H), 8.87 (d, J = 2.0 Hz, 1H), 9.69 (br s, 2H); ¹³C NMR (75 MHz, DMSO-d₆): δ 32.3, 40.3, 42.4, 45.0, 119.3, 119.6, 122.0, 129.8, 135.4, 140.0, 147.9, 149.1, 159.3, 162.6; [ES+ MS] m/z 361 (MH⁺). |
| 29 | 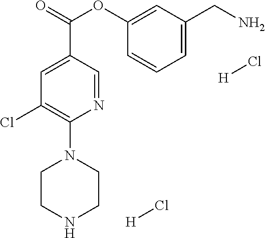 | [3-(aminomethyl)phenyl] 5-chloro-6-piperazin-1-yl- pyridine-3-carboxylate;dihydrochloride Yield: 64%; ¹H NMR (300 MHz, DMSO-d₆): δ 3.19-3.26 (m, 4H), 3.76-3.82 (m, 4H), 4.06 (q, J = 5.7 Hz, 2H), 7.31 (td, J = 1.8, 7.7 Hz, 1H), 7.44-7.55 (m, 3H), 8.34 (d, J = 2.0 Hz, 1H), 8.60 (br s, 3H), 8.89 (d, J = 2.0 Hz, 1H), 9.67 (br s, 2H); ¹³C NMR (75 MHz, DMSO-d₆): δ 41.7, 42.4, 45.0, 119.0, 119.6, 122.0, 122.3, 126.7, 129.8, 135.9, 140.0, 147.9, 150.3, 159.3, 162.5; [ES+ MS] m/z 347 (MH⁺). |
| 30 | 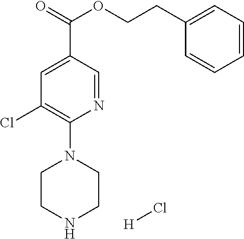 | 2-phenylethyl 5-chloro-6-piperazin-1-yl-pyridine-3- carboxylate;hydrochloride Yield: 88%; ¹H NMR (300 MHz, DMSO-d₆): δ 3.02 (t, J = 6.7 Hz, 2H), 3.19-3.22 (m, 4H), 3.69-3.72 (m, 4H), 4.47 (t, J = 6.6 Hz, 2H), 7.19-7.32 (m, 5H), 8.08 (d, J = 1.9 Hz, 1H), 8.65 (d, J = 1.9 Hz, 1H), 9.53 (br s, 2H); ¹³C NMR (75 MHz, DMSO-d₆): δ 34.3, 42.4, 45.0, 65.5, 119.7, 120.1, 126.4, 128.4, 128.9, 133.0, 139.4, 147.1, 158.9, 163.5; [ES+ MS] m/z 446 (MH⁺). |
| 31 | 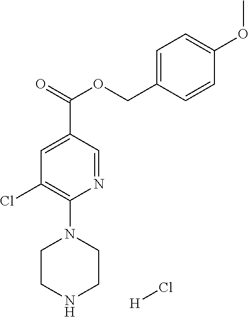 | (4-methoxyphenyl)methyl 5-chloro-6-piperazin-1-yl- pyridine-3-carboxylate;hydrochloride Yield: 54%; ¹H NMR (300 MHz, DMSO-d₆): δ 3.17-3.24 (m, 4H), 3.67-3.73 (m, 4H), 3.75 (s, 3H), 5.27 (s, 2H), 6.94 (d, J = 8.6 Hz, 2H), 7.40 (d, J = 8.2 Hz, 2H), 8.14 (d, J = 1.5 Hz, 1H), 8.71 (d, J = 1.5 Hz, 1H), 9.50 (br s, 2H); ¹³C NMR (75 MHz, DMSO-d₆): δ 42.4, 45.0, 55.1, 66.2, 113.9, 119.7, 120.1, 127.7, 130.0, 139.5, 147.3, 159.0, 159.3, 163.6; [ES+ MS] m/z 462 (MH⁺). |
| 32 | 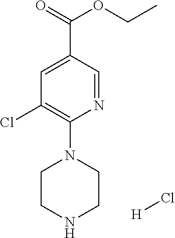 | ethyl 5-chloro-6-piperazin-1-yl-pyridine-3- carboxylate; hydrochloride Yield: 100%; ¹H NMR (300 MHz, DMSO-d₆): δ 1.32 (t, J = 7.1 Hz, 3H), 3.18-3.24 (m, 4H), 3.70-3.73 (m, 4H), 4.32 (q, J = 7.1 Hz, 2H), 8.16 (d, J = 2.0 Hz, 1H), 8.71 (d, J = 2.0 Hz, 1H), 9.60 (br s, 2H); ¹³C NMR (75 MHz, DMSO-d₆): δ 14.6, 42.8, 45.5, 61.5, 120.2, 120.8, 139.9, 147.6, 159.4, 164.1; [ES+ MS] m/z 270 (MH⁺). |
| 33 | 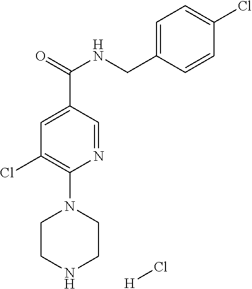 | 5-chloro-N-[(4-chlorophenyl)methyl]-6-piperazin-1-yl- pyridine-3-carboxamide;hydrochloride Yield: 82%, ¹H NMR (300 MHz, DMSO-d₆): δ 3.15-3.26 (m, 4H), 3.58-3.69 (m, 4H), 4.44 (d, J = 5.8 Hz, 2H), 7.30-7.41 (m, 4H), 8.30 (d, J = 2.1 Hz, 1H), 8.75 (d, J = 2.1 Hz, 1H), 9.34 (t, J = 5.6 Hz, 1H), 9.55 (br s, 2H); ¹³C NMR (75 MHz, DMSO-d₆): δ 41.9, 42.4, 45.2, 120.3, 124.6, 128.2, 129.2, 131.4, 138.1, 138.4, 145.5, 158.0, 163.3; [ES+ MS] m/z 365 (MH⁺). |
| 34 | 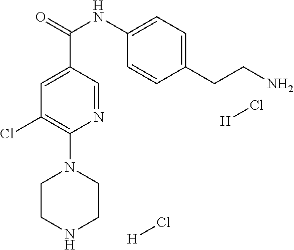 | N-[4-(2-aminoethyl)phenyl]-5-chloro-6-piperazin-1-yl- pyridine-3-carboxamide;dihydrochloride Yield: 99%; ¹H NMR (300 MHz, DMSO-d₆): δ 2.83-3.05 (m, 4H), 3.18-3.25 (m, 4H), 3.66 (t, J = 4.5 Hz, 4H), 7.24 (d, J = 8.4 Hz, 2H), 7.75 (d, J = 8.5 Hz, 2H), 8.14-8.19 (m, 3H), 8.42 (d, J = 2.0 Hz, 1H), 8.84 (d, J = 2.1 Hz, 1H), 9.61 (br s, 2H), 10.47 (s, 1H); ¹³C NMR (75 MHz, DMSO-d₆): δ 32.4, 39.9, 42.4, 45.2, 120.1, 120.6, 125.2, 128.8, 132.8, 137.5, 138.5, 146.0, 158.1, 162.3; [ES+ MS] m/z 360 (MH⁺). |
| 35 | 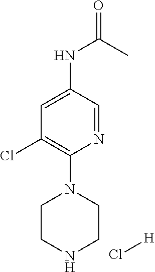 | N-(5-chloro-6-piperazin-1-yl-3- pyridyl)acetamide;hydrochloride Yield: 84; ¹H NMR (300 MHz, DMSO-d₆): δ 2.05 (s, 3H), 3.15-3.24 (m, 4H), 3.36-3.42 (m, 4H), 8.19 (d, J = 2.3 Hz, 1H), 8.41 (d, J = 2.2 Hz, 1H), 9.40 (s, 2H), 10.47 (s, 1H); ¹³C NMR (75 MHz, DMSO-d₆): δ 23.7, 42.6, 45.9, 121.6, 129.5, 132.4, 136.6, 152.3, 168.8; [ES+ MS] m/z 255 (MH⁺). |
| 36 | 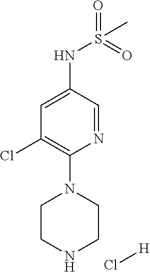 | N-(5-chloro-6-piperazin-1-yl-3-pyridyl)methane sulfonamide;hydrochloride Yield: 22%; ¹H NMR (300 MHz, DMSO-d₆): δ 3.03 (s, 3H), 3.15-3.23 (m, 4H), 3.29-3.47 (m, 4H), 7.70 (d, J = 2.4 Hz, 1H), 8.14 (d, J = 2.4 Hz, 1H), 9.51 (s, 2H), 10.04 (s, 1H); ¹³C NMR (75 MHz, DMSO-d₆): δ 39.6, 42.5, 45.8, 121.8, 130.9, 132.0, 138.8, 153.7; [ES+ MS] m/z 291 (MH⁺). |
| 37 | 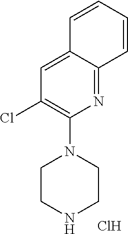 [SMILES: Clc1cc2ccccc2nc1N1CCNCC1 ; Cl] | 3-chloro-2-piperazin-1-yl-quinoline; hydrochloride Yield: 72%; ¹H NMR (300 MHz, DMSO-d₆): δ 3.22-3.28 (m, 4H), 3.63-3.68 (m, 4H), 7.49 (t, J = 7.2 Hz, 1H), 7.70 (t, J = 7.2 Hz, 1H), 7.80-7.89 (m, 2H), 8.49 (s, 1H), 9.71 (br s, 2H) ppm; ¹³C NMR (75 MHz, DMSO-d₆): δ 42.4, 45.9, 121.5, 125.6, 125.7, 126.9, 127.0, 130.3, 138.3, 144.4, 155.5 ppm; [ES+ MS] m/z 248 (MH⁺). |
| 37′ | 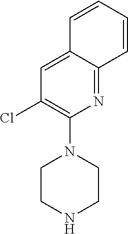 [SMILES: Clc1cc2ccccc2nc1N1CCNCC1] | 3-chloro-2-piperazin-1-yl-quinoline Purified by reverse phase chromatography (MeCN 0.1% formic acid/water 0.1% formic acid 20/80-100/0); Yield: 14%; ¹H NMR (300 MHz, DMSO-d₆): δ 3.00-3.05 (m, 4H), 3.38-3.43 (m, 4H), 7.46 (ddd, J = 1.2, 6.9, 8.0 Hz, 1H), 7.67 (ddd, J = 1.4, 6.9, 8.4 Hz, 1H), 7.78 (d, J = 8.4 Hz, 1H), 7.84 (d, J = 8.1 Hz, 1H), 8.44 (s, 1H) ppm; [ES+ MS] m/z 248 (MH⁺). |
| 38 | 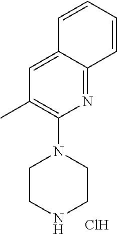 [SMILES: Cc1cc2ccccc2nc1N1CCNCC1 ; Cl] | 3-methyl-2-piperazin-1-yl-quinoline; hydrochloride Yield: 100%; ¹H NMR (300 MHz, DMSO-d₆): δ 2.47 (s, 3H), 3.29-3.34 (m, 4H), 3.81-3.85 (m, 4H), 7.53 (ddd, J = 0.9, 7.2, 8.0 Hz, 1H), 7.75 (ddd, J = 1.1, 7.1, 8.3 Hz, 1H), 7.89 (dd, J = 0.7, 8.0 Hz, 1H), 8.24 (d, J = 8.2 Hz, 1H), 8.35 (s, 1H), 9.81 (br s, 2H) ppm; ¹³C NMR (75 MHz, DMSO-d₆): δ 19.1, 42.5, 46.4, 122.0, 124.0, 125.6, 125.8, 127.2, 130.7, 139.3, 142.6, 157.1 ppm; [ES+ MS] m/z 228 (MH⁺). |
| 39 | 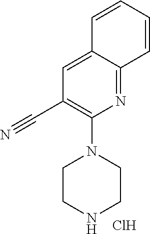 [SMILES: N#Cc1cc2ccccc2nc1N1CCNCC1 ; Cl] | 2-piperazin-1-ylquinoline-3-carbonitrile; hydrochloride Yield: 59%; ¹H NMR (300 MHz, DMSO-d₆): δ 3.25-3.30 (m, 4H), 3.80-3.86 (m, 4H), 7.50-7.56 (m, 1H), 7.75-7.87 (m, 2H), 7.94 (d, J = 8.0 Hz, 1H), 8.98 (s, 1H), 9.52 (br s, 2H) ppm; ¹³C NMR (75 MHz, DMSO-d₆): δ 42.3, 45.6, 99.0, 117.2, 122.6, 125.6, 127.1, 128.4, 133.4, 147.1, 147.2, 156.9 ppm; [ES+ MS] m/z 239 (MH⁺). |
| 40 | 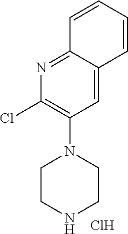 [SMILES: Clc1nc2ccccc2cc1N1CCNCC1 ; Cl] | 2-chloro-3-piperazin-1-yl-quinoline;hydrochloride Yield: 100%; ¹H NMR (300 MHz, DMSO-d₆): δ 3.29-3.47 (m, 8H), 7.60-7.74 (m, 2H), 7.90 (d, J = 7.7 Hz, 1H), 8.00 (d, J = 7.1 Hz, 1H), 8.14 (s, 1H), 9.34 (br s, 2H) ppm; [ES+ MS] m/z 248 (MH⁺). |
| 41 | 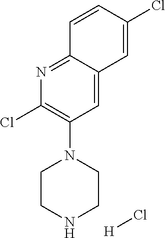 | 2,6-dichloro-3-piperazin-1-yl-quinoline;hydrochloride Yield: 63%; ¹H NMR (300 MHz, DMSO-d₆): δ 3.27-3.37 (m, 8H), 7.69 (dd, J = 2.4, 8.9 Hz, 1H), 7.91 (d, J = 8.9 Hz, 1H), 8.11 (s, 2H), 9.49 (s, 2H) ppm; ¹³C NMR (75 MHz, DMSO- d₆): δ 42.8, 47.8, 125.7, 125.9, 128.8, 129.4, 129.5, 131.8, 141.5, 142.9, 147.4 ppm; [ES+ MS] m/z 282 (MH⁺). |
| 42 | 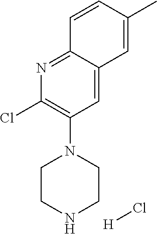 | 2-chloro-6-methyl-3-piperazin-1-yl- quinoline;hydrochloride Yield: 94%; ¹H NMR (300 MHz, DMSO-d₆): δ 2.49 (s, 3H), 3.29-3.33 (m, 8H), 7.53 (dd, J = 1.8, 8.6 Hz, 1H), 7.74-7.75 (m, 1H), 7.79 (d, J = 8.6 Hz, 1H), 8.01 (s, 1H), 9.37 (s, 2H) ppm; ¹³C NMR (75 MHz, DMSO-d₆): δ 21.2, 43.0, 48.0, 126.0, 126.0, 127.1, 127.9, 131.1, 137.1, 141.8, 142.0, 145.9 ppm; [ES+ MS] m/z 262 (MH⁺). |
| 43 | 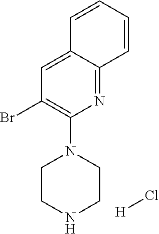 | 3-bromo-2-piperazin-1-yl-quinoline;hydrochloride Yield: 100%; ¹H NMR (300 MHz, DMSO-d₆): δ 3.21-3.30 (m, 4H), 3.59-3.65 (m, 4H), 7.49 (t, J = 7.5 Hz, 1H), 7.71 (t, J = 7.5 Hz, 1H), 7.84 (dd, J = 7.9, 15.7 Hz, 2H), 8.69 (s, 1H), 9.70 (br s, 2H) ppm; ¹³C NMR (75 MHz, DMSO-d₆): δ 42.5, 46.4, 111.6, 125.7, 126.3, 126.9, 127.0, 130.4, 142.1, 144.7, 156.3 ppm; [ES+ MS] m/z 292 (MH⁺). |
| 44 | 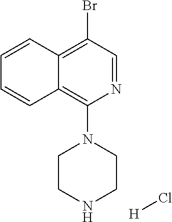 | 4-bromo-1-piperazin-1-yl-isoquinoline;hydrochloride Yield: 100%; ¹H NMR (300 MHz, DMSO-d₆): δ 3.30-3.35 (m, 4H), 3.58-3.62 (m, 4H), 7.74 (t, J = 7.5 Hz, 1H), 7.92 (t, J = 7.7 Hz, 1H), 8.05 (d, J = 8.2 Hz, 1H), 8.22 (d, J = 8.3 Hz, 1H), 8.35 (s, 1H), 9.72 (s, 2H); ¹³C NMR (75 MHz, DMSO- d₆): δ 42.5, 47.8, 112.6, 122.0, 126.0, 126.3, 128.2, 132.2, 135.6, 140.7, 159.5 ppm; [ES+ MS] m/z 292 (MH⁺). |
| 45 | 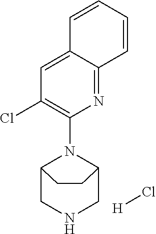 | 3-chloro-2-(3,8-diazabicyclo[3.2.1]octan-8- yl)quinoline;hydrochloride Yield: 93%; ¹H NMR (300 MHz, DMSO-d₆): δ 2.07-2.13 (m, 4H), 3.18-3.34 (m, 4H), 4.69-4.72 (m, 2H), 7.45 (ddd, J = 1.2, 6.9, 8.0 Hz, 1H), 7.67 (ddd, J = 1.4, 6.7, 8.4 Hz, 1H), 7.75 (d, J = 8.3 Hz, 1H), 7.84 (dd, J = 0.7, 8.2 Hz, 1H), 8.47 (s, 1H), 9.40 (br s, 1H), 9.96 (br s, 1H) ppm; ¹³C NMR (75 MHz, DMSO-d₆): δ 25.7, 47.5, 55.1, 120.7, 125.0, 125.4, 126.8, 126.9, 130.2, 138.1, 144.6, 153.1 ppm ppm; [ES+ MS] m/z 274 (MH⁺). |
| 46 | 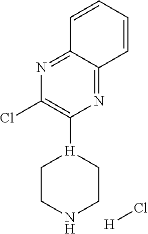 | 2-chloro-3-piperazin-1-yl-quinoxaline;hydrochloride Purification by reverse phase chromatography (MeOH/H₂O 10/90-100/0); Yield: 42%; ¹H NMR (300 MHz, DMSO-d₆): δ 3.26-3.29 (m, 4H), 3.73-3.76 (m, 4H), 7.67 (td, J = 6.9, 1.5 Hz, 1H), 7.78 (td, J = 1.5, 6.9 Hz, 1H), 7.85-7.93 (m, 2H), 9.65 (br s, 2H) ppm; ¹³C NMR (75 MHz, DMSO-d₆): δ 42.2, 45.5, 126.9, 127.4, 128.1, 130.7, 137.9, 139.2, 141.2, 151.7 ppm; [ES+ MS] m/z 249 (MH⁺). |
| 47 | 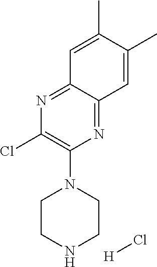 | 2-chloro-6,7-dimethyl-3-piperazin-1-yl- quinoxaline;hydrochloride Yield: 76%; ¹H NMR (300 MHz, DMSO-d₆): δ 2.40 (s, 3H), 2.41 (s, 3H), 3.25-3.29 (m, 4H), 3.65-3.70 (m, 4H), 7.66 (s, 1H), 7.68 (s, 1H), 9.53 (s, 1H) ppm; ¹³C NMR (75 MHz, DMSO-d₆): δ 19.5, 19.8, 42.3, 45.6, 126.1, 126.4, 136.8, 137.9, 138.3, 140.2, 140.9, 151.2 ppm; [ES+ MS] m/z 277 (MH⁺). |
| 48 | 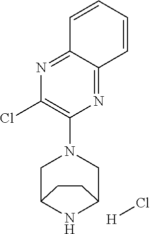 | 2-chloro-3-(3,8-diazabicyclo[3.2.1]octan-3- yl)quinoxaline;hydrochloride Yield: 85%; ¹H NMR (300 MHz, DMSO-d₆): δ 1.94-2.21 (m, 4H), 3.47-3.51 (m, 2H), 3.94 (d, J = 14.0 Hz, 2H), 4.16-4.21 (m, 2H), 7.67 (ddd, J = 1.6, 6.9, 8.3 Hz, 1H), 7.77 (ddd, J = 1.5, 6.9, 8.4 Hz, 1H), 7.86 (dd, J = 1.2, 8.3 Hz, 1H), 7.92 (dd, J = 1.2, 8.3 Hz, 1H), 9.58 (br s, 2H) ppm; ¹³C NMR (75 MHz, DMSO-d₆): δ 24.9, 50.8, 53.9, 126.8, 127.4, 128.1, 130.7, 137.9, 139.2, 140.8, 151.9; [ES+ MS] m/z 275 (MH⁺). |
| 49 | 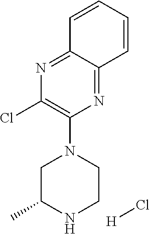 | 3-[(3R)-3-methylpiperazin-1-yl]quinoxalin-2- ol;hydrochloride Purification by reverse phase chromatography (MeOH/H₂O 10/90-100/0); Yield: 24%; ¹H NMR (300 MHz, DMSO-d₆): δ 1.04 (d, J = 6.3 Hz, 3H), 2.52-2.61 (m, 1H), 2.84-3.02 (m, 4H), 3.80-3.92 (m, 2H), 7.61 (ddd, J = 1.5, 6.9, 8.3 Hz, 1H), 7.73 (ddd, J = 1.6, 7.0, 8.4 Hz, 1H), 7.83 (ddd, J = 0.5, 1.5, 8.3 Hz, 1H), 7.88 (ddd, J = 0.5, 1.5, 8.3 Hz, 1H) ppm; ¹³C NMR (75 MHz, DMSO-d₆): δ 19.3, 44.9, 49.3, 49.9, 55.9, 126.7, 127.3, 127.4, 130.5, 137.4, 139.6, 141.3, 152.4 ppm; [ES+ MS] m/z 263 (MH⁺). |
| 50 | 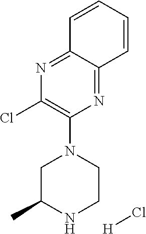 | 2-chloro-3-[(3S)-3-methylpiperazin-1- yl]quinoxaline;hydrochloride Purification by reverse phase chromatography (MeOH/H₂O 10/90-100/0); Yield: 32%; ¹H NMR (300 MHz, DMSO-d₆): δ 1.04 (d, J = 6.3 Hz, 3H), 2.57 (dd, J = 10.4, 12.1 Hz, 1H), 2.80-3.04 (m, 4H), 3.79-3.97 (m, 2H), 7.61 (ddd, J = 1.3, 6.9, 8.2 Hz, 1H), 7.73 (ddd, J = 1.3, 6.9, 8.2 Hz, 1H), 7.82 (dd, J = 0.8, 8.4 Hz, 1H), 7.86 (dd, J = 0.8, 8.3 Hz, 1H) ppm; ¹³C NMR (75 MHz, DMSO-d₆): δ 19.2, 44.8, 49.2, 49.8, 55.8, 126.7, 127.3, 127.4, 130.5, 137.3, 139.6, 141.3, 152.4 ppm; [ES+ MS] m/z 263 (MH⁺). |
| 51 | 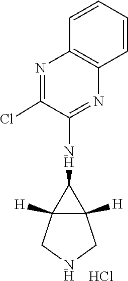 [SMILES: Clc1nc2ccccc2nc1N[C@H]1[C@@H]2CNC[C@@H]21 ; Cl] | N-[(1R,5S)-3-azabicyclo[3.1.0]hexan-6-yl]-3-chloro- quinoxalin-2-amine;hydrochloride Yield: 99%; ¹H NMR (300 MHz, DMSO-d₆): δ 2.08-2.10 (m, 2H), 2.98-3.00 (m, 1H), 3.35-3.50 (m, 4H), 7.45 (ddd, J = 1.5, 7.0, 8.2 Hz, 1H), 7.62-7.79 (m, 4H), 9.38 (s, 1H), 9.49 (s, 1H) ppm; ¹³C NMR (75 MHz, DMSO-d₆): δ 23.9, 32.1, 46.5, 125.2, 125.8, 127.5, 130.3, 135.9 , 137.6, 140.6, 148.8 ppm; [ES+ MS] m/z 261 (MH⁺). |
| 52 | 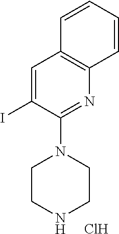 [SMILES: Ic1cc2ccccc2nc1N1CCNCC1 ; Cl] | 3-iodo-2-piperazin-1-yl-quinoline;hydrochloride Yield: 79%; ¹H NMR (300 MHz, DMSO-d₆): δ 3.25-3.30 (m, 4H), 3.52-3.56 (m, 4H), 7.49 (ddd, J = 0.9, 7.0, 8.0 Hz, 1H), 7.71 (ddd, J = 1.2, 6.9, 8.1 Hz, 1H), 7.79-7.86 (m, 2H), 8.91 (s, 1H), 9.51 (br s, 2H) ppm; ¹³C NMR (75 MHz, DMSO-d₆): δ 42.5, 47.1, 87.6, 125.5, 126.7, 127.2, 130.4, 145.2, 149.3, 158.7 ppm; [ES+ MS] m/z 340 (MH⁺). |
| 53 | 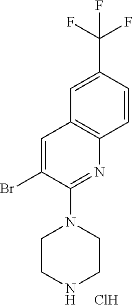 [SMILES: FC(F)(F)c1ccc2nc(N3CCNCC3)c(Br)cc2c1 ; Cl] | 3-bromo-2-piperazin-1-yl-6-(trifluoromethyl)quinoline; hydrochloride Yield: 92%; ¹H NMR (300 MHz, DMSO-d₆): δ 3.25-3.30 (m, 4H), 3.67-3.72 (m, 4H), 7.94-7.96 (m, 1H), 8.36-7.37 (m, 1H), 8.86 (s, 1H), 9.59 (br s, 2H) ppm; ¹³C NMR (75 MHz, DMSO-d₆): δ 42.4, 46.1, 112.6, 124.2 (q, ¹J_{CF} = 272 Hz), 125.1 (q, ³J_{CF} = 2.3 Hz), 125.2 (q, ²J_{CF} = 32 Hz), 125.6 (q, ²J_{CF} = 3.0 Hz), 128.5, 142.9, 146.3, 158.0 ppm; [ES+ MS] m/z 360 (MH⁺). |
| 54 | 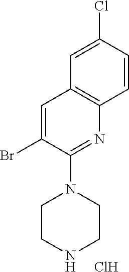 [SMILES: Clc1ccc2nc(N3CCNCC3)c(Br)cc2c1 ; Cl] | 3-bromo-6-chloro-2-piperazin-1-yl-quinoline; hydrochloride Yield: 80%; ¹H NMR (300 MHz, DMSO-d₆): δ 3.23-3.28 (m, 4H), 3.59-3.63 (m, 4H), 7.71 (d, J = 8.5 Hz, 1H), 7.80 (d, J = 8.8 Hz, 1H), 7.99 (s, 1H), 8.66 (s, 1H), 9.60 (s, 2H) ppm; ¹³C NMR (75 MHz, DMSO-d₆): δ 42.4, 46.2, 112.7, 125.5, 126.8, 129.1, 129.5, 130.6, 141.2, 143.4, 156.7 ppm; [ES+ MS] m/z 326 (MH⁺). |
| 55 | 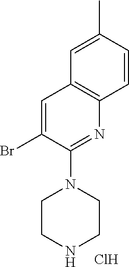 [SMILES: Cc1ccc2nc(N3CCNCC3)c(Br)cc2c1 ; Cl] | 3-bromo-6-methyl-2-piperazin-1-yl- quinoline;hydrochloride Yield: 56%; ¹H NMR (300 MHz, DMSO-d₆): δ 2.45 (s, 3H), 3.22-3.28 (m, 4H), 3.54-3.59 (m, 4H), 7.53-7.74 (m, 3H), 8.57 (s, 1H), 9.54 (s, 2H) ppm; ¹³C NMR (75 MHz, DMSO- d₆): δ 20.9, 42.5, 46.4, 111.6, 125.6, 126.2, 126.9, 132.3, 135.0, 141.3, 143.2, 155.7 ppm; [ES+ MS] m/z 306 (MH⁺). |
| 56 | 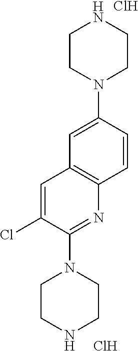 [SMILES: Cl ; Clc1cc2cc(N3CCNCC3)ccc2nc1N1CCNCC1 ; Cl] | 3-chloro-2,6-di(piperazin-1 yl)quinoline;dihydrochloride Yield: 99%; ¹H NMR (300 MHz, DMSO-d₆): δ 3.22-3.27 (m, 8H), 3.47-3.57 (m, 8H), 7.24 (d, J = 2.2 Hz, 1H), 7.57 (dd, J = 2.4, 9.3 Hz, 1H), 7.72 (d, J = 9.2 Hz, 1H), 8.30 (s, 1H), 9.51 (br s, 4H) ppm; ¹³C NMR (75 MHz, DMSO-d₆): δ 42.4, 42.5, 45.5, 46.0, 109.0, 122.0, 122.8, 126.8, 128.0, 136.8, 139.9, 147.5, 153.8 ppm; [ES+ MS] m/z 332 (MH⁺). |
| 57 | 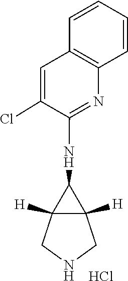 [SMILES: Clc1cc2ccccc2nc1N[C@H]1[C@@H]2CNC[C@@H]21 ; Cl] | N-[(1R,5S)-3-azabicyclo[3.1.0]hexan-6-yl]-3-chloro- quinolin-2-amine;hydrochloride Yield: 98%; ¹H NMR (300 MHz, DMSO-d₆): δ 2.21-2.24 (m, 2H), 3.23-3.26 (m, 1H), 3.35-3.42 (m, 2H), 3.69-3.79 (m, 2H), 7.47 (t, J = 7.5 Hz, 1H), 7.74 (t, J = 7.5 Hz, 1H), 7.86 (d, J = 7.7 Hz, 1H), 8.19 (d, J = 6.3 Hz, 1H), 8.58 (s, 2H), 9.16 (s, 1H), 10.07 (s, 1H) ppm; ¹³C NMR (75 MHz, DMSO- d₆): δ 24.5, 32.3, 46.5, 117.6, 121.3, 122.2, 124.7, 127.6, 131.4, 138.4, 151.5 ppm; [ES+ MS] m/z 260 (MH⁺). |
| 58 | 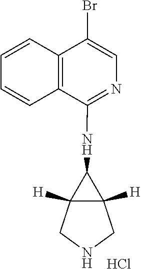 [SMILES: Brc1cnc(N[C@H]2[C@@H]3CNC[C@@H]32)c2ccccc12 ; Cl] | N-[(1R,5S)-3-azabicyclo[3.1.0]hexan-6-yl]-4-bromo- isoquinolin-1-amine;hydrochloride Yield: 99%; ¹H NMR (300 MHz, DMSO-d₆): δ 2.34-2.36 (m, 2H), 3.09-3.11 (m, 1H), 3.36-3.45 (m, 2H), 3.76-3.84 (m, 2H), 7.81-7.87 (m, 1H), 8.05-8.08 (m, 3H), 8.86 (d, J = 8.2 Hz, 1H), 9.26 (s, 1H), 9.88 (s, 1H), 10.06 (s, 1H) ppm; ¹³C NMR (75 MHz, DMSO-d₆): δ 24.2, 31.5, 46.4, 105.9, 119.2, 126.1, 126.2, 129.1, 134.3, 134.9, 153.2 ppm; [ES+ MS] m/z 304 (MH⁺). |
| 59 | 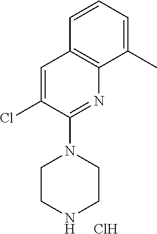 [SMILES: Cc1cccc2cc(Cl)c(N3CCNCC3)nc12 ; Cl] | 3-chloro-8-methyl-2-piperazin-1-yl- quinoline;hydrochloride Purification by reverse phase chromatography (MeOH/H₂O 10/90-100/0); Yield: 50%; ¹H NMR (300 MHz, DMSO-d₆): δ 2.63 (s, 3H), 3.27-3.31 (m, 4H), 3.66-3.70 (m, 4H), 7.38 (dd, J = 7.2, 7.9 Hz, 1H), 7.56 (td, J = 1.0, 6.8 Hz, 1H), 7.70 (d, J = 7.9 Hz, 1H), 8.45 (s, 1H), 9.45 (br s, 2H) ppm; ¹³C NMR (75 MHz, DMSO-d₆): δ 17.3, 42.3, 45.8, 121.0, 124.8, 125.3, 125.6, 130.2, 134.7, 138.5, 143.3, 154.3 ppm; [ES+ MS] m/z 262 (MH⁺). |
| 60 | 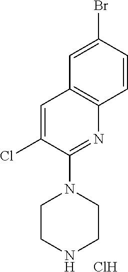 [SMILES: Clc1cc2cc(Br)ccc2nc1N1CCNCC1 ; Cl] | 6-bromo-3-chloro-2-piperazin-1-yl- quinoline;hydrochloride Yield: 100%; ¹H NMR (300 MHz, DMSO-d₆): δ 3.23-3.28 (m, 4H), 3.63-3.67 (m, 4H), 7.73 (d, J = 9.0 Hz, 1H), 7.80 (dd, J = 2.0, 8.9 Hz, 1H), 8.40 (d, J = 1.7 Hz, 1H), 8.47 (s, 1H), 9.59 (s, 2H) ppm; ¹³C NMR (75 MHz, DMSO-d₆): δ 42.4, 45.7, 117.8, 122.5, 126.9, 128.8, 129.2, 133.1, 137.3, 143.3, 155.8 ppm; [ES+ MS] m/z 326 (MH⁺). |
| 61 | 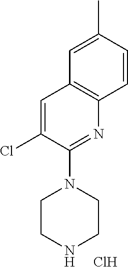 | 3-chloro-6-iodo-2-piperazin-1-yl- quinoline;hydrochloride Yield: 100%; ¹H NMR (300 MHz, DMSO-d₆): δ 3.23-3.29 (m, 4H), 3.62-3.66 (m, 4H), 7.57 (d, J = 8.8 Hz, 1H), 7.93 (dd, J = 1.7, 8.8 Hz, 1H), 8.31 (d, J = 1.4 Hz, 1H), 8.43 (s, 1H), 9.52 (br s, 2H) ppm; ¹³C NMR (75 MHz, DMSO-d₆): δ 42.4, 45.7, 90.7, 122.1, 127.5, 129.0, 135.2, 137.1, 138.4, 143.6, 155.8 ppm; [ES+ MS] m/z 374 (MH⁺). |
| 62 | 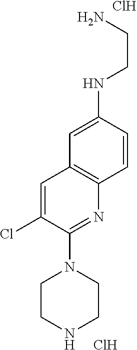 [SMILES: Cl ; NCCNc1ccc2nc(N3CCNCC3)c(Cl)cc2c1 ; Cl] | N′-(3-chloro-2-piperazin-1-yl-6-quinolyl)ethane-1,2- diamine; dihydrochloride Yield: 97%; ¹H NMR (300 MHz, DMSO-d₆): δ 3.02-3.08 (m, 2H), 3.22-3.26 (m, 4H), 3.39 (t, J = 1.4 Hz, 2H), 3.48-3.52 (m, 4H), 6.84 (d, J = 2.1 Hz, 1H), 7.21 (dd, J = 2.4, 9.1 Hz, 1H), 7.61 (d, J = 9.1 Hz, 1H), 8.18 (s, 1H), 8.32 (br s, 3H), 9.53 (br s, 2H) ppm; ¹³C NMR (75 MHz, DMSO-d₆): δ 37.5, 40.7, 42.6, 46.2, 101.9, 121.9, 121.9, 127.7, 127.8, 135.6, 138.5, 145.5, 152.2 ppm; [ES+ MS] m/z 306 (MH⁺). |
| 63 | 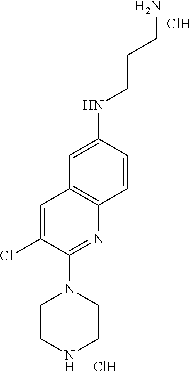 [SMILES: NCCCNc1ccc2nc(N3CCNCC3)c(Cl)cc2c1 ; Cl ; Cl] | N′-(3-chloro-2-piperazin-1-yl-6-quinolyl)propane-1,3- diamine; dihydrochloride Yield: 77%; ¹H NMR (300 MHz, DMSO-d₆): δ 1.94-2.01 (m, 2H), 2.88-2.95 (m, 2H), 3.23-3.30 (m, 6H), 3.53-3.58 (m, 4H), 7.15-7.19 (m, 1H), 7.43 (d, J = 8.5 Hz, 1H), 7.71 (d, J = 9.0 Hz, 1H), 8.18 (br s, 3H), 8.31 (s, 1H), 9.59 (br s, 2H) ppm; ¹³C NMR (75 MHz, DMSO-d₆): δ 25.3, 36.6, 42.5, 46.1, 122.1, 122.9, 127.1, 128.0, 136.6, 139.8, 141.9, 153.2 ppm; [ES+ MS] m/z 320 (MH⁺). |
| 64 | 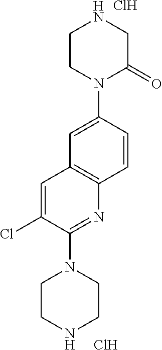 [SMILES: Cl ; O=C1CNCCN1c1ccc2nc(N3CCNCC3)c(Cl)cc2c1 ; Cl] | 1-(3-chloro-2-piperazin-1-yl-6-quinolyl)piperazin-2- one;dihydrochloride Purification by reverse phase chromatography (MeOH/H₂O 10/90-100/0); Yield: 74%; ¹H NMR (300 MHz, DMSO-d₆): δ 3.24-3.29 (m, 4H), 3.55-3.66 (m, 6H), 3.87-3.90 (m, 2H), 3.98-4.02 (m, 2H), 7.68 (dd, J = 1.3, 9.0 Hz, 1H), 7.81 (d, J = 1.3 Hz, 1H), 7.84 (d, J = 9.0 Hz, 1H), 8.53 (s, 1H), 9.57 (br s, 2H), 10.24 (br s, 2H) ppm; ¹³C NMR (75 MHz, DMSO- d₆): δ 39.6, 42.4, 45.0, 45.8, 46.2, 122.0, 122.7, 125.6, 127.7, 128.7, 138.0, 138.4, 143.1, 155.7, 162.2 ppm; [ES+ MS] m/z 346 (MH⁺). |
| 65 | 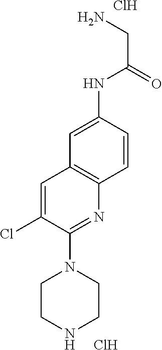 [SMILES: Cl ; NCC(=O)Nc1ccc2nc(N3CCNCC3)c(Cl)cc2c1 ; Cl] | 2-amino-N-(3-chloro-2-piperazin-1-yl-6- quinolyl)acetamide;dihydrochloride Yield: 100%; ¹H NMR (300 MHz, DMSO-d₆): δ 3.24-3.29 (m, 4H), 3.58-3.62 (m, 4H), 3.84-3.91 (m, 2H), 7.79 (d, J = 9.0 Hz, 1H), 7.85 (dd, J = 1.9, 9.0 Hz, 1H), 8.25 (d, J = 1.8 Hz, 1H), 8.36 (s, 3H), 8.48 (s, 1H), 9.56 (br s, 2H), 11.22 (br s, 1H) ppm; ¹³C NMR (75 MHz, DMSO-d₆): δ 41.1, 42.5, 45.9, 114.4, 122.2, 123.4, 126.0, 127.9, 135.6, 137.7, 141.5, 154.7, 165.1 ppm; [ES+ MS] m/z 346 (MH⁺). |
| 66 | 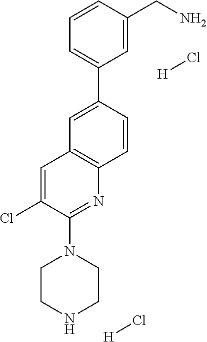 | [3-(3-chloro-2-piperazin-1-yl-6- quinolyl)phenyl]methanamine;dihydrochloride Yield: 91%; ¹H NMR (300 MHz, DMSO-d₆): δ 3.25-3.30 (m, 4H), 3.65-3.70 (m, 4H), 4.11 (q, J = 5.6 Hz, 2H), 7.53-7.58 (m, 2H), 7.77-7.82 (m, 1H), 7.91 (d, J = 8.8 Hz, 1H), 8.02 (s, 1H), 8.09 (dd, J = 2.1, 8.8 Hz, 1H), 8.24 (d, J = 1.9 Hz, 1H), 8.50 (s, 1H), 8.64 (br s, 3H), 9.65 (br s, 2H) ppm; ¹³C NMR (75 MHz, DMSO-d₆): δ 42.2, 42.4, 45.8, 122.0, 124.4, 125.9, 126.7, 127.7, 1 128.3, 129.1, 129.3, 135.0, 136.5, 138.3, 139.3, 144.1, 155.7 ppm; [ES+ MS] m/z 353 (MH⁺). |
| 67 | 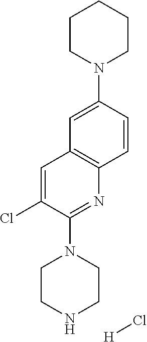 | 3-chloro-2-piperazin-1-yl-6-(1- piperidyl)quinoline;hydrochloride Yield: 92%; ¹H NMR (300 MHz, DMSO-d₆): δ 1.64-1.73 (m, 2H), 1.96-2.06 (m, 4H), 3.23-3.29 (m, 4H), 3.51-3.56 (m, 4H), 3.64-3.69 (m, 4H), 7.91 (d, J = 9.0 Hz, 1H), 8.09-8.26 (m, 2H), 8.48 (s, 1H), 9.62 (s, 2H) ppm; ¹³C NMR (75 MHz, DMSO-d₆): δ 21.3, 23.2, 42.4, 45.7, 54.9, 118.1, 122.6, 125.4, 125.4, 128.7, 138.1, 141.4, 143.3, 155.9 ppm; [ES+ MS] m/z 331 (MH⁺). |
| 68 | 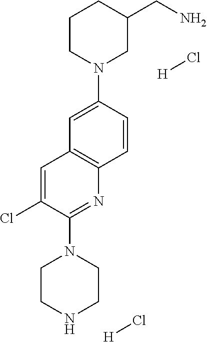 | [1-(3-chloro-2-piperazin-1-yl-6-quinolyl)-3- piperidyl]methanamine;dihydrochloride Yield: 100%; ¹H NMR (300 MHz, DMSO-d₆): δ 1.23-1.34 (m, 1H), 1.80-1.93 (m, 3H), 2.22-2.31 (m, 1H), 2.76-2.83 (m, 2H), 2.90-3.10 (m, 2H), 3.23-3.28 (m, 4H), 3.57-3.69 (m, 5H), 3.85-3.91 (m, 1H), 7.66-7.87 (m, 3H), 8.29 (br s, 3H) 8.34 (s, 1H), 9.60 (s, 2H) ppm; ¹³C NMR (75 MHz, DMSO- d₆): δ 23.0, 26.7, 33.1, 41.5, 42.5, 45.9, 51.2, 54.1, 109.6, 122.2, 123.2, 123.2, 126.4, 128.1, 137.2, 141.0, 154.4 ppm; [ES+ MS] m/z 360 (MH⁺). |
| 69 | 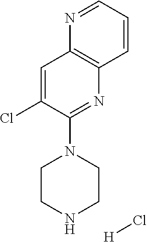 | 3-chloro-2-piperazin-1-yl-1,5-naphthyridine; hydrochloride Yield: 28%; ¹H NMR (300 MHz, DMSO-d₆): δ 3.27-3.32 (m, 4H), 3.66-3.70 (m, 4H), 7.75 (dd, J = 4.3, 8.5 Hz, 1H), 8.23- 8.27 (m, 1H), 8.51 (d, J = 0.5 Hz, 1H), 7.75 (dd, J = 1.5, 4.3 Hz, 1H), 9.32 (s, 2H) ppm; ¹³C NMR (75 MHz, DMSO- d₆): δ 42.5, 45.8, 124.9, 125.3, 135.3, 138.5, 140.3, 140.9, 149.4, 155.8 ppm; [ES+ MS] m/z 249 (MH⁺). |
| 70 | 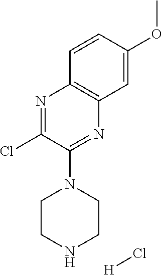 | 2-chloro-6-methoxy-3-piperazin-1-yl-quinoxaline; hydrochloride Yield: 78%; ¹H NMR (300 MHz, DMSO-d₆): δ 3.22-3.32 (m, 4H), 3.71-3.74 (m, 4H), 3.92 (s, 3H), 7.25 (d, J = 2.7 Hz, 1H), 7.30 (dd, J = 2.8, 9.0 Hz, 1H), 7.81 (d, J = 9.0 Hz, 1H), 9.57 (s, 2H) ppm; ¹³C NMR (75 MHz, DMSO-d₆): δ 42.2, 45.5, 55.9, 105.8, 120.1, 128.4, 133.5, 138.0, 141.0, 151.9, 160.9 ppm; [ES+ MS] m/z 279 (MH⁺). |
| 71 | 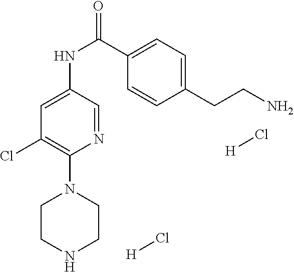 | 4-(2-aminoethyl)-N-(5-chloro-6-piperazin-1-yl-3- pyridyl)benzamide;dihydrochloride Yield: quantitative; ¹H NMR (300 MHz, DMSO-d₆): δ 2.98- 3.10 (m, 4H), 3.17-3.25 (m, 4H), 3.41-3.47 (m, 4H), 7.43 (d, J = 8.2 Hz, 2H), 8.01 (d, J = 8.2 Hz, 2H), 8.22-8.27 (m, 3H), 8.39 (d, J = 2.3 Hz, 1H), 8.70 (d, J = 2.2 Hz, 1H), 9.49 (brs, 2H), 10.68 (s, 1H) ppm; ¹³C NMR (75 MHz, DMSO-d₆): δ 32.8, 39.5, 42.6, 45.9, 121.3, 128.2, 128.8, 130.9, 132.2, 132.3, 138.0, 141.7, 152.7, 165.3 ppm; [ES+ MS] m/z 360 (MH⁺). |
| 72 | 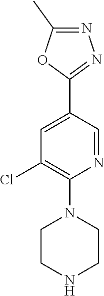 [SMILES: Cc1nnc(-c2cnc(N3CCNCC3)c(Cl)c2)o1] | 2-(5-chloro-6-piperazin-1-yl-3-pyridyl)-5-methyl-1,3,4- oxadiazole Purification by reverse phase chromatography (MeOH/H₂O 10/90-100/0); Yield : 62%; ¹H NMR (300 MHz, DMSO-d₆): δ 2.58 (s, 3H), 3.10-3.16 (m, 4H), 3.56-3.62 (m, 4H), 8.25 (d, J = 2.0 Hz, 1H), 8.75 (d, J = 2.1 Hz, 1H) ppm; ¹³C NMR (75 MHz, DMSO-d₆): δ 10.6, 43.3, 46.4, 114.5, 120.7, 136.6, 143.9, 158.3, 161.3, 164.0 ppm; [ES+ MS] m/z 280 (MH⁺). |
| 73 | 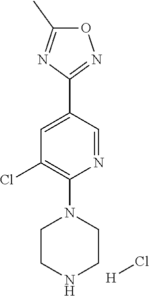 | 3-(5-chloro-6-piperazin-1-yl-3-pyridyl)-5-methyl-1,2,4- oxadiazole;hydrochloride Purification by reverse phase chromatography (MeOH/H₂O 10/90-100/0); Yield: 66%; ¹H NMR (300 MHz, DMSO-d₆): δ 2.68 (s, 3H), 3.22-3.27 (m, 4H), 3.62-3.67 (m, 4H), 8.26 (d, J = 2.1 Hz, 1H), 8.79 (d, J = 2.0 Hz, 1H), 9.15 (brs, 2H) ppm; ¹³C NMR (75 MHz, DMSO-d₆): δ 12.0, 42.6, 45.3, 117.5, 121.1, 137.1, 144.4, 158.1, 164.9, 177.8 ppm; [ES+ MS] m/z 280 (MH⁺). |
| 74 | 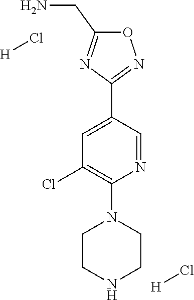 | [3-(5-chloro-6-piperazin-1-yl-3-pyridyl)-1,2,4-oxadiazol- 5-yl]methanamine;dihydrochloride Purification by reverse phase flash chromatography (MeOH/water 10/90-100/0); Yield: 46%; ¹H NMR (300 MHz, DMSO-d₆): δ 3.19-3.26 (m, 4H), 3.67-3.71 (m, 4H), 4.57 (brs, 2H), 8.28 (d, J = 2.0 Hz, 1H), 8.81 (d, J = 2.0 Hz, 1H), 9.10 (brs, 3H), 9.59 (brs, 2H) ppm; ¹³C NMR (75 MHz, DMSO-d₆): δ 34.6, 42.5, 45.2, 116.6, 121.1, 137.2, 144.6, 158.5, 165.0, 174.5 ppm; [ES+ MS] m/z 295 (MH⁺). |
| 75 | 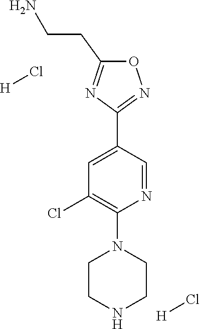 | 2-[3-(5-chloro-6-piperazin-1-yl-3-pyridyl)-1,2,4- oxadiazol-5-yl]ethanamine;dihydrochloride Yield: 92%; ¹H NMR (300 MHz, DMSO-d₆): δ 3.18-3.26 (m, 4H), 3.28-3.45 (m, 4H), 3.65-3.70 (m, 4H), 8.32 (d, J = 2.0 Hz, 1H), 8.40 (brs, 3H), 8.83 (d, J = 2.0 Hz, 1H), 9.59 (brs, 2H) ppm; ¹³C NMR (75 MHz, DMSO-d₆): δ 24.3, 35.6, 42.4, 45.2, 117.2, 121.0, 137.3, 144.6, 158.2, 164.8, 177.3 ppm; [ES+ MS] m/z 309 (MH⁺). |
| 76 | 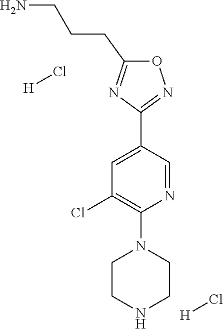 | 3-[3-(5-chloro-6-piperazin-1-yl-3-pyridyl)-1,2,4- oxadiazol-5-yl]propan-1-amine; dihydrochloride Yield: quantitative; ¹H NMR (300 MHz, DMSO-d₆): δ 2.11 (p, J = 7.5 Hz, 2H), 2.92-2.98 (m, 2H), 3.12-3.27 (m, 6H), 3.63- 3.71 (m, 4H), 8.15 (brs, 3H), 8.26 (d, J = 2.0 Hz, 1H), 8.79 (d, J = 2.0 Hz, 1H), 9.48 (brs, 2H) ppm; ¹³C NMR (75 MHz, DMSO-d₆): δ 23.0, 23.7, 37.9, 42.5, 45.2, 117.4, 121.0, 137.2, 144.5, 158.2, 164.8, 179.8 ppm; [ES+ MS] m/z 323 (MH⁺). |
| 77 | 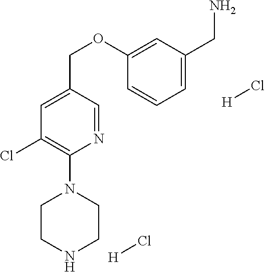 | [3-[(5-chloro-6-piperazin-1-yl-3- pyridyl)methoxy]phenyl]methanamine;dihydrochloride Yield: 79%; ¹H NMR (300 MHz, DMSO-d₆): δ 3.17-3.25 (m, 4H), 3.47-3.54 (m, 4H), 3.98 (q, J = 5.8 Hz, 2H), 5.10 (s, 2H), 7.00-7.10 (m, 2H), 7.24 (s, 1H), 7.34 (t, J = 7.9 Hz, 1H), 7.97 (d, J = 1.9 Hz, 1H), 8.37 (d, J = 2.0 Hz, 1H), 8.48 (brs, 3H), 9.41 (brs, 2H) ppm; ¹³C NMR (75 MHz, DMSO-d₆): δ 42.0, 42.5, 45.6, 65.8, 114.6, 115.5, 121.4, 121.5, 128.2, 129.8, 135.7, 139.2, 145.7, 156.6, 158.1 ppm; [ES+ MS] m/z 333 (MH⁺). |
| 78 | 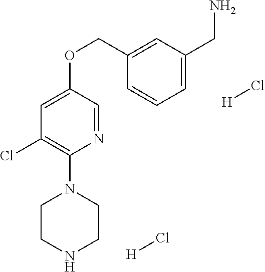 | [3-[(5-chloro-6-piperazin-1-yl-3- pyridyl)oxymethyl]phenyl]methanamine;dihydrochloride Yield: 67%; ¹H NMR (300 MHz, DMSO-d₆): δ 3.13-3.22 (m, 4H), 3.31-3.37 (m, 4H), 3.98-4.06 (m, 2H), 5.16 (s, 2H), 7.42-7.55 (m, 3H), 7.59 (brs, 1H), 7.72 (d, J = 2.5 Hz, 1H), 8.10 (d, J = 2.5 Hz, 1H), 8.57 (brs, 3H), 9.55 (brs, 2H) ppm; ¹³C NMR (75 MHz, DMSO-d₆): δ 42.0, 42.6, 46.1, 70.2, 122.7, 126.0, 127.9, 128.4, 128.7, 128.8, 133.5, 134.5, 136.6, 151.2, 151.3 ppm; [ES+ MS] m/z 333 (MH⁺). |
| 93 | 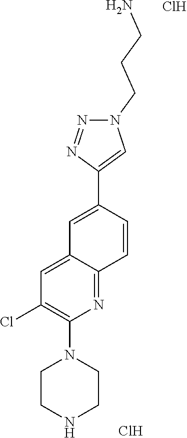 [SMILES: NCCCn1cc(-c2ccc3nc(N4CCNCC4)c(Cl)cc3c2)nn1 ; Cl ; Cl] | 3-[4-(3-chloro-2-piperazin-1-yl-6-quinolyl)triazol-1- yl]propan-1-amine;dihydrochloride Yield: 100%; ¹H NMR (300 MHz, DMSO-d₆): δ 2.22 (p, J = 7.1 Hz, 2H), 2.84 (q, J = 6.5 Hz, 2H), 3.20-3.33 (m, 4H), 3.60-3.71 (m, 4H), 4.59 (t, J = 6.8 Hz, 2H), 7.88 (d, J = 8.8 Hz, 1H), 8.15 (dd, J = 1.9, 8.7 Hz, 1H), 8.26 (brs, 3H), 8.37 (d, J = 1.8 Hz, 1H), 8.55 (s, 1H), 8.78 (s, 1H), 9.62 (brs, 2H) ppm; ¹³C NMR (75 MHz, DMSO-d₆): δ 27.7, 36.2, 42.5, 45.9, 46.9, 122.1, 122.2, 122.5, 126.0, 127.76, 127.80, 127.9, 138.3, 144.2, 145.7, 155.6 ppm; [ES+ MS] m/z 372 (MH⁺). |
| 94 | 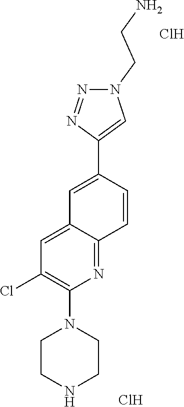 [SMILES: NCCn1cc(-c2ccc3nc(N4CCNCC4)c(Cl)cc3c2)nn1 ; Cl ; Cl] | 2-[4-(3-chloro-2-piperazin-1-yl-6-quinolyl)triazol-1- yl]ethanamine;dihydrochloride Yield: 100%; ¹H NMR (300 MHz, DMSO-d₆): δ 3.23-3.33 (m, 4H), 3.41 (q, J = 5.8 Hz, 2H), 3.62-3.70 (m, 4H), 4.76 (t, J = 6.1 Hz, 2H), 7.90 (d, J = 8.7 Hz, 1H), 8.15 (dd, J = 1.8, 8.7 Hz, 1H), 8.34-8.42 (m, 4H), 8.58 (s, 1H), 8.80 (s, 1H), 9.52 (brs, 2H) ppm; ¹³C NMR (75 MHz, DMSO-d₆): δ 38.4, 42.5, 45.9, 47.1, 122.1, 122.5, 122.7, 126.0, 127.7, 127.80, 127.84, 138.3, 144.2, 145.9, 155.6 ppm; [ES+ MS] m/z 358 (MH⁺). |
| 95 | 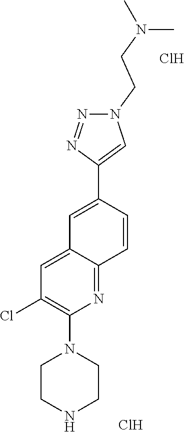 [SMILES: CN(C)CCn1cc(-c2ccc3nc(N4CCNCC4)c(Cl)cc3c2)nn1 ; Cl ; Cl] | 2-[4-(3-chloro-2-piperazin-1-yl-6-quinolyl)triazol-1-yl]- N,N-dimethyl-ethanamine;dihydrochloride Yield: 100%; ¹H NMR (300 MHz, DMSO-d₆): δ 2.82 (d, J = 4.6 Hz, 6H), 3.21-3.32 (m, 4H), 3.62-3.77 (m, 6H), 4.97 (t, J = 6.5 Hz, 2H), 7.89 (d, J = 8.8 Hz, 1H), 8.14 (dd, J = 1.9, 8.7 Hz, 1H), 8.37 (d, J = 1.8 Hz, 1H), 8.56 (s, 1H), 8.85 (s, 1H), 9.66 (brs, 2H), 11.06 (brs, 1H) ppm; ¹³C NMR (75 MHz, DMSO-d₆): δ 42.4, 42.5, 44.5, 45.9, 54.9, 122.1, 122.6, 122.7, 126.0, 127.5, 127.81, 127.84, 138.3, 144.2, 146.0, 155.6 ppm; [ES+ MS] m/z 386 (MH⁺). |
| 96 | 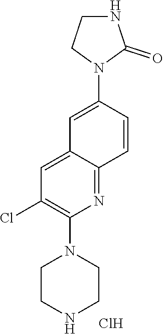 [SMILES: O=C1NCCN1c1ccc2nc(N3CCNCC3)c(Cl)cc2c1 ; Cl] | 1-(3-chloro-2-piperazin-1-yl-6-quinolyl)imidazolidin-2- one;hydrochloride Yield: 87%; ¹H NMR (300 MHz, DMSO-d₆): δ 3.22-3.33 (m, 4H), 3.43-3.48 (m, 2H), 3.53-3.62 (m, 4H), 3.91-3.96 (m, 2H), 7.67 (d, J = 2.6 Hz, 1H), 7.76 (d, J = 9.3 Hz, 1H), 8.32 (dd, J = 2.5, 9.3 Hz, 1H), 8.40 (s, 1H), 9.35 (brs, 2H) ppm; ¹³C NMR (75 MHz, DMSO-d₆): δ 36.5, 42.6, 44.7, 46.1, 111.0, 121.96, 122.00, 126.2, 127.3, 137.3, 138.4, 140.3, 154.1, 158.9 ppm; [ES+ MS] m/z 332 (MH⁺). |
| 97 | 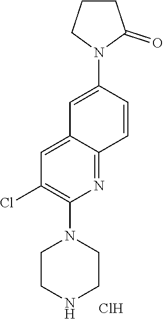 [SMILES: O=C1CCCN1c1ccc2nc(N3CCNCC3)c(Cl)cc2c1 ; Cl] | 1-(3-chloro-2-piperazin-1-yl-6-quinolyl)pyrrolidin-2- one;hydrochloride Yield: 100%; ¹H NMR (300 MHz, DMSO-d₆): δ 2.11 (p, J = 7.5 Hz, 2H), 2.55 (t, J = 7.9 Hz, 2H), 3.21-3.32 (m, 4H), 3.57- 3.64 (m, 4H), 3.93 (t, J = 7.0 Hz, 2H), 7.81 (d, J = 9.2 Hz, 1H), 7.96 (d, J = 2.4 Hz, 1H), 8.23 (dd, J = 2.5, 9.2 Hz, 1H), 8.46 (s, 1H), 9.46 (brs, 2H) ppm; ¹³C NMR (75 MHz, DMSO- d₆): δ 17.4, 32.4, 42.5, 46.0, 48.3, 114.8, 122.1, 123.3, 125.8, 127.3, 136.9, 137.8, 141.4, 154.8, 174.2 ppm; [ES+ MS] m/z 331 (MH⁺). |
| 98 | 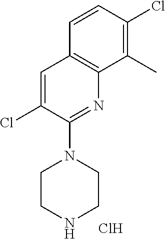 [SMILES: Cc1c(Cl)ccc2cc(Cl)c(N3CCNCC3)nc12 ; Cl] | 3,7-dichloro-8-methyl-2-piperazin-1-yl- quinoline;hydrochloride Yield: 100%; ¹H NMR (300 MHz, DMSO-d₆): δ 2.68 (s, 3H), 3.26-3.32 (m, 4H), 3.68-3.73 (m, 4H), 7.50 (d, J = 8.7 Hz, 1H), 7.73 (d, J = 8.7 Hz, 1H), 8.49 (s, 1H), 9.45 (br s, 2H) ppm; ¹³C NMR (75 MHz, DMSO-d₆): δ 14.1, 42.3, 45.6, 121.1, 124.2, 125.7, 126.2, 132.1, 134.3, 138.5, 143.7, 155.0 ppm; [ES+ MS] m/z 296 (MH⁺). |
| 99 | 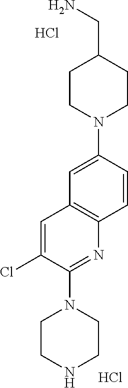 [SMILES: NCC1CCN(c2ccc3nc(N4CCNCC4)c(Cl)cc3c2)CC1 ; Cl ; Cl] | [1-(3-chloro-2-piperazin-1-yl-6-quinolyl)-4- piperidyl]methanamine;dihydrochloride Yield: 100%; ¹H NMR (300 MHz, DMSO-d₆): δ 1.62-1.75 (m, 2H), 1.96-2.04 (m, 2H), 2.76-2.81 (m, 2H), 3.20-3.29 (m, 6H), 3.58-3.63 (m, 4H), 3.72-3.80 (m, 2H), 7.80-7.95 (m, 3H), 8.20 (br s, 3H), 8.40 (s, 1H), 9.51 (br s, 2H) ppm; [ES+ MS] m/z 360 (MH⁺). |
| 100 | 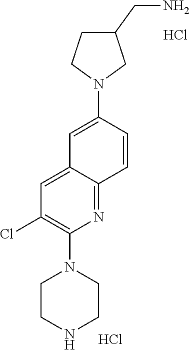 [SMILES: NCC1CCN(c2ccc3nc(N4CCNCC4)c(Cl)cc3c2)C1 ; Cl ; Cl] | [1-(3-chloro-2-piperazin-1-yl-6-quinolyl)pyrrolidin-3- yl]methanamine;dihydrochloride Yield: 100%; ¹H NMR (300 MHz, DMSO-d₆): δ 1.79-1.93 (m, 1H), 2.14-2.27 (m, 1H), 2.63-2.74 (m, 1H), 2.89-2.96 (m, 2H), 3.16-3.56 (m, 12H), 6.72 (d, J = 2.4 Hz, 1H), 7.18 (dd, J = 2.5, 9.2 Hz, 1H), 7.72 (d, J = 9.1 Hz, 1H), 8.23 (s, 1H), 8.33 (br s, 3H), 9.59 (br s, 2H) ppm; ¹³C NMR (75 MHz, DMSO-d₆): δ 28.7, 36.5, 41.3, 42.6, 46.2, 46.3, 51.3, 103.4, 119.3, 122.2, 127.5, 127.7, 136.1, 137.4, 145.0, 152.0 ppm; [ES+ MS] m/z 346 (MH⁺). |
| 101 | 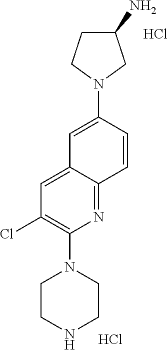 [SMILES: N[C@@H]1CCN(c2ccc3nc(N4CCNCC4)c(Cl)cc3c2)C1 ; Cl ; Cl] | (3R)-1-(3-chloro-2-piperazin-1-yl-6-quinolyl)pyrrolidin- 3-amine;dihydrochloride Yield: 100%; ¹H NMR (300 MHz, DMSO-d₆): δ 2.13-2.22 (m, 1H), 2.29-2.41 (m, 1H), 3.22-3.27 (m, 4H), 3.35-3.66 (m, 8H), 3.93-4.00 (m, 1H), 6.77 (d, J = 2.5 Hz, 1H), 7.20 (dd, J = 2.6, 9.2 Hz, 1H), 7.71 (d, J = 9.1 Hz, 1H), 8.28 (s, 1H), 8.56 (br s, 3H), 9.55 (br s, 2H) ppm; ¹³C NMR (75 MHz, DMSO-d₆): δ 29.2, 42.6, 45.6, 46.2, 49.5, 51.4, 103.7, 119.2, 122.1, 127.5, 127.9, 136.0, 137.7, 144.7, 152.2 ppm; [ES+ MS] m/z 332 (MH⁺). |
| 102 | 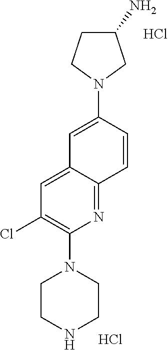 [SMILES: N[C@H]1CCN(c2ccc3nc(N4CCNCC4)c(Cl)cc3c2)C1 ; Cl ; Cl] | (3S)-1-(3-chloro-2-piperazin-1-yl-6-quinolyl)pyrrolidin- 3-amine;dihydrochloride Yield: 95%; ¹H NMR (300 MHz, DMSO-d₆): δ 2.11-2.22 (m, 1H), 2.29-2.42 (m, 1H), 3.22-3.27 (m, 4H), 3.35-3.66 (m, 8H), 3.92-4.00 (m, 1H), 6.77 (d, J = 2.4 Hz, 1H), 7.21 (dd, J = 2.5, 9.2 Hz, 1H), 7.72 (d, J = 9.2 Hz, 1H), 8.28 (s, 1H), 8.57 (br s, 3H), 9.57 (br s, 2H) ppm; ¹³C NMR (75 MHz, DMSO-d₆): δ 29.2, 42.6, 45.6, 46.2, 49.5, 51.4, 103.8, 119.3, 122.1, 127.5, 127.9, 136.0, 137.7, 144.7, 152.2 ppm; [ES+ MS] m/z 332 (MH⁺). |
| 103 | 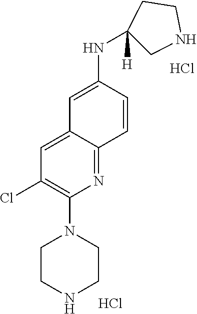 [SMILES: Clc1cc2cc(N[C@@H]3CCNC3)ccc2nc1N1CCNCC1 ; Cl ; Cl] | 3-chloro-2-piperazin-1-yl-N-[(3R)-pyrrolidin-3- yl]quinolin-6-amine;dihydrochloride Yield: 99%; ¹H NMR (300 MHz, DMSO-d₆): δ 1.90-2.00 (m, 1H), 2.20-2.31 (m, 1H), 3.08-3.15 (m, 1H), 3.22-3.38 (m, 6H), 3.44-3.52 (m, 5H), 4.13-4.17 (m, 1H), 6.77 (d, J = 2.5 Hz, 1H), 7.19 (dd, J = 2.5, 9.1 Hz, 1H), 7.61 (d, J = 9.1 Hz, 1H), 8.20 (s, 1H), 9.46-9.65 (br s, 4H) ppm; ¹³C NMR (75 MHz, DMSO-d₆): δ 30.1, 42.6, 43.5, 46.2, 49.2, 51.8, 102.3, 121.9, 122.0, 127.7, 127.9, 136.0, 138.5, 144.9, 152.3 ppm; [ES+ MS] m/z 332 (MH⁺). |
| 104 | 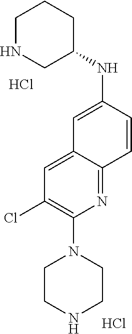 [SMILES: Cl ; Clc1cc2cc(N[C@H]3CCCNC3)ccc2nc1N1CCNCC1 ; Cl] | 3-chloro-2-piperazin-1-yl-N-(3-piperidyl)quinolin-6- amine;dihydrochloride Yield: 100%; ¹H NMR (300 MHz, DMSO-d₆): δ 1.50-1.62 (m, 1H), 1.70-1.83 (m, 1H), 1.87-2.04 (m, 2H), 2.71-2.79 (m, 1H), 2.83-2.93 (m, 1H), 3.14-3.39 (m, 6H), 3.49-3.52 (m, 4H), 3.78-3.87 (m, 1H), 6.89 (d, J = 2.4 Hz, 1H), 7.23 (dd, J = 2.5, 9.1 Hz, 1H), 7.62 (d, J = 9.1 Hz, 1H), 8.12 (s, 1H), 9.36 (br s, 2H), 9.48 (br s, 2H) ppm; ¹³C NMR (75 MHz, DMSO-d₆): δ 20.3, 27.7, 42.6, 42.9, 46.3, 102.6, 121.9, 122.0, 127.7, 127.9, 135.9, 138.4, 144.3, 152.3 ppm; [ES+ MS] m/z 346 (MH⁺). |
| 105 | 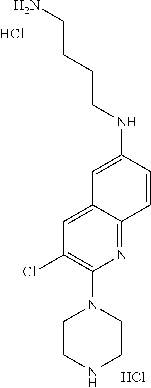 [SMILES: Cl ; NCCCCNc1ccc2nc(N3CCNCC3)c(Cl)cc2c1 ; Cl] | N′-(3-chloro-2-piperazin-1-yl-6-quinolyl)butane-1,4- diamine;dihydrochloride Yield: 96%; ¹H NMR (300 MHz, DMSO-d₆): δ 1.67-1.72 (m, 4H), 2.74-2.83 (m, 2H), 3.15-3.28 (m, 6H), 3.49-3.53 (m, 4H), 6.93 (s, 1H), 7.31 (dd, J = 2.6, 9.0 Hz, 1H), 7.63 (d, J = 9.0 Hz, 1H), 8.04 (br s, 3H), 8.24 (s, 1H), 9.45 (br s, 2H) ppm; [ES+ MS] m/z 334 (MH⁺). |
| 106 | 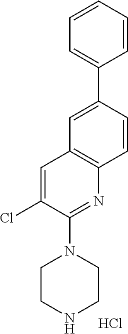 [SMILES: Clc1cc2cc(-c3ccccc3)ccc2nc1N1CCNCC1 ; Cl] | 3-chloro-6-phenyl-2-piperazin-1-yl- quinoline;hydrochloride Yield: 73%; ¹H NMR (300 MHz, DMSO-d₆): δ 3.26-3.30 (m, 4H), 3.64-3.68 (m, 4H), 7.38-7.42 (m, 1H), 7.49-7.52 (m, 2H), 7.76-7.80 (m, 2H), 7.88 (d, J = 8.8 Hz, 1H), 8.04 (dd, J = 2.1, 8.8 Hz, 1H), 8.19 (d, J = 2.0 Hz, 1H), 8.53 (s, 1H), 9.52 (br s, 2H) ppm; ¹³C NMR (75 MHz, DMSO-d₆): δ 42.5, 45.9, 121.8, 124.3, 126.0, 126.9, 127.6, 127.8, 129.1, 129.2, 137.0, 138.4, 139.1, 144.0, 155.5 ppm; [ES+ MS] m/z 324 (MH⁺). |
| 107 | 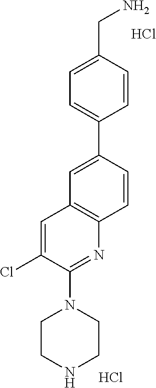 [SMILES: NCc1ccc(-c2ccc3nc(N4CCNCC4)c(Cl)cc3c2)cc1 ; Cl ; Cl] | [4-(3-chloro-2-piperazin-1-yl-6- quinolyl)phenyl]methanamine;dihydrochloride Yield: 85%; ¹H NMR (300 MHz, DMSO-d₆): δ 3.26-3.30 (m, 4H), 3.64-3.69 (m, 4H), 4.08 (s, 2H), 7.64 (d, J = 8.4 Hz, 2H), 7.84 (d, J = 8.3 Hz, 2H), 7.89 (d, J = 8.8 Hz, 1H), 8.06 (dd, J = 2.1, 8.8 Hz, 1H), 8.22 (d, J = 2.0 Hz, 1H), 8.52 (s, 1H), 8.58 (s, 2H), 9.56 (s, 3H) ppm; ¹³C NMR (75 MHz, DMSO-d₆): δ 41.8, 42.5, 45.8, 121.9, 124.4, 126.0, 126.9, 127.7, 129.1, 129.7, 133.6, 136.3, 138.3, 139.2, 144.1, 155.6 ppm; [ES+ MS] m/z 353 (MH⁺). |
| 108 | 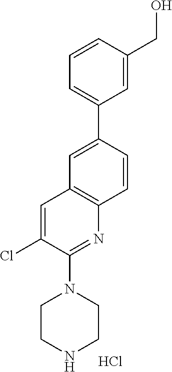 [SMILES: OCc1cccc(-c2ccc3nc(N4CCNCC4)c(Cl)cc3c2)c1 ; Cl] | [3-(3-chloro-2-piperazin-1-yl-6- quinolyl)phenyl]methanol;hydrochloride Yield: 100%; ¹H NMR (300 MHz, DMSO-d₆): δ 3.26-3.32 (m, 4H), 3.63-3.68 (m, 4H), 4.60 (s, 2H), 7.36 (d, J = 7.6 Hz, 1H), 7.48 (t, J = 7.6 Hz, 1H), 7.64 (d, J = 7.8 Hz, 1H), 7.74 (s, 1H), 7.89 (d, J = 8.8 Hz, 1H), 8.03 (dd, J = 2.1, 8.8 Hz, 1H), 8.18 (d, J = 2.0 Hz, 1H), 8.55 (s, 1H), 9.46 (br s, 2H) ppm; ¹³C NMR (75 MHz, DMSO-d₆): δ 42.5, 45.8, 62.8, 121.4, 124.2, 124.9, 125.2, 125.9, 126.0, 127.6, 128.9, 129.2, 137.2, 138.4, 138.9, 143.5, 144.0, 155.5 ppm; [ES+ MS] m/z 354 (MH⁺). |
| 109 | 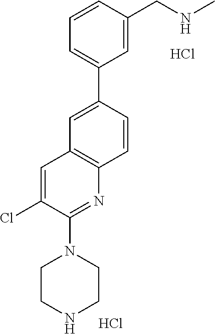 [SMILES: CNCc1cccc(-c2ccc3nc(N4CCNCC4)c(Cl)cc3c2)c1 ; Cl ; Cl] | 1-[3-(3-chloro-2-piperazin-1-yl-6-quinolyl)phenyl]-N- methyl-methanamine;dihydrochloride Yield: 82%; ¹H NMR (300 MHz, DMSO-d₆): δ 2.55 (t, J = 5.4 Hz, 3H), 3.25-3.30 (m, 4H), 3.65-3.70 (m, 4H), 4.19 (t, J = 5.5 Hz, 2H), 7.55-7.58 (m, 2H), 7.80-7.85 (m, 1H), 7.91 (d, J = 8.8 Hz, 1H), 8.08 (d, J = 2.1 Hz, 1H), 8.10 (dd, J = 2.1, 8.8 Hz, 1H), 8.25 (d, J = 2.1 Hz, 1H), 8.50 (s, 1H), 9.58 (br s, 2H), 9.62 (br s, 2H) ppm; ¹³C NMR (75 MHz, DMSO-d₆): δ 31.9, 42.5, 45.9, 51.1, 122.0, 124.5, 125.9, 127.2, 127.7, 128.7, 129.2, 129.3, 129.4, 132.4, 136.4, 138.3, 139.4, 144.1, 155.7 ppm; [ES+ MS] m/z 367 (MH⁺). |
| 110 | 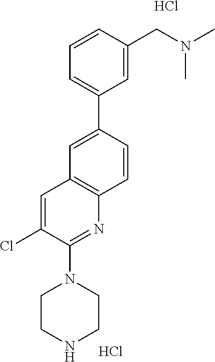 [SMILES: Cl ; CN(C)Cc1cccc(-c2ccc3nc(N4CCNCC4)c(Cl)cc3c2)c1 ; Cl] | 1-[3-(3-chloro-2-piperazin-1-yl-6-quinolyl)phenyl]-N,N- dimethyl-methanamine;dihydrochloride Yield: 71%; ¹H NMR (300 MHz, DMSO-d₆): δ 2.72 (s, 3H), 2.73 (s, 3H), 3.26-3.31 (m, 4H), 3.65-3.70 (m, 4H), 4.37 (d, J = 5.4 Hz, 2H), 7.58-7.61 (m, 2H), 7.84-7.88 (m, 1H), 7.90 (d, J = 8.8 Hz, 1H), 8.12 (dd, J = 2.1, 8.8 Hz, 1H), 8.13 (d, J = 2.0 Hz, 1H), 8.27 (d, J = 1.9 Hz, 1H), 8.49 (s, 1H), 9.61 (br s, 2H), 11.24 (br s, 1H) ppm; ¹³C NMR (75 MHz, DMSO- d₆): δ 41.6, 42.5, 45.9, 59.4, 122.0, 124.6, 125.9, 127.7, 127.7, 129.2, 129.5, 129.8, 130.3, 131.4, 136.3, 138.3, 139.6, 144.1, 155.7 ppm; [ES+ MS] m/z 381 (MH⁺). |
| 111 | 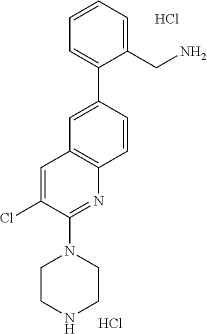 [SMILES: NCc1ccccc1-c1ccc2nc(N3CCNCC3)c(Cl)cc2c1 ; Cl ; Cl] | [2-(3-chloro-2-piperazin-1-yl-6- quinolyl)phenyl]methanamine;dihydrochloride Yield: 99%; ¹H NMR (300 MHz, DMSO-d₆): δ 3.26-3.32 (m, 4H), 3.66-3.70 (m, 4H), 3.93-3.98 (m, 2H), 7.37-7.41 (m, 1H), 7.46-7.56 (m, 2H), 7.72-7.80 (m, 2H), 7.89 (d, J = 8.7 Hz, 1H), 7.93 (d, J = 1.7 Hz, 1H), 8.53 (s, 1H), 8.63 (br s, 3H), 9.61 (br s, 2H) ppm; ¹³C NMR (75 MHz, DMSO-d₆): δ 39.2, 42.4, 45.9, 122.0, 125.5, 127.1, 127.3, 128.1, 128.4, 128.9, 130.3, 131.5, 131.6, 136.6, 138.3, 140.6, 143.8, 155.9 ppm; [ES+ MS] m/z 353 (MH⁺). |
| 112 | 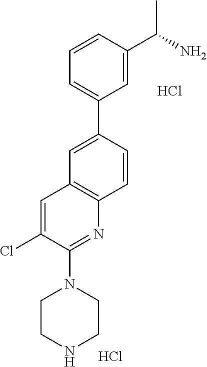 [SMILES: C[C@H](N)c1cccc(-c2ccc3nc(N4CCNCC4)c(Cl)cc3c2)c1 ; Cl ; Cl] | (1S)-1-[3-(3-chloro-2-piperazin-1-yl-6- quinolyl)phenyl]ethanamine; dihydrochloride Yield: 100%; ¹H NMR (300 MHz, DMSO-d₆): δ 1.60 (d, J = 6.8 Hz, 3H), 3.25-3.30 (m, 4H), 3.65-3.69 (m, 4H), 4.46-4.52 (m, 1H), 7.54-7.57 (m, 2H), 7.77-7.81 (m, 1H), 7.91 (d, J = 8.8 Hz, 1H), 8.06 (s, 1H), 8.11 (dd, J = 2.1, 8.8 Hz, 1H), 8.27 (d, J = 2.0 Hz, 1H), 8.51 (s, 1H), 8.73 (br s, 3H), 9.62 (br s, 2H) ppm; ¹³C NMR (75 MHz, DMSO-d₆): δ 20.8, 42.5, 45.9, 50.1, 122.0, 124.5, 125.6, 126.0, 126.3, 126.7, 127.7, 129.2, 129.5, 136.5, 138.3, 139.5, 140.3, 144.1, 155.7 ppm; [ES+ MS] m/z 367 (MH⁺). |
| 113 | 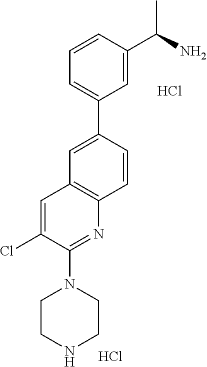 [SMILES: C[C@@H](N)c1cccc(-c2ccc3nc(N4CCNCC4)c(Cl)cc3c2)c1 ; Cl ; Cl] | (1R)-1-[3-(3-chloro-2-piperazin-1-yl-6- quinolyl)phenyl]ethanamine;dihydrochloride Yield: 100%; ¹H NMR (300 MHz, DMSO-d₆): δ 1.60 (d, J = 6.8 Hz, 3H), 3.25-3.30 (m, 4H), 3.65-3.69 (m, 4H), 4.44-4.52 (m, 1H), 7.55-7.57 (m, 2H), 7.77-7.81 (m, 1H), 7.91 (d, J = 8.8 Hz, 1H), 8.06 (s, 1H), 8.11 (dd, J = 2.1, 8.8 Hz, 1H), 8.27 (d, J = 2.0 Hz, 1H), 8.51 (s, 1H), 8.72 (br s, 3H), 9.61 (br s, 2H) ppm; ¹³C NMR (75 MHz, DMSO-d₆): δ 20.8, 42.5, 45.9, 50.1, 122.0, 124.5, 125.6, 126.0, 126.3, 126.7, 127.7, 129.2, 129.5, 136.5, 138.3, 139.5, 140.3, 144.1, 155.7 ppm; [ES+ MS] m/z 367 (MH⁺). |
| 114 | 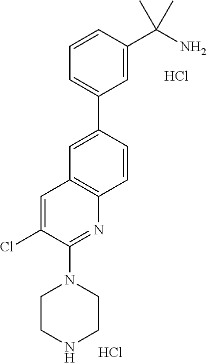 [SMILES: CC(C)(N)c1cccc(-c2ccc3nc(N4CCNCC4)c(Cl)cc3c2)c1 ; Cl ; Cl] | 2-[3-(3-chloro-2-piperazin-1-yl-6- quinolyl)phenyl]propan-2-amine;dihydrochloride Yield: 97%; ¹H NMR (300 MHz, DMSO-d₆): δ 1.72 (s, 6H), 3.26-3.31 (m, 4H), 3.64-3.69 (m, 4H), 7.53-7.61 (m, 2H), 7.75-7.80 (m, 1H), 7.91 (d, J = 8.8 Hz, 1H), 8.09 (s, 1H), 8.15 (dd, J = 2.0, 8.8 Hz, 1H), 8.33 (d, J = 1.8 Hz, 1H), 8.50 (s, 1H), 8.89 (br s, 3H), 9.57 (br s, 2H) ppm; ¹³C NMR (75 MHz, DMSO-d₆): δ 27.7, 42.5, 45.9, 55.6, 121.9, 123.9, 124.4, 124.7, 126.0, 126.2, 127.6, 129.3, 129.4, 136.6, 138.3, 139.3, 143.7, 144.1, 155.7 ppm; [ES+ MS] m/z 381 (MH⁺). |
| 115 | 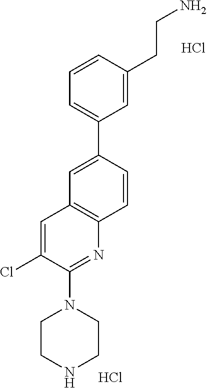 [SMILES: NCCc1cccc(-c2ccc3nc(N4CCNCC4)c(Cl)cc3c2)c1 ; Cl ; Cl] | 2-[3-(3-chloro-2-piperazin-1-yl-6- quinolyl)phenyl]ethanamine;dihydrochloride Yield: 100%; ¹H NMR (300 MHz, DMSO-d₆): δ 2.98-3.05 (m, 2H), 3.08-3.16 (m, 2H), 3.26-3.31 (m, 4H), 3.65-3.70 (m, 4H), 7.31 (d, J = 7.6 Hz, 1H), 7.48 (t, J = 7.6 Hz, 1H), 7.65- 7.70 (m, 2H), 7.89 (d, J = 8.8 Hz, 1H), 8.07 (dd, J = 2.1, 8.8 Hz, 1H), 8.21 (d, J = 2.0 Hz, 1H), 8.23 (br s, 3H), 8.53 (s, 1H), 9.60 (br s, 2H) ppm; ¹³C NMR (75 MHz, DMSO-d₆): δ 33.0, 39.6, 42.5, 45.9, 121.9, 124.4, 125.3, 126.0, 127.3, 127.6, 128.2, 129.3, 129.4, 136.9, 138.3, 139.5, 144.0, 155.6 ppm; [ES+ MS] m/z 367 (MH⁺). |
| 116 | 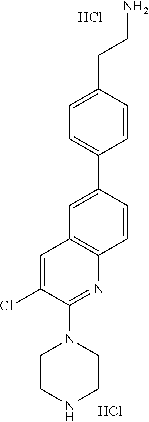 [SMILES: NCCc1ccc(-c2ccc3nc(N4CCNCC4)c(Cl)cc3c2)cc1 ; Cl ; Cl] | 2-[4-(3-chloro-2-piperazin-1-yl-6- quinolyl)phenyl]ethanamine;dihydrochloride Yield: 93%; ¹H NMR (300 MHz, DMSO-d₆): δ 2.94-3.00 (m, 2H), 3.02-3.11 (m, 2H), 3.25-3.30 (m, 4H), 3.64-3.68 (m, 4H), 7.41 (d, J = 8.3 Hz, 2H), 7.75 (d, J = 8.2 Hz, 2H), 7.88 (d, J = 8.8 Hz, 1H), 8.03 (dd, J = 2.0, 9.0 Hz, 1H), 8.18 (d, J = 2.0 Hz, 1H), 8.21 (s, 3H), 8.53 (s, 1H), 9.61 (s, 2H) ppm; ¹³C NMR (75 MHz, DMSO-d₆): δ 32.6, 39.6, 42.5, 45.9, 121.9, 124.1, 126.0, 127.0, 127.6, 129.1, 129.5, 136.7, 137.1, 137.6, 138.4, 143.9, 155.6 ppm; [ES+ MS] m/z 367 (MH⁺). |
| 117 | 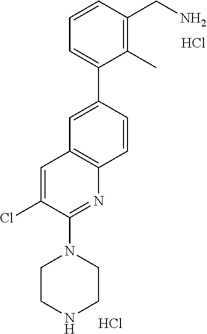 [SMILES: Cc1c(CN)cccc1-c1ccc2nc(N3CCNCC3)c(Cl)cc2c1 ; Cl ; Cl] | [3-(3-chloro-2-piperazin-1-yl-6-quinolyl)-2-methyl- phenyl]methanamine;dihydrochloride Yield: 56%; ¹H NMR (300 MHz, CD₃OD): δ 2.30 (s, 3H), 3.49-3.51 (m, 4H), 3.75-3.77 (m, 4H), 4.30 (s, 2H), 7.34 (dd, J = 1.6, 7.5 Hz, 1H), 7.40 (t, J = 7.5 Hz, 1H), 7.47 (dd, J = 1.6, 7.5 Hz, 1H), 7.65 (dd, J = 1.9, 8.8 Hz, 1H), 7.71 (d, J = 1.7 Hz, 1H), 7.94 (d, J = 8.8 Hz, 1H), 8.38 (s, 1H) ppm; ¹³C NMR (75 MHz, CD₃OD): δ 16.6, 42.0, 44.5, 47.4, 124.0, 127.2, 127.5, 127.8, 129.8, 131.9, 133.0, 133.3, 135.5, 139.6, 140.5, 143.5, 145.2, 157.3 ppm; [ES+ MS] m/z 367 (MH⁺). |
| 118 | 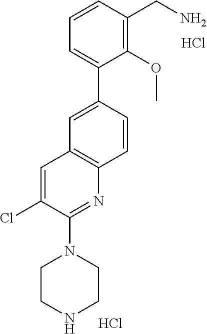 [SMILES: COc1c(CN)cccc1-c1ccc2nc(N3CCNCC3)c(Cl)cc2c1 ; Cl ; Cl] | [3-(3-chloro-2-piperazin-1-yl-6-quinolyl)-2-methoxy- phenyl]methanamine;dihydrochloride Yield: 29% over 2 steps; ¹H NMR (300 MHz, DMSO-d₆): δ 3.24-3.29 (m, 4H), 3.33 (s, 3H), 3.63-3.69 (m, 4H), 4.09- 4.11 (m, 2H), 7.32 (t, J = 7.5 Hz, 1H), 7.49 (d, J = 7.2 Hz, 1H), 7.59 (d, J = 7.5 Hz, 1H), 7.87-7.92 (m, 2H), 8.04 (br s, 1H), 8.56 (br s, 4H), 9.59 (br s, 2H); ¹³C NMR (75 MHz, DMSO-d₆): δ 37.5, 42.9, 46.3 , 61.2, 122.3, 125.0, 126.2, 126.9, 127.6, 128.5, 130.2, 131.5, 132.1, 133.8, 135.1, 138.8, 144.3, 156.0, 156.2 ppm; [ES+ MS] m/z 383 (MH⁺). |
| 119 | 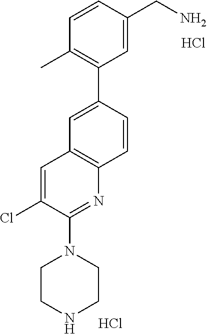 [SMILES: Cc1ccc(CN)cc1-c1ccc2nc(N3CCNCC3)c(Cl)cc2c1 ; Cl ; Cl] | [3-(3-chloro-2-piperazin-1-yl-6-quinolyl)-4-methyl- phenyl]methanamine;dihydrochloride Yield: 33% over 2 steps; ¹H NMR (300 MHz, DMSO-d₆): δ 2.27 (s, 3H), 3.25-3.34 (m, 4H), 3.62-3.69 (m, 4H), 4.04 (m, 2H), 7.36-7.39 (m, 1H), 7.42-7.46 (m, 2H), 7.69-7.72 (m, 1H), 7.86-7.90 (m, 2H), 8.41 (br s, 3H), 8.54 (s, 1H), 9.45 (br s, 2H) ppm; ¹³C NMR (75 MHz, DMSO-d₆): δ 19.9, 41.8, 42.4, 45.8, 121.9, 125.4, 126.6, 126.9, 128.2, 130.1, 130.7, 131.4, 131.7, 135.2, 137.7, 138.1, 140.4, 143.6, 155.6 ppm; [ES+ MS] m/z 367 (MH⁺). |
| 120 | 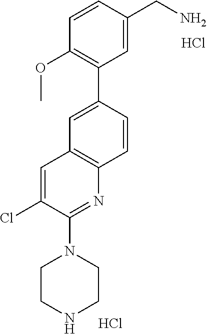 [SMILES: COc1ccc(CN)cc1-c1ccc2nc(N3CCNCC3)c(Cl)cc2c1 ; Cl ; Cl] | [3-(3-chloro-2-piperazin-1-yl-6-quinolyl)-4-methoxy- phenyl]methanamine;dihydrochloride Yield: 87%; ¹H NMR (300 MHz, DMSO-d₆): δ 3.23-3.30 (m, 4H), 3.63-3.69 (m, 4H), 3.81 (s, 3H), 4.01 (d, J = 5.4 Hz, 2H), 7.19 (d, J = 8.5 Hz, 1H), 7.51 (dd, J = 1.9, 8.9 Hz , 1H), 7.60 (d, J = 1.9 Hz, 1H), 7.85 (s, 2H), 8.01 (s, 1H), 8.50 (br s, 4H), 9.65 (s, 2H) ppm; ¹³C NMR (75 MHz, DMSO-d₆): δ 41.6, 42.4, 45.9, 55.9, 111.9, 121.7, 125.5, 126.4, 126.5, 126.9, 128.7, 130.2, 131.7, 131.9, 134.9, 138.2, 143.6, 155.6, 156.3 ppm; [ES+ MS] m/z 383 (MH⁺). |
| 121 | 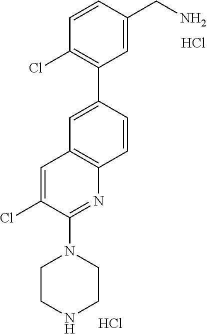 [SMILES: NCc1ccc(Cl)c(-c2ccc3nc(N4CCNCC4)c(Cl)cc3c2)c1 ; Cl ; Cl] | [4-chloro-3-(3-chloro-2-piperazin-1-yl-6- quinolyl)phenyl]methanamine;dihydrochloride Yield: 59% over 2 steps; ¹H NMR (300 MHz, DMSO-d₆): δ 3.28 (m, 4H), 3.68 (m, 4H), 4.10 (q, J = 5.3 Hz, 2H), 7.55- 7.60 (m, 1H), 7.66 (d, J = 8.4 Hz, 1H), 7.71 (d, J = 1.8 Hz, 1H), 7.77 (dd, J = 1.4, 8.6 Hz, 1H), 7.90 (d, J = 8.6 Hz, 1H), 7.97 (d, J = 1.4 Hz, 1H), 8.56 (s, 1H), 8.61 (br s, 3H), 9.59 (br s, 2H) ppm; ¹³C NMR (75 MHz, DMSO-d₆): δ 41.3, 42.4, 45.8, 122.0, 125.3, 126.9, 127.4, 130.1, 131.4, 132.5, 133.7, 135.3, 138.3, 138.9, 143.9, 155.9 ppm; [ES+ MS] m/z 387 (MH⁺). |
| 122 | 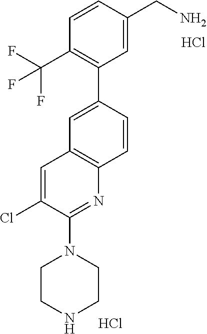 [SMILES: NCc1ccc(C(F)(F)F)c(-c2ccc3nc(N4CCNCC4)c(Cl)cc3c2)c1 ; Cl ; Cl] | [3-(3-chloro-2-piperazin-1-yl-6-quinolyl)-4- (trifluoromethyl)phenyl]methanamine;dihydrochloride Yield: 69%; ¹H NMR (300 MHz, DMSO-d₆): δ 3.26-3.32 (m, 4H), 3.66-3.71 (m, 4H), 4.17 (q, J = 5.2 Hz, 2H), 7.64 (dd, J = 1.3, 8.6 Hz, 1H), 7.68-7.70 (m, 1H), 7.76-7.81 (m, 1H), 7.84 (d, J = 1.5 Hz, 1H), 7.89 (d, J = 8.6 Hz, 1H), 7.94 (d, J = 8.3 Hz, 1H), 8.58 (s, 1H), 8.70 (br s, 3H), 9.58 (br s, 2H) ppm; ¹³C NMR (75 MHz, DMSO-d₆): δ 41.6, 42.8, 45.8, 122.2, 124.0 (q, ¹J_{CF} = 272 Hz), 125.0, 126.5 (q, ³J_{CF} = 3.6 Hz), 126.7, 126.7 (q, ³J_{CF} = 4.1 Hz), 126.8 (q, ²J_{CF} = 28 Hz), 128.7, 130.9, 132.8, 135.8, 138.3, 138.6, 139.9, 143.9, 156.0 ppm; [ES+ MS] m/z 421 (MH⁺). |
| 123 | 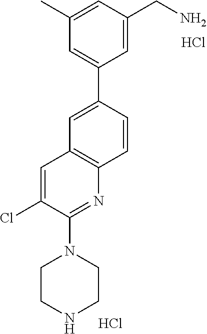 [SMILES: Cc1cc(CN)cc(-c2ccc3nc(N4CCNCC4)c(Cl)cc3c2)c1 ; Cl ; Cl] | [3-(3-chloro-2-piperazin-1-yl-6-quinolyl)-5-methyl- phenyl]methanamine;dihydrochloride Yield: 90%; ¹H NMR (300 MHz, DMSO-d₆): δ 2.41 (s, 3H), 3.22-3.32 (m, 4H), 3.62-3.69 (m, 4H), 4.01-4.09 (m, 2H), 7.33-7.35 (m, 1H), 7.61-7.63 (m, 1H), 7.79-7.82 (m, 1H), 7.90 (d, J = 8.5 Hz, 1H), 8.08 (d, J = 8.3 Hz, 1H), 8.22 (s, 1H), 8.50 (s, 1H), 8.60 (br s, 3H), 9.65 (br s, 2H) ppm; ¹³C NMR (75 MHz, DMSO-d₆): δ 21.1, 42.2, 42.4, 45.8, 121.9, 124.3, 124.9, 125.9, 127.3, 127.6, 128.9, 129.2, 134.8, 136.6, 138.3, 138.5, 139.3, 144.0, 155.6 ppm; [ES+ MS] m/z 367 (MH⁺). |
| 124 | 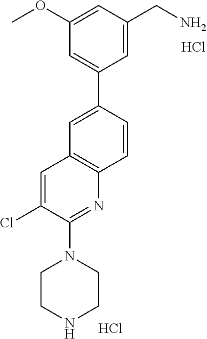 [SMILES: COc1cc(CN)cc(-c2ccc3nc(N4CCNCC4)c(Cl)cc3c2)c1 ; Cl ; Cl] | [3-(3-chloro-2-piperazin-1-yl-6-quinolyl)-5-methoxy- phenyl]methanamine;dihydrochloride Yield: 78%; ¹H NMR (300 MHz, DMSO-d₆): δ 3.23-3.31 (m, 4H), 3.63-3.70 (m, 4H), 3.87 (s, 3H), 4.04-4.10 (m, 2H), 7.19-7.21 (m, 1H), 7.31-7.33 (m, 1H), 7.56-7.58 (m, 1H), 7.89 (d, J = 8.8 Hz, 1H), 8.08 (dd, J = 2.0, 8.8 Hz, 1H), 8.24 (d, J = 1.9 Hz, 1H), 8.50 (s, 1H), 8.63 (br s, 3H), 9.94 (br s, 2H) ppm; ¹³C NMR (75 MHz, DMSO-d₆): δ 42.2, 42.4, 45.9, 55.5, 112.1, 114.0, 119.9, 122.0, 124.6, 125.9, 127.6, 129.2, 136.3, 136.4, 138.4, 140.7, 144.2, 155.7, 160.0 ppm; [ES+ MS] m/z 383 (MH⁺). |
| 125 | 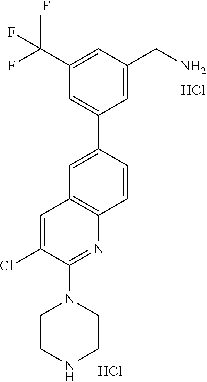 | [3-(3-chloro-2-piperazin-1-yl-6-quinolyl)-5- (trifluoromethyl)phenyl]methanamine;dihydrochloride Yield: 52% over 2 steps; ¹H NMR (300 MHz, DMSO-d₆): δ 3.26-3.31 (m, 4H), 3.66-3.71 (m, 4H), 4.23 (q, J = 5.5 Hz, 2H), 7.94 (d, J = 9.1 Hz, 1H), 7.95-7.96 (m, 1H), 8.12-8.13 (m, 1H), 8.18 (dd, J = 1.9, 8.8 Hz, 1H), 8.38 (d, J = 1.7 Hz, 2H), 8.52 (s, 1H), 8.72 (br s, 3H), 9.58 (br s, 2H) ppm; ¹³C NMR (75 MHz, DMSO-d₆): δ 41.7, 42.5, 45.8, 122.1, 123.0 (q, ³J_{CF} = 3.6 Hz), 124.1 (q, ¹J_{CF} = 272 Hz), 125.0 (q, ³J_{CF} = 3.6 Hz), 125.1, 125.9, 127.8, 129.0, 130.0 (q, ²J_{CF} = 32.0 Hz), 132.0, 134.8, 136.5, 138.4, 140.3, 144.4, 155.9 ppm; [ES+ MS] m/z 421 (MH⁺). |
| 126 | 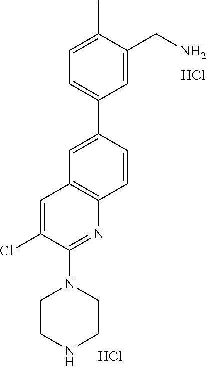 [SMILES: Cc1ccc(-c2ccc3nc(N4CCNCC4)c(Cl)cc3c2)cc1CN ; Cl ; Cl] | [5-(3-chloro-2-piperazin-1-yl-6-quinolyl)-2-methyl- phenyl]methanamine;dihydrochloride Yield: 78%; ¹H NMR (300 MHz, DMSO-d₆): δ 2.41 (s, 3H), 3.23-3.31 (m, 4H), 3.56 (s, 2H), 3.63-3.68 (m, 4H), 7.37 (d, J = 8.1 Hz, 1H), 7.70 (dd, J = 1.6, 8.0 Hz, 1H), 7.90 (d, J = 8.8 Hz, 1H), 7.96 (d, J = 1.5 Hz, 1H), 8.11 (dd, J = 1.9, 8.8 Hz, 1H), 8.24 (d, J = 1.9 Hz, 1H), 8.47 (s, 1H), 8.63 (br s, 3H), 9.62 (br s, 2H) ppm; ¹³C NMR (75 MHz, DMSO-d₆): δ 18.6, 42.5, 45.9, 66.4, 122.0, 124.0, 126.0, 126.5, 127.6, 127.9, 129.1, 131.1, 133.1, 136.3, 136.5, 136.8, 134.2, 144.0, 155.6 ppm; [ES+ MS] m/z 367 (MH⁺). |
| 127 | 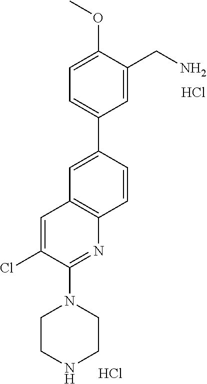 [SMILES: COc1ccc(-c2ccc3nc(N4CCNCC4)c(Cl)cc3c2)cc1CN ; Cl ; Cl] | [5-(3-chloro-2-piperazin-1-yl-6-quinolyl)-2-methoxy- phenyl]methanamine;dihydrochloride Yield: 88%; ¹H NMR (300 MHz, DMSO-d₆): δ 3.24-3.31 (m, 4H), 3.62-3.70 (m, 4H), 3.90 (s, 3H), 4.05 (m, 2H), 7.21 (d, J = 8.4 Hz, 1H), 7.81 (d, J = 8.4 Hz, 1H), 7.88 (d, J = 8.8 Hz, 1H), 7.95 (s, 1H), 8.06 (d, J = 8.8 Hz, 1H), 8.17 (s, 1H), 8.47 (br s, 4H), 9.65 (br s, 2H) ppm; ¹³C NMR (75 MHz, DMSO- d₆): δ 37.9, 42.9, 46.3, 56.3, 112.1, 122.4, 122.9, 123.9, 126.5, 128.1, 128.7, 129.4, 131.6, 136.8, 138.6, 144.2, 155.9, 157.5 ppm; [ES+ MS] m/z 383 (MH⁺). |
| 128 | 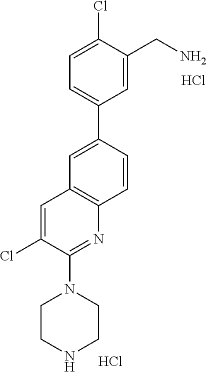 [SMILES: NCc1cc(-c2ccc3nc(N4CCNCC4)c(Cl)cc3c2)ccc1Cl ; Cl ; Cl] | [2-chloro-5-(3-chloro-2-piperazin-1-yl-6- quinolyl)phenyl]methanamine;dihydrochloride Yield: 87%; ¹H NMR (300 MHz, DMSO-d₆): δ 3.26-3.30 (m, 4H), 3.64-3.69 (m, 4H), 4.20-4.31 (m, 2H), 7.64-7.68 (m, 1H), 7.81-7.86 (m, 1H), 7.90-7.94 (m, 1H), 8.10-8.15 (m, 1H), 8.18-8.20 (m, 1H), 8.29-8.31 (m, 1H), 8.48 (s, 1H), 8.78 (br s, 3H), 9.56 (br s, 2H) ppm; ¹³C NMR (75 MHz, DMSO- d₆): δ 40.0, 42.4, 45.7, 121.9, 124.5, 125.8, 127.7, 128.1, 128.9, 129.0, 129.9, 132.1, 135.1, 138.1, 138.2, 144.1, 155.7 ppm; [ES+ MS] m/z 387 (MH⁺). |
| 129 | 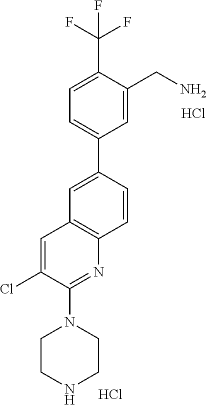 [SMILES: NCc1cc(-c2ccc3nc(N4CCNCC4)c(Cl)cc3c2)ccc1C(F)(F)F ; Cl ; Cl] | [5-(3-chloro-2-piperazin-1-yl-6-quinolyl)-2- (trifluoromethyl)phenyl]methanamine;dihydrochloride Yield: 31% over 2 steps; ¹H NMR (300 MHz, DMSO-d₆): δ 3.26-3.33 (m, 4H), 3.66-3.72 (m, 4H), 4.25-4.29 (m, 2H), 7.89-7.98 (m, 2H), 7.99-8.05 (m, 1H), 8.21 (d, J = 8.3 Hz, 1H), 8.37-8.40 (m, 1H), 8.42-8.45 (m, 1H), 8.50 (s, 1H), 8.90 (br s, 3H), 9.52 (br s, 2H) ppm; ¹³C NMR (75 MHz, DMSO- d₆): δ 38.6, 42.5, 45.8, 122.1, 125.5, 125.8, 126.0 (q, ²J_{CF} = 30 Hz), 126.8, 127.0 (q, ³J_{CF} = 5.2 Hz), 127.8 (q, ¹J_{CF} = 274 Hz), 127.9, 129.0, 129.3, 132.9, 134.7, 138.4, 143.2, 144.6, 156.0 ppm; [ES+ MS] m/z 421 (MH⁺). |
| 130 | 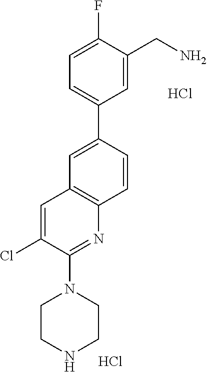 [SMILES: NCc1cc(-c2ccc3nc(N4CCNCC4)c(Cl)cc3c2)ccc1F ; Cl ; Cl] | [5-(3-chloro-2-piperazin-1-yl-6-quinolyl)-2-fluoro- phenyl]methanamine;dihydrochloride Yield: 99%; ¹H NMR (300 MHz, DMSO-d₆): δ 3.25-3.30 (m, 4H), 3.65-3.69 (m, 4H), 4.15 (q, J = 5.4 Hz, 2H), 7.42 (d, J = 9.2 Hz, 1H), 7.82-7.89 (m, 1H), 7.91 (d, J = 8.8 Hz, 1H), 8.08 (dd, J = 2.0, 8.8 Hz, 1H), 8.16 (dd, J = 2.0, 7.0 Hz, 1H), 8.23 (d, J = 1.8 Hz, 1H), 8.48 (s, 1H), 8.71 (br s, 3H), 9.60 (br s, 2H) ppm; ¹³C NMR (75 MHz, DMSO-d₆): δ 35.7, 42.5, 45.8, 116.0 (d, ²J_{CF} = 22.3 Hz), 121.7 (d, ²J_{CF} = 15.3 Hz), 122.0, 124.3, 125.9, 127.7, 129.0 (d, ³J_{CF} = 9.5 Hz), 129.0, 130.1 (⁴J_{CF} = 2.7 Hz), 135.5 (⁴J_{CF} = 2.7 Hz), 135.6, 138.2, 144.0, 155.7, 160.0 (d, ¹J_{CF} = 248 Hz) ppm; [ES+ MS] m/z 371 (MH⁺). |
| 131 | 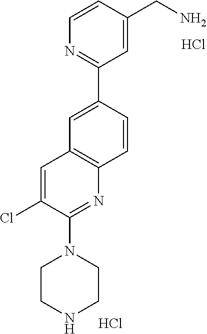 [SMILES: NCc1ccnc(-c2ccc3nc(N4CCNCC4)c(Cl)cc3c2)c1 ; Cl ; Cl] | [2-(3-chloro-2-piperazin-1-yl-6-quinolyl)-4- pyridyl]methanamine;dihydrochloride Yield: 43%; ¹H NMR (300 MHz, DMSO-d₆): δ 3.24-3.34 (m, 4H), 3.64-3.72 (m, 4H), 4.19 (d, J = 5.2 Hz, 2H), 7.51 (d, J = 4.6 Hz, 1H), 7.94 (d, J = 8.8 Hz, 1H), 8.32 (s, 1H), 8.43 (d, J = 8.3 Hz, 1H), 8.59-8.67 (m, 5H), 8.75 (d, J = 4.5 Hz, 1H), 9.30 (br s, 2H) ppm; [ES+ MS] m/z 354 (MH⁺). |
| 132 | 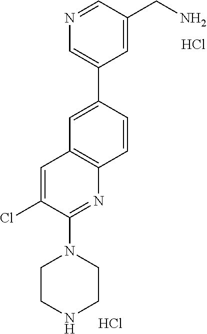 [SMILES: NCc1cncc(-c2ccc3nc(N4CCNCC4)c(Cl)cc3c2)c1 ; Cl ; Cl] | [5-(3-chloro-2-piperazin-1-yl-6-quinolyl)-3- pyridyl]methanamine;dihydrochloride Yield: 25%; ¹H NMR (300 MHz, DMSO-d₆): δ 3.26-3.33 (m, 4H), 3.68-3.75 (m, 4H), 4.27-4.36 (m, 2H), 7.94-8.01 (m, 1H), 8.18-8.25 (m, 1H), 8.45-8.51 (m, 2H), 8.85-9.08 (m, 5H), 9.27 (s, 1H), 9.58 (br s, 2H) ppm; ¹³C NMR (75 MHz, DMSO-d₆): δ 37.6, 42.4, 45.8, 122.3, 125.7, 125.8, 128.1, 128.8, 131.3, 132.6, 136.2, 138.4, 141.0, 142.4, 143.9, 144.8, 156.2 ppm; [ES+ MS] m/z 354 (MH⁺). |
| 133 | 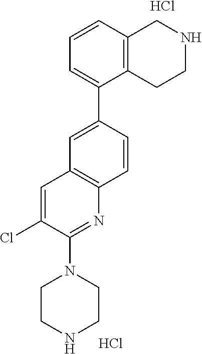 [SMILES: Cl ; Clc1cc2cc(-c3cccc4c3CCNC4)ccc2nc1N1CCNCC1 ; Cl] | 3-chloro-2-piperazin-1-yl-6-(1,2,3,4- tetrahydroisoquinolin-5-yl)quinoline;dihydrochloride Yield: 97%; ¹H NMR (300 MHz, DMSO-d₆): δ 2.82-2.88 (m, 2H), 3.24-3.30 (m, 6H), 3.65-3.69 (m, 4H), 4.31-4.34 (m, 2H), 7.25-7.42 (m, 3H), 7.62 (dd, J = 1.9, 8.6 Hz, 1H), 7.80 (d, J = 1.7 Hz, 1H), 7.89 (d, J = 8.6 Hz, 1H), 8.57 (s, 1H), 9.62 (br s, 2H), 9.76 (br s, 2H) ppm; ¹³C NMR (75 MHz, DMSO-d₆): δ 23.8, 40.4, 42.4, 43.6, 45.9, 121.9, 125.6, 126.5, 126.7, 126.8, 127.1, 128.8, 129.8, 130.0, 131.3, 137.0, 138.3, 140.4, 143.7, 155.8 ppm; [ES+ MS] m/z 379 (MH⁺). |
| 134 | 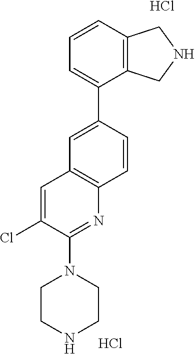 [SMILES: Cl ; Clc1cc2cc(-c3cccc4c3CNC4)ccc2nc1N1CCNCC1 ; Cl] | 3-chloro-6-isoindolin-4-yl-2-piperazin-1-yl- quinoline;dihydrochloride Purification by reverse phase flash chromatography (MeOH/water 10/90-100/0); Yield: 30%; ¹H NMR (300 MHz, DMSO-d₆): δ 3.26-3.31 (m, 4H), 3.65-3.69 (m, 4H), 4.57- 4.65 (m, 4H), 7.46-7.55 (m, 3H), 7.84 (dd, J = 2.0, 8.7 Hz, 1H), 7.91 (d, J = 8.7 Hz, 1H), 8.01 (d, J = 1.9 Hz, 1H), 8.59 (s, 1H), 9.55 (br s, 2H), 10.22 (br s, 2H) ppm; ¹³C NMR (75 MHz, DMSO-d₆): δ 42.4, 45.9, 49.7, 49.9, 122.0, 122.4, 125.8, 126.0, 127.7, 128.4, 129.3, 130.1, 132.9, 135.6, 135.8, 136.3, 138.4, 144.0, 155.9 ppm; [ES+ MS] m/z 365 (MH⁺). |
| 135 | 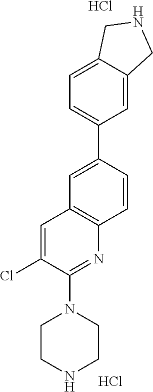 [SMILES: Cl ; Clc1cc2cc(-c3ccc4c(c3)CNC4)ccc2nc1N1CCNCC1 ; Cl] | 3-chloro-6-isoindolin-5-yl-2-piperazin-1-yl- quinoline;dihydrochloride Yield: 100%; ¹H NMR (300 MHz, DMSO-d₆): δ 3.24-3.30 (m, 4H), 3.63-3.70 (m, 4H), 4.53-4.60 (m, 4H) 7.50-7.56 (m, 1H), 7.74-7.92 (m, 3H), 8.00-8.04 (m, 1H), 8.18 (s, 1H), 8.52 (s, 1H), 9.57 (br s, 2H), 10.23 (br s, 2H) ppm; ¹³C NMR (75 MHz, DMSO-d₆): δ 43.0, 46.3, 50.2, 50.3, 121.9, 122.4, 124.1, 124.9, 126.4, 127.5, 128.2, 129.6, 135.1, 136.7, 136.8, 138.8, 139.9, 144.5, 156.1 ppm; [ES+ MS] m/z 365 (MH⁺). |
| 136 | 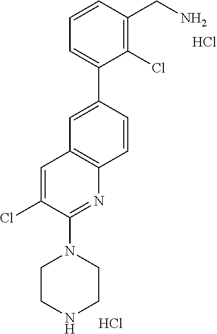 [SMILES: NCc1cccc(-c2ccc3nc(N4CCNCC4)c(Cl)cc3c2)c1Cl ; Cl ; Cl] | [2-chloro-3-(3-chloro-2-piperazin-1-yl-6- quinolyl)phenyl]methanamine;dihydrochloride Yield: 43% over 2 steps; ¹H NMR (300 MHz, DMSO-d₆): δ 3.23-3.33 (m, 4H), 3.64-3.70 (m, 4H), 4.21 (q, J = 4.8 Hz, 2H), 7.45-7.58 (m, 2H), 7.68-7.76 (m, 2H), 7.87-7.92 (m, 2H), 8.57 (s, 1H), 8.73 (br s, 3H), 9.59 (br s, 2H) ppm; ¹³C NMR (75 MHz, DMSO-d₆): δ 40.0, 42.4, 45.8, 121.9, 124.4, 126.8, 127.2, 127.4, 129.9, 131.4, 131.5, 131.7, 132.7, 135.7, 138.3, 139.9, 143.9, 155.9 ppm; [ES+ MS] m/z 387 (MH⁺). |
| 137 | 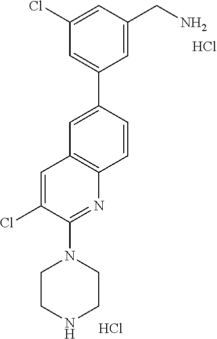 [SMILES: NCc1cc(Cl)cc(-c2ccc3nc(N4CCNCC4)c(Cl)cc3c2)c1 ; Cl ; Cl] | [3-chloro-5-(3-chloro-2-piperazin-1-yl-6- quinolyl)phenyl]methanamine;dihydrochloride Yield: 78% over 2 steps; ¹H NMR (300 MHz, DMSO-d₆): δ 3.23-3.33 (m, 4H), 3.64-3.70 (m, 4H), 4.14 (br s, 2H), 7.64- 7.67 (m, 1H), 7.86-7.88 (m, 1H), 7.91 (d, J = 8.7 Hz, 1H), 8.00-8.03 (m, 1H), 8.11 (dd, J = 1.3, 9.1 Hz, 1H), 8.30 (d, J = 1.3 Hz, 1H), 8.49 (s, 1H), 8.65 (br s, 3H), 9.53 (br s, 2H) ppm; ¹³C NMR (75 MHz, DMSO-d₆): δ 41.7, 42.6, 45.8, 122.1, 124.9, 125.8, 126.3, 126.5, 127.8, 128.0, 128.9, 133.8, 134.9, 137.2, 138.4, 141.3, 144.4, 155.8 ppm; [ES+ MS] m/z 387 (MH⁺). |

### Example 79: 1-(3-chloro-5-iodo-2-pyridyl)piperazine

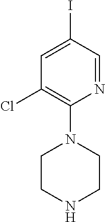 [SMILES: Clc1cc(I)cnc1N1CCNCC1]

A dry 10 mL tube was charged with CuI (0.07 eq), sodium iodide (2.0 eq), Example 18 (0.10 mmol, 1 eq). The tube was purged with argon for 30 min then 1,4-dioxane (2 mL) and (1R,2R)—N1,N2-dimethylcyclohexane-1,2-diamine (0.1 eq) were added. The mixture was heated at 110° C. for 3 days. The product was purified by normal phase flash chromatography (DCM/MeOH 100/0-90/10) and by reverse phase chromatography (MeOH/H₂O 95/5-100/0) to give the title compound. Yield: 63%; ¹H NMR (300 MHz, CD₂Cl₂): δ 2.93-2.97 (m, 4H), 3.24-3.28 (m, 4H), 7.85 (d, J=2.0 Hz, 1H), 8.31 (d, J=2.0 Hz, 1H) ppm; ¹³C NMR (75 MHz, CD₂Cl₂): δ 46.3, 50.7, 81.8, 123.4, 146.1, 151.9, 158.3 ppm; [ES+MS] m/z 324 (MH⁺).

### Example 80: N-[5-chloro-6-(4-methylpiperazin-1-yl)-3-pyridyl]acetamide

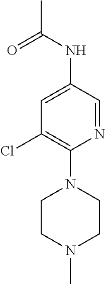 [SMILES: CC(=O)Nc1cnc(N2CCN(C)CC2)c(Cl)c1]

Intermediate 17 (0.7 mmol, 1 eq) and iron (9.5 eq) were dissolved in acetic acid (9.0 mL). The mixture was heated at 50° C. overnight, cooled to room temperature, washed with EtOAc and purified by flash chromatography (DCM/MeOH/NEt₃ 100/0/0-90/05/05) to give the title compound. Yield: 12%; ¹H NMR (300 MHz, CD₂Cl₂): δ 2.13 (s, 3H), 2.31 (s, 3H), 2.54 (t, J=4.9 Hz, 4H), 3.28 (t, J=4.7 Hz, 4H), 7.38 (s, 1H), 8.06 (d, J=2.4 Hz, 1H), 8.13 (d, J=2.4 Hz, 1H) ppm; ¹³C NMR (75 MHz, CD₂Cl₂): δ 24.4, 46.2, 49.5, 55.3, 122.7, 130.2, 131.4, 137.6, 155.4, 168.8 ppm; [ES+MS] m/z 269 (MH⁺).

### Example 81: 3-chloro-2-(4-methylpiperazin-1-yl)quinoline

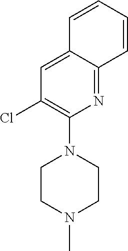 [SMILES: CN1CCN(c2nc3ccccc3cc2Cl)CC1]

2,3-dichloroquinoline (0.25 mmol, 1 eq), methylpiperazine (1.5 eq) and NEt₃ (1.3 eq) in 1 mL DMF was stirred for 2 days at 110° C. The reaction was purified by flash chromatography (cyclohexane/EtOAc 100/0-90/10) to give the title compound. Yield: 39%; ¹H NMR (300 MHz, CD₂Cl₂): δ 2.34 (s, 3H), 2.61 (t, J=4.9 Hz, 4H), 3.50 (t, J=4.7 Hz, 4H), 7.38 (ddd, J=1.2, 6.8, 8.1 Hz, 1H), 7.57-7.67 (m, 2H), 7.80 (d, J=8.1 Hz, 1H), 8.05 (s, 1H); ¹³C NMR (75 MHz, CD₂Cl₂): 46.3, 49.6, 55.3, 123.0, 125.1, 126.0, 126.8, 127.7, 129.9, 137.8, 145.9, 157.3; [ES+MS] m/z 262 (MH⁺).

### Example 82: 2-methoxy-3-piperazin-1-yl-quinoxaline; 2,2,2-trifluoroacetic acid

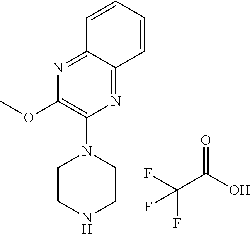

In a flask containing Intermediate 69 (0.08 mmol, 1 eq.) in 2.3 mL of dry DCM, 232 μL of TFA (39.5 eq) were added. The mixture was stirred at room temperature for 1 h 30. The reaction was evaporated under reduced pressure and the solid rinsed several times with DCM to give the title compound. Yield: 88%; ¹H NMR (300 MHz, DMSO-d₆): δ 3.24-3.32 (m, 4H), 3.84 (t, J=5.0 Hz, 4H), 4.05 (s, 3H), 7.44-7.54 (m, 2H), 7.67-7.73 (m, 2H), 8.93 (br s, 2H); ¹³C NMR (75 MHz, DMSO-d₆): δ 42.5, 44.2, 53.9, 115.6 (q, J=291.4 Hz), 125.9, 126.1, 126.3, 126.8, 136.2, 137.4, 146.0, 150.5, 158.4 (q, J=35.9 Hz); [ES+MS] m/z 245 (MH⁺).

### Example 83: 2-chloro-3-(3,6-diazabicyclo[3.1.1]heptan-3-yl)quinoxaline; trifluoromethanesulfonic acid

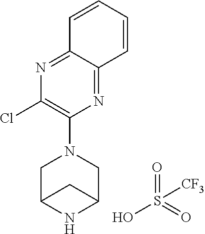

To a solution of Intermediate 32 (0.14 mmol, 1 eq) in 1.5 mL of dry DCM were added dropwise at 0° C. 2,6-lutidine (6 eq) and TMSOTf (3 eq). The solution was stirred at room temperature for 3 h 30. The solvent was evaporated under reduced pressure. The crude was purified by reverse phase flash chromatography (MeCN/H₂O 10/90-100/0) to give the title compound. Yield: 76%; ¹H NMR (300 MHz, DMSO-d₆): δ 1.91 (dd, J=5.2, 10.0 Hz, 1H), 2.79-2.88 (m, 1H), 4.24 (d, J=13.2 Hz, 2H), 4.46-4.57 (m, 4H), 7.57 (ddd, J=1.8, 6.6, 8.4 Hz, 1H), 7.70-7.81 (m, 2H), 7.88 (ddd, J=0.7, 1.4, 8.3 Hz, 1H), 8.12 (br s, 1H), 9.36 (br s, 1H); ¹³C NMR (75 MHz, DMSO-d₆): δ 27.8, 49.1, 58.2, 126.0, 126.7, 127.2, 130.9, 136.7, 136.8, 139.2, 149.6; [ES+MS] m/z 261 (MH⁺).

### Examples 84-92

The appropriate Boc-protected compound (0.10-0.32 mmol, 1 eq) was dissolved in dry DCM (2.9-9.4 mL) and TFA (40 eq) were added. The mixture was stirred at room temperature for 40 min-2 h 30. The reaction was evaporated under reduced pressure and the solid was rinsed with DCM and with diethyl ether to give the desired product.

| | | |
|---|---|---|
| 84 | 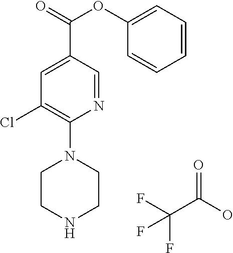 | Phenyl 5-chloro-6-piperazin-1-yl-pyridine-3- carboxylate; 2,2,2-trifluoroacetic acid Yield: 95%; ¹H NMR (300 MHz, DMSO-d₆): δ 3.28 (t, J = 4.8 Hz, 4H), 3.73 (t, J = 4.9 Hz, 4H), 7.26-7.36 (m, 3H), 7.45-7.52 (m, 2H), 8.36 (d, J = 2.0 Hz, 1H), 8.89 (d, J = 2.0 Hz, 1H), 8.97 (brs, 2H) ppm; ¹³C NMR (75 MHz, DMSO-d₆): δ 42.6, 45.2, 119.5, 119.6, 121.8, 126.2, 129.6, 140.1, 147.9, 150.3, 159.2, 162.5 ppm; [ES+ MS] m/z 318 (MH⁺). |
| 85 | 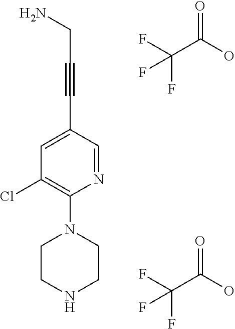 | 3-(5-chloro-6-piperazin-1-yl-3-pyridyl)prop-2- yn-1-amine; 2,2,2-trifluoroacetic acid Yield: 58%; ¹H NMR (300 MHz, DMSO-d₆): δ 3.22-3.28 (m, 4H), 3.51-3.58 (m, 4H), 4.02 (s, 2H), 7.92 (d, J = 2.0 Hz, 1H), 8.33 (d, J = 2.0 Hz, 1H), 8.43 (brs, 3H), 8.98 (brs, 2H) ppm; ¹³C NMR (75 MHz, DMSO-d₆): δ 29.1, 42.7, 45.4, 81.3, 85.5, 112.6, 120.7, 141.3, 148.6, 156.3, 158.1 (q, J = 31.5 Hz, TFA) ppm; [ES+ MS] m/z 251 (MH⁺). |
| 86 | 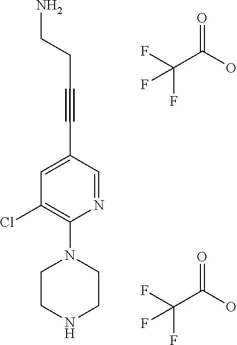 | 4-(5-chloro-6-piperazin-1-yl-3-pyridyl)but-3- yn-1-amine; 2,2,2-trifluoroacetic acid Yield: 94%; ¹H NMR (300 MHz, DMSO-d₆): δ 2.77 (t, J = 6.9 Hz, 2H), 3.05 (dd, J = 6.3, 12.3 Hz, 2H), 3.21-3.27 (m, 4H), 3.48-3.53 (m, 4H), 7.96 (d, J = 1.9 Hz, 1H), 8.02 (brs, 3H), 8.32 (d, J = 1.9 Hz, 1H), 9.00 (brs, 2H) ppm; ¹³C NMR (75 MHz, DMSO-d₆): δ 17.8, 37.7, 42.7, 45.5, 78.1, 88.9, 114.5, 120.7, 141.4, 148.4, 155.8, 158.3 (q, J = 31.5 Hz, TFA) ppm; [ES+ MS] m/z 265 (MH⁺). |
| 87 | 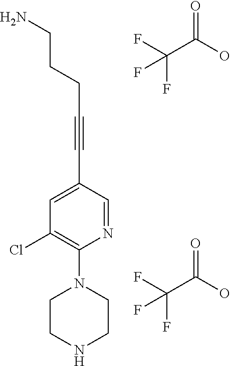 | 5-(5-chloro-6-piperazin-1-yl-3-pyridyl)pent-4- yn-1-amine; 2,2,2-trifluoroacetic acid Yield: 96%; ¹H NMR (300 MHz, DMSO-d₆): δ 1.81 (p, J = 7.4 Hz, 2H), 2.55 (t, J = 7.1 Hz, 2H), 2.89-2.96 (m, 2H), 3.20-3.28 (m, 4H), 3.46-3.52 (m, 4H), 7.81 (brs, 3H), 7.91 (d, J = 2.0 Hz, 1H), 8.29 (d, J = 2.0 Hz, 1H), 8.89 (brs, 2H) ppm; ¹³C NMR (75 MHz, DMSO-d₆): δ 16.0, 26.1, 38.0, 42.7, 45.5, 76.7, 92.2, 114.9, 120.9, 141.2, 148.3, 155.7 ppm; [ES+ MS] m/z 279 (MH⁺). |
| 88 | 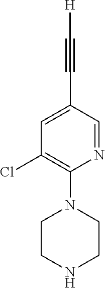 [SMILES: C#Cc1cnc(N2CCNCC2)c(Cl)c1] | 1-(3-chloro-5-ethynyl-2-pyridyl)piperazine Purification by reverse phase chromatography (MeCN 0.1% formic acid/water 0.1% formic acid 5/95-100/0); Yield: 59%; ¹H NMR (300 MHz, DMSO-d₆): δ 3.05-3.13 (m, 4H), 3.39-3.47 (m, 4H), 4.38 (s, 1H), 7.96 (d, J = 2.0 Hz, 1H), 8.34 (d, J = 2.0 Hz, 1H) ppm; ¹³C NMR (75 MHz, DMSO-d₆): δ 43.5, 46.9, 79.3, 83.6, 113.0, 120.5, 141.4, 148.8, 156.6 ppm; [ES+ MS] m/z 222 (MH⁺). |
| 89 | 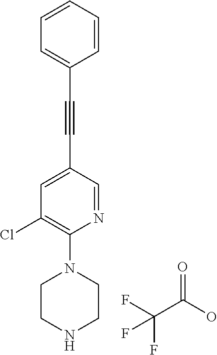 | 1-[3-chloro-5-(2-phenylethynyl)-2- pyridyl]piperazine; 2,2,2-trifluoroacetic acid Yield: 89%; ¹H NMR (300 MHz, DMSO-d₆): δ 3.22-3.30 (m, 4H), 3.53-3.58 (m, 4H), 7.42-7.47 (m, 3H), 7.53-7.59 (m, 2H), 8.08 (d, J = 2.0 Hz, 1H), 8.44 (d, J = 2.0 Hz, 1H), 8.88 (brs, 2H) ppm; ¹³C NMR (75 MHz, DMSO-d₆): δ 42.7, 45.5, 85.0, 92.0, 114.2, 115.7 (q, J = 291.7 Hz, TFA), 120.8, 121.8, 128.9, 129.2, 131.4, 141.2, 148.4, 156.0, 158.3 (q, J = 36.2 Hz, TFA) ppm; [ES+ MS] m/z 298 (MH⁺). |
| 90 | 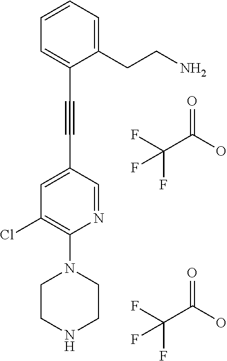 | 2-[2-[2-(5-chloro-6-piperazin-1-yl-3- pyridyl)ethynyl]phenyl]ethanamine; 2,2,2- trifluoroacetic acid Yield: 85%; ¹H NMR (300 MHz, DMSO-d₆): δ 3.11 (s, 4H), 3.21-3.33 (m, 4H), 3.53-3.61 (m, 4H), 7.31-7.46 (m, 3H), 7.56 (dd, J = 1.2, 7.5 Hz, 1H), 8.03 (brs, 3H), 8.12 (d, J = 2.0 Hz, 1H), 8.51 (d, J = 2.0 Hz, 1H), 9.08 (brs, 2H) ppm; ¹³C NMR (75 MHz, DMSO-d₆): δ 32.0, 39.1, 42.7, 45.5, 89.0, 89.9, 114.1, 120.8, 121.6, 127.4, 129.5, 132.4, 139.0, 141.1, 148.6, 156.1, 158.3 (q, J = 30.4 Hz, TFA) ppm; [ES+ MS] m/z 341 (MH⁺). |
| 91 | 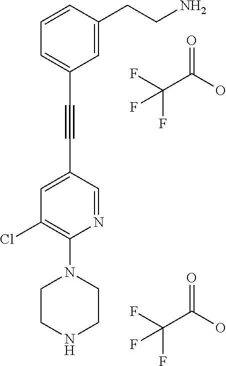 | 2-[3-[2-(5-chloro-6-piperazin-1-yl-3- pyridyl)ethynyl]phenyl]ethanamine; 2,2,2- trifluoroacetic acid Yield: 78%; ¹H NMR (300 MHz, DMSO-d₆): δ 2.89 (t, J = 7.5 Hz, 2H), 3.04-3.13 (m, 2H), 3.23- 3.29 (m, 4H), 3.52-3.58 (m, 4H), 7.34 (td, J = 1.7, 7.2 Hz, 1H), 7.40 (d, J = 7.4 Hz, 1H), 7.44 (t, J = 1.8 Hz, 1H), 7.47 (d, J = 1.6 Hz, 1H), 7.94 (brs, 3H), 8.06 (d, J = 2.0 Hz, 1H), 8.43 (d, J = 2.0 Hz, 1H), 9.04 (brs, 2H) ppm; ¹³C NMR (75 MHz, DMSO-d₆): δ 32.7, 39.7, 42.7, 45.5, 85.1, 91.9, 114.1, 120.8, 122.0, 129.2, 129.7, 129.8, 131.6, 138.1, 141.1, 148.4, 156.0, 158.2 (q, J = 30.9 Hz, TFA) ppm; [ES+ MS] m/z 341 (MH⁺). |
| 92 | 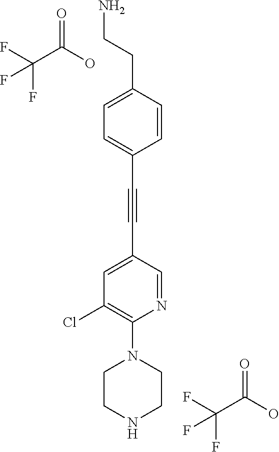 | 2-[4-[2-(5-chloro-6-piperazin-1-yl-3- pyridyl)ethynyl]phenyl]ethanamine; 2,2,2- trifluoroacetic acid Yield: 71%; ¹H NMR (300 MHz, DMSO-d₆): δ 2.87-2.92 (m, 2H), 3.01-3.14 (m, 2H), 3.21-3.30 (m, 4H), 3.51-3.59 (m, 4H), 7.34 (d, J = 8.3 Hz, 2H), 7.53 (d, J = 8.2 Hz, 2H), 7.87 (brs, 3H), 8.07 (d, J = 1.9 Hz, 1H), 8.43 (d, J = 1.9 Hz, 1H), 8.90 (brs, 2H) ppm; ¹³C NMR (75 MHz, DMSO-d₆): δ 32.9, 39.6, 42.8, 45.5, 85.0, 91.9, 114.2, 120.2, 120.9, 129.3, 131.7, 138.6, 141.2, 148.4, 156.0 ppm; [ES+ MS] m/z 341 (MH⁺). |

### Part B—Biological Activity of Compounds According to the Invention

### Material and Methods

Strains, Media and antibiotics

Escherichia coli BW25113 (CGSC 7636) and its derivatives E. coli ΔtolC (JW5503-1, ΔtolC732::kan, CGSC 11430), E. coli ΔacrA (JW0452-3, ΔacrA748::kan, CGSC 11843) and E. coli ΔacrB (JW0451-2, ΔacrB747::kan, CGSC 8609) were obtained from E. coli Genetic Stock Center (CGSC, New Haven, Connecticut) and originated from the Keio Collection (Baba T et al, Mol Sys Biol, 2006). Klebsiella pneumoniae (LMG 2095/ATCC 13883) and K. pneumoniae (ATCC43816), Pseudomonas aeruginosa (PAO1), Acinetobacter baumannii (LMG 1025, ATCC 17978), were obtained from ATCC or BCCM/LMG Bacteria Collection. All bacteria were grown and tested in cation-adjusted Mueller-Hinton broth (CAMHB; BD Difco) at 37° C. For conservation, log phase bacteria were frozen (−80° C.) in CAMHB supplemented with 15% glycerol.

Commercially available molecules, including antibiotics and efflux pump inhibitors, were purchased from various vendors, including Sigma Aldrich, Carbosynth Limited, Fisher Scientific, Euromedex. Pyridomycin was extracted and purified from Dactylosporangium fulvum, as previously described (Hartkoorn R C et al, EMBO Mol Med, 2012).

### Assay 1

Measurement of Growth Inhibition of E. coil Strains by Sub Inhibitory Concentrations of Pyridomycin in Combination with Examples 1-137

For the evaluation of the dose dependent activity of examples 1-137, their ability to boost the antibacterial activity of sub-inhibitory concentrations pyridomycin (an antibiotic that is a good AcrA/B-TolC substrate) was measured. Briefly, E. coli BW25113 were diluted from frozen or growing stocks to an OD₆₀₀ of 0.0004-0.001 in CAMHB. The bacterial suspension was then used as such (no—antibiotic control to evaluate EPI activity), or spiked with pyridomycin at a final concentration of 8 μg/mL. Bacteria were then added to microplates carrying serial dilutions of the compounds examples 1-137, and incubated for (5 h, 37° C.). E. coli BW25113 viability was evaluated using the resazurin reduction assay, and measured by fluorescence (POLARstar Omega, BMG Labtech: Ex: 530 nm Em: 590 nm). EC₅₀ were defined as the compound concentration that prevented 50% of resazurin turnover compared to the non-treated bacteria. Similar assays were also performed in reverse, with a standard dose of EPI (typically 500 μM of compound example 14′ or 100 μM of compound example 37) and a serial dilution of pyridomycin.

### Assay 2

Measurement of Growth Inhibition of A. baumannii Strains by Sub Inhibitory Concentrations of Chloramphenicol in Combination with Examples 1-137

For the evaluation of the dose dependent activity of examples 1-137, their ability to boost the antibacterial activity of sub-inhibitory concentrations chloramphenicol was determined. Briefly, A. baumannii LMG 1025 (ATCC 17978) were diluted from frozen or growing stocks to an OD₆₀₀ of 0.001 in CAMHB. The bacterial suspension was then used as such (no-antibiotic control to evaluate EPI activity), or spiked with chloramphenicol at a final concentration of 10 μg/mL. Bacteria were then added to microplates carrying serial dilutions of the compounds examples 1-137, and incubated for (5 h or 24 h, 37° C.). Bacterial viability was evaluated using the resazurin reduction assay, and measured by fluorescence (POLARstar Omega, BMG Labtech: Ex: 530 nm Em: 590 nm). EC₅₀ were defined as the compound concentration that prevented 90% of resazurin turnover compared to the non-treated bacteria.

### Assay 3

Measurement of growth Inhibition of A. baumannii strains by sub Inhibitory concentrations of novobiocin In combination with examples 1-137

For the evaluation of the dose dependent activity of examples 1-137, their ability to boost the antibacterial activity of sub-inhibitory concentrations novobiocin was determined. Briefly, A. baumannii LMG 1025 (ATCC 17978) were diluted from frozen or growing stocks to an OD₆₀₀ of 0.001 in CAMHB. The bacterial suspension was then used as such (no—antibiotic control to evaluate EPI activity), or spiked with novobiocin at a final concentration of 5 μg/mL. Bacteria were then added to microplates carrying serial dilutions of the compounds examples 1-137, and incubated for (5 h or 24 h, 37° C.). Bacterial viability was evaluated using the resazurin reduction assay, and measured by fluorescence (POLARstar Omega, BMG Labtech: Ex: 530 nm Em: 590 nm). EC₅₀ were defined as the compound concentration that prevented 90% of resazurin turnover compared to the non-treated bacteria.

### Assay 4

Measurement of the Impact of EPI Example 37 on the Boosting of Antibiotic Activity in E. coli, A. baumannii, K. pneumoniae and P. aeruginosa.

To screen for the spectrum of antibiotics boosted by compound example 37, a panel of antibiotics (Erythromycin (ERY), Azithromycin (AZY), Tetracycline (TET), Novobiocin (NOV), Chloramphenicol (CM), Fusidic Acid (FUS), Ciprofloxacin (CIP), Linezolid (LIN), Triclosan (TRC), Pyridomycin (PYR), Streptomycin (STP), Kanamycin (Cm), Genetamicin (Gm), Cefepime (CEP), Ceftazidime (CAZ), Aztreonam (AZT), Oxacillin (OXA), Piperacillin (PPC), Ampicillin (AMP)) were added in a dose response dilutions by acoustic technology (Echo® 550, Labcyte Inc) to a destination microwell plates (typically 384 well plates). Bacterial suspensions were then prepared in CAMHB from a growing pre-culture, (E. coli [OD600=0.001], A. baumannii [OD600=0.001], K. pneumoniae [OD600=0.001] and P. aeruginosa [OD600=0.001]), and added to the microplate using a ViaFill. Microplate containing bacterial cultures were then grown (5 h or 24 h, 37° C.) and bacterial viability determined by either resazurin reduction or OD600.

### Results

The MIC₉₀ of pyridomycin alone on E. coli BW25113 is 12.5-25 μg/mL.

The MIC₉₀ of chloramphenicol alone on A. baumannii LMG 1025 is 25-100 μg/mL.

The MIC₉₀ of novobiocin alone on A. baumannii LMG 1025 is 12.5 μg/mL.

The results of EPI (examples 1-137) antibiotic boosting activity are provided in the Tables below.

| | EC₅₀ on | EC₉₀ on | EC₉₀ on |
|---|---|---|---|
| | E. coli | A. baumannii | A. baumannii |
| Example | with 8 μg/mL of | with 10 μg/mL of | with 5 μg/mL of |
| number | pyridomycin | chloramphenicol | novobiocin |
| 1 | + | | |
| 2 | ++ | | |
| 3 | ++ | | |
| 4 | ++ | | |
| 5 | + | | |
| 6 | + | + | |
| 7 | + | | |
| 8 | ++ | ++ | |
| 9 | ++ | + | |
| 10 | ++ | | |
| 11 | ++ | + | |
| 12 | ++ | | |
| 13 | + | | |
| 14 | ++ | + | |
| 14′ | ++ | + | |
| 15 | + | + | |
| 16 | | + | |
| 17 | ++ | + | |
| 18 | ++ | | |
| 19 | ++ | | |
| 20 | ++ | + | |
| 21 | ++ | + | |
| 22 | ++ | + | |
| 23 | + | + | |
| 24 | ++ | + | |
| 25 | ++ | + | |
| 26 | + | + | |
| 27 | + | + | |
| 28 | +++ | | |
| 29 | +++ | | |
| 30 | ++ | + | |
| 31 | ++ | + | |
| 32 | ++ | | |
| 33 | ++ | + | |
| 34 | ++ | | |
| 35 | + | | |
| 36 | + | | |
| 37 | +++ | ++ | |
| 37′ | +++ | | |
| 38 | ++ | + | |
| 39 | ++ | + | |
| 40 | ++ | | |
| 41 | ++ | + | |
| 42 | ++ | + | |
| 43 | +++ | + | |
| 44 | + | ++ | |
| 45 | + | + | |
| 46 | +++ | + | |
| 47 | +++ | | |
| 48 | ++ | + | |
| 49 | ++ | + | |
| 50 | ++ | | |
| 51 | | + | |
| 52 | +++ | + | |
| 53 | ++ | + | |
| 54 | +++ | + | |
| 55 | +++ | + | |
| 56 | +++ | | |
| 57 | | + | |
| 58 | ++ | + | + |
| 59 | +++ | + | |
| 60 | ++ | | |
| 61 | +++ | | |
| 62 | +++ | | |
| 63 | +++ | | |
| 64 | ++ | | |
| 65 | +++ | | |
| 66 | +++ | ++ | ++ |
| 67 | ++ | | |
| 68 | +++ | | |
| 69 | ++ | | |
| 70 | ++ | | |
| 71 | ++ | | |
| 72 | ++ | | |
| 73 | ++ | | |
| 74 | ++ | | |
| 75 | +++ | | |
| 76 | ++ | | |
| 77 | ++ | | |
| 78 | ++ | | |
| 79 | ++ | | |
| 80 | ++ | | |
| 81 | ++ | | |
| 82 | ++ | + | |
| 83 | + | + | |
| 84 | ++ | | |
| 85 | ++ | | |
| 86 | ++ | | |
| 87 | +++ | | |
| 88 | ++ | | |
| 89 | ++ | | |
| 90 | +++ | | ++ |
| 91 | +++ | | ++ |
| 92 | +++ | | |
| 93 | +++ | | |
| 94 | +++ | | |
| 95 | +++ | | |
| 96 | +++ | | |
| 97 | +++ | | |
| 98 | +++ | ++ | |
| 99 | +++ | | |
| 100 | +++ | | |
| 101 | +++ | | |
| 102 | +++ | | |
| 103 | ++ | | |
| 104 | +++ | | |
| 105 | +++ | | ++ |
| 106 | ++ | ++ | |
| 107 | +++ | | ++ |
| 108 | +++ | | |
| 109 | +++ | ++ | ++ |
| 110 | +++ | | ++ |
| 111 | +++ | | ++ |
| 112 | +++ | | ++ |
| 113 | +++ | | |
| 114 | +++ | | |
| 115 | +++ | | |
| 116 | +++ | | ++ |
| 117 | +++ | | ++ |
| 118 | +++ | | ++ |
| 119 | +++ | | ++ |
| 120 | +++ | | ++ |
| 121 | +++ | | ++ |
| 122 | +++ | | |
| 123 | +++ | | ++ |
| 124 | +++ | | ++ |
| 125 | +++ | | ++ |
| 126 | +++ | | ++ |
| 127 | +++ | | ++ |
| 128 | +++ | | ++ |
| 129 | +++ | | ++ |
| 130 | +++ | ++ | ++ |
| 131 | +++ | | |
| 132 | +++ | | |
| 133 | +++ | | ++ |
| 134 | +++ | ++ | |
| 135 | +++ | | ++ |
| 136 | +++ | | |
| 137 | +++ | | |

≤5 μM = +++
>5 μM to ≤100 μM = ++
>100 μM to <500 μM = +

| | E. coli | K. pneumoniae | K. pneumoniae | A. baumannii | P. aeruginosa |
|---|---|---|---|---|---|
| | BW25113 | ATCC43816 | ATCC13883 | LMG | PAO1 |
| | Fold Boosted MIC | Fold Boosted MIC | Fold Boosted MIC | Fold Boosted MIC | Fold Boosted MIC |
| | By 100 μM | By 100 μM | By 100 μM | By 100 μM | By 100 μM |
| | example 37 | example 37 | example 37 | example 37 | example 37 |
| Erythromycin (ERY) | ** | ** | *** | — | —/* |
| Azithromycin (AZY) | nt | ** | ** | nt | *** |
| Tetracycline (TET) | * | — | * | — | — |
| Novobiocin (NOV) | *** | ** | *** | — | —/* |
| Chloramphenicol (CM) | *** | * | ** | * | — |
| Fusidic Acid (FUS) | *** | ** | 2-4 | — | —/* |
| Ciprofloxacin (CIP) | *** | *** | *** | — | — |
| Linezolid (LIN) | *** | *** | *** | * | * |
| Triclosan (TRC) | * | *** | *** | * | — |
| Pyridomycin (PYR) | *** | *** | *** | — | — |
| Streptomycin (STP) | — | — | — | — | * |
| Kanamycin (Cm) | — | — | — | — | — |
| Genetamicin (Gm) | — | — | — | — | — |
| Cefepime (CEP) | — | * | — | nt | nt |
| Ceftazidime (CAZ) | — | — | — | — | — |
| Aztreonam (AZT) | — | — | 1- | — | — |
| Oxacillin (OXA) | *** | *** | *** | — | * |
| Piperacillin (PPC) | ** | * | * | nt | — |
| Ampicillin (AMP) | * | — | — | — | — |

* = 2-3.9 fold
** = 4-7.9 fold
*** = 8 fold +
— = no change
nt = not tested

1. A compound of formula (I): 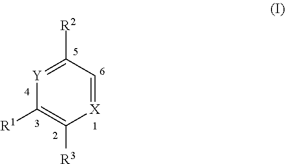
In which:
X can be chosen from:
CH; and
N;
Y can be chosen from:
CH; and
N;
X and Y not being CH at the same time;
R¹ can be chosen from:
a halogen atom;
a —(C₁-C₃)alkyl group;
a —(C₁-C₃)halogenoalkyl group;
a —(C₁-C₃)alkoxy group; or
a nitrile group;
R² can be chosen from:
a halogen atom, the fluorine atom being excluded;
a —(C₁-C₆)alkyl group optionally substituted by one heteroatom chosen from O, N, or S;
a —(C₁-C₆)alkenyl group optionally substituted by one heteroatom chosen from O, N, or S;
a —(C₂-C₆)alkynyl group optionally substituted by one heteroatom chosen from O, N, or S;
a —(C₁-C₃)alkoxy group;
a —(C₁-C₃)halogenoalkyl group;
a —COORa group;
a —N(H)Rb-Ra group;
a —(C₁-C₆)alkynyl-(C₆-C₁₀)aryl group optionally substituted by a —(C₁-C₃)alkyl group, said —(C₁-C₃)alkyl group being optionally substituted by a —NRR′ group;
a CONH—(C₆-C₁₀)aryl group optionally substituted by a —(C₁-C₃)alkyl group, said —(C₁-C₃)alkyl group being optionally substituted by a —NRR′ group;
a —(C₁-C₃)alkyl-O—(C₆-C₁₀)aryl group, said group being optionally substituted by a (C₁-C₃)alkyl group optionally substituted by a —NRR′ group;
a —O—(C₁-C₃)alkyl-(C₆-C₁₀)aryl group, said group being optionally substituted by a (C₁-C₃)alkyl group optionally substituted by a —NRR′ group;
a (5-6 membered)heteroaryl group having at least one heteroatom chosen from O, N, or S; said group being optionally substituted by a (C₁-C₃)alkyl group optionally substituted by a —NRR′ group;
a —CONH—(C₁-C₃)alkyl-(C₆-C₁₀)aryl group, said group being optionally substituted by a halogen atom, a methyl group or a methoxy group; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optionally a bridge of 0 to 2 carbon atoms between 2 members of the heterocycle;
a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁸ group;
Ra is chosen from:
a —(C₁-C₆)alkyl group;
a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
a phenyl group optionally substituted by a —(C₁-C₃)alkyl group, said —(C₁-C₃)alkyl group being substituted by a —NRR′ group; or
a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
carbonyl; and
SO₂;
R³ is chosen from:
a heterocycle comprising 4 to 10 members with at least one N; said heterocycle comprising optionally a bridge of 0 to 2 carbon atoms between 2 members of the heterocycle and being optionally substituted by a —(Cr C₃) alkyl group or a —NRR′ group; and
a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
or its pharmaceutically acceptable salts or optical isomers, racemates, diastereoisomers, enantiomers or tautomers;
with the exclusion of the following compounds:
1-[3-chloro-5-(trifluoromethyl)-2-pyridyl]piperazine;
1-[3-chloro-5-(trifluoromethyl)-2-pyridyl]piperazine hydrochloride;
1-(5-bromo-3-chloro-2-pyridyl)-4-methyl-piperazine;
1-[2-chloro-4-(trifluoromethyl)phenyl]piperazine hydrochloride;
1-[3-chloro-5-(trifluoromethyl)-2-pyridyl]piperidin-4-amine;
1-[3-chloro-5-(trifluoromethyl)-2-pyridyl]-1,4-diazepane;
methyl 5-chloro-6-piperazin-1-yl-pyridine-3-carboxylate hydrochloride;
1-(3-chloro-5-methyl-2-pyridyl)piperazine;
1-(5-bromo-3-chloro-2-pyridyl)piperazine;
2-chloro-3-piperazin-1-yl-quinoxaline hydrochloride;
1-[3-bromo-5-(trifluoromethyl)-2-pyridyl]-4-methyl-piperazine;
3-methyl-2-piperazin-1-yl-quinoline hydrochloride;
2-piperazin-1-ylquinoline-3-carbonitrile hydrochloride;
3-[(3R)-3-methylpiperazin-1-yl]quinoxalin-2-ol hydrochloride;
2-chloro-3-(4-ethylpiperazin-1-yl)quinoxaline;
3-(5-chloro-6-piperazin-1-yl-3-pyridyl)-5-methyl-1,2,4-oxadiazole;
(1R,5S)—N-[3-chloro-5-(trifluoromethyl)-2-pyridyl]-3-azabicyclo[3.1.0]hexan-6-amine;
2-methoxy-3-piperazin-1-yl-quinoxaline hydrochloride;
2-chloro-6,7-dimethyl-3-piperazin-1-yl-quinoxaline;
3-Cyan-2-(4-methyl-piperazino)-5-(pyrid-4-yl)-pyridin;
méthyl 5-chloro-6-4-methylpiperazin-1-yl) nicotinate;
(1-[3-chloro-5-(trifluoromethyl)-2-pyridinyl]-4-methylpiperazine;
1-(3,5-dichloro-2-pyridyl)piperazine; hydrochloride; 2-methoxy-3-(4-methylpiperazin-1-yl)quinoxaline;
2-ethoxy-3-(4-methylpiperazin-1-yl)quinoxaline;
2-methyl-3-(4-methylpiperazin-1-yl)quinoxaline;
2-ethyl-3-(4-methylpiperazin-1-yl)quinoxaline;
2-(4-methylpiperazin-1-yl)-3-(trifluoromethyl)quinoxaline;
2-bromo-3-(4-methylpiperazin-1-yl)quinoxaline;
2-chloro-3-(4-methyl-1,4-diazepan-1-yl)quinoxaline;
3,6-dichloro-2-(4-methylpiperazin-1-yl)quinoxaline;
2,6,7-trichloro-3-(4-methylpiperazin-1-yl)quinoxaline;
1-(3-chloroquinoxalin-2-yl)-N-methyl-pyrrolidin-3-amine
2-chloro-3-[(3S)-3-methylpiperazin-1-yl]quinoxaline hydrochloride; and
4-bromo-1-piperazin-1-yl-isoquinoline.

2. A compound according to claim 1, wherein:
R¹ can be chosen from:
a halogen atom;
a —(C₁-C₃)alkyl group;
a —(C₁-C₃)halogenoalkyl group; or
a —(C₁-C₃)alkoxy group; and/or
R² can be chosen from:
a halogen atom, the fluorine and chlorine atoms being excluded;
a —(C₁-C₆)alkyl group optionally substituted by one heteroatom chosen from O, N, or S;
a —(C₁-C₆)alkenyl group optionally substituted by one heteroatom chosen from O, N, or S;
a —(C₂-C₆)alkynyl group optionally substituted by one heteroatom chosen from O, N, or S;
a —(C₁-C₃)alkoxy group;
a —(C₂-C₃)halogenoalkyl group;
a —COORa group;
a —N(H)Rb-Ra group;
a —(C₁-C₆)alkynyl-(C₆-C₁₀)aryl group optionally substituted by a —(C₁-C₃)alkyl group, said —(C₁-C₃)alkyl group being optionally substituted by a —NRR′ group;
a CONH—(C₆-C₁₀)aryl group optionally substituted by a —(C₁-C₃)alkyl group, said —(C₁-C₃)alkyl group being optionally substituted by a —NRR′ group;
a —(C₁-C₃)alkyl-O—(C₆-C₁₀)aryl group, said group being optionally substituted by a (C₁-C₃)alkyl group optionally substituted by a —NRR′ group;
a —O—(C₁-C₃)alkyl-(C₆-C₁₀)aryl group, said group being optionally substituted by a (C₁-C₃)alkyl group optionally substituted by a —NRR′ group;
a (5-6 membered)heteroaryl group having at least one heteroatom chosen from O, N, or S; said group being optionally substituted by a (C₁-C₃)alkyl group optionally substituted by a —NRR′ group;
a —CONH—(C₁-C₃)alkyl-(C₆-C₁₀)aryl group, said group being optionally substituted by a halogen atom, a methyl group or a methoxy group; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optionally a bridge of 0 to 2 carbon atoms between 2 members of the heterocycle;
a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁸ group;
the other substituents being as defined in claim 1, and provided that:
X and Y are not N at the same time if R¹ is halogen and R³a piperazine, and
R² is not COOCH₃ if R¹ is halogen and R³a piperazine.

3. A compound according to claim 1, wherein:
R¹ is a halogen atom;
R² can be chosen from:
a —(C₂-C₆)alkynyl group optionally substituted by one heteroatom chosen from O, N, or S; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optionally a bridge of 0 to 2 carbon atoms between 2 members of the heterocycle;
a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-6 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁰ group;
the other substituents being as defined in claim 1, and provided that:
X and Y are not N at the same time if R¹ is halogen and R³a piperazine.

4. A compound according to claim 1 wherein,
X can be chosen from:
CH; and
N;
Y can be chosen from:
CH; and
N;
X and Y not being CH or N at the same time.

5. A compound according to claim 1, wherein:
R³ is chosen from a heterocycle comprising 4 to 10 members with at least one N; in particular a piperazine; said heterocycle comprising optionally a bridge of 0 to 2 carbon atoms between 2 members of the heterocycle and being optionally substituted by a —(C₁-C₃) alkyl group or a —NRR′ group.

6. A compound according to claim 1, wherein:
X is N and Y is CH; and/or
R¹ is chosen from chlorine, bromine and iodine; and/or
R² is chosen from:
a iodine;
a —(C₂-C₆)alkynyl group, especially pentynyl group, optionally substituted by a —NRR′ group;
a —(C₂-C₆)alkynyl-(C₆-C₁₀)aryl group, especially ethynyl-phenyl, optionally substituted by a —(C₁-C₃)alkyl group, said —(C₁-C₃)alkyl group being optionally substituted by a —NRR′ group;
a —COORa group;
a (5-6 membered)heteroaryl group having at least one heteroatom chosen from O, N, or S, especially oxadiazol; said group being optionally substituted by a (C₁-C₃)alkyl group, especially ethyl, optionally substituted by a —NRR′ group; or
R² forms together with the carbon atom in position 6, a fused phenyl optionally substituted by
a halogen atom, especially chlorine, bromine and iodine;
one or more —(C₁-C₄)alkyl groups, especially methyl group;
a (4-10 membered)heterocycle having at least one N, especially a piperazine, a pyrrolidine and a imidazolidine, said heterocycle being optionally substituted by a (C₁-C₃)alkyl group, especially methyl, a NRR′ group or a carbonyl; said —(C₁-C₃)alkyl group being optionally substituted by a NRR′ group;
a (C₆-C₁₀)aryl group, especially phenyl, said group being optionally substituted by one or more of a halogen atom, especially fluorine and chlorine, a methyl group optionally substituted by three fluorine atoms, a methoxy group, a (C₁-C₃)alkyl group, optionally substituted by a —NRR′ group or a OH;
a NH-heterocycle comprising 4 to 10 members with a least one N, especially a piperazine;
a (5-12 membered)heteroaryl, especially pyridine and triazole, optionally substituted by (C₁-C₃)alkyl group optionally substituted by a —NRR′ group;
a NR⁷R⁸ group; and/or
Ra is chosen from:
a phenyl group optionally substituted a —(C₁-C₃)alkyl group being substituted by a NRR′ group;
R and R′, identical or different are chosen from —(C₁-C₃)alkyl group, especially methyl, and H; and/or
R³ is a piperazine optionally substituted by a methyl; and/or
R⁷ and R⁸, identical or different are chosen from H, —(C₁-C₄)alkyl group, especially ethyl, propyl and butyl, and —CO—(C₁-C₃)alkyl group, especially —CO—CH₂—; said —(C₁-C₃)alkyl group and —CO—(C₁-C₃)alkyl group being optionally substituted by —NH₂.

7. A compound according to claim 1, wherein:
X is N and Y is CH; and/or
R¹ is chosen from chlorine, bromine and iodine; and/or
R² is chosen from:
a iodine;
a —(C₂-C₆)alkynyl group, especially pentynyl group, optionally substituted by a —NRR′ group;
a —COORa group;
a (5-6 membered)heteroaryl group having at least one heteroatom chosen from O, N, or S, especially oxadiazol; said group being optionally substituted by a (C₁-C₃)alkyl group, especially ethyl, optionally substituted by a —NRR′ group; or
R² forms together with the carbon atom in position 6, a fused phenyl optionally substituted by
a halogen atom, especially chlorine, bromine and iodine;
one or more —(C₁-C₄)alkyl groups, especially methyl group;
a (4-10 membered)heterocycle having at least one N, especially a piperazine;
a (C₆-C₁₀)aryl group, especially phenyl, said group being optionally substituted by a (C₁-C₃)alkyl group, especially methyl, optionally substituted by a —NRR′ group;
a NR⁷R⁸ group; and/or
Ra is chosen from:
a phenyl group optionally substituted a —(C₁-C₃)alkyl group being substituted by a NRR′ group;
R and R′ are H; and/or
R³ is a piperazine optionally substituted by a methyl; and/or
R⁷ and R⁸, identical or different are chosen from H, —(C₁-C₃)alkyl group, especially ethyl and propyl, and —CO—(C₁-C₃)alkyl group, especially —CO—CH₂—; said —(C₁-C₃)alkyl group and —CO—(C₁-C₃)alkyl group being optionally substituted by —NH₂.

8. A compound according to claim 1, wherein said compound is chosen from:
1-(5-bromo-3-chloro-2-pyridyl)piperazine hydrochloride;
1-(3-chloro-5-iodo-2-pyridyl)piperazine;
1-(3-chloro-5-methyl-2-pyridyl)-4-methyl-piperazine;
1-(3-chloro-5-methyl-2-pyridyl)piperazine hydrochloride;
N-[5-chloro-6-(4-methylpiperazin-1-yl)-3-pyridyl]acetamide;
N-(5-chloro-6-piperazin-1-yl-3-pyridyl)acetamide hydrochloride;
N-(5-chloro-6-piperazin-1-yl-3-pyridyl)methane sulfonamide hydrochloride;
methyl 5-chloro-6-(4-methylpiperazin-1-yl)pyridine-3-carboxylate;
ethyl 5-chloro-6-piperazin-1-yl-pyridine-3-carboxylate hydrochloride;
benzyl 5-chloro-6-piperazin-1-yl-pyridine-3-carboxylate hydrochloride;
2-phenylethyl5-chloro-6-piperazin-1-yl-pyridine-3-carboxylate hydrochloride;
3-phenylpropyl5-chloro-6-piperazin-1-yl-pyridine-3 carboxylate hydrochloride;
p-tolylmethyl 5-chloro-6-piperazin-1-yl-pyridine-3-carboxylate hydrochloride;
(4-chlorophenyl)methyl 5-chloro-6-piperazin-1-yl-pyridine-3-carboxylate hydrochloride;
(4-methoxyphenyl)methyl 5-chloro-6-piperazin-1-yl-pyridine-3-carboxylate hydrochloride;
2-(4-chlorophenyl)ethyl 5-chloro-6-piperazin-1-yl-pyridine-3-carboxylate hydrochloride;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
3-bromo-2-piperazin-1-yl-6-(trifluoromethyl)quinoline hydrochloride;
3-bromo-6-chloro-2-piperazin-1-yl-quinoline hydrochloride;
1-[2-chloro-4-(trifluoromethyl)phenyl]-4-methyl-piperazine;
2-chloro-3-piperazin-1-yl-quinoline hydrochloride;
1-[3-bromo-5-(trifluoromethyl)-2-pyridyl]piperazine hydrochloride;
3-iodo-2-piperazin-1-yl-quinoline hydrochloride;
1-[5-iodo-3-(trifluoromethyl)-2-pyridyl]piperazine hydrochloride;
1-(3-chloro-5-iodo-2-pyridyl)piperazine;
3-chloro-2-(1,4-diazepan-1-yl)quinoline;
2-chloro-3-(3,8-diazabicyclo[3.2.1]octan-3-yl)quinoxaline hydrochloride;
N-[(1R,5S)-3-azabicyclo[3.1.0]hexan-6-yl]-3-chloro-quinolin-2-amine hydrochloride;
1-[3-chloro-5-(trifluoromethyl)-2-pyridyl]piperidin-4-amine;
1-(3-chloro-5-methoxy-2-pyridyl)piperazine hydrochloride;
(1R,5S)—N-[3-chloro-5-(trifluoromethyl)-2-pyridyl]-3-azabicyclo[3.1.0]hexan-6-amine formic acid;
3-(4-chlorophenyl)propyl 5-chloro-6-piperazin-1-yl-pyridine-3-carboxylate hydrochloride;
[3-(aminomethyl)phenyl] 5-chloro-6-piperazin-1-yl-pyridine-3-carboxylate dihydrochloride;
5-chloro-N-[(4-chlorophenyl)methyl]-6-piperazin-1-yl-pyridine-3-carboxamide hydrochloride;
N-[4-(2-aminoethyl)phenyl]-5-chloro-6-piperazin-1-yl-pyridine-3-carboxamide dihydrochloride;
2,6-dichloro-3-piperazin-1-yl-quinoline hydrochloride;
2-chloro-6-methyl-3-piperazin-1-yl-quinoline hydrochloride;
3-chloro-2-(3,8-diazabicyclo[3.2.1]octan-8-yl)quinoline hydrochloride;
2-chloro-6,7-dimethyl-3-piperazin-1-yl-quinoxaline hydrochloride;
N-[(1R,5S)-3-azabicyclo[3.1.0]hexan-6-yl]-3-chloro-quinoxalin-2-amine hydrochloride;
3-bromo-6-methyl-2-piperazin-1-yl-quinoline hydrochloride;
3-chloro-2,6-di(piperazin-1 yl)quinoline dihydrochloride;
2-methoxy-3-piperazin-1-yl-quinoxaline 2,2,2-trifluoroacetic acid;
2-chloro-3-(3,6-diazabicyclo[3.1.1]heptan-3-yl)quinoxaline trifluoromethanesulfonic acid; and
2-chloro-3-(4-ethylpiperazin-1-yl)quinoxaline hydrochloride;
4-(2-aminoethyl)-N-(5-chloro-6-piperazin-1-yl-3-pyridyl)benzamide dihydrochloride;
phenyl 5-chloro-6-piperazin-1-yl-pyridine-3-carboxylate 2,2,2-trifluoroacetic acid;
2-(5-chloro-6-piperazin-1-yl-3-pyridyl)-5-methyl-1,3,4-oxadiazole;
3-(5-chloro-6-piperazin-1-yl-3-pyridyl)-5-methyl-1,2,4-oxadiazole hydrochloride;
3-(5-chloro-6-piperazin-1-yl-3-pyridyl)prop-2-yn-1-amine 2,2,2-trifluoroacetic acid;
5-(5-chloro-6-piperazin-1-yl-3-pyridyl)pent-4-yn-1-amine 2,2,2-trifluoroacetic acid;
4-(5-chloro-6-piperazin-1-yl-3-pyridyl)but-3-yn-1-amine 2,2,2-trifluoroacetic acid;
3-chloro-8-methyl-2-piperazin-1-yl-quinoline hydrochloride;
3-chloro-6-iodo-2-piperazin-1-yl-quinoline hydrochloride;
6-bromo-3-chloro-2-piperazin-1-yl-quinoline hydrochloride;
3-chloro-2-piperazin-1-yl-1,5-naphthyridine hydrochloride;
2-chloro-6-methoxy-3-piperazin-1-yl-quinoxaline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
1-(3-chloro-2-piperazin-1-yl-6-quinolyl)piperazin-2-one dihydrochloride;
2-amino-N-(3-chloro-2-piperazin-1-yl-6-quinolyl)acetamide dihydrochloride;
[3-(5-chloro-6-piperazin-1-yl-3-pyridyl)-1,2,4-oxadiazol-5-yl]methanamine dihydrochloride
2-[3-(5-chloro-6-piperazin-1-yl-3-pyridyl)-1,2,4-oxadiazol-5-yl]ethanamine dihydrochloride;
3-[3-(5-chloro-6-piperazin-1-yl-3-pyridyl)-1,2,4-oxadiazol-5-yl]propan-1-amine dihydrochloride;
[3-[(5-chloro-6-piperazin-1-yl-3-pyridyl)methoxy]phenyl]methanamine dihydrochloride;
[3-[(5-chloro-6-piperazin-1-yl-3-pyridyl)oxymethyl]phenyl]methanamine dihydrochloride;
3-chloro-2-piperazin-1-yl-N-(4-piperidyl)quinolin-6-amine dihydrochloride;
1-(3-chloro-2-piperazin-1-yl-6-quinolyl)piperidin-4-amine dihydrochloride;
3-chloro-6-(1,4-diazepan-1-yl)-2-piperazin-1-yl-quinoline dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
1-(3-chloro-2-piperazin-1-yl-6-quinolyl)azetidin-3-amine dihydrochloride;
3-chloro-6-(2,6-diazaspiro[3.3]heptan-2-yl)-2-piperazin-1-yl-quinoline dihydrochloride;
3-chloro-6-(3,8-diazabicyclo[3.2.1]octan-8-yl)-2-piperazin-1-yl-quinoline dihydrochloride;
3-chloro-6-(3,8-diazabicyclo[3.2.1]octan-3-yl)-2-piperazin-1-yl-quinoline dihydrochloride;
3-chloro-2-piperazin-1-yl-6-(1-piperidyl)quinoline hydrochloride;
4-(3-chloro-2-piperazin-1-yl-6-quinolyl)morpholine hydrochloride;
[1-(3-chloro-2-piperazin-1-yl-6-quinolyl)-3-piperidyl]methanamine dihydrochloride;
2-[1-(3-chloro-2-piperazin-1-yl-6-quinolyl)-3-piperidyl]ethanamine dihydrochloride;
[1-(3-chloro-2-piperazin-1-yl-6-quinolyl)pyrrolidin-2-yl]methanamine dihydrochloride
[1-(3-chloro-2-piperazin-1-yl-6-quinolyl)-2-piperidyl]methanamine dihydrochloride;
[3-(3-chloro-2-piperazin-1-yl-6-quinolyl)phenyl]methanamine dihydrochloride;
1-(3-chloro-5-ethynyl-2-pyridyl)piperazine;
3,7-dichloro-8-methyl-2-piperazin-1-yl-quinoline hydrochloride;
3-chloro-6-phenyl-2-piperazin-1-yl-quinoline hydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
3-chloro-2-piperazin-1-yl-N-[(3S)-3-piperidyl]quinolin-6-amine dihydrochloride;
N′-(3-chloro-2-piperazin-1-yl-6-quinolyl)butane-1,4-diamine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
1-[3-chloro-5-(2-phenylethynyl)-2-pyridyl]piperazine 2,2,2-trifluoroacetic acid;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride;
2-[3-(3-chloro-2-piperazin-1-yl-6-quinolyl)phenyl]propan-2-amine dihydrochloride;
2-[5-(3-chloro-2-piperazin-1-yl-6-quinolyl)triazol-1-yl]-N,N-dimethyl-ethanamine dihydrochloride;
[1-(3-chloro-2-piperazin-1-yl-6-quinolyl)imidazol-2-yl]methanamine dihydrochloride;
2-[1-(3-chloro-2-piperazin-1-yl-6-quinolyl)imidazol-2-yl]ethanamine dihydrochloride;
[1-(3-chloro-2-piperazin-1-yl-6-quinolyl)imidazol-4-yl]methanamine dihydrochloride;
2-[1-(3-chloro-2-piperazin-1-yl-6-quinolyl)imidazol-4-yl]ethanamine dihydrochloride;
[3-(3-chloro-2-piperazin-1-yl-6-quinolyl)imidazol-4-yl]methanamine dihydrochloride;
2-[3-(3-chloro-2-piperazin-1-yl-6-quinolyl)imidazol-4-yl]ethanamine dihydrochloride;
[4-(3-chloro-2-piperazin-1-yl-6-quinolyl)-1,2,4-triazol-3-yl]methanamine dihydrochloride;
2-[4-(3-chloro-2-piperazin-1-yl-6-quinolyl)-1,2,4-triazol-3-yl]ethanamine dihydrochloride;
3-(2-aminoethyl)-1-(3-chloro-2-piperazin-1-yl-6-quinolyl)pyrrolidin-2-one dihydrochloride;
1-(2-aminoethyl)-3-(3-chloro-2-piperazin-1-yl-6-quinolyl)imidazolidin-2-one dihydrochloride;
1-(3-chloro-2-piperazin-1-yl-6-quinolyl)-3-[2-(dimethylamino)ethyl]pyrrolidin-2-one dihydrochloride;
1-(3-chloro-2-piperazin-1-yl-6-quinolyl)-3-[2-(dimethylamino)ethyl]imidazolidin-2-one dihydrochloride;
[5-(3-chloro-2-piperazin-1-yl-6-quinolyl)-1,2,4-oxadiazol-3-yl]methanamine dihydrochloride;
2-[5-(3-chloro-2-piperazin-1-yl-6-quinolyl)-1,2,4-oxadiazol-3-yl]ethanamine dihydrochloride;
[3-(3-chloro-2-piperazin-1-yl-6-quinolyl)-1,2,4-oxadiazol-5-yl]methanamine dihydrochloride;
2-[3-(3-chloro-2-piperazin-1-yl-6-quinolyl)-1,2,4-oxadiazol-5-yl]ethanamine dihydrochloride;
[5-(3-chloro-2-piperazin-1-yl-6-quinolyl)-1,3,4-oxadiazol-2-yl]methanamine dihydrochloride;
2-[5-(3-chloro-2-piperazin-1-yl-6-quinolyl)-1,3,4-oxadiazol-2-yl]ethanamine dihydrochloride;
[4-(3-chloro-2-piperazin-1-yl-6-quinolyl)oxazol-2-yl]methanamine dihydrochloride;
2-[4-(3-chloro-2-piperazin-1-yl-6-quinolyl)oxazol-2-yl]ethanamine dihydrochloride;
[4-(3-chloro-2-piperazin-1-yl-6-quinolyl)thiazol-2-yl]methanamine dihydrochloride;
2-[4-(3-chloro-2-piperazin-1-yl-6-quinolyl)thiazol-2-yl]ethanamine dihydrochloride;
[4-(3-chloro-2-piperazin-1-yl-6-quinolyl)-1H-imidazol-2-yl]methanamine dihydrochloride;
2-[4-(3-chloro-2-piperazin-1-yl-6-quinolyl)-1H-imidazol-2-yl]ethanamine dihydrochloride;
[3-(3-chloro-2-piperazin-1-yl-6-quinolyl)-1H-1,2,4-triazol-5-yl]methanamine dihydrochloride;
2-[3-(3-chloro-2-piperazin-1-yl-6-quinolyl)-1H-1,2,4-triazol-5-yl]ethanamine dihydrochloride;
6-[3-(aminomethyl)phenyl]-N-[(1S,5R)-3-azabicyclo[3.1.0]hexan-6-yl]-3-chloro-quinolin-2-amine dihydrochloride;
[3-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride;
[4-(3-chloro-2-piperazin-1-yl-6-quinolyl)-2-pyridyl]methanamine dihydrochloride;
[6-(3-chloro-2-piperazin-1-yl-6-quinolyl)-2-pyridyl]methanamine dihydrochloride;
[2-(3-chloro-2-piperazin-1-yl-6-quinolyl)-4-pyridyl]methanamine dihydrochloride;
[6-(3-chloro-2-piperazin-1-yl-6-quinolyl)pyrazin-2-yl]methanamine dihydrochloride;
3-chloro-6-imidazol-1-yl-2-piperazin-1-yl-quinoline hydrochloride; and
(1S,4S)-2-[3-chloro-5-(trifluoromethyl)-2-pyridyl]-2,5-diazabicyclo[2.2.1]heptane formic acid.

9. A compound according to claim 1, wherein said compound is chosen from:
1-(3-chloro-5-iodo-2-pyridyl)piperazine;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
2-chloro-3-piperazin-1-yl-quinoxaline hydrochloride;
3-iodo-2-piperazin-1-yl-quinoline hydrochloride;
[3-(aminomethyl)phenyl] 5-chloro-6-piperazin-1-yl-pyridine-3-carboxylate dihydrochloride;
3-bromo-6-chloro-2-piperazin-1-yl-quinoline hydrochloride;
3-bromo-6-methyl-2-piperazin-1-yl-quinoline hydrochloride;
3-chloro-2,6-di(piperazin-1 yl)quinoline dihydrochloride;
3-chloro-6-iodo-2-piperazin-1-yl-quinoline hydrochloride;
6-bromo-3-chloro-2-piperazin-1-yl-quinoline hydrochloride;
2-[3-(5-chloro-6-piperazin-1-yl-3-pyridyl)-1,2,4-oxadiazol-5-yl]ethanamine dihydrochloride;
2-chloro-6,7-dimethyl-3-piperazin-1-yl-quinoxaline hydrochloride;
5-(5-chloro-6-piperazin-1-yl-3-pyridyl)pent-4-yn-1-amine 2,2,2-trifluoroacetic acid;
3-chloro-8-methyl-2-piperazin-1-yl-quinoline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
[3-(3-chloro-2-piperazin-1-yl-6-quinolyl)phenyl]methanamine dihydrochloride;
2-amino-N-(3-chloro-2-piperazin-1-yl-6-quinolyl)acetamide dihydrochloride.
[1-(3-chloro-2-piperazin-1-yl-6-quinolyl)-3-piperidyl]methanamine dihydrochloride;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3,7-dichloro-8-methyl-2-piperazin-1-yl-quinoline hydrochloride;
N′-(3-chloro-2-piperazin-1-yl-6-quinolyl)butane-1,4-diamine dihydrochloride;
3-chloro-2-piperazin-1-yl-N-[(3S)-3-piperidyl]quinolin-6-amine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
2-[3-(3-chloro-2-piperazin-1-yl-6-quinolyl)phenyl]propan-2-amine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride; and
[2-(3-chloro-2-piperazin-1-yl-6-quinolyl)-4-pyridyl]methanamine dihydrochloride.

10. A pharmaceutical composition comprising a compound of formula (I) as defined in claim 1, and a pharmaceutically acceptable excipient.

11. A method for treating a bacterial infection, comprising administering to a subject in need thereof, a compound of formula (I): 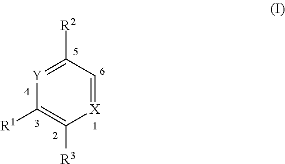
In which:
X can be chosen from:
CH; and
N;
Y can be chosen from:
CH; and
N;
R¹ can be chosen from:
a halogen atom;
a —(C₁-C₃)alkyl group;
a —(C₁-C₃)halogenoalkyl group;
a —(C₁-C₃)alkoxy group;
a nitrile group; or
R¹ forms together with Y a fused phenyl in positions 3 and 4;
R² can be chosen from:
a halogen atom, the fluorine atom being excluded;
a —(C₁-C₆)alkyl group optionally substituted by one heteroatom chosen
from O, N, or S;
a —(C₂-C₆)alkenyl group optionally substituted by one heteroatom chosen
from O, N, or S;
a —(C₂-C₆)alkynyl group optionally substituted by one heteroatom chosen
from O, N, or S;
a —(C₁-C₃)alkoxy group;
a —(C₁-C₃)halogenoalkyl group;
a —COORa group;
a —N(H)Rb-Ra group;
a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
a CONH—(C₆-C₁₀)aryl group optionally substituted by a —(C₁-C₃)alkyl group, said —(C₁-C₃)alkyl group being optionally substituted by a —NRR′ group;
a —(C₁-C₃)alkyl-O—(C₆-C₁₀)aryl group, said group being optionally substituted by a (C₁-C₃)alkyl group optionally substituted by a —NRR′ group;
a —O—(C₁-C₃)alkyl-(C₆-C₁₀)aryl group, said group being optionally substituted by a (C₁-C₃)alkyl group optionally substituted by a —NRR′ group;
a (5-6 membered)heteroaryl group having at least one heteroatom chosen from O, N, or S; said group being optionally substituted by a (C₁-C₃)alkyl group optionally substituted by a —NRR′ group;
a —CONH—(C₁-C₃)alkyl-(C₆-C₁₀)aryl group, said group being optionally substituted by a halogen atom, a methyl group or a methoxy group; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optional a bridge of 0 to 2 carbon atoms between 2 members of the heterocycle;
a (C₆-C₁₀)aryl group, optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁸ group;
Ra is chosen from:
a —(C₁-C₆)alkyl group;
a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
a phenyl group optionally substituted by a —(C₁-C₃)alkyl group, said —(C₁-C₃)alkyl group being substituted by a —NRR′ group; or
a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
carbonyl; and
SO₂;
R³ is chosen from:
a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group;
or its pharmaceutically acceptable salts or optical isomers, racemates, diastereoisomers, enantiomers or tautomers.

12. The method according to claim 11, wherein:
R¹ can be chosen from:
a halogen atom;
a —(C₁-C₃)alkyl group;
a —(C₁-C₃)halogenoalkyl group;
a —(C₁-C₃)alkoxy group; or
a nitrile group;
R² can be chosen from:
a halogen atom, the fluorine atom being excluded;
a —(C₁-C₆)alkyl group optionally substituted by one heteroatom chosen from O, N, or S;
a —(C₁-C₆)alkenyl group optionally substituted by one heteroatom chosen from O, N, or S;
a —(C₂-C₆)alkynyl group optionally substituted by one heteroatom chosen from O, N, or S;
a —(C₁-C₃)alkoxy group;
a —(C₁-C₃)halogenoalkyl group;
a —COORa group;
a —N(H)Rb-Ra group;
a —(C₂-C₆)alkynyl-(C₆-C₁₀)aryl group optionally substituted by a —(C₁-C₃)alkyl group, said —(C₁-C₃)alkyl group being optionally substituted by a —NRR′ group;
a CONH—(C₆-C₁₀)aryl group optionally substituted by a —(C₁-C₃)alkyl group, said —(C₁-C₃)alkyl group being optionally substituted by a —NRR′ group;
a —(C₁-C₃)alkyl-O—(C₆-C₁₀)aryl group, said group being optionally substituted by a (C₁-C₃)alkyl group optionally substituted by a —NRR′ group;
a —O—(C₁-C₃)alkyl-(C₆-C₁₀)aryl group, said group being optionally substituted by a (C₁-C₃)alkyl group optionally substituted by a —NRR′ group;
a (5-6 membered)heteroaryl group having at least one heteroatom chosen from O, N, or S; said group being optionally substituted by a (C₁-C₃)alkyl group optionally substituted by a —NRR′ group;
a —CONH—(C₁-C₃)alkyl-(C₆-C₁₀)aryl group, said group being optionally substituted by a halogen atom, a methyl group or a methoxy group; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optionally a bridge of 0 to 2 carbon atoms between 2 members of the heterocycle;
a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-12 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁸ group;
Ra is chosen from:
a —(C₁-C₆)alkyl group;
a —(C₁-C₃)alkyl-phenyl group optionally substituted by a halogen atom, a —(C₁-C₄)alkyl group optionally substituted by one to three fluorine atoms, a —(C₁-C₃)alkoxy group optionally substituted by one to three fluorine atoms;
a phenyl group optionally substituted by a —(C₁-C₃)alkyl group, said —(C₁-C₃)alkyl group being substituted by a —NRR′ group; or
a —(C₁-C₃)alkyl-(5-6 membered)heteroaryl optionally substituted by a halogen atom, a methyl group optionally substituted by one to three fluorine atoms, a methoxy group optionally substituted by one to three fluorine atoms;
R and R′, identical or different are chosen from —(C₁-C₃)alkyl group and H;
Rb is chosen from:
carbonyl; and
SO₂;
R³ is chosen from:
a heterocycle comprising 4 to 10 members with at least one N; said heterocycle comprising optionally a bridge of 0 to 2 carbon atoms between 2 members of the heterocycle and being optionally substituted by a —(C₁-C₃) alkyl group or a —NRR′ group; and
a —NH-heterocycle, said heterocycle comprising 4 to 10 members with at least one N; said heterocycle comprising optionally a bridge of 0 to 2 carbon atoms between 2 members of the heterocycle;
R⁷ and R⁸, identical or different are chosen from H, —(C₁-C₆)alkyl group and —CO—(C₁-C₆)alkyl group; said —(C₁-C₆)alkyl group and —CO—(C₁-C₆)alkyl group being optionally substituted by —NRR′ group.

13. The method according to claim 11 wherein:
R¹ is a halogen atom; and/or
R² can be chosen from:
a —(C₂-C₆)alkynyl group optionally substituted by one heteroatom chosen from O, N, or S; or
R² forms together with the carbon atom in position 6, a fused phenyl or (5-6 membered)heteroaryl in positions 5 and 6; said fused heteroaryl having at least one heteroatom chosen from O, N, or S; and said fused phenyl or heteroaryl being optionally substituted by one or more of:
one or more halogen atoms,
one or more —(C₁-C₄)alkyl groups,
one or more —(C₁-C₃)halogenoalkyl groups,
one or more —(C₁-C₃)alkoxy groups optionally substituted by one to three fluorine atoms,
a (4-10 membered)heterocycle having at least one N, said heterocycle comprising optionally a bridge of 0 to 2 carbon atoms between 2 members of the heterocycle and being optionally substituted by one or more of a —(C₁-C₃) alkyl group, a NRR′ group or a carbonyl; said —(C₁-C₃) alkyl group being optionally substituted by a NRR′ group;
a NH-heterocycle comprising 4 to 10 members with a least one N, said heterocycle comprising optionally a bridge of 0 to 2 carbon atoms between 2 members of the heterocycle;
a (C₆-C₁₀)aryl group optionally substituted by one or more of a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a (5-6 membered)heteroaryl optionally substituted by a halogen atom, a methyl group optionally substituted by one to three fluorine atoms, a methoxy group optionally substituted by one to three fluorine atoms, a (C₁-C₃)alkyl group optionally substituted by a —NRR′ group or a OH;
a NR⁷R⁸ group; and/or
X can be chosen from:
CH; and
N;
Y can be chosen from:
CH; and
N;
X and Y not being CH or N at the same time; and/or
R³ is chosen from a heterocycle comprising 4 to 10 members with at least one N; in particular a piperazine; said heterocycle comprising optionally a bridge of 0 to 2 carbon atoms between 2 members of the heterocycle and being optionally substituted by a —(C₁-C₃) alkyl group or a —NRR′ group.

14. The method according to claim 11, wherein said compound is chosen from:
1-[3-chloro-5-(trifluoromethyl)-2-pyridyl]-4-methyl-piperazine;
1-[3-chloro-5-(trifluoromethyl)-2-pyridyl]piperazine;
1-[3-chloro-5-(trifluoromethyl)-2-pyridyl]piperazine hydrochloride;
1-(5-bromo-3-chloro-2-pyridyl)-4-methyl-piperazine;
1-(5-bromo-3-chloro-2-pyridyl)piperazine hydrochloride;
1-(3-chloro-5-iodo-2-pyridyl)piperazine;
1-(3-chloro-5-methyl-2-pyridyl)-4-methyl-piperazine;
1-(3-chloro-5-methyl-2-pyridyl)piperazine hydrochloride;
N-[5-chloro-6-(4-methylpiperazin-1-yl)-3-pyridyl]acetamide;
N-(5-chloro-6-piperazin-1-yl-3-pyridyl)acetamide hydrochloride;
N-(5-chloro-6-piperazin-1-yl-3-pyridyl)methane sulfonamide hydrochloride;
methyl 5-chloro-6-(4-methylpiperazin-1-yl)pyridine-3-carboxylate;
methyl 5-chloro-6-piperazin-1-yl-pyridine-3-carboxylate hydrochloride;
ethyl 5-chloro-6-piperazin-1-yl-pyridine-3-carboxylate hydrochloride;
benzyl 5-chloro-6-piperazin-1-yl-pyridine-3-carboxylate hydrochloride;
2-phenylethyl5-chloro-6-piperazin-1-yl-pyridine-3-carboxylate hydrochloride;
3-phenylpropyl5-chloro-6-piperazin-1-yl-pyridine-3 carboxylate hydrochloride;
p-tolylmethyl 5-chloro-6-piperazin-1-yl-pyridine-3-carboxylate hydrochloride;
(4-chlorophenyl)methyl 5-chloro-6-piperazin-1-yl-pyridine-3-carboxylate hydrochloride;
(4-methoxyphenyl)methyl 5-chloro-6-piperazin-1-yl-pyridine-3-carboxylate hydrochloride;
2-(4-chlorophenyl)ethyl 5-chloro-6-piperazin-1-yl-pyridine-3-carboxylate hydrochloride;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
3-bromo-2-piperazin-1-yl-6-(trifluoromethyl)quinoline hydrochloride;
3-bromo-6-chloro-2-piperazin-1-yl-quinoline hydrochloride;
1-[2-chloro-4-(trifluoromethyl)phenyl]-4-methyl-piperazine;
1-[2-chloro-4-(trifluoromethyl)phenyl]piperazine hydrochloride;
2-chloro-3-piperazin-1-yl-quinoline hydrochloride;
2-chloro-3-piperazin-1-yl-quinoxaline hydrochloride;
1-[3-bromo-5-(trifluoromethyl)-2-pyridyl]-4-methyl-piperazine;
1-[3-bromo-5-(trifluoromethyl)-2-pyridyl]piperazine hydrochloride;
3-iodo-2-piperazin-1-yl-quinoline hydrochloride;
3-methyl-2-piperazin-1-yl-quinoline hydrochloride;
2-piperazin-1-ylquinoline-3-carbonitrile hydrochloride;
1-[5-iodo-3-(trifluoromethyl)-2-pyridyl]piperazine hydrochloride;
1-(3-chloro-5-iodo-2-pyridyl)piperazine;
1-[3-chloro-5-(trifluoromethyl)-2-pyridyl]-1,4-diazepane;
3-chloro-2-(1,4-diazepan-1-yl)quinoline;
2-chloro-3-(3,8-diazabicyclo[3.2.1]octan-3-yl)quinoxaline hydrochloride;
3-[(3R)-3-methylpiperazin-1-yl]quinoxalin-2-ol hydrochloride;
2-chloro-3-[(3S)-3-methylpiperazin-1-yl]quinoxaline hydrochloride;
N-[(1R,5S)-3-azabicyclo[3.1.0]hexan-6-yl]-3-chloro-quinolin-2-amine hydrochloride;
1-[3-chloro-5-(trifluoromethyl)-2-pyridyl]piperidin-4-amine;
1-(3-chloro-5-methoxy-2-pyridyl)piperazine hydrochloride;
(1R,5S)—N-[3-chloro-5-(trifluoromethyl)-2-pyridyl]-3-azabicyclo[3.1.0]hexan-6-amine formic acid;
3-(4-chlorophenyl)propyl 5-chloro-6-piperazin-1-yl-pyridine-3-carboxylate hydrochloride;
[3-(aminomethyl)phenyl] 5-chloro-6-piperazin-1-yl-pyridine-3-carboxylate dihydrochloride;
5-chloro-N-[(4-chlorophenyl)methyl]-6-piperazin-1-yl-pyridine-3-carboxamide hydrochloride;
N-[4-(2-aminoethyl)phenyl]-5-chloro-6-piperazin-1-yl-pyridine-3-carboxamide dihydrochloride;
2,6-dichloro-3-piperazin-1-yl-quinoline hydrochloride;
2-chloro-6-methyl-3-piperazin-1-yl-quinoline hydrochloride;
3-chloro-2-(3,8-diazabicyclo[3.2.1]octan-8-yl)quinoline hydrochloride;
2-chloro-6,7-dimethyl-3-piperazin-1-yl-quinoxaline hydrochloride;
N-[(1R,5S)-3-azabicyclo[3.1.0]hexan-6-yl]-3-chloro-quinoxalin-2-amine hydrochloride;
3-bromo-6-methyl-2-piperazin-1-yl-quinoline hydrochloride;
3-chloro-2,6-di(piperazin-1 yl)quinoline dihydrochloride;
2-methoxy-3-piperazin-1-yl-quinoxaline 2,2,2-trifluoroacetic acid;
2-chloro-3-(3,6-diazabicyclo[3.1.1]heptan-3-yl)quinoxaline trifluoromethanesulfonic acid;
2-chloro-3-(4-ethylpiperazin-1-yl)quinoxaline hydrochloride;
4-(2-aminoethyl)-N-(5-chloro-6-piperazin-1-yl-3-pyridyl)benzamide dihydrochloride;
phenyl 5-chloro-6-piperazin-1-yl-pyridine-3-carboxylate 2,2,2-trifluoroacetic acid;
3-(5-chloro-6-piperazin-1-yl-3-pyridyl)-5-methyl-1,2,4-oxadiazole hydrochloride;
3-(5-chloro-6-piperazin-1-yl-3-pyridyl)prop-2-yn-1-amine 2,2,2-trifluoroacetic acid;
5-(5-chloro-6-piperazin-1-yl-3-pyridyl)pent-4-yn-1-amine 2,2,2-trifluoroacetic acid;
4-(5-chloro-6-piperazin-1-yl-3-pyridyl)but-3-yn-1-amine 2,2,2-trifluoroacetic acid;
3-chloro-8-methyl-2-piperazin-1-yl-quinoline hydrochloride;
3-chloro-6-iodo-2-piperazin-1-yl-quinoline hydrochloride;
6-bromo-3-chloro-2-piperazin-1-yl-quinoline hydrochloride;
3-chloro-2-piperazin-1-yl-1,5-naphthyridine hydrochloride;
2-chloro-6-methoxy-3-piperazin-1-yl-quinoxaline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
1-(3-chloro-2-piperazin-1-yl-6-quinolyl)piperazin-2-one dihydrochloride;
2-amino-N-(3-chloro-2-piperazin-1-yl-6-quinolyl)acetamide dihydrochloride;
[3-(5-chloro-6-piperazin-1-yl-3-pyridyl)-1,2,4-oxadiazol-5-yl]methanamine dihydrochloride;
2-[3-(5-chloro-6-piperazin-1-yl-3-pyridyl)-1,2,4-oxadiazol-5-yl]ethanamine dihydrochloride;
3-[3-(5-chloro-6-piperazin-1-yl-3-pyridyl)-1,2,4-oxadiazol-5-yl]propan-1-amine dihydrochloride;
[3-[(5-chloro-6-piperazin-1-yl-3-pyridyl)methoxy]phenyl]methanamine dihydrochloride;
[3-[(5-chloro-6-piperazin-1-yl-3-pyridyl)oxymethyl]phenyl]methanamine dihydrochloride;
3-chloro-2-piperazin-1-yl-N-(4-piperidyl)quinolin-6-amine dihydrochloride;
1-(3-chloro-2-piperazin-1-yl-6-quinolyl)piperidin-4-amine dihydrochloride;
3-chloro-6-(1,4-diazepan-1-yl)-2-piperazin-1-yl-quinoline dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
1-(3-chloro-2-piperazin-1-yl-6-quinolyl)azetidin-3-amine dihydrochloride;
3-chloro-6-(2,6-diazaspiro[3.3]heptan-2-yl)-2-piperazin-1-yl-quinoline dihydrochloride
3-chloro-6-(3,8-diazabicyclo[3.2.1]octan-8-yl)-2-piperazin-1-yl-quinoline dihydrochloride;
3-chloro-6-(3,8-diazabicyclo[3.2.1]octan-3-yl)-2-piperazin-1-yl-quinoline dihydrochloride;
3-chloro-2-piperazin-1-yl-6-(1-piperidyl)quinoline hydrochloride;
4-(3-chloro-2-piperazin-1-yl-6-quinolyl)morpholine hydrochloride;
[1-(3-chloro-2-piperazin-1-yl-6-quinolyl)-3-piperidyl]methanamine dihydrochloride;
2-[1-(3-chloro-2-piperazin-1-yl-6-quinolyl)-3-piperidyl]ethanamine dihydrochloride;
[1-(3-chloro-2-piperazin-1-yl-6-quinolyl)pyrrolidin-2-yl]methanamine dihydrochloride;
[1-(3-chloro-2-piperazin-1-yl-6-quinolyl)-2-piperidyl]methanamine dihydrochloride;
[3-(3-chloro-2-piperazin-1-yl-6-quinolyl)phenyl]methanamine dihydrochloride;
1-(3-chloro-5-ethynyl-2-pyridyl)piperazine;
3,7-dichloro-8-methyl-2-piperazin-1-yl-quinoline hydrochloride;
3-chloro-6-phenyl-2-piperazin-1-yl-quinoline hydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
3-chloro-2-piperazin-1-yl-N-[(3R)-pyrrolidin-3-yl]quinolin-6-amine dihydrochloride;
3-chloro-2-piperazin-1-yl-N-[(3S)-3-piperidyl]quinolin-6-amine dihydrochloride;
N′-(3-chloro-2-piperazin-1-yl-6-quinolyl)butane-1,4-diamine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride;
1-(3-chloro-2-piperazin-1-yl-6-quinolyl)imidazolidin-2-one hydrochloride;
1-[3-chloro-5-(2-phenylethynyl)-2-pyridyl]piperazine 2,2,2-trifluoroacetic acid;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride;
2-[3-(3-chloro-2-piperazin-1-yl-6-quinolyl)phenyl]propan-2-amine dihydrochloride;
2-[5-(3-chloro-2-piperazin-1-yl-6-quinolyl)triazol-1-yl]-N,N-dimethyl-ethanamine dihydrochloride;
[1-(3-chloro-2-piperazin-1-yl-6-quinolyl)imidazol-2-yl]methanamine dihydrochloride;
2-[1-(3-chloro-2-piperazin-1-yl-6-quinolyl)imidazol-2-yl]ethanamine dihydrochloride;
[1-(3-chloro-2-piperazin-1-yl-6-quinolyl)imidazol-4-yl]methanamine dihydrochloride;
2-[1-(3-chloro-2-piperazin-1-yl-6-quinolyl)imidazol-4-yl]ethanamine dihydrochloride;
[3-(3-chloro-2-piperazin-1-yl-6-quinolyl)imidazol-4-yl]methanamine dihydrochloride;
2-[3-(3-chloro-2-piperazin-1-yl-6-quinolyl)imidazol-4-yl]ethanamine dihydrochloride;
[4-(3-chloro-2-piperazin-1-yl-6-quinolyl)-1,2,4-triazol-3-yl]methanamine dihydrochloride;
2-[4-(3-chloro-2-piperazin-1-yl-6-quinolyl)-1,2,4-triazol-3-yl]ethanamine dihydrochloride;
3-(2-aminoethyl)-1-(3-chloro-2-piperazin-1-yl-6-quinolyl)pyrrolidin-2-one dihydrochloride;
1-(2-aminoethyl)-3-(3-chloro-2-piperazin-1-yl-6-quinolyl)imidazolidin-2-one dihydrochloride;
1-(3-chloro-2-piperazin-1-yl-6-quinolyl)-3-[2-(dimethylamino)ethyl]pyrrolidin-2-one dihydrochloride;
1-(3-chloro-2-piperazin-1-yl-6-quinolyl)-3-[2-(dimethylamino)ethyl]imidazolidin-2-one dihydrochloride;
[5-(3-chloro-2-piperazin-1-yl-6-quinolyl)-1,2,4-oxadiazol-3-yl]methanamine dihydrochloride;
2-[5-(3-chloro-2-piperazin-1-yl-6-quinolyl)-1,2,4-oxadiazol-3-yl]ethanamine dihydrochloride;
[3-(3-chloro-2-piperazin-1-yl-6-quinolyl)-1,2,4-oxadiazol-5-yl]methanamine dihydrochloride;
2-[3-(3-chloro-2-piperazin-1-yl-6-quinolyl)-1,2,4-oxadiazol-5-yl]ethanamine dihydrochloride;
[5-(3-chloro-2-piperazin-1-yl-6-quinolyl)-1,3,4-oxadiazol-2-yl]methanamine dihydrochloride;
2-[5-(3-chloro-2-piperazin-1-yl-6-quinolyl)-1,3,4-oxadiazol-2-yl]ethanamine dihydrochloride;
[4-(3-chloro-2-piperazin-1-yl-6-quinolyl)oxazol-2-yl]methanamine dihydrochloride;
2-[4-(3-chloro-2-piperazin-1-yl-6-quinolyl)oxazol-2-yl]ethanamine dihydrochloride;
[4-(3-chloro-2-piperazin-1-yl-6-quinolyl)thiazol-2-yl]methanamine dihydrochloride;
2-[4-(3-chloro-2-piperazin-1-yl-6-quinolyl)thiazol-2-yl]ethanamine dihydrochloride;
[4-(3-chloro-2-piperazin-1-yl-6-quinolyl)-1H-imidazol-2-yl]methanamine dihydrochloride;
2-[4-(3-chloro-2-piperazin-1-yl-6-quinolyl)-1H-imidazol-2-yl]ethanamine dihydrochloride;
[3-(3-chloro-2-piperazin-1-yl-6-quinolyl)-1H-1,2,4-triazol-5-yl]methanamine dihydrochloride;
2-[3-(3-chloro-2-piperazin-1-yl-6-quinolyl)-1H-1,2,4-triazol-5-yl]ethanamine dihydrochloride;
6-[3-(aminomethyl)phenyl]-N-[(1S,5R)-3-azabicyclo[3.1.0]hexan-6-yl]-3-chloro-quinolin-2-amine dihydrochloride;
[3-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride;
[4-(3-chloro-2-piperazin-1-yl-6-quinolyl)-2-pyridyl]methanamine dihydrochloride;
[6-(3-chloro-2-piperazin-1-yl-6-quinolyl)-2-pyridyl]methanamine dihydrochloride;
[2-(3-chloro-2-piperazin-1-yl-6-quinolyl)-4-pyridyl]methanamine dihydrochloride;
[6-(3-chloro-2-piperazin-1-yl-6-quinolyl)pyrazin-2-yl]methanamine dihydrochloride;
3-chloro-6-imidazol-1-yl-2-piperazin-1-yl-quinoline hydrochloride; and
(1S,4S)-2-[3-chloro-5-(trifluoromethyl)-2-pyridyl]-2,5-diazabicyclo[2.2.1]heptane formic acid.

15. The method according to claim 11, wherein said compound is chosen from:
1-(3-chloro-5-iodo-2-pyridyl)piperazine;
[4-(2-aminoethyl)phenyl] 5-chloro-6-piperazin-1-yl-pyridine-3-carboxylate dihydrochloride;
3-chloro-2-(4-methylpiperazin-1-yl)quinoline;
3-chloro-2-piperazin-1-yl-quinoline;
3-chloro-2-piperazin-1-yl-quinoline hydrochloride;
3-bromo-2-piperazin-1-yl-quinoline hydrochloride;
2-chloro-3-piperazin-1-yl-quinoxaline hydrochloride;
3-iodo-2-piperazin-1-yl-quinoline hydrochloride;
[3-(aminomethyl)phenyl] 5-chloro-6-piperazin-1-yl-pyridine-3-carboxylate dihydrochloride;
3-bromo-6-chloro-2-piperazin-1-yl-quinoline hydrochloride;
3-bromo-6-methyl-2-piperazin-1-yl-quinoline hydrochloride;
3-chloro-2,6-di(piperazin-1 yl)quinoline dihydrochloride;
3-chloro-6-iodo-2-piperazin-1-yl-quinoline hydrochloride;
6-bromo-3-chloro-2-piperazin-1-yl-quinoline hydrochloride;
2-[3-(5-chloro-6-piperazin-1-yl-3-pyridyl)-1,2,4-oxadiazol-5-yl]ethanamine dihydrochloride;
2-chloro-6,7-dimethyl-3-piperazin-1-yl-quinoxaline hydrochloride;
5-(5-chloro-6-piperazin-1-yl-3-pyridyl)pent-4-yn-1-amine 2,2,2-trifluoroacetic acid;
3-chloro-8-methyl-2-piperazin-1-yl-quinoline hydrochloride;
N′-(3-chloro-2-piperazin-1-yl-6-quinolyl)ethane-1,2-diamine dihydrochloride;
N′-(3-chloro-2-piperazin-1-yl-6-quinolyl)propane-1,3-diamine dihydrochloride;
[3-(3-chloro-2-piperazin-1-yl-6-quinolyl)phenyl]methanamine dihydrochloride;
2-amino-N-(3-chloro-2-piperazin-1-yl-6-quinolyl)acetamide dihydrochloride.
[1-(3-chloro-2-piperazin-1-yl-6-quinolyl)-3-piperidyl]methanamine dihydrochloride;
2-[2-[2-(5-chloro-6-piperazin-1-yl-3-pyridyl)ethynyl]phenyl]ethanamine 2,2,2-trifluoroacetic acid;
2-[3-[2-(5-chloro-6-piperazin-1-yl-3-pyridyl)ethynyl]phenyl]ethanamine 2,2,2-trifluoroacetic acid;
2-[4-[2-(5-chloro-6-piperazin-1-yl-3-pyridyl)ethynyl]phenyl]ethanamine 2,2,2-trifluoroacetic acid;
3,7-dichloro-8-methyl-2-piperazin-1-yl-quinoline hydrochloride;
N′-(3-chloro-2-piperazin-1-yl-6-quinolyl)butane-1,4-diamine dihydrochloride;
3-chloro-2-piperazin-1-yl-N-[(3S)-3-piperidyl]quinolin-6-amine dihydrochloride;
[1-(3-chloro-2-piperazin-1-yl-6-quinolyl)-4-piperidyl]methanamine dihydrochloride;
[1-(3-chloro-2-piperazin-1-yl-6-quinolyl)pyrrolidin-3-yl]methanamine dihydrochloride;
(3R)-1-(3-chloro-2-piperazin-1-yl-6-quinolyl)pyrrolidin-3-amine dihydrochloride;
(3S)-1-(3-chloro-2-piperazin-1-yl-6-quinolyl)pyrrolidin-3-amine dihydrochloride;
[2-(3-chloro-2-piperazin-1-yl-6-quinolyl)phenyl]methanamine dihydrochloride;
[4-(3-chloro-2-piperazin-1-yl-6-quinolyl)phenyl]methanamine dihydrochloride;
[3-(3-chloro-2-piperazin-1-yl-6-quinolyl)phenyl]methanol hydrochloride;
1-[3-(3-chloro-2-piperazin-1-yl-6-quinolyl)phenyl]-N-methyl-methanamine dihydrochloride;
1-[3-(3-chloro-2-piperazin-1-yl-6-quinolyl)phenyl]-N,N-dimethyl-methanamine dihydrochloride;
2-[3-(3-chloro-2-piperazin-1-yl-6-quinolyl)phenyl]propan-2-amine dihydrochloride;
(1S)-1-[3-(3-chloro-2-piperazin-1-yl-6-quinolyl)phenyl]ethanamine dihydrochloride;
(1R)-1-[3-(3-chloro-2-piperazin-1-yl-6-quinolyl)phenyl]ethanamine dihydrochloride;
2-[3-(3-chloro-2-piperazin-1-yl-6-quinolyl)phenyl]ethanamine dihydrochloride;
2-[4-(3-chloro-2-piperazin-1-yl-6-quinolyl)phenyl]ethanamine dihydrochloride;
3-chloro-2-piperazin-1-yl-6-(1,2,3,4-tetrahydroisoquinolin-5-yl)quinoline dihydrochloride;
3-chloro-6-isoindolin-4-yl-2-piperazin-1-yl-quinoline dihydrochloride;
3-chloro-6-isoindolin-5-yl-2-piperazin-1-yl-quinoline dihydrochloride
[5-(3-chloro-2-piperazin-1-yl-6-quinolyl)-2-fluoro-phenyl]methanamine dihydrochloride;
[3-(3-chloro-2-piperazin-1-yl-6-quinolyl)-2-methyl-phenyl]methanamine dihydrochloride;
[3-(3-chloro-2-piperazin-1-yl-6-quinolyl)-4-methoxy-phenyl]methanamine dihydrochloride;
[5-(3-chloro-2-piperazin-1-yl-6-quinolyl)-3-pyridyl]methanamine dihydrochloride;
3-[4-(3-chloro-2-piperazin-1-yl-6-quinolyl)triazol-1-yl]propan-1-amine dihydrochloride;
2-[4-(3-chloro-2-piperazin-1-yl-6-quinolyl)triazol-1-yl]ethanamine dihydrochloride;
2-[4-(3-chloro-2-piperazin-1-yl-6-quinolyl)triazol-1-yl]-N,N-dimethyl-ethanamine dihydrochloride;
1-(3-chloro-2-piperazin-1-yl-6-quinolyl)pyrrolidin-2-one hydrochloride;
1-(3,8-dichloro-2-piperazin-1-yl-6-quinolyl)pyrrolidin-2-one hydrochloride
1-(3-chloro-2-piperazin-1-yl-6-quinolyl)imidazolidin-2-one hydrochloride;
[3-(3-chloro-2-piperazin-1-yl-6-quinolyl)-4-(trifluoromethyl)phenyl]methanamine dihydrochloride;
[3-(3-chloro-2-piperazin-1-yl-6-quinolyl)-5-(trifluoromethyl)phenyl]methanamine dihydrochloride;
[5-(3-chloro-2-piperazin-1-yl-6-quinolyl)-2-(trifluoromethyl)phenyl]methanamine dihydrochloride;
[3-(3-chloro-2-piperazin-1-yl-6-quinolyl)-4-methyl-phenyl]methanamine dihydrochloride;
[3-(3-chloro-2-piperazin-1-yl-6-quinolyl)-5-methyl-phenyl]methanamine dihydrochloride;
[5-(3-chloro-2-piperazin-1-yl-6-quinolyl)-2-methyl-phenyl]methanamine dihydrochloride;
[3-(3-chloro-2-piperazin-1-yl-6-quinolyl)-2-methoxy-phenyl]methanamine dihydrochloride;
[3-(3-chloro-2-piperazin-1-yl-6-quinolyl)-5-methoxy-phenyl]methanamine dihydrochloride;
[5-(3-chloro-2-piperazin-1-yl-6-quinolyl)-2-methoxy-phenyl]methanamine dihydrochloride;
[2-chloro-3-(3-chloro-2-piperazin-1-yl-6-quinolyl)phenyl]methanamine dihydrochloride;
[4-chloro-3-(3-chloro-2-piperazin-1-yl-6-quinolyl)phenyl]methanamine dihydrochloride;
[3-chloro-5-(3-chloro-2-piperazin-1-yl-6-quinolyl)phenyl]methanamine dihydrochloride;
[2-chloro-5-(3-chloro-2-piperazin-1-yl-6-quinolyl)phenyl]methanamine dihydrochloride; and
[2-(3-chloro-2-piperazin-1-yl-6-quinolyl)-4-pyridyl]methanamine dihydrochloride.

16. The method of claim 11 wherein administering the compound of formula (I) is performed in combination with an antibiotic.

17. The method according to claim 16, wherein the compound of formula (I) is a Gram-negative bacteria efflux pump inhibitor.

18. The method according to claim 16 wherein the administering is sufficient to prevent and/or treat antibiotic resistant (innate or acquired) Gram-negative bacteria.

19. The method according to claim 18, wherein said Gram-negative bacteria is chosen from E. coli., K. pneumoniae and other Enterobacteriaceae, A. baumannii, P. aeruginosa, Neisseria gonorrhoeae and Shigella species.
